SEQUENCE LISTING (330 sequences)

SEQ ID NO: 1 - DNA, 3215 bp, Homo sapiens
  source 1..3215: mol_type = genomic DNA
aggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 2 - DNA, 119001 bp, Homo sapiens
  source 1..119001: mol_type = genomic DNA
aaagaaaaattaaaaaaactttattttaacttctagttcccttctttgtacttgagcagctttccctccttaagaatacagacctagaacatatgcaatatcactatcaatattatgtgtaattaaaagttcattggatgtttactgtgttcaaggcattttaaggagtgacaagagttaaacatatagttgtaattcaaaatgacaacgaaattagtttacagttttctttttttgtaggtagtaagaaatcatctccccctattgaggaataccaatatagaaaaggcaaaactttaaatatgaatgaactgtttcataataacataagttcttcttgatttccattgtcacatccaaatttgaaggctatttctaacacagctgggttctacctttttccttctcactctttaccacacccaatctgtgaggcttcagacacaaactgctaattcaggagacaattgtgccttctgtaacagtttctgctaaattgtctcagctctgccacttaaaatagctaggtgatctcagcatatcaccaaaactcttggagctcagtttctctgtctataaaagttacataaaatgtaattgatctgcttgttatgactaaataacatagtacattagtcctttgccaaaggactaacaaattaccaaataaaagtttggaatcatgttaaacgtttataagaagtgcaactgtccagaaataattctctcacattggtctgttgtaatgagacctaaaatatctcattttatttacctctttgacttaaagcactaggtctcaaggaggtcatggttatactataaatatgtcatgtgaaataatatattaaataattgttgtaatactctattgagatactagttgtaaagaggcacaatggaaaacttatactattaacagtagtaaaaagaaacaacaaaaagcaataaaaaacaaaacacccattcatgcaacgacatgaacgaacctcacaaatattatactgagtaaaagaagtcagacaaatataaaacaaagtttatactacgtgattagatctttatgacattctagaatatgcacatgaaggtacaaggtaactgtctggaatgatgaaaatgtcctgtgtcttcaaaatagtgtgggttacactaatgcatggctttttcaaaactgatttaaagggacacaacatctgagcatttccctaggtgtaaattacactgcaattttaaagaatcatctaatgatattgtggttatttttaaacagtccttaaattttgtggatgcatactgaatgtttacagctgaaaagatatatataaagcttgaatttggtaaaaaaaaaaaaaaaagagggaggattggtagtgataaagtgagtggacttatggatgagacatgatcagccatgcattgaaaaaatgtaaaagttggatgatcttcacatgagagtcctttattctgtctacttttgcatatgtttgaatatttcccataacaaaaagttgaaaatagagtgatcacatgagttaatctcctaatttacaaaaaagaaaactggaaacagaaggagaacaaaacttgttcaaggtctcaaagccagacagcaaactagctcccaagtccaaccttcttgctctggtcctaagcaaacaaaaaatattaatatgagctactgcattaaggaaagtctgcttttccaaagggcagaccaatagttcaaggaagagtttaaataataaatatttgtgatcttactttcatgcttttctattttccactgaacacatatgcattatcttctatatgtcttttatgtataatcatttgcttcctgttccttgtggttttaaagttgttttgtatgtttaaatttgattttactcaaatttcagaacccaaattagcgcaagaatcagacaaagcataactttctataaatataaaaacaattaaaaaaaaaacatacagcaaaaacgagttgttgtttcccccctcctcttccagtgcttaactaatcttccgaatccaggcacagaaagcaaaggctttctgctagtgggaggagcttgcttctccattctggtgtgatccaggaacagctgtcttccagctctgaaagaggtgaaaatgtgttaagcgatgcaaaaattgtcttgaagttcgcgtgtgtatgtctgtgtgcatgtgcgtgtggtgggtggggggagagaaaagggggtgtcaattctgagggcaacgagaatcagaagtcagaaaggtgagtggtgtgtagcatctccctttcagaaggggctgaagaagaaattggatatgatggtccggtaggctaaatcacgctggatttgtctcccagataaagggaggtctgcaaagtaagtcccatttctagagcgaaaagccttaggaccgcttgttttagacggctggggaatatttattccttgttccactgatgggaaaatcagcgtctggcaggcgctgattggtggaaaggaaaatggtgatagtggcgtggaaagaggatttgctgagccttctcctgcctcctcaacctgtgactcttccttagtagtctccctttcaccctcaggaccctttccggctcttcctagattaagagcaaacgaaaaccttgaagatatttgaactaaagcgacccctaacgttgtaacctgtgaccgtgattaaatttcagcgatgcgagggcaaagcgctctcggcggtgcggtgtgagccacctcccggcgctgcctgtctcctccagcagctccccaagggataggctctgcccttggtggtcgaccctcaggccctcggctctcccagggcgactctgacgaggggtagggggtggtccccgggaggacccagaggaaaggcggggacaagaagggaggggaaggggaaagaggaagaggcatcatccctagcccaaccgctcccgatctccacaagagtgctcgtgaccctaaacttaacgtgaggcgcaaaagcgcccccactttcccgccttgcgcggccaggcaggcggctggagttgatggctcaccccgcgccccctgccccatccccatccgagatagggacgaggagcacgctgcagggaaagcagcgagcgccgggagaggggcgggcagaagcgctgacaaatcagcggtgggggcggagagccgaggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaagagagaggcggggaggagtcggagttgtggagaagcagagggactcaggtaagtacctgtggatctaaacgggcgtctttggaaatcctggagaacgccggatgggagacgaatggtcgtgggcaccgggagggggtggtgctgccatgaggacccgctgggccaggtctctgggaggtgagtacttgtccctttggggagcctaaggaaagagacttgacctggctttcgtcctgcttctgatattcccttctccacaagggctgagagattaggctgcttctccgggatccgcttttccccgggaaacgcgaggatgctccatggagcgtgagcatccaacttttctctcacataaaatctgtctgcccgctctcttggtttttctctgtaaagtaagcaagctgcgtttggcaaataatgaaatggaagtgcaaggaggccaagtcaacaggtggtaacgggttaacaagtgctggcgcggggtccgctagggtggaggctgagaacgccccctcgggtggctggcgcggggttggagacggcccgcgagtgtgagcggcgcctgctcagggtagatagctgagggcgggggtggatgttggatggattagaaccatcacacttgggcctgctgtttgcctgagtttgaaccacaccccgagtgagcagttagttctgttgcctacgcctttccaccatcaacctgttagccttcttctgggattcatgttaaggatacccctgaccctaagcctccagcttccatgcttctaactcatactgttaccctttagaccccgggaatttaaaaaaggggttaatcttttcatgcaactccacttctgaaatgcagtaataacaactcagaggattcatcctaatccgtggttaggtggctagacttttactagccaagatggatgggagatgctaaatttttaatgccagagctaaaaatgtctgctttgtccaatggttaaatgagtgtacacttaaaagagtctcacactttggagggtttctcatgatttttcagtgttttttgtttatttttccccgaaagttctcattcaaagtgtattttatgttttccagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggtaggtaaaccccaaatgtcagtttggtgcttgttcatgagtgatgggttaggataatcaatactctaaatgctggtagttctctctcttgattcatttttgcatcattgcttgtcaaaaaggtggactgagtcagaggtatgtgtaggtaggtgaatgtgaacgtgtgtatttgagctaatagtaaaaaatgcgactgtttgcttttccagatttttaattttgccctaatatttatgactttttaaaaatgaatgtttctgtacctacataattctatttcagagaacagttttaaaaactcatagtcttttaaaaaataatcaagaatattcttaagaatcaaaatcattgatggatctgtgatttcttttaccatcatgaaaaatgtttgtcaattttaatccattctgatttttaaaatatgactttgatatgcccctgtgatgtgtataaagagacctatttgtggccctaaaatggaaagaacagattagtctttgatagagttacttcatgtgatcatttggtctctgtgaacactgaggacagagaaaagtgcttgagggctgctactaatctctcagaaacatttgtatagttcatccatcaaatgacacacatactaaaagaataaagaaattgatgcttattacctacttgttcctaaagttccaccttggggtatacacccaaactctgactctcttttctgtaacttgaactgtattcaattgagtgttattttacaaaccactttgaattccttggaaaagaatagacacacactctcatccacaggcatagacacacacactcaacacagacacattgcccattcttcctctcttctttctcctctgagctttttcacattctctggtggcaactatagcagtaagagtcacaggatgaacagtcaggtggaggatgaccacattgagttgcctagctgaaacatgtgctccgtctatgtctgcaaagtgaaagaaagctacactatctcttcaacatagatcagtgggggaaattttatacttgggatgatttatatgaatgcatctcatcaaagttcacaacacatttttttttcagttttttattttcagtttttagagtcagggccttgctctgtcgcccaggctggactgcagtgatgctatcatagctcactgcatccttgaattcctgggctcaagtcatgcccccacctcagcctcctgagtagccaggattataggcatgtgccactgcctcattatttagacttttcttatgttgacttaatcttcccacaaatcttcaattaaattactttttttctaccttaaaacatattttcagaaagtcattgaaatagggtgttacaagaggaaaaaattgatgagttaattttaaatattttatgaagtgtgaattatacctttttagatggaatttggaatactgaatcagtgacatgcagtttatcaatatctttccgtttgtcctcagatttccaagttctgcaagcacaagtttctttgacttagttaccttttaactgttcattgaaatcattttcaatgtctctcatggcatttaacacatagcacattctataaattttttattggttacattctgagttctaattgagagttgaacttacacacagaatttaagataaaaaatgaccatgtgaagacacaatagtatagtccagggattggcaaaattttgggtaaggaatcagatagcacgtattttaagccatgagatctatgtcttggccaggtgccgtggctcaggtctttaatcccagcactttgagagcccgaggctggtggatcacttgagcccaggggtttgagaccagcctgggccacatggtgaaaccctgtgtctacaaacaacgcaaaaattagccgggtatggtagcatgcatgtgtattgccagctacccaggaggctgaggtaggaggatggcttgagccatacagctcactgcagaggttgcagtgagctgagatcgagccactgcactccagcctgggtggcagagtgataccctgtctaaaaaaaagaaaaaaaaatctatgtctcaattctgctgttgaagtgtgaaggtagtcataaacaataactagtgtggctgtgtcccaataaaacttcatttatcaaaacaggtggtgggctggaattgtcttgtatgttgtagcttgctgactactgatagagtggaaagaacatgcactaatcacacaaaccaaagttttagttgagactacatcacttatcacctttagggtcttggggaagcgtacttaacatctctgagcatcacttccctgattagtaaaaaatatgatttagaaaactgcaactaccttgcagtttttgtgggaatgtcataataagacaggacatatgaataattgagcacacttttatatataggaaccatggttattattatcaaataaactctccaacggaataattactttgccaacacgttttccatttattcttttatccttcattacataactagtttgaaagattggaggcgaccaaagaccattttataatttcacttatggctgaagatgtttggtagaagcctcataagaaaagtaatctcattcctttataagaatatacttttaacaactactttttaactcattgaagaactaccttaatgatcagtgttatttttatgggttttgttccctccatttttgttatctgcgtacaccaattttcaatcaacatacttcaatttaatagacaaaaatttcttcaaatgactcagaaattaattagatctaaatccaaaagcagaaagatttaattatctttatataatgctcagtaatataaatgcaataaatacaagaaaatgatgatctttgagtgtcttccaatgccactctgctcaataagcagcagtggccatcagtgaaattgatagcaaattctcaagtcaaaatgtgcttcacctcactaagctgacaaagtcaacataacatgcacaacagggataactgagttctcaaaactctcaggtattacttctgaccttcttctccactctgtgctcttttgaggttgggaagacaagatagggtgtgtgtgggacacctccgctcagggaagccatcagctctggtgtccctacagcatttataccttgctagtcacataaccacttggcacctattttgtaggtgtacgttatcaattacagattactcataaattaaaggctaaccatcaattacagattattagtaaataattatgacctcaaagaacaactgattggtttgatacatggtaaccttatgaggactctcatttatctcgtttttttaagttatatacctatctctttggggttgcactacaaaaatataaaatatgttgcataagatatttataaaaaataattaattataagttctaatggtgtggtttagtggcattcttttttttttctttttttctgagatagggtctcaatctgtcatttcactccaggctgaagtgcagtggtgtgatctcggctcactgcaacctccgcctcctgggttcaagttattctcctgactcagcctcctgagtagctgaaattacaggcatgcaccaccatgcccggctaatttttgtatttttagtagagatggggtttcaccatgttagccaggatggtctcgaactcctgatctcatcatccccgacctcggcctcccaaaatgctgggattacaggcgtgagccattgcacccggcctagtggcattcttttttaaaaataaatttaattgtgtatatttagggtatgcaacatgatgctatcagatacattagacactaaaaaattactatattgaagcaaattaatatattcataatctctcatagttaccttttttgttgtttttgtggcaagggcagctaaaatccacttatttatcatgaatctcaaatatagtacaattttatcacctacagtcctcatacattagatctgtacactttttcatcttacacatctgctacttgcttggatcctatggcctatatgtccctattttctacctacttttccacccctattaaccctgttttttacgtagtctctgtatatttgaattttgtttcaagcttccacatatatgtgagataatgtaatatttttctttctgtgtttggcttatttcacttagcataattttgtctgggttcatccatgttgtaaatggtaggatcttgtttttttagggctgactgatattccattgtatctatgtaccacaatctttttatctacctatctatcagtagacactttagttgtggctattatgtttttctttttttcttttttggagacagggtcttgctgtcacccaggctgcaatggagtggtgttatcatagctcactgtaacctcaaacttctgggctcaagagatcctcctgccttggcctcccaagtagctgagactacaggcatacattaccatgcctggctaatttttaatattttttgtagatatagcatctcactctgttgcccagactggtctcaaactcctaattcaaatttagaatagagtatgacaattctgtaaaatataaaaaacatgtccactccgtataggaagttatacaatgagaagaagacaaacactatttacattactcttgataagttttttacaaagaaataaaacactttaatttctaatgttttaaattctggtttgctaaataaataaatattagttttagtgtttttaaaattccttatatagttataagtgatcttcctgcctcagcctcccaaagcactgggattccaagcaagagccactgtgttggggcccttggaaacagatatgctgaaatcttttcttgtggatctacacccagaagagggattgctgggtcatatgctactctatttttaatttttcttttatttttagtgaatatgtaataattgtatataattgtgggatccagaattatatttccatacatgtatacaatgtgtgataatcaaattagggtaattaacatatccattacctgaaacatttatcattcctttgtggtgggaacagtaaaaattaaaaattctctcttctagatttttgaacatatgcaataaactattgttaagtatatcaccctacagtactacagaatgctagaactcattcctcatatttggctccaatttcatattctttaaccaacctctccatatcctcccctccctcttaccgttgtcagcctctaataatcataattctactctctacttctatctcattgtctttgatttagaatatgtttcataatttaaccaaaggtcaaattcttaggtactgctaaggcaaagaacaaagatcgcattccagctgttagacatttcttactactagtcatttttaagacaacatggggtgcaggtggtgaggatgagagatagagattgaaacatattctcttaaatatcagctgttctcactctgcatagttccagcacaaacaaattccaggtactatggttagttaaataacaccagccactaacaacacaattcaaatttctgttaccacagtataccgaaagtcattgcataaagtacaaactttgctgctaactcttcagccttcaaatcattacataaataacagaaacccattataatcagtgacaaaaccacagcacttctttcaaagctttttggagattggttgcttcacatctgttatgcagttcatacagacagcaatgcccggacttgtgtggccacattgtctcccagtggtgagcccatgtgatgtttcacgaaaatgcgcaatcaaaagaggaaactggccagcaaagatgaaagagtagcaaacaaaggaagtgaaacattctggaagtaaaatttgaatcaaacataagttgatgtatacaggaagtagctaccctgaggatgttgtcactgctgcaattcaggagactctaaatatgcagtcagaggaacgtagtgaggtgaaggtatccgtataatggggaaagaggttgtgataaagagtgaaggtgtcccagaggaagtgttgctgaaaaatacaccttatgttaaatacactgtcagtatatcatgacattaaagtgcaaatgataacattttgtaaactgatccaaacttaaaaaggagtatgataattctgtaaaacataaaaatcatgccgattccataaattatacagtgtgaattacactgaaaaatccaacattagagaggatatgaatacaattttttacaagcataattttaataatacacataataattatttgtattcaagtttagtaatgttcaaggtttggaagaaattctgatcctgtgtagagaccctagtttgaatgtgcttatagcctattattacatgtgtaatgttacataaattacttaactcggatttttaatttcatcagctatttaaaatgggcataatataactatattaaatggctgttatgaagattaaataagatgatatgtaaaatgtgttttttgtttgtttgtttgtttgtctgtttgtttttttgagacagagtcttgctctgttacccaggctggagtgcagtggcacaatcttggctcactgcaagttctgcctcccgagttcatgccattctcctgcctcagcccctcccaagtagctgggactacaggcacccgccaccacgcctggctaattttttgtatttttggtagagatggggtttcaccatattagccaggatggtctcgatctcctgacctcgtgatctgcccacctcggcctcccaaattgctgggattacaggcatgagccactgcgcccagcctaaaatgttttttttacataatgggtgttcagcacatgttaaagccttctctccatccttcttcccttttgtttcatgggttgactgatctgtctctagtgctgtacttttaaagcttctacagttctgaattcaaaattatcttctcactgggccccggtgttatctcattcttttttctcctctgtaagttgacatgtgatgtgggaacaaaggggataaagtcattattttgtgctaaaatcgtaattggagaggacctcctgttagctgggctttcttctatttattgtggtggttactggagttccttcttctagttttaggatatatatatatatttttttctttccctgaagatataataatatatatacttctgaagattgagatttttaaattagttgtattgaaaactagctaatcagcaatttaaggctagcttgagacttatgtcttgaatttgtttttgtaggctccaaaaccaaggagggagtggtgcatggtgtggcaacaggtaagctccattgtgcttatatccaaagatgatatttaaagtatctagtgattagtgtggcccagtattcaagattcctatgaaattgtaaaacaatcactgagcattctaagaacatatcagtcttattgaaactgaattctttataaagtatttttaaataggtaaatattgattataaataaaaaatatacttgccaagaataatgagggctttgaattgataagctatgtttaatttatagtaagtgggcatttaaatattctgaccaaaaatgtattgacaaactgctgacaaaaataaaatgtgaatattgccataattttaaaaaaagtaaaatttctgttgattacagtaaaatattttgaccttaaattatgttgattacaatattcctttgataattcagagtgcatttcaggaaacacccttggacagtcagtaaaatgtttattgtatttatctttgtattgttatggtatagctatttgtacaaatattattgtgcaattattacatttctgattatattattcatttggcctaaatttaccgagaatttgaacaagtcaattaggtttacaatcaagaaatatcaaaaatgatgaaaaggatgataatcatcatcagatgttgaggaagatgaggatgagagtgccagaaatagagaaatcaaaggagaaccaaaatttaacaaattaaaagcccacagacttgctgtaattaagttttctgttgtaagtactccacgtttcctggcagatgtggtgaagcaaaagatataatcagaaatataatttatataatcggaaagcattaaacacaatagtgcctatacaaataaaatgttcctatcactgacttctaaaatggaaatgaggacaatgatatgggaatcttaatacagtgttgtggatatgactaaaaacacaggagtcagatcttcttggttcaacttcctgcttactccttaccagctgtgtgttttttgcaagattcttcacctctgtgtgatttagcttcctcatctataaaataattcagtgaattaatgtacacaaaacatctggaaaacaaaagcaaacaatatgtattttataagtgttacttatagttttatagtgaactttcttgtgcaacatttttacaactagtggagaaaaatatttctttaaatgaatacttttgatttaaaaatcagagtgtaaaaataaaacagactcctttgaaactagttctgttagaagttaattgtgcacctttaatgggctctgttgcaatccaacagagaagtagttaagtaagtggactatgatgccttctagggacctcctataaatatgatattgtgaagcatgattataataagaactagataacagacaggtggagactccactatctgaagacggtcaacctagatgaatggtgttccatttagtagttgaggaagaacccatgaggtttagaaagcagacaagcatgtggcaagttctggagtcagtggtaaaaattaaagaacccaactattactgtcacctgatgatctaatggagactgtggagatgggctgcatttttttagtcttttccagaatgccaaaatgtaaacacatatctgtgtgtgtgtgtgtgtgtgtgtgtgtgtgcgtgtgtgtgagagagagagagagactgaagtttgtacaattagacattttataaaatgttttctgaaggacagtggctcacaatcttaagtttctaacattgtacaatgttgggagactttgtatactttattttctctttagcgtattaaggaatctgagatgtcctacagtaaagaaatttgcattacatagttaaaatcagggttattcaaactttttgattattgaaaactttcttcattagttactagggttgaatgaaactagtgttccacagaaaactatgggaaatgttgctaggcagtaaggacatggtgatttcagcatgtgcaatatttacagcgattgcacccatggaccaccctggcagtagtgaaataaccaaaaatgctgtcataactagtatggctatgagaaacacattgggataaatcggctgctatcataatcattcctctcccacatcagataaatgaattaactttttgaatagggttatttaatataaagtgcttaagtctaattatgagaagaaataagataattacacttcaatggttaaagagagggagaataatttgcatattatgcctgatgtaaaatgtttattatgggtacatattaagtgctaactaattgttaattgttcttgctacaagtcttaatgcagggaaacaagaaattattacatagtacctaatattatcttctaatattaaagaaacaatttcccctaaattcatcccattagctttttttttttcggtggggcaggggagaaatacagacttcagtaaacttgggctgggaactttctacctacaaagttcaaataaaataaattatcctagttagataatatcaatgaaaaatccaccaacttaaatcctggctgtttgatctcaggaaattatttcagttatcaacttaatgcatcatattatagaaatatatgaaaatgtgtttaattaaacttactgaatgatatgttttttcaggtactttaaaaataaactatgatataaagttacctatttttcatgcaagtatagtataaagaaatttctaacactggagattttctgaaggttttgattcttataaatttattacatcataatgaacaaaactaattttcaacatattatgatttaaatttccttagtaaattgtttcaaatttattttctttaaatccatatttacatatgtatatttaaatatacatatttacttgtataacaattcaaaaccatatattaattttataattttgtttaatgtcaaaggttagatttggctatatctattctaaaagttggtatcacatttcctttttggaattttatttttaaagtagctaaagtcaaatataaacctattatttatattaatgcagacattagaggtagacactaaattcattttagtatattctaaattatttattatctactatgaaataatataaagaaaaataaagcagaatccctgatttcaaagaactcaattgccgaaaaacagttaccatttattagacccaaaatgtactaatatgagtgtgtctcttttccttttgttttgtcacccgtcatttggaatgtcagtgagtagagagatagtgtgaaaggccctcaaggggaaaaatagaggttaaaggtcagcagagaccctactagagaaatcagttctacagaaatgtttttaaatgtgtcgattattgctacatgtacactctgtcattttgtaatgtagccattttatttatgattataataataaaacaacaaaattataataatgtgtagagtacattttactgtgcagtgtattgcattaaaactagattaaaatttatacatatataaaaggctatctagatattataaaatttatggctggatctgtaaaaaattcaaaacctatttttaatctcgctttgagattttataacaagaaaatgttcgtttcaagcaaaattttcaattcacgtccttgaaaaggaaaaaaatgacaacttgaaacacataattgactatttttaaaggatcaacatttcagaaatgttttaaaacataagattttcagtacagcttttcgctggcatttaaatcgaactttgaattgtaaatagctcttgctcttaaggagacatcagccatatccttagaagtggcacggagttgttaggtagttgtacaaaattctagcctaaaagacaaatagggagcaacactactgtggaccgtttctggtcttgggctgtgtggctatgtcaggcttgcccacattgcctgtactaaggagaaagcctcttgtccttacagacccccttagcttacatagtctatttgaaaacaaattgctttgtccacaccatttaaatattggcttcaggccaggcgcggtggctcacgcctgttatcccagcactttgggaggctgaggcgggcagatcacgaggtcaggagatcgagaccatcctggctaacacggtgaaaccctgtctctactaaaaatataaaaaaattagccgggtgtggtggcgcgcacctgtagtcccagctgctggggaggctgaggcaggagaatggcctgaacccgggagtcggagtttgcagtgagccgacatcgtgccactgcactccagcctgggtgacagagcaagactccgtctcaaaataaataaataaataaataaataagtaaatattggcttcttcaactggtgagatgaaacctatacaatagtcatgtgaatagcactaaacagctgacatggtgtaactcctctcagactgaggcttatctggggagtacaaagcatgtcaagaaaatgtgccttcatttccttagatgagtgtccccatcctccactctcctccactgttctcctctctgcttctatgatatcaacttttctttttctttagattccacatgagtgagatcatgtggttgtttgcctttctgtttctggcttatttaactgaacaagaaagtttttgacatgaaattaaacttctgcttgtaaactcaattcaaactatttacactgtcttctcaaaaatgttaacttattttaataaatctactgaatgaccgtatctcattttgttttatgaaaagaaattgtaagggtgctcaatagcctcttcattttcatactgtctagctcctgtgctcctattaaaattactgcaaatttagctttttaagaaccctttgtttcactacctgaagttctataaaaagatccaagttccttcacaaccgtttcttatgctgttattcgtacatatgtgataataccacgtctgaacacgtagataataagtaggggctgggtgcggtggatcatgcctataatcctagcactttgggaggctaaggcgggtggatcacctgaggttaggagttcgagaccggcctggccaacatgatgaaaccctgtttctactaaaaatacaaataataataataataataattagccaggtgtggttgtgggcacctgtaatcccagctactcgggagactgaggcaggagaatagcttgaactcaggaggcggaggttgctgtgagctgagattgtgccattgcattccagcctgaacaacaagaatgaaactccatctcaaataaataaataaatagaagtatgtattgtgttgcttagaaggtgtggtggaaattaacttgctgagtgagatcaaaggattggcactgaattgaaataaagaaatattcatgctgagtctggttcaaatataactgcacctgtaagaattgctttctgtaaactttccatagtataaaccaaatccaaatcactcatggctttacattcctgatcgttaaacttgaagcactttttaatactgcatgactttagccaaaatatcttagccaagattcaatgtttggttgaaccacactcacttggacatcttggtggcttttgtttcttctgaccactcagttatctatggcatgtgtagatacaggtgtatggaagccgatggctagtggaagtggaatgattttaagtcactgttattctaccaccctttaatctgttgttgctctttatttgtaccagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggtatggctgtgtacgttttgtgttacatttataagctggtgagattacggttcattttcatgtgaggcctggaggcaggagcaagatacttactgtggggaacggctacctgaccctccccttgtgaaaaagtgctacctttatattggtcttgcttgtttcaggcattaacccagataaatgccatgcaaattttataattattatgattgtttcaatttctggaagaaagttaatgaaacaaaaaatgtagtaaaatgccaaaggaacagtgacatttcagaaagaatgagggctttcatgttaattgtaagtcttggaatttctcttccttggagtaacaaatccctttgtgcctaatttcctaatttccaaaataaagttcttttacttatttctttatagtgacatcatctcttattaaatggcatatctgcatattacataacagttcattgccaaatacatatttgtgggaaatgagagacttaaaatacataccaaccagagatatagttttgaggtagattttaaaattctgagaagaattttgactgaatttttttgacaaacatgggacacgaataagattataccaaagatattataactttcattttaaatatggaactaatacagtatgaggtgtcaacaacgttgaagtttcacaaacatcaccactacaacagcaaaataatttttgctttttccctgccacaatgacctccttgctatttcttgaataaatcaagcatacccttgccctgacacgttcttggggaggcctgccctaatctatataaaattggagccattcttctcacctctggtattcccagtctccctactttttttccttctttctttctttttctttttctttctttctttccttctttctctctttcctttctttcttttcccttccttccttcctttctcccttccttccttcctccctctctccctcccttccttcctccctttctttctttctcttttttctttcttgcttccttccttccttctttccttttctttctttttcctttctttgccaaagtgttattcacctttaaatataatacataatgtgcttactttaatgtatgatttttattttatttctcccttctagaatgtaggcaccatgagagtgaaatatatttattttgttcattgatatttcacaagtgtctgggagagtttccaacttacagtagacaattaacaaacatttattaaattaaggagggaaggaagtgagtaagcacaacaactttcatttctgggtcttttataatcatatgcttagtataagaacagtgctattcagctatccaaaagttacaatcaaaatgattttggatgaatatcttgaaaattgtgagaaagaagttttatttgctggcaaactattctgggttgtttccacttcatgtaatcctaagtagcagccttaccttgatagcccattaaaactctgataataaaaaggcagaacaaaaatatctgtgatatatttagatttactacatgtacttacatgtctagtgtctggtgcaatggatgctaatgatggcaaatccttactgggcttctagtgaagttcttcagctaatgtttgaatgcatggttggtcatggtggtacccctttgtacaaaatatgcttttcaaataatcttattagggataataattatattaattcctggtttccatctaaaattttaattctatttatagcttcgtaagatttcacaagttaagagggacctcagattaaattagtacacaggcaattaatcagttttgtgtctccgacccttttcacgggctaatagaagctatagaccctcttagcttcagaaaaatgcgcactcacatacgcacatcaaagagcttaatgggaagtccattgacagaccctctgttcagatcaatcttctgattgtagagatgaggaaacagaaatctacagaggaagtgggtagtccaagattgcacagtcatttggaatagactggacaccagtagtacttttccagccactatatcacttccccaagcacttcctcaaaacttaccttcctttgggtctttatacattcagttatggacaactagatttaactagaggattttattgcttcagaatattaagcaacagggaaacatgtaccgtcttttattcacctgcatttaaggcatacaatataaattgcaaatggagcatgaaagtgcttaatcttttacaaaactgggtttgctttccacccatctaaaaatacttctatttattttaatatttaaagcagaaatctaagtgatgtgacaaaattaatcatttggagatatttcccttataggtagtatagtttcttactgatttctaatatgaaaatgaagccatagaacctagaaattgcagcatagttgtggaaataaacattggactgagagtgaaaatggctagtcttcctctctgctcatacaccacctgactggataacctttcgcagatctcctaaaagtctttctcataaaatgaggaagctctactagaaaattgttgaagtctaatttagcaataaagttctgagtttctataataattcaaagaatactctaataaatgtctgcaattgtggtcacatctatgggatgctaaaaaatctggatggtttcaatgaaagtatttaatttgttcattatgaactttgaaataatttatttcattttttaaactttgatcaaaatgaccctggtaaatagaaataagcaaactctttttgcttgaaatgcttattaatgactgcattgagacactcattcatcattcaagaaagaatgtttgctcacactgtgccagaaacttggaggaagagggatgtgacaagtaggggtactggatgtctagcttgtagaagtggattaatggctctgcttttaagatcaggaacactgaaagggagtaatggcaccggttttcacctttcatgccctttgagggtatctggtccatcaccctctagttgatgagggagggaaagttccctctcccttcacaaataggtggaaattaaatgacataattctgaacaaccaataaatcgagagtaaatcaaagcagatacctgttttgttaatttgatcatatgaatgtagctgcccttagtaataatttctaagtataagactagttaaaggacaaatgagttatcttgaattataagattttgttttacagaacaatattaactcttgtgtttagtacattagaataatagatcttttgatccatatttttactcatgtgcacataagaagttatcagtcatacaattcatttcttgaagttcatacctttcattggcagagtagaaacaggttaaaagtgcacaggcagaaattttaagtgcaaagcaacagtgatgttatatagagaaaatttatatttcctacttctattgaagaagaaagatctgcttgttctaagaatattgtacaaagaaagtgacttgaatcagcgttattctgtaatgctactatgcgtgcagtgtggagtagccactagaacacttggtctatcccagctcctcaacagtgtcttgcttgtggctggtgctcaaataaatccttgctgaactaatgagcatctctttcatgccacatggaatgctctaaaagagttggatcctgaagtttttatatttttgtaattttctggagttttagagagcaaaagtcctgaataaactgtgaagccactgcctgacaaataatacagcagtcagcttcgttatcatatcccattgagacacgacttatctacatgatgattaatagttttcacgcaagaaataagcttgaaatgtctgttgccttggatacttaaaacatccaggttcagcgatgttatttattgttgttcaaaatcagaatgaagttcctaagcaatgccattttggaaaaattacatcaatatattatgaacaactttttttaaatcttgatttcaaatggattgacacgtgtatattctgtaataatcctgacttaattcataaaaggatagctagccagttgtgtgctagatgaataaaaaaaaagcaggttttaaaatgtcaggtttgacattgtgaatataatatctaagtatccttttactcatttcctttgacttactatggctgtcatgttgggcttcatgaaaatttatttttaaacacttgagtgttatggaccctctgattaaatgattaatcagatgatgtatgttgccatcagctgaatcatttaatgttgatttcacaaacaagcacaggtcacaggcaacatttcagatttctttgaagaagcacacacaggtcacaggcataatcttaaaataattttataacaaggtagtaataagagatgtcaggactggagaaatattttaatttatagtaagctttccccttaagtgtctaataattgttaatataatacattgcctcaaataattaaaagtttggttcttgtccttgtgcttgacttcagaagataaccagatgactattaggtatatttagacctaaattaaaagctttgagacacaatgaattgcctgatttgtatttgtgtttcgagtggcatatactattactggcactataatcttagattaaagcatactgtgattattaaagaaaaatttaagattgatttgtttctaaaggtatgtaacagtgacattttgcaatgtggtatgtaaaagttggtatttctcactcatatgagagcccactaatggtacataaactgtccccacttagaaacacaattattatggcctttctttgtatctgacaaaatttcactgggttcaagatggatgaatagtgaattctaatgacccttaatcctgtaaggttctaggtgggaaagtactctgtaattatgtataaaattataaggaaaataggcttactgctatgttttcattaaaaatcattaactgagtacttaatatgtgccagacactcagctgggcaccatgagaaatacaaaactgagtaacatatgggtggctcctgccttcaagaaatgggcagttcaggccgggagactgacatatttaccctgggaaaaagggagcagctgtggtctctgagaacaatatggtttgttacaagtatatatccatcatggaaaaaaagagatttatcttagaaatgagagaggctgatgctctcaataaatatcatacattaaattgtgtttttgtcagtagactgaaattacctcacatacacgcacagatagtagccatgatattttagctgcttagatatagagacaaatacttccacccaaatcttaggatcagtggttaatagtctgtaagcattacaatcccacaacatatgcatgactatacatccaattttaatattcaaagaactgattgcgatgatagttttgtttgtcaaagaaatgtattataggatgagtgggatagaactgcatcacgttacaccaacaaataggtttaaatcatatttgtgcacttcccttgttccttcataaatgtttaacatagcttaaaattctgtggactgcaacgtgagagcaatgaccacacttctgtgaacccatttttactgtgcatgtgctaacgtctattgttagtattccttcacttgcaaagatggcatgataattttgctggtttcattaatgagatactgttaaatgtaggatgacttcaaacttagttgtattgtaaaattatttttaattgtatacatttaagttgtacagcatgatgttttgagatacttatctttatttatatatatatataatatacacacgtatataaaagtgattcctacattgaagcaaattaacatacccatcatcatatggttatctttgcttttttactatcagtgcctaaaatctactttcttgaaaaattaccagtatgcactacaatattattaacaataatcttcatgttgtacattagatctttagacttactcatcttacatgacttaggtttgtttttacctctactaccatctgagccatatttccactttgtaatttgataataaacttggaaaaatagcacttatatgtttaggtgacgggcataaataggataagatgtgtttatatattattccatatatcttgtctccaactacaatgataaacaacctgtttgtccctaaaaagtaagaaataacttgacttttctgccccttcaagcataggctgttagcttttaagttttagggagacattgatgatgctatttgctttatcaagaggaaattgtcaaaagaggtcttttggttctcaaactattcaaagtatttaaaaatcaggacaaaatatgtttacgtgatattcaagggtacagaaatgaggtaaatgagatgccaattgtatttgtcatgcaaatatataattacgtgtatgagagttagatgatacatctcatcaatttaattgttcttctacaaggagaaaatgaacaatttgtcaactcgtatatgaagtaatttttataagaaattttattaaaacttttaacaacatttggatttttaagttgcaatttaaatatccccttctaccaggtgattctggaatcactaagcagttacttgtgaaaattccaaagtagcatttaattcttattaatgtcatagtgaatactaatgcaaagaatactgagccagaaattatgcttgttgaataaatagattatttattgaacaagtaagtgaaaaaatggaaataaagaacggatatatattttatcttcctgcttagatgtgggactgtcctacttttctctggtgttcacaacaacaatatgataaatctaattggaattcagttcataggaatgaattcagttacattatggattgtgatgaataatgtacacttttaatttaatgaaatcaaatagattttaactatctatgcttacaatggggtgacataagtctgacaatccttaatatcaagtcatctccaattcacatgtatacacactttttttctatttggctattgggaatcctcacaaaaatcgaaaattgccctttcagtgtacgttacggtatttcatgccacacagattttctgaggttgtacatacagctttgccttgaggttccaatttttgctcagtggattgagtatatattatttgctatatatcagaagaggcatgtgcttcctacttatgtcaggtaactttgggattaatataattgtcctacaaagcatagatagatagaaatacttcatccttaatttctaatattatgacatatctaaagtaggcacctttaaaagttaatctccactaaatactaatgactgcttatagtggcaattcatctttcatggtagtcctcctacaaaggtatactaacatttatgagtttgaaacaaaggcaattcacaagtgttctgctagagatggtctatatctgctgtttgatccagcatgatggccagctggccctcctgtgcatgacggctcgtggtttaactgcaccattttgtttggtcatatacagggaaaacatggcatggtgtggagggcatgggcttgaattcagggaacagagagttggtcttctctctctcactctactggatgatgtcatctcccctctctaagcatgagttttcttatctgtgaaataaaaatgttgaattaaatgagttcaaaatgctttcagtctgtgtttaatagcttgaatcttaagacaatgtattcaattatgcgttgccagatccctggcaactcatgtaacctttctaaaccatagctactcatctgtaactggccagccaactgcccagggttggagtgtgaatgaaataagataatgcagacaaaagatttttaaaaattgtagtgcattatacagttgtaatattttgccaagaacttacattttctctaagaagtgtgtcgatacatgatcacagaaaatcttttccatattcctttgtagtttgatgatattaagtaagtaaattgtataacacaaagagggaaaagcatcactgaacatgccgttttatttagctaaataaaatgtaatcactattagttttcctctgatttccccaaagtcatgtgattccattgagtattatgcacatggtataattagaatggattctctgctcaaataattttgggaaacatttaaattaacaaagtttaaaagtatctctgttaagctgaagcaaatctcaaaggccttaatattgtatgtaagaggaatagttaccatctttcctaatgcctctttgacgccaaacccatggagaatagttctaggtgttcagtaaaacacagatttgggatgccacaggttaattggaactgtcccctgcaatccttttctctttttcttaataatggctgattgcaggtcctagatgaaagacatttagagagattatcaggactcagcatcccatatcagaatccattcttttatagtcattttctgttacatttcttgggacaacaccaaagaaatgaccatcttcattcacataggctttgtaccaaatgctgacaaagatccttggtgacctagatgggggcaggtctaagtagattgcagctgtaaaattggctgatgaatgatctcagccccttttactcacactcaaaggcaggacagtccattaaggggaaggagggcagagtttttccttaggccaattccctatgccagaactttttagaatggaagcatttccagaggagaaacaaccccaagcacagttcaaagccccctcctcccaagttcatttgaaagtgggatggtttatctgcaaagggggaaaagatgagggatagggacgggaatatccctacccttcagagagtctggtttcatcctgcacttttactgcacagccacaaatgccttggggtgaatctacaatatgatacatcatatggtctaaacgtgcctggctgatcctctctaatacttcaggggtctaaaagggataacatgctctcctgttactcaccgactctgtccgccatatttcacccagccagccactgccttcacttccgtccgaggcctaatctgagcccatgggaaacctaagaacccctaccacaactgcctcaactcttgggaatcagggtgtatgggggtgacaggaagtgagcatacattctccaacttgatatgtcagcccccacgtctgtatgaatgtttgctcacactgtgactgccggccttgctcctcaggctgcatcctaccagggagtaagacccaagtccttcctgctttcagacaacaccaagcctcatgagtccccactcagaggaaggaccagagacaaactctaatgttccactaatacttcccttcttattactttccttgaaaatcccttctccctctttctttttatacttcgctaatgaaaggtaatgaaagggtctggcacttggaatttagaattgatacatggtttttaacccgcggacgtattccacaataacccttgcatcttctactaagatgtgggctaggaagggaccagccagttcccagggtcacagtgcctcagctgatgtttcatattttcagcaactttatgttagagatgtccatcaatcagaacaatatggttagagaataaactaataaaagtcatttttgaggacatgttggaagtctatcaaaagcattgaaattatgcatgctctgaccagtcgcatgtctaagaatttaaatatgatcataagtttaaatatgaagatgtttatcacagaattgattataaaacaaaattgaaaaaaatagtgctagaagtttgatcatagggacctcattaaatgcattatggttgatccatgcagtggtttgctgaacagccattaaaatgttgtagaataattattaatggtgtggaaggatgctattgttgcagtatgtgaaaagaacaaattacaaagcagtttgtgcagcataatatttttattttttaaaaacctgtatgtggcttatgtacatataaagacgtggaataaatgcacaaggtactcagtttttctcagtgaagcccattttgcattttgggctgggtaattcttcgctgtggagaactctcattcattgtaggatgtttacaagccctgggccttacctctttaacgccagtaggcacccccagcatggcaacaagcacaaaatggtctctctcatattgcccttgaggaaattttgcaactaagtaactattactgggtcctagattacagtctggattattgcgttcctttcttatttttattttctccaattccctttaataagcatgtactggattcataaaaaaacaacataaatggtaattacaatattccgcactggttaaaacttatgtaaataagcattctgctgctttagccacaattgcaatttatgctccttctctttcttaagttcccagttcccacgtacattcattcgactgattcaaaagtcattttagcttgatagactcttaaaagttagagttatcatttctgctatttattctttcaattatccatttgtccacccatccatctgatccattttgttgatgcatgctgtgtataaaatactacaccagcctggtgcggtggctcacgcctgtaattccaggactttgggaggccaaggcgggtggatcacctgaagtcaggtgtttgagaccagcctggccaacgtggaaaaaccctgtctctactaaaaatacaaaaattagccaggcatggtggcagacgactctaatcccagctacttaggaggctgaaccaggagaatcgctcgaacccaggagatggagtttgcagtgagctgagatcatgccaatacactccagcctgggtgacagagcaagactccgtctcaaaaacaaacaaaaaaaatacaatgccaagcatcataaaaaatatagtgatatataagacctatttgttgtgctctaggcattgacatctagctgtcaaccattaatatgtgtaggagtctatctatcaatattatggactgtgcttgaagacttcttccccaatctttttctcttcccattaagtttgaagtgaggttttctgagtgaagtatcatagtacatacagtctcattatttttcaaaaatctctggttatagtacatttctttcctttatcccctttgttcccaactatcaaaccattttggatatccagtattggtatccagtattattaaaaagcaaaacagagaactattaacaaaaaaatttgtaggagtaattggttgtatggtatccagtactattagatagtaaatcagaaaattattaacaaaaattttagacgaataatggattgtcttgcccaagtgaattgagtgatttagttgttctttcatttttagcaagtacagctgatcatttgaggccttactcattgtttgattttgcaaattcttactattataaatgttttgggctctgagaaagctgttgtcttaatctgtttgtgctgttataacaaaatacatgagactgggtaatttacaaacaacagaaatttatttctcatagctctggaggctgggaactccaagatcaaggcatttgtcttcaggttcagtatctggcgagggccggttctctactcccaagatggtgtcttgtcactgtatcctccagagggccaaatgctgtgttctcacatggtagagagatagaaagggccaactcactccctcaaggcctttcataatgttaccaattccacttgtcagggctctgcccccgtgactttattacctctgcaaggccccaccacttaatactatcacgttggttattacgatttatcacatgaatttcgaccatactagttgccatcctttcattttcatatatccttaaaactttgcctttctcattttaatgtactttatccacagtatgccaacttttcgatacttttgttaacctgtctgacgatatataggaaactgtaaaagtgcagtttttgatacactctttagctgcccgtttacttctactgtcgttagagaaccccatccatagtgcatgtgtttattttgtgtatgaacaaagactttatatatagtttgggtcatttttattcattagtgcttcccttataatctctgaataccattttattagtacatactgctattcttaatagtaactagcatgcctgatcatcccaaatgtctaggttcacattttaaaataagttatatctttgggcttaacagtttattgaaaggtaacaaggattgagtcatagttgtatgtttttggaagtagaattcaactgtaaatagaaattggttgtttagatctcactatatatgaaaaaatgaaggctttaggagaaaatctccccaaagtacccatttttcatgtgataaatatcatgaaatgatttgagaaaaaaatgtatatttgttacagctaacaaatatttgtgttttttattcttcatggagagaatgaaatttcttctcttctttacacatttctttttcttattagaaactaattggtgcctttataaaaattaactgcagagcactaacgtgtatatataagtattatgtagggtgtagggtatgttcagggtatggtgtgtgtgtgtgtgtgtgtgtgtgtgtgtgtgtgtagctgtgtgtgtatataatgaaatatatggtagtgttgtttcagaaatctgcttggtcttcccagagttcattcatcttataaattcatctacattgatctctatttttggaatccatgaaatgttttttggcagtacttcctttaatatagtgtgctggaaatctggaaatttctagccagattagttacaaaaaattagccagtggttttgcactctctatagaatcaaggcccaaggcctactcttgttactcagggccttgttttatctggcctctttcttttcagccatatagctctcaaatactcaacaaaattcttcattctaggtagacaagtatcttcaaaatacttcccaattatctaataactgtcttaccactaagaaggcttttatgtctcctgtctgaattttatccatgcaaaaaagtccagcccaagcctccagaactccaaaaagttatccctaactgctgaaacacagtaatttcactatgtgaaatttcactttggtctcctagcatttgcagatataccatacatatccttgatccttttcctttcataccttttatatctaacccttaagctaataattttacctacactgtaattcaaaatgtatccccagtcttaccatgtctcccttctctactgttaccaccctaggctaggccttcatcatttctcacctggactccttccctaacctctgaactgatctgcctgcttccacttagacacccaacctagtccattcttgagcagtcggaataattcttttaagaaagaaaccagatcacatccccctctgctcccaaccatccagtgacctcttatcatacatagaatgaaatgcaaatctttactgtgttttaaaggccctacattatctggacctcagtaacttcttacttcctatcccttttctccttgtatgccaccctccaactacactctaactacactgtctttttccctgttcttcagacctgccaaccatattttcactgctcaattaatatgtagaaaatgaattgtttgttaaatgtagactgtttccttcttaaagcaaagataaatgacattgtcttcaaaaacaactaactgcccagaattcctgattttaattttaaaaagacaaactgcaagaatgtgttaaacagtaaggaaacaattcactacttcagaattctatatgatttcactgcacgttagtaattttgtatattatagaatatgagggtattctaataaacttaactctatgctgtatacttatcatgatagctcattttcttatatgtttataacagcactacttattgtacatggatacgtgggaaataaattaattttctccttaagaacaaagcaaccatttcactcatgagataaatcttgaagatttaaaaactacttataattaattatacattattcatataatgttaagtattttcttagtaaaccacataatttagaatggcaattggacagatgggcagaaccacatgcatccactattaggcagttggtgagcataagatgccagaaagaagattaggaatatcaaggcagggagcttccgatcgctcttgaaaacattgacccttcactcctcactctccacgatgcatttcctttgaaaagtaatgccttccaaaacaaagttctctgttttatatctaaacttactcaatagtttctcatggttattgatatataaaaaataaagtaaaatgtttaggcagaccaaaagaagaatttccccctccctctgccttttatgccaaggtgacagctatgaaatgtacagtacgtttcctctgcaaggaatgtagcagtgttccattgcaagaagatgagagggagagaaaggttgcacgctgaggaatatagtgtcatttgtcactgcctagactcatcagctgtgtggaactctgagaggcaccaggcttctttatttatttcttcagaaacttcagcaaaaaagatttcattaggagcagagaaaaatgtgaaaaacgaattagcttttgtgatggggagtagtcatctctgaatattgatcaagattaagagggttgtcttcgtaacttcttttatccatagtctatactgatttaactagaaaactaatttcaggtggtatttcgggtgtggcagatctttatagtaaatgaagaatctagtcaaatctactgaaaaactctgcttactttaatgtttgatctggttgaaaccattttagcttaacaatccttcctctgaaacagggaatcaattgatatcctacagcaaaattatgtggaagggccattagcttcacatccaatgcaaattttgcctgtgtttactcttccccaatccaaaatatatcagatcctagatgccagtgaaatcgtttgagctagatggcttgagggtcatagcttttttcatttcctgttctcagacctcttataattgatagaataaaatcagaagagccctagagctgtcccacctattctgcctcacaaaagtagaagtaatggcaaccactatcatagggatcatgctcacctttttcttaccagacaaatttggatattagcttgaaattaataccttccttaaaatgttggaatttggttatatgcgaaattttgctctatttattcattatattttgtatggaattatttttgccctatattttcacttaagtgttctctacccaagattttaattgaacccaaatcagccagacacacagacatggattttgctgccaccaaggttaattcttcttttaaagttaacttttaaaatttggtaaaatatagctttgaaaatttgcattcgtctagtgtttgttatgtatttcccccttttgtttgattatatgtctatatttttcttgtagaaattgatttttaacctgctttttatgttagcttttatgagcttctgtctgaattctgaatatgtctttcttaatgtcttctaaatgtttctttctggattattaaaagatttattaggcttttaataattatatttgttaccttagggaatgtgtttgaaaatattttaaatggaattgccagttaacacagcattgaactttttcttgttagagatacattgttttctaggcattttattgggagagaagttagtatgatataatgtctttggctgatattaactcttctaagatgcattgtttctgagaacaccattgtctgatttcattcagggaaatttcacacaagccagtagagtcaatacttttttcaagacctgttaattgatatatataaaaacttgccattgtttacatgcccatttcagatcctttatgtgacctaagctagaaatgcattttaacagcatttgtttttccaaaaatatttatttatttatttattatagagatagcgtctctctatgttgcccaggctggcctcgaactcctgggctcaagcaattctcctgcctcggcctcccaacagtgctgggatacaggtgtgagccattgtgccaggcccttgtttttattttttttgaacattgtattttgaaaggggtttgaaggtgatccctagatagcaaccagtaatgattcgagcagcaaaacaatctaaaaagtaattttataagaaaatgcagaacataaatgagcccataaaaaattatattaggttctatttacattactaccttctttcacatgtaatatttcactaacatttaatgaatttctgtgcagtgccatataccattatgaattctaggatagaagaatgagtgagaaatgttcttaggccttaggaagaaggaacaagcatctctgtgtaatagttatttcaactcttcttttacacctcattcccatattaaatctcagaaaagctaaagtaatagctatcccagatctattttagactccagacacttacttcaatgtcttgttctccttatcagactggaatcattccaaacctcttaacttctgggcaaccatgataatgcgacagaaaggacactaaatctgtcgcaaatttatcttgatattctatccagtcttacttggtactgaaggtcacaagtaaaataaggtggttgttttttgtttgtttttttttttttgacagaagagaaaagaacactgtgagcacagagtgaatgtctaacattgattcttgagtagcaggaattctctatgcgagaggatctctatgcaaaaagatctcatattctagcacaatttaaggatctctatgcaaagatatcccatattttagcattatcaataagctatggggtaatatattgtatgtggtgtggcttgaattctagaaatttgatttctagaaatggtccctgtagttaaggatatataatgtggccgtctccagttttctatgaggaataggaaaatactatcattattagctgtgtgaccatggacaacttgcttcgttcttcagttgcatcatctgtataaaataagaataagaaaatttacatctgcaaggtgtgatggagatcacatgggataattgtggtcccagagcctggcacaaaagggcttaatatttataatcctccccatttctccgtatactctaaaggaagtttattgcttatcaaattgtgccgtggttagttgtacagcttccctgccaaattgtaaactccaacactaatgtgacgttacattttatatagtgctatgattttcaaattgtttgcataatttcaaatacacagtaaattgctttttattagtataattattgctattgtcaatattattattacaacagcttcacagtaagatgggcagaaaaaaatttaatttccattttacaaatgcacttttgaggctcacagaagtcaaatagaccaaagtcacagggctagtgagggacccagaagaaacaaattgtaattcactgattccaagttcagtggttgccttactgcatcataaaggctattacacaatccaggtgtatcatatgattcttgtctatatattcatacatatcagaaaaagtgttctactcaaaattgctagcaatcaacagatactgatagtcattagtacttaaatctttatcaaatgaaatattaatacccatgaaagagaggacaatgaaaggtttgtatcatttgtatgtcacaagtcaacttttttcaatcactcattattagtttaactgtaaaaaattatttacatttagcgtgaaactttcctgtattctcaacatatttccttcggtagaaaagcaaacctccagttctctgttctttgcttggatacttgccagtttgtaactcagctatcaaacagtaaagctcacaaaacacttattaaaatgactaaaatccaaaacaccaagagcacagcatgctggtgagatgtggagcaacaagaactttcattcattcactaatgctggcaatacaaaatggtacagtaactttggaagataggttgacaatttcttacgaagctaaactatacttaacatatatatttgtccattttcacagtgctaaaaagaagttcccgagactgggaaatttataaaggaaagaggtttatttaattgactcacagctcagcatggctgaggaggcctcagaaagcttataatcatggtggaaggagaaggggaagcaaggcacctacttcacaaggtgacaggaaggagaatgaatgcaggaggaactaccaaacacataaaaccattagctctcgtgagaactcactcgctatcatgagaacagcatgggggaaacagctctcatgatctagttacctccacctggtctctcccttgacatgtggggattatggggattataattcaagatgagatttgggtggggacacaaagcctaaccatatcaccatatgatccaaaatcatgctacatgatattcacccaaaggaaatgtaaactgtgtccacaccaaaacctgcacatgcacgtttatagcagctttattcataattgccaaaacttggaagcaaccaagatgttcctcaataggtgaatgaacaaaaagactggcacatgtactcaatggaatattattcagtgataaaaagaaatgagctatcaagccacaaaaacacatggagaaaacttaggtacgtaagccagtttgaaaggttgcattctatatgattccaatatatgacattctgaaagagacaaaattctggagacagtaaaaagatcagtgattgcctggggctctgagaaagtgcagagggatgaatgggtgaagcacatggcatgtttaggacagtgaaactattctctatgatactgtcatggtggatacatgaccttatacctttgttaaaactcagaattttacaatacagagtgaattctaatataaactatggactttagttgtaataaggtatcaatgttatttcataagttttaataatgtaccacactaatgcaaaattataataataggggaattgggggaagggtaatggagtatatgggaatgcactgtaatctcagtacaattattccacaaacctaaaacttctttcaaaaatacaagctattggtcaggtgtgatggcttataccagtaatctcagcactttgggaagtcaagaccctcagatcacttgaggccaggagttcgagaccagcctggccaacatggtgaaatcctgtctctactaaaaatacaaaaaaaaaaaaagaaagaaagaaaagaaagaaagaacagaagaaataaaagaaagaaaggaaagaaagaaagaagaaaagaaagaaagagaaagagagaaagaaagaaggaaagaaagaaacagaaagagagaaagaaagaaagaaaaagaaagaaagaaagaaagaaagaaaagaaagacagatgcggttgctcatgcttgtaatcacaactactcgggagactgaggcatgagaatcgcctgaactcagaaggtggaggttgcagtagggtgagattacgccactgcactccagcctgggtgacagagcaaggctctgtctcaaaaaaaaaaaaaaaaagctattaaaaatatgtaaagctcagtctagatacagtaccagaatagtaggaactttatttcacctgtcctacaaattatggttgtgtgccacttgggtaaaactcagaatccaaatatgtgaatgtaagatttatggggaaattatttgtatttcaaaataatccttaatgaatgcactccttctaaagtagccattaataaagcagttaatgtttcatttaattatagattaatgtacataagatatgccaggaatgcaattaggaactgggaagggggtgttattctaataacttccacatagcattgtgagacattttctgctttcttcaaatttcatttaattacattttaaacaaatatttttgtgagcctattatatagtccttcgctagcactgaggagacatgctttgtgaccttggtgatttcacattcaaatttccctttcacctacactcttccttgttttttcatgcctgtgtagattgtaaattcttcctcagattaagacattttattcacctttgtaacatccacagtatctagcacaatcagtgccttcaaaaacaattggcctcaagaattgattgactcaatgagtgactgaaagactaaattaataagtacacatctatttgtacttccctgcttacttataaggtatgacaatgaaatactgagacagttatacattacttacggactcaatctcatttctttacaatctctattcttcttttttgagtataatgttattttacaattccactaacttgtcactctttattataaattcatatctccatttcacctgagaataataaaggcaaggaagtattttaaatgatcttgttttttataactagcattcattgagcaaatcaaagtatgaaaataatataggtgtcagtgattattataaagttgtatgcacaaaacattccaatgattggggccaatacagagaaaacatctcaatatttggaattttgcttttctgtaaatactttgatatgtacttacatcatatcaattataactcctgctgaaaacaaacagtgcacacaaatttggtagttggaggagactttataaagggactaattacgaaggtttagaccgggttaggaaaaacacacggaatagtgcaatactttaggatggcaacagcgagcaccgttataaccactaggccaaaatgaactaaatgaacagggagattaccatttatcagaaaaagagggagaaaggaaggagagatgaccaagcaagtcctatgtgaagacggctgcctgacttgagctgtgtgatctttggactgataccacctgcctgcactggcctagcagggcgagaatagtcaatatctggaaaatggatcacctgaccttactttcctccctccctgtttcctctttgtggtgtttccactggccaaactcacagcgtagacaaaaggagtgcattgatgtagcagtggttctaatccagggccaattgtgctcccagggaacattagtggttatcacagctcaggggaggaagggagaggagtggagtgctactatgattcactgagggatttttttaaacatctacaatgcacaggacatccttccacaacaaagtatccagttaaaaaatgtcattactgccaaggttgaaaaaccgtggtgtagtcagtacaattcatcttctccaggcacagtgcaggagtggggtggagtgtctgaaggggaagaaggaagaaaccagcacaccccacaaaagtaaccaatgcaaataccaaataggaaaagacagcacttaaaatacaaaagtctcaggaatatatctgatagtgttttatggaatttattaaaatttagcctggagtgagtaatatttagcaagccaggtttgtctttagagaaatccttgtggggtttatacaaggatttattaacaaagggcacacacaatactcatattacagtcagtctggttatgtaaaacatgggcaagaatgtaataggacaatgtgatgtattcacaaaggattttaggactacacagataatcctctaatgctttcacttacgtactatgaaaggctatagtttgcatagtgatatagccacgtaagatagtaaacttgacattcatgcagctatacatgtttgcacacaccaggatgcatgccctttctacctggttgattttttattcttttattaatctctaatttattccccagaacactctccataaaaactttctcacaacttaaatctttaatctattgtgtggatttctgactcattctccaagcttttcctcttccctccgcaatgccttatagtcttatgactatttatccctttgcctacatttctagccagatctcttgcctgatacacactctcatatttctctttgcacgctacacatttttatttagatatcacactactactttgatttcaacaggtctcagtttaacttaatttttccttcaagcaaggagtcccttcatatcagttatcaccattggcaccagaatttttcttatgacttcccatgacctacaatataaaccatataaatcactgatgcctccatagttccctccctctcaaatttagccataagatgattttaggatccttgttttttccaatctctctttcattctctcccccatctcttccattatgaaggtttggataggacacaactcatgcctagattagtgcaatagatgctgagcctgtgcagcggtagtttagctttctctcctggttaactttaactgccacatatatcacttcacacgtcatttttcattcaaacgtatttaactggctcttcattcataagaagctggaatttgtcgtttgactgatattttaaagattttatattttttctccatcctcgttctaatgttgtatcttgtgtcatttgttcattcataaacttaagacttagctaaccactgagcatccaggaaattcagtatctatcatgtgaattctctaatactggttgatccattgtcaccagagcatagcaggcttctcctgcctttatgtatgtttgtcatatagttcatgcctaaaattctttcttaaatcttaaattcctaagatacacacttttgcccaagatcacagtaatctctgccataatctctgctggaatctgttcactgtgttgctcctgctaaacttcttacagatgactttttttctttttggtttccctggtatctagtataatttcttatataggtactcaataaatgtttcctgttgatctctacacctactctgtacaataccatagtgactagacacatgttgctatcaagcatttcaaaagtagctagcctgagttgagatataggggtaaaatacacaacagatttcaagacatattatgaaaaaaacccataaaatttctcagtaatttttttatagattacatgtagaaactataacattttgaataagttgtatcaaataaaatataaaattcacccggttctttttaatttgttaaatgtggtggctagaaaatttaaaattacataattggctcacagaataattataatggatggtattgctttagatcaagtttgtctaacccgtggcccatgggccacaagcggcccaggatggttttgaatgagatccaacacaaatgtgtgaacttccttaaaacattatgaattttttgtttgttttgtttttgtttttttctcatcagctatcatgagtgttagtgtattttatgcatggctcaagacaattaattcttcttcaaatatggcccagggaagccaaaagactggacaaccctgctttagatagtaaagcatatgagtagttaatgtgtactataagcagtgtgatctgatagactatttaatgttgtttgatggtacattattcaagtcgattattatgtctacctatgcagtttaacgacggtaatgagagagggcagcttgattacaggtcttatcttttgactaacttgctaggccacctgagaaggacccaaattatctgaatgcttaactcaactaatttgtattcacttgaagaatttcaaggatgtttatatgccatcaacttgctttaaattttttctctcagtgaaaatttttcttaaaatgagtatgtggtattcaaatttatccttgttttctatgattatcttttcatagcactgtggtttccaggaacctttttttttttgagatgcattctacatgtaactattgcacagtttgcatgtagtaaggttcattattcttctacttttccaaacacctggcatgtttacttgaggttggtacaccttgtatcccagattttgctgtttttaacttaaatattgaatattttgattaaacattatggaaagtttaaatgggtcaagaaaaatagcttttcttcccatgaagaacaatacggcataggagttaagagcatagatttaaagtcagaaaacctgtgctgcctacttgtgcaaagtcacttacatgctgtacttctgtttcttcatctgtaagttctacccctaggtatttacttaagattaatggaagcatatgttcatacaatgacttgtacagaattattcacgatagcattactcttaatagctctaactggtaacaacacaataatcaatcaacaattgtgctgtattcatacagcagaatactacttagcaacaaaaatggaatggactactgataacctcaacaacatggatgaatctcaaaactatcatgctgtgtgatgccaggcacaaatcagtacatactataattccagaaaagacaaatgtcatccatggtaacaacaagatccatgcttgctggaggtagaggcatcagttcagtcattcaggaagctgattccaagatggtgttagaattacaaccatccacaagagatttattgcaggcaatagctatgaaaggtagaaagagaacaggagaaaaaccaggcaaggaaaaaccacaatgtagttgtgatatcacttcaaagggaggcagaaggaaggagaattgggtaggaatagccacagattacagtgcagttacaagaaagtcttggcttccaacaaaggttacttgttgaggagtcatgcattaggcagacatgtctgggctgtagtttccttgctgctcccagtcattggctggaggccagtctgggttcctgtgctgtggtggatcccattgctgctgcagcaggaggccaatagcactcctggcagctaattggagagaaaagatccaagaggtgtaccttcatggctacccccatggggctggggtggaggtggaggagaaggagaaggaattaactagaaaaaggcacaaaggaaaattggggaaaataatgaagatatatgatttctcaattgtggtggtcgttacatgggtttattaatgcatcaaaactcaagaaatgtacatttaaaatgagtgcatatgattgtaagtgaattatacctcaatatagttaattttttaaaaatcatagatttctttatatttaatgcatgaacataaacctaagacactcctccactccaaaacttaattaccttgtgatcagcagagcagaaggtactttgtgatatataggtagagaagatgaagtcttgtgacatttaacaagggacaggaaaatggaccttgtcctaagttaccaaactgcaaaaatatcacctacaaaggctattcataacatacattttcaagggggttacaatatttgcctactataaaattttggatctgtaaaggggttaaattatttgtgcaggggaataaacatcaaagaaacattaagaggtccagagaagtaaaataggaagggtcttttggctagaggagatatttaactttcagaacatgtggaattaagttgtattgattatgatctgatcttcttccccctaaatttgatcctcttcctgtaatctattgtttccatcatcttcaactcttccctttccctctcccttgtccctcagttctagtcaatcacaaagtcctacagtttcactttctgtataccttatttctggaattcatctctagacttcaaaatatatatatatatatttttttttttgagatggagtctcgctctgttgcccaggctggagtgccgtggtgcaatctcagctcacagcagcctctgccacccaggttcaagcgattctcctagttcagcctcctgagtagctgggattacaggcatctgccaccacgcctggttaatttttgtattttcagtagagatggggtttcgccatgttggccaggctgatctcgaactcctgacctcaggtgatccacccgcgtcagcctcccaaagtgctggaattacaggtgtgagccactgcttccagcccaaaatatcttaagtagataattgcacgactaatctctgcttttctctcccagcagccttccaaattcatgtctcacagctgacagagttgttcctgccttcagattcatgacctggctctgtgttctagctcaggctttctctctcatatcacctcttgcctctctgttgcccccatattttcccctctggttggttggtgctcctttggaaccctctgcatatcttttcaagaatattatgacttattatgcctataaactttgtttaattatttatttctaaaatttgacagggaactttccgaaggcaggtattgtgtctttctcatttaaaagcaaattctcgcctggcatggtggctcatgcctgtaatcccacactttgggaggctaaggtggacagatcacttgagcctaggagttcatgaccagcctgggcaacacagttagaccaaaaaaaaaatatatacgaaaattagcctggcatggtggcacacccccgtagtctcagctagtctggtagctgaggtgagaggatcacttgagcctggatggttgaggttgcagtgagctgtgattgtatcactgcactccagcctgggcaaaaaagtaagatcctgtctcaaaaaaaaaaaaaaaaaaaattagtgaatcctcagtgtttaaaaagtccataaacatactaaacatagaagacctccaaatgaaattaatcaattattatttagtgggttgcttctcttttgttttaatatagttttaacaaagagtaaaagttatgatctttttatatgtaaaataaataatgccgggtttgacataaattttaggaaaactagagacgctacttcctaaaaattttctttctataatcttcctaaatatttttccataaagtacaaaataatagaaaaaaattaagagattgagtatcctttcaggaagtgatatgacaaatagggttcgagaactatttgaattctcaccacttttcataagggcagatctcaagttaaatttttctattcgaatttaaatgactttcactggaataccattacagaaaagcttctgtgtttagatggcaatatggagtttcttttcttggaatattaattgaaggagaagtcttaattttttaagtctatatctccgtatatatttgaacctattttatatgttagtccttctctttagtaaccttcatccacagtgaacaagatttacccttacctttaagcagtagcggctactttatgtgaagtgaacagctgctttttttatctgcatctagacatcaagtagtccagagtcctttctaacaccctagcaatagaagtaagaatattttgaccattccatgacttgatgatacttctagtaataatactgtattattaaaaacaaacaaacctttgtgcagtggtaattgaagcagttccttgggaacatgtattaagtactttttagcagttaagtccactctctgtaggttaaggaatatttaaataaaataatgtggcaaatgagttcaagatgataaatgcgatgagaactaaaacagctttaattttatgtgggaaataaatagaggaaaagtacattacagggctcctggacttatttctttcttcaaagtgtttctcctagcgaatattattactattttttctcttaagtaaaaaatacacaaagtatgaatctacacaggataataatattgaagttaaggatgatgtctcctccttcactctccaaaatactatttacttggcttcatggaaatctctctcactccaattccaccgtgtcaactgaggtcttctgttctttctctccctatagcatattcctgttacataaatcctaaactgtgtcgtgttagtcacacactgtaacctctagataagcgcctgtccagaggttctcaatcagagccttgcaaatatgtattaaatcaatgggtcatcttcagtgtctcagtgggcccttggatatgttttgcagactgctgtgagtatgtagggatgtccagtatcgagggaagtgtggatggctttcattggttcttatagggctgaagaacacatagagcagtaagcacttctactgtagggagagatcgagcttctcccatccccactgctggcaccaccaccaccctacaccccattttgagttctgaaagtgaatccttgagaaagaacacacaaaacaaccatcataatagtgggcacagctgtgggtggtagaataacattcccaagcttcttttcctacacatgattaatattaattcagcaaacatttattcagctcctacttttaaacaggcactattctaggtactaaagacatagaggcaaagcatacaagactctgcctttgtgaaacaattaagaaataagtaaaaagaaaagaaacagaaaaggcaatttggatagtgtcaggtgctataaagaaaacaaaatgccattttaataaataataataatacaatgttttcatactatgtgctagacactatgctagtaggtatttatagacataacctcaattaatcctcaaaatggcatgttgatatcaataccccaagtttacatatgagacttaagatgtctgagtatattcccccaggtaacaattaatatgcacaataaaactttttgctcattcatttattaacctatgttgattgagtacctattttgtgtcaggcatcattttaaggcacctggatatagttatgaacaaacaaataaaaatctctgccctcaaataattaatatctcacagaggttaggcaaaatataatcagaaaataagtataacgtataggatgccagatcatgaaagaagctatgaatggcatcaagaagctggaaaaggcaaggagacagattttctcctagagtctccaaaacagaacacagtcctgccgacaccttaactttaggctagtgagacccctattggacttcagacttacaatcccacaatgtaataaatttgtggtaattcagtaggggaacaatagaaaactaatacgatatcaaaacaaattatatcatagaacaagaaaatgtaattgtgacaaataatacctacaaaaatgttgtaaatgctaggcaaataatgtgtttaaagcacttaggccaatgttcaacgtaaagtaattcatgctataatatcatcatcatcattaccaatatttaggggctctaacaaatgatgtacgtgtaagcagatgtaagaaaatttccttgctgaagaggaggtattaatagagtatataacaatagataacaaattccaaataaaggcaaactaaatgttttattggattaaatttaattttaaaaactacaagaggccgggcgcggtggctcacgcctataatcccagcactttggaaggctgaggtgggtggatcacgaggtcaggagatcgagaccatcctggccaacatggtgaaacgctgtctctactaaaaatacaaaaattagctgggcctggtggcgcgtgcctgtaatctcagctatttgggaggctgaggcaagagaatcacttgaacaaccaaggagtcggaggttgcagtgagccaagattgtgccactgcactccagcctggcaacagagtgagatcccgtctcaacaacaacaacaacaacaacaacaacaacaacaacaacaacaacaaaactgtgagatccatggtgggcttttaagaggaaaatgcaagctaaggtttgtttagactctgagtactgcatgtgtaaaaataaaggcatgatgaaaagatcaagagattagagtgatactttttatctactagtgtcagagtcatgaccaggggattggctatgagaatacataagctgtgccaggagtaatccaaggagattgtttcaatttggaagagtgtccacagaatgattctcatactagacgttgggctattgtaaagaaagttggtaggtactccatcgctaggatcatatcagggagaaattgaacaggatggccctaatgaccctgttgtacccctagcttatggattaggcaagtcacttctactcgtataccctgtttccccatttgtaaataagaggatgtgttactctaaggatctctaagattctttgcagttgttaaattgcatagctctccactgattccatggtggaaatttgctattctattacaaatattctaaatgtatgagatatcagacatactcatttaaaaaacaaaatacaaaaaataagtattctacaaataaacacagataatgtttaaattctatatgtctttgtttctcttcagaagcatccaaaatacaaaccatctaagaggcaagaaaatgtcgtgatgttcctagtgcaagttaaaaagatttgctttcctcaagtcggaaagcccttctcatttttgaggtttttttcttcttttttttttcaagtgaaagcattttggaggagtcaatatccatctttaaaggtagccaggtcacatgtatacatatgtaactaacctgcacaatgtgcacatgtaccctaaaacttaaagtataatttaaaaaaaaagaatttaaataaaaaaagaaaatcagagagaaaaaaaaaaagatgcatgtgcaccctgatactaccatccatagtgatacggtttggctttgtgtccccacccaaatctcatcttgaattgtaacccccatgtgttgagggagggaccttatgggaggtgattggatcatgggggtagtttctccatgctgttctcatgatagtgaatgagttctcataagatctaatggtttaaaatcatggcacttccttttgctctctctttctcctgccatgtgaggtgtgccttgcttccccttccccttctgctatgattgtaagtttcctgaggcctcctcagctatgcagaacggtgagtcaattaaacttctttctttataaaaaaaaaaaaaaaaaaaaaggtagccaggtaaaaattacttgtttccaggacattttcacctgaaagaagcattgtcatataacatagaagcaagaaatccagtagtgggggttatttaaaaatagctggaaaatttcaatcagcatgagtttgaagcaacaatttatcatcaccttttatggtgggtggggttaagaacatttcagcgggcaaagtggtggtgatggggaagagacaccaggggaggtgattcccattgcattgctttgtaaacagaggcacaggttcttcatttttgtcacacaaaatcacagctatgcagaatttattaatttattcttctgagacaagaaaaaagccaccaaaggaaaccaacagcttgctcctctcacactgggggaaccatatgagagacttatctatccctgactttaattttgacctgaggagagctcctcttaaggaaaacaaattaattcaatgactatactacttaatcattgacctttatttaataagagatttttccataggatatgctgagctgtctcacttacatcagttgtgtctcctgaggtgggtgacaggagaccacaaatattgcatagcacacaaatcgttaatagcagctgtataccaaaccattacctaaatatgtagagtacaattcattctcactaatgtcagagagcatgctataaaatggtgaatccggacagctgaagatactgaataataacctctattttgaacaagtttacagtgttccaatcagtaattaaattgatacctgatgaatatatgtgtgtgtatgtattcatagcagagatggttttcctgagataaggattttgttattcggataggctgctgctggaattgtccttctacccttgtttctttgtccttagtcatcactcatacctctttccactcttctgccatcacttttgtcaccaaagtcatggtcctttccccgccgattgctgctgcaggtctagggcaccaagacttaggcagcactcaccatgtgccaagaactggaccacaggtaccatccagcattgctcatggagactctgtccctttctgtaggacaccctccttttagctagcaacccctccaccacctagagcctctggacctctcattttaatattaagaactaggaaaacttaccgctgagaataactagtacaactagaactggtagagaaatctgggtctcttgggaatggatttttaggctttattgattagaggtgtattaataatgcagtgttatagtttcatgacataacgaataaaaaagttcattttggacttgcctttcagctccctaggagctaaaagacgtatttaatgtaacttgtgtggtggaaataagttcttttttcaggcaaaagatgtgcaaacccatctggggaagaaacattaaaaactaaggagacagtgtcctagataactatgttcttttcctgttttagtctaaaataatgattagttttcttatatatcttcatttgtcttggttccttttagcccaatttaataatattattgcagatattgatgaaaacctttaccttcctcttaattcatcaaagtacttgataaaatttatacatagtacattaattgggaggtttttatgagattaattaatataatgaactgatgttgaaattatttaaaacctgaattattattgtattaagtaggacacttaatacagttaatcagttctgtctttattcatttgtgagaatttttggcaagctattgtgaatattcagggaagggaatgtatttttagcaggaatcttatacctcctacatagaaatgaagcatttactgaaacatccatgaaacaaaatgtttctgaatgtgtactatacacttgttataagccccttttcttctgtagctatattttggagaaaaatctttgctttgacaaaaaaaattatgttgacttacacatatattttataactaagcagtgtttggtttgtgataaaggatacaaaaatataaaaatgttcagcacacgtaagtaaggccttgttgacagtgtgagttatgctactggatactcaaaaggaacattcagtgttctcaggtggtctctagactgtctcaagcctaggaagatattttataagcaaaggaataagagaaggaagattcagatttaatccaagtgaagaattcagttttgtgtgccttatcctgttattttgagaggcagccaaaagatgctggtcagcaaggagaattgtaagttgggcagccaactctgatttctcaacctcttagctgttttcttaaactcagaatttttaatgaatttaaatgtccatatcaggtagactttggggatgcttttaccagtgattttcagaatgttactttctggcatttcttttcacgtagcattatattaaaaatgaattcattcatccaccttcccttgtccttactaattttccctcctactcccttcccccttgttcttgccatggggacatgcaaacactggtggttgatgtctgagcaaggctgctgacagggggaggaaggagatgtcaagcagaggtcaatggcagtgtgcccagcagcctaggaagtaggagggaaaagagagagagacagagatggtggatgaaagagaaagccaggatgattatggtggttatgatacttgtcatgctgaacacccaattgagcacccaataagcacataataatttaatcatcctctggcttggatggcagtgttctatcagtgttgacttcctggttgtgacagttttacagtgttagtgtagaagagaatccttgctttagagaggtacttactgaagtacttagggttaatgcaccattgtgctggaaaaagatacgcacacacacgcacacacacacacacacacactctcacacacacgcacaaatacatccatgtgttaggcagagggagcaaatgaggtaaaatgttaacaattaggaattctgggtgaagtggatagagggactctttgactgttcttgaaacttctctatacatttgatctgtttcaaattcttcagaaaatcaaactacaaaaacttaattcatttagtgaacatctactgaacatctgtatattaaatagtgttaaatgaatgtcaattaaaatgctcaaacacagtagaggttgattctcattcacataagtccatggtaggtgtttttggcaggtgggtgagtttctcccttagggagattgaggaacccagactcctcccaagttgcagccccaccgtcttctgaggggatgcatccatacccacttcgaagtagcatacattatttcctttctcattcctttggataccagccacaatttattcaaggtagacagaaaattgtagtatatagccatatgccctgacaaagaagggagaacagattttggtggacaactagcaaactctgatacaatctgttattaagcactgtgtgtggatagatgctaactagaaggagattatcttcccttcagcaaatataaactgaatgccgtttatttggttgaaactaagctagatcatgggagtatagaaattttataagaagacatagtcacttctgtcagtgagctcaagaagaattagtatgcggaatgtaatcatacctacagggggcttgtgccacttaagtaaaatgaaacattattttgagtacaatttagcaataaatgtactacgagatcattaaaaatcatgtttgaatgttattgtgtcaaggatgggaaaaagacttttgggttgtagacttgataattatagttaaaaacagtttttattcttgtttagtcttattttttatgtttaaacatatttatacttgctaacatttatacttgctaagtaaagactgtttttacaaccatgacaagaacaaaacatattagtaatgcaaatgccacatttcctacaatcaactaatcacactaacatatttgcatggaagaatcactgggattgatctggccacgtgtgtagtcatgcccaaaatgtgaagtccatctgttttgcaattttttttaaccactgttatccaaatgctccttggattttttttattagtggatatattttggaggtcagacaccctcttggctagatcatcacctttataacaaatatatatactattctcatggaaatatatttagacattgccctactgggaatttttttcaagtaattaatgtacagcttgtgcaacagcttgatcttggcttcatggaaataattcactcttagcagcatctaatgccacaaagcatttatggatgtcagctcagaacttacttttatttatctctgagttacttttttttttttttttttgagacagagtctcactctgtctttggcttgtccctaacctcttaacagacttaatattaagctccatttcactcagtcgttctgttgtcatataaatgagacattctacaagcatagtttttagtttctgccagagcatcatacaacattgtgagctatgatgaagataaagacctagagaagatatttaatatgaagttcattatctaatatttggtatgtgtggcaaaatagcaatctactgcttggttctgctgtaatctatttacccacccatcccatctttctttcaatttaaaaggataatgattttagtcacgattatacataaacccattaccataggcaataaacaatggggcaaaccattggtcccatagttggagtgtggtctgaagtgtgttttggtggagagagatctatgtctggagatagctaacatggatttggatcccagatctgctcctacctgttgctgtgcctgtgaccaaatcatgtgatctctctggtttcagtttacttgtgaataaagtaaataccttcatcaacacctgtttttgaatacaatgtttttctgtaatttttgcttcttataatgttataatgatcatccttacatctaaatcttggtttacattttcatcaattcttttggaaagattggagaagtaaattttggagatgtatgtcggctattaaaaatgtttaattttttaattaaaaattaaaacgttgaaaaatcctgatgcaaaataaatgcattatgcttagtgaactcttctcatttcgaagtttattcaccttcttgtttttgcaagtttcctgaaaaatgcatataaagtcactaagttagcagaactttataaaattatataactatatataatcttttgatatcagtgaagccagctgatcctatagaaataatgtaggaattataatcactagcacataatttaagagtcctgtggtcttattcatgttatttaccctctctgaatcttacatatagtaagagggttattatacataatatgtgtacatgtatacaggtaagtaagtatatatgcttatgtgtaaaagcagagttattgtgagagtcaaatggaaatgtgaaagtactttgtagttttttattactattattaatttttaataaaatggtaacattcatttaataatcattagttttaacttcagattgtactggatttcctctagtatttcttaagattagtgaataaagtatttctcctaataaatatattgactactgtctttcgatcaaacatattaggtatatttttacagtagcatcaggcagtgaaaatttgaagctctttatagaggactgatttatgatgaaaaggaataacatgaacaaatggaattatatgaagcttccccagaaatatctaagaggggccaattttaagaaatatctgacttctttttcatggacatttcaaaataaacctaactcatatggtacagtttttaagagggaaaagaaaaaaccatctgagaatctctggaattctgccgaaagtatcacttggcattttattctaccttctggatgcagttgattgacagtagtgttatgatgccaggggtatagtgactagaaaaagaaaaccagggaattcagtgttcttgctcatgaagaacagcttggttctttaaaaacaatgagattttgccaccccatctcacaaacctatgatttgtgagaacaatcccttttgtgttgcaagacttttacatttctcttcccacactatattagaagaataaacattgcttcataagtaccgattgatagtctcatttcatatttttaaaatagagttactttaaggttaaatttttcatgtagattaaaatgactaagtaaccattcacatatttcaaataaaatatatttttactacaaaaggaaaataactagattcttaagtgttatagtcaagtgtaattgagtaatatgaattctaaatgaatttctaagatctgctcagctttcactactttaggaaggaacaacttaagaaaaattttaataaagatatctcttcacacacatggcagtgttgtacttagagaacatgacccaaaattttttatgactgcatattgaattcctgatactcttgggaagctccaaaagcaccagtggagtttccagatgtaactgtggctgcagacccgccagtcccggtgttggaagggatcattataggctcttgtgtgcagactcatcttcagacccagaggaattaaataacttgcccaaagtcgcacaactttctcatggtaggttgggcactagaataaatattgctttttcttaagagttttagcctccgtattatgaaatcttctatgttctgctgatgatatctcctttcttcatctgttttctatttttaagcaatggaaatacaaacttgcaactccccatttccaacacaacttagaaaaaacaatatttaaagaaaaaattacaggcatctcatctcctttacctgacagatgcttgatagtaatggcctctagatagggatgacatctaatataaatgtgtcctttcaagtcaagctttctctgttcattagtagaaatattgtatatcaagtgtgcaaaaattttcttcaacagggagctttgtttccctccttttattataacaatctgagctttgtggtcccagggtctcctagtgcctgtctttaggtctgtttattcacatgaagaaagcatgtcatatagtattatctaagactcaggctgcttatgcatgatgacagaagggttcccaggcacaaacattcatccatgcattcatccatccacctattcatccattgatttggctgataattattgactactgttgagttgccctcagatttagtttctgtccttctgccatggggaaatatggggttaagccacaacatactcttctcttctttttctgcaccttcttagtatatttagttccattttgtctagccctgcctctgacttctttgttgtacttcaggttttttatcattgaaagttatttctggatcatagatcattctcttggtcactttgcttgttcacttataaaattaattcagaaaaaatgacccacagtaattaccgtaaatcacagaccataaactataatactgtatattgtattatagtacagaaatatttatactttaaaatgttttaaatatagatattataaaaagatatgtctcatataagtaatataaatacttttttattacctcttctctccctattctccaggccagtgttttaaaaatccatctttatatgtccatcctggaaaaaactcatgatcataaatgagtttctcaatagagtttataagcccacagttgaaacacaattgtcttagcatccatttagttgtcatacttttaagatttaatggcaaatattatgttttgtttcttcaaaagaaatattttaaaattttagtaaaggcagttagagaaggtagagataatggactgtttaatcctacttttcatcccacaagtgaacaaaaaaatgataaaacatttttcccaaaatgtagctttaactatacttaaatttggactaaaatgggagatatcttttctactattgaaaagccgtgtctgtagattaatgctaaaatcgggtgtaaaagcaaaatttgtttggcttgattgccaatggcccattcatttggctacagaaacaatagcacatagcaacagataatgatgtgagatcacctagctcaagtaagagtgtctgatccgtcaaaaatatatacatcaagattcaaaagaaatgtgtgttttctcaagtcatctctgtaaaaatacattaaatagaggaatagaagtttgactttgaaaatacattgcagacccaatccgtctttcctattttctggtgaaaagtatcaaatatgtggaacctggaactgctattctccttcttaaaaatctttcttaatattctattgataactggtgcaagcctaactttttgtcttacccgattcttctcacaccaaagtgataggaccttcaggtagcctttggatagaagataaataataatttaactattgatggaagttagtattagaattagacttggaagtctatggaataaaatgattctacaacaatttgtacttcagacattagtataacaaaacatgtttgcccgtgcatgcggaaacaaccaatttcatgtggatgcttatattcacaaaggagtaaccacctggggtttcccactgttgctccagagaaaactagcagcaggagaacttctctgaaggtatcaagacatctttaaaaaacacttgttaagtgttggttcagctaaagcagggagttttcagttagtaatggcttttaaaaattaaaacaagtttagcatgtaggtcattaaccttgaatcactgtcatgattattattaaccatctgttctcaaatcgaaagatatttttcttttctagatcacatttattctcacattgctcaatttcactatatatcaagacatgaaaactgtaaaaatcacaccttctacattattatttttattgaaaaattcctaatgaaacagtgcgctctgggatagagaaaggaactaactgacattttgcttcttaacttgtttttatgcaagttctaagtggtttctggccatgtacataaaagacaaatatctggaaaaaaaactagcagaagtcagttatttggctctatctactttgagaattatgttatataaatgttaggaaattttttgtaatattcttatttagaaatgaaatataaaaagttttaaaaatatctaaggacagtatacagtcctaaagtaaagctgttaggtaaatgctacacaatcctcttattacagagtcacttacctgagaatataagaagagggcctcttgtttaagagtaaatgtgagctgcaatcaggattctgcactcatttggacacttagttttgtttttccatgactggtgttgcctgttactgagacacctacctgtcatgtgaccacagcttatgttacaatgtgtctagtcagacttagagatgtgtgaaagagcagtacctagacgggaaactatgggtctataaaggttttgccttcttgggcggagttcaaactaggaagccacaaaacttccagttgcattttcacagattaatgaaatatattttacacttttcctgaaagatattttatttgtgcaaaccttgttacaaagtacagccagttgattaatcgatgaagtgatttgtagtggattcttatattttgtgtaagggtatatgtgaggccctatatatgaggctttctatataatgaagtataattcagttcagcatttcaattcagcaatcacttattgggcctctactcagttgccttcagggctttataatttaattgataaagggaggttaattaattaattataacaacagatcgcttaatagtgtaactactaatttaattaatgacaaataacaatacattaaaagaaatgcattaataaaaataatatattggtgttatagacaataattttctgattaactttattattattatttcaatagcttttggggagcaggtggtttttggttatatggagaagttgtttaggtatgatttctgagattttggtacactcataacctgagcagcatacactgcacccaatgtgtagtctttcattcctcaccttcctcccacccttcccctcaagtctccagagtccattatatcattcttatgcctttgcatcctttagtttaggtggcagttataaatgagaacatgtaatgtttggttttccactcctgagttacttcacttagaataatggtctccaactctatctacgtagctacaaatgccattattttgttcctttttatggctgagtagtattccatagcatccacacacacccccctatgctttatatatatatgtaaatatatcacattttctttatccactcattggttgatgggtatttaggctggttccatatttttgcaattgtgaattgtgcagctataaacatgcatgtgcaagtgtctttttcatataatgacttcttttcctctgggtagatacctaggagtgggatcgctggaacaaatgattgttctacttttagttctttaaggaatctccataacttttccatggtggttgtactagtttacattcctaccagcagtgtaaaaaaatgttccctttttaccacttccatgccaacgtttatttttttattttttaattatggcaattcttgcaggagtaaggtggtatcacattgtggttttgatttgcatttccctggtcattaaagatgttgagcattttttcatatgtttgttggctgtttgtctatcttcttttgagaattgtctattcatgtccttagcccactttttgataggattatttgttttttcttactgatttgtttgagttccttgtagattctggatattagtcctttgtcagatggatagtttgcagatatttctcccattctgtgggttgtctgtttactctgatgattatttcttttgctgtgcagaagctttatagttttaggtcccatctatttatcttttttgttgttgttgcatttgcttttggtttcttggtcatgaactctttgcttaagccagtgtctagaagagttttaccaatgttatcttctataatttttaaggttttgggtcttagatttaagtctttgatccatcttgagtggatttttgtataagttgagagatgaggatccagcttcattcttctacatgtggcttgccaattatcccaacaccatttgttgaataggatgtcctttccccaccttatgtttttgtttgctttgttgaagatcagttggctgtaagtatttagctttatttctggattttctattctgctccattgatctacatgtctatttttatagtagtaccatgctgttttcctaactatagtcttgtagtatagtttgaagttgggtaatctagtgcctccagatttgttattttttgcttagtcttgctttggctgtatgggctgttgttttgttccatgtgaattttaagattttttttcttgttctttgaagaatgatggtggcattttgatgggagtcgcattgaatttatagattgtttttggcagtgtgctcattttcacaatattgattctgccaatccatgaataagggatgtgttttcattagtttctgttgtctgtgatttctttcagcaatattttgtagttttcctgtagagatcttccacctctttggttaggtatattcctaagcattttttttttttgcagctgttgtaaaaaggctcaagttcttaatttgattctcagttttgttgctgttggtgtatagcactggtactgatttgtgtacattgattttgtatctggaaactttactgaattaacttatcagatctaggagctttttggatgagtctttaggttttctaggtatacaaacatatcatcggcaaagagcaacagtttgacttcctctttagcagtttggatgctctttatttctttctcttgtctgattgctctggctaggatttccagtactatgttgaatagaagtggtgaaagcaggcattcttgtcttattccagttctcgggggaaatgctttcaaattttcccccgttcaatataatgttggctgtgggtttgtcataagtggcttttattaccttaaggtgtgtatcttatatgccagttttgctgagggttttaatcataaagcaatactgaattttgtcaaatgctttttctgcatctattgagtttatcatatgatttttgtttttactcctgcttatatggtgtatcacatttattgacttgcatatgttaaagcaaccctgcatccccggtatgaaacccacctgatcatggtggattatctttttgatatgctgctggattcatttagctagtattttattgaggatttttacatctctgttcatcagggatattggtctgtagttttctttttttgttatgtccttttctggttttgatattagggtaatactggcttcatagaatgatttagggaggattccctctgtctctatcttttggaacagtttcaatagaatttgtaccaatttttctttgaatttctgatagcattcacctgtgaatccatctggtcctagactttttttgtttcctgacattttttctattattgtttcactctcactatgcattattggtctgttaataatttctatttcttcctgttttaatctaggaggtttgtatatatgcaggaatttgtccatctcttcttggttttctagtttgtgtacgtaaatgtgttcacagtagtcttgaataatcttttttatttctgtggtatcagttgtagtatctcccatttcatttctaattgagcttgtttagatcttttttcttgttttcttggttaatcttgccaatggtctattgattttgtttatcttttcaaagaagcaggtttttgtttcatttatcttttgtattgtattttgtgtttcaattttatttatttatttatttatttttatttttattttttgagatggagtctcactcttgttacccaggctggaatgcaacagtatgatcttggctcactgcaacatctgccttccaggttcaagtgattctcttgcctcagctgcccgagtagctgggactacaggtgcctgccaccacacctggctaatttttgtatttttagtagagacggggtttcaccatgttggccaggcaggtctcaaactcctgacttatggtgatccgcctgccttggcctcccaaagtgctgcgattacaggtgtgagccaccacactaagactcaattttatttatttctattctgatctttgttatttcttttcttctgctgggtttgggtttgctttgtcttgtttttccagttcctagaggtgtaagctcagattgtctatttgtgctctttcagactttttgatgtagatatttaatgctatgaactttgctcttaacatggcttttgctgtatcccagaggttgtgataggttttgtcattattattgttgaattcaaatatttttaaaattttcatctttcttgatttcattgttgacccaaagatcattcaggagcagattattcgatttccatgtatttgtatagttttgagggtttcttttggagttaatttttaattttattccactgtggtctgagagaatacttgatataattttgattttcttaaatttattgagacttgttcatatggtctgtcttggagaatattccatgtgttgatgaaaaggatgtagttgttgggtaggattttttgtaaatatctgttaagtccatttgttctagggtatagtttaagtccatgtttctttgttgactttctgtcttgatgacctgtctagtgctgtcagtggagtactgaagtcccccactattattgtgttgctgtctatctcatgtcttaggtctagtagtgattgctttataaatttgggagcccaagtgttagatgcatatacacttaagattgtaaatttttcctgttgaactaattattttatcattatataatgtctctctttgtcttttttaattgttgttgctttaaaatcttttttgtctgatataagaattgctattctttctcactttgagtttccatttgcatggaatatctttttccacccctttaccttaagtttatgtgagtccttacgtgttaggtgagtctcttgaagacagcagatacttggttgatggatttttatccattctgccattctgtatcttttaagtggagcatttaggccatttacattcaacattagtattgaggtatgaggtactgttctattcatcatgatagttgttgcctcaataccttcttgttgttgctgttgttaattgtgttattattttatgggtcctgttaaatttatgctttaaggaggttctattttgatgtattcaagttactgtttcaagatttagagctccttttagcatttctcagtgctggcttggtagtggcaaattcagcatttgtttgtctgaaaaagactttatctctctttcatttatgaagcttagtttcactggatacaaaattcttggctgataattattttgtttaagaggctaaatatagggcccaatctcttctggctagcagggtttatgctgagaaatctgctattaatctgctatgttttcttttataggatacctgatgcttttgcctcacagctcttaagattctttccttcatcttgactttagacaacctgatggctgtgtgcccaggtggtaatctttttgcattgaatttcccaggtgttctttgtgcttcttatatttggatatctagatctctagcaagactaggaagtttttcttgattattccctcaaataagtccttaatgaccccactatataacatgaaatatctgttattggtactgaggtgctggccacaaacaattctgtgtgtcctgaaaactcttcagaatattcgtcatctttagcacttgttatcttagtgtttgggcttggcttagagtgatacatctcataacagggcaacagaaagaaccaggaaccaagatttatataacataagtcagtaaaactagaggcaccagaggtttacatttacattaggttacattttctaacaggtagcaaagcacatgaatgaagttcagtggaaggccttcctcaggaatccagtaaaaaccaaacatacacacacacacacggacatccgtgaggcaggaagggatgtccactatagtacagacaagcatcctggaaggccatcaaggagtaggtgggtttcagttgcctcaggaatgtggcatggacccaaactaagtgagtacagatacttgtcattgaggagaagattcaaaatagcatcctaggtgtaaaaactgaggcacctggggcaggggaactaggtctctggaatgttggcttaaaagcacccctctcaggaaaggcctcatatgccatgcagggggttatatatgtgttgtgggacacagatggcaaggagataattctatgcaccaggctccactactaacaggtaaacagaccaacattaacagagacttaggtaaaaaggtaggtgcccagtggtcagttctcaggcacttccaagatgcacctaacagaaatgtaacttggtgtctattgtgtcctaggtctaacaactgaagagaagtgaattagtacctcttgtggacagagaaacaggggcagagacccattacaaagctgtctcagataggcatttgaagctgtttaagtatgtagaggcttaagtcaggctggttctgaaatgtgagagagggttaagcttcatgggaaatcagcagggtagtttgctattttttattataaccaatctcacaatagtttgggacatcaaatatcaaattgttgggaatatttatccatattagtctttttgccactaatatttaaaaatagtttacaatatacaacaaaaagttgtaaaatttccatctccacttaatcgatcttatgtaacccatacaatacatcaaatgtcctttccccactttatgtttttatttgctttgtcaaagatcacttggctgttagcatttgggtttatttctaggttctctattctgttttattggtctgtgtgcctatttttataccagtgccatgctgttttggtgactatggccttatagtatagtttgaaagcaggtaatgtgatgcctccagatttttctttttgcttaatcttgctttggctatgtgggctcttttttggttccatatgaattttaggattgttttttctagttctgtgaagaatgatggtggtattttgatgggaattgcatttaattgtagatttctcttggcagtattacccaggcttttcttattttggcaccctgtgctgctgtctccttttccttctttctgcttctcttaaccaactgttacctacacttcaatactttctgagggcaattcatcctccagtaagtctccctgaatcttctcttccttccctggcttattatatatccttcctcttggttcccatagcacctatgcacacttctgtcattgcacttgccaatttgttttataatgatctgctcatctgtctcctcacttagactatgagctcactgagagcaatggctgttgcattcaccttatatcctcaacaccattctgaaggcaagagaaagaatacccagaggtggagctgggaagctggttgtccaagtagtgaatgactctagtttgaattgaactctatagccagtgggcaatgtggatgtgttgacagttttttaacaggggactagtgaaaacacattttgggtttagaaaaaattgcaagtctgatgacatacataggagaagagattagagataggaatttcacttcagaaatttaaccacaagagcaagtgacagatcacggaagtctgaaccagactataaatgtgagaatagagaaaaaagttaacaatttgggtgtgaaagggcgagggagagaggtgtgaagaatgactaagtgtggatctgtttttaaggattgaatggaaatttgagcattttagctaatcaggcctaatattgagcaaagcaaaactcttgcaaattgttatttcaagtgtgggctgagaaaatgaaaaaatataaattctcacgttataacctcttccgtgtgtctgatttgatagaatccagccccattgcctccaaattccattgcatcttagaccagcaaacacaagtgaattctacttaaccccagaattctgtatgaaaatcttactgcctttttttttctaatcatgtgtcaaagtgtgggaagaacttttatttatgttttaataaattgtcagtataaccatttttacttgaaaatattataatttttcaagtaaacaaattgtttctctaagttgaaaattttatgatggaataaaagtatttttcctcaaaacacatagaaattttacaacaatattttagagttaactaaatgtttctttagtagtttagtcacttaaaaagtgatatgattatgaaaatacttaaactttgtcttttaactatttctaataatgctattggtataatttcatatttttatactgatcttttctccaaactttagtaaaacatacttctgtaaacccctgcccacaaaactgaagtccacatttacttctgaatgactgataagtttgtaaaagtatgcatgaatttcgttattaaattaaagtttttattatattttatgcacaatggtataaattattaaattaattttcaagcttatagaacattgataaagattgtcattagaaaaccctgagttgattgttatacattacataacctttcattggtggattagtgaatatgttatagggtgaccatgaatccaaagaatcaaagctggctacagcaaacagagggtcaaaaggatatggaactatgcatgatccagcaaaacactcaatatctgttttcctggaatgttaaaagacaaagaagaaaacttggggaacactagatgcatatagttctggttctttaagaataaaaatatgggccgggcccggtggctcatgcctgtaatcccagcactttgtgggaggccaaggcgggtggatcacaaggttaggagttcaagaccagccaggccaacatagtgaaaccctgtctctactaaaaatacaaaaaaaaattacaaaaaaaatacaaaaaaaaaaatagccaggtgtggtgacaggcacctgtattcccagctacttgggaggctgaggcaggagaatcacttgaacccgggaggcagaggttgcagtgagccaagatagtgccactgtgctccagcctgggtgacatagtgagactctgtctcaaaaaaaaaaaaaagaataaaaacaagaatggtcagagtcctagtaccttgtccagtgtagtgctgccttgagattgcattgcaatctgtctgagagatagtaaaagaaagtgataccttccttagccctgtttctctttagactatgctttcccctctccaagttaatatctctcagtctaaagcctgggaaaaggtgccaattttgtttttctttcttcctcacacctcctagaagttacactgggacactattacttttttccaggctttggccatgtgtattgttttggagagtcaacttccttttttctttcattctgcaaatagttttgagctgtcactctgtactaggtgctataaaacttacaggtgcattttacatgcctatttcctataggccacgatttaacaaaatgttcataaatgagaattaggagtgcatgtattgaatcaccacacattaactgaacagctttcattggccagagactatattgacagtggagattcaaagataaactagagaaatctcatgcttaaataactttctataataaattatataagagaagtaggttcagggatcttgggagctcagaagcaggatgagttaaacaaaagttggattttgcctttagcttggtttcattatcctgaaggaagagcctgaaatatagtgtagggtgcaagtagtatatgtgggtggcaatctcgggaaacaggagcatgtgatgaataaggagaaaaagccaatataaaggtactgcattgagggcaatgagggctctaattctctgcaccttctcaagcattgtgcagattggttttctggattatcagcctgaaggacaaaacgaagaaacagccattagctcctgtctcccattgtctgagagctgccactaggatattaacttcctgaaattctgcagaaatctcctcttactttggcactggagatgcccatacgcagaaagcaaaaaggcacagcatatttaaggaagctcataagaaacagtgcatccagaagtggcgagaattggaggaatggacatgagactctaagaaccagcgcctttgatgttccttttgatctgttatgtagctcttcttgtacacaggtgagcaaaggcatgctggacaaatggattcacatgtgctaaagcatggggcaaaaaccacatattaattcaggaaaagacaagatgcgtggccctctctgtctctgtctaagggtgaattaaagaggggatatatgtacagagtggcagggcaggacttgagataagaaggctaggtgggtgctctcatgctagtagcattatagtacaggtgatgagaagctcctgaagaatcatcttaacatttgtattttagagcaacagtattgagttctgacttagagacagcaaaactaaagacagaaagactattttgattattaatgatgtagatataagaatatcgtcaatgtgaactaaagcatgaagctacttatgatatatcattaaaaggatttaactgattggagacaaacgagagggatggggaaaagaattcatttgtttttagttgctctttttttcctacttattcctttgttccgagtgtgaataaactttgtaaacttttatactaaaacattctgctcattcatacttatttctttgatgaaacaaggaaacccttgtatagttataaacgtgtgaatcaatttaaatattaggaaatttttttaaataaagctagttttctgaaggggaaaaacttggttcaattttttgctggcaatctgctttgtgatttttgaacatgatatctacatctagactcatgttttgctagctggaattttttttcaaattaacgctaccattattatatgctttactatttagcttttgcagccttggaaatctatgattaatacaaataattctctatggcaattttaaaaatacatgtaaaagccttcaatctacattgctactgtgtcgtagcacaaaaaaagaaaatgtgatcaaattttaataaaatctacaatttattcccttctaaatacagtcctagctcaggagaaaggaagctatttgtatttttcagaatcaaatttccctaaatgaatatagagaaagaattataactgaaatattgttgaaacagtggtcatctcaaatctgaaggtcattccaaaaaagtttctgagttttcattgcctcaatctaaaagttggcctttttggtaatagatgaaagtaaaataattgaaagggtctgttgcagttttggaatatcttgaaaatatagtagagtgaagccttcttcccttaaataaaagacaagttgctgattgttttctttctagccagataagaataatgccttctttctcttgttagtcttaacacctcacttgttactatgtgtcagaaaggcgagacaccataaatggagatactactgatggaggtcatctgacatggggctggtaggcagtgggaagactggtatggacacaggtggcttaggggttggggaatgatatggaactaaggaaatgataattagcagaacccagtgtgcatgtgtgtgcattcgtgtgtccgtgtatgtgtgtactgtagcacaatgcaagaaagaaaaaacaaggcagacttttcataatttcagggataaataaatcctttatcacttcatgtagaatattggctacttggaggtatatctaaacgtaaatatataactatataactacatgctaattaaaaacatacaaagaagaagtgcctaaagaattacaacagaaagtggcatagtgattattagagttaatataatataaataaggccaggcatggtggctcatgcctataatcccagcacttttggaggtcaagttgcagggatcacttgaggacaggggatagagacaagcctagccaacatggtgaaacccatctctactaaaaatacagaaattagctgggtgtggtgatgggcgctggtaatcccagctactcaagaaactgaagcaggagaattgcttgaacccggaagctggggctgcagtgagccaagatcgcgcactgcactccagactgggtgacagagaaagacccggtctcaaaaaattaaaaaatagtataaataatatttcaaaacacaagtctgttaagataaaaggtacagaggaatggtgagatgacttttttatttgtgtgataagggactgttttctgtgattgtgagaaagaccaggagttaagaaaaagtggccatcaataaatcagccacttatggggaagaaccataaaccactctcagatgaaatacaaatgcagtcattatttaatattattggaatatttgtattagtttttggtatgtgctgctagtgctggtacattttagtagtcaattaatattttgttaatcttaatttctaactaaattccagagtgaaatggaaataataatgaaaaaattttatttacaaaacagattttgtttttttctgttaagaatgatacacagttgtccttcagtagccataggggattggtttcaggacctcccttgggtactaaaatctgcagatgcctaagcccctgttataaaatggcttagtatttgtatataacctatgcacatcctctcatatactttcaatcaggggtccccaaccccagggccatgaccagtactggtccatagcctgttaggctgttcgataccaggctgcacagcaagagctgagctcctcctcctgtcagctcagtggtggcattagattgccataggagcacgaaccctattgtgaactgcacatgtgagggatctaggttgtgcgctccttatgagaatctaatgataaatgtaatgtgcttgaatcatcccaaaaccattccccttcccctcaccatccctgtccgtggaaacatttcttccagaaaaccagtccctggtgccagaaaggttggggactgctgctttaaataatctctagattactgataatgcccaatacaatgtaaattctatgtaaatagtttttatactatattgtttagagaataatgaaaagaaaaagtctacatgttcagtttaagtgttgataagtgtgtagagaaaagggaacccttgtacattgttggtggaaatatagattggtgcagtcattatggacaatagtacggaggttcctaaagaaattaaaattagaattacctaagacccagcaatccctcctctggatgtacccaaaggaaataaaatcatcacctcataaagatatctgcactgctatattcattgcagcattatttacagtagccaagatatggaaaccacctaggtatgtgttggtgcatgaatggataaaagaaactgtggtatatgtatatacaatggaatattattcagccttaaaaaaggagaagaccctgtcatttgccacaacatgcatggacctggaggatattaagctgtgggaaataagtccaacacacatccacacacaaaattgcataatctcacttatatgtggaatctaaaaagaaaaagttcaaatataaagttagaataaaacagtggttaccggccggatgtggtagctcacgcctgtaatcctagccctttgggaagccgaggtgggtgaatcacctgaggtcaggagttcaagaccagcctgaccaacatggtgaaatcctgtttctactaaaagtacaaaaattagccgggcatagtggcaggtgcctgtaatcccagctactcaggcagttgagaaaggagaatcacttgaactcaggaggcataggttgcagtgagccgagatggcgccacttcactccagcctgggcaaaagagcaaaactctgtctcaaaataaaaaaacaaaaaacacagtccacacactggttaccatgagtgaggtggcagggaggagattgggagatgtagatctaaggatacaaagtagcagatatgtaggaggaactaaaaagctgacatgcaggatgacaactatagttagtaatagtgtattgtattcaggatttttgctaattgagtagattatagctgctcttgccacaggggaaaaagtgggtaactacgtgagatagacaatggatgtgttaatttttgtcactataataaccttttcaccatatacattcatcttataacagcatgttgtttactgtaaatatatacaataaaatttatttttaaatatctgagtatgatttgatgatttgtgaaaatagagtgaattataataattttaaatgtaagttaatgttattagaaaagaaacagaaagaacataccacacagaaagtctgtctgaaggatctttgttttctccaccaatacaagtgttcattgattcagaggtggattatgagatatgaccataaaacaaaaatttcaagggaaatatattttattcaatgaaaaattctcaacacaactgttatatgccagtaaacactatatcttttaaataacaggtcatatctattatatttaaaattcaaggagagactacattagagatgctattagatcaacttctaatttcaaagatttctaagatatggaacagttactccttatacaaattaaaaaagcaaatgctgaagaaattcagctacatggatacaccatgaggtggaaagatgctccataactcttagttaaactgcactaattacacataaaaggaaaatgtttcatttcactgtaatttggaaaccaaagaaagaaaagactgaatttttacatactgttaaagagattgcgtatctgttctaagtttaagacagaggcaaaatgtattttattcatttgtcctgcaccgtttagaaataaaattcaacttccttttaattttttttaagaataaaaaactcagtctaaggaaagtcttaaagttttcattttaagtgatccactgttctagaagtttaatattttgtttaaaatgtttatgttctgtattccaccaagtctagttttaaaacaaaacaaacaacaacaaaatacttctctaacttggagtttaaggtgaaagaaaccaattacgtggtttggaaatgtcacacttttcatctcttttttaaaaaaatttttaattcaggacagaaattgtatggatttagtgtaagtcttgggatctcacaagtgtcagtatttcactctcctccatatcttgatagcaataacttgaaataggatctcagtagctcaagcaatactgggctctgagagttggttaaaaattatttggctgagcgcctgttgctgagggaagaactaatctcgagcatatttttggagccaaataccaaattgtttgtgcttagcaacacagcaccaggcttgcccttcagaatgattctagaccaaatgccagaaatgctctggttctgactacagagttctattcacaaatgacaggaggcaagaggtcctcctcactttcagaagaaaggtcctttgctttcttagtcaatggtaggaaaaccattgtggttttcattgcattacataatttttaaggtgattacttcaataagaagtgctctgtgtatatgtgtgtttatagacgcattttttaaacactggagaatttctgaaagtagtacaaaccttgtaatgtcaagtagatgtgggaaaaagggagtttacaacattctctcctgacattgctctcctttggcatctgcatttttaaaatgttaaaaatgtttaaaaacgtgtgcttaacacttaatttggtgatagttgctgttaccaaggcaactctgtaactccacccagataaaaataaatcttgaagatgagtttctgtgtctctgagcaaatatttttgtgaatagtagaagcagagaaagttaaagatacctgagcttttgatctttactagttttatagatatgtttatagttatacatttttattcatacattttagataaataactttgtaaagcaattgattcttcttgtaaaaatcaagtatattcttaatagactgataaactttctttttttgagacagagtcttgctctattgcccaggctggaatacagtgccatgatcttggctcactgcaacctacctctgcctcctgggttcaagcaattctcctgcctcagcctcttgagtagctgagattacaggtgcatggtaccacaccccactaatttttgtattcttagtagagatggggttttgccattttggccaggctctgagaaactttttaaggtctcttttgcagccagctatttgtctaccttatttcattcttaatctcactagccaatattttttctgtttaagtgctttcagcaaatattaaatgcttgtgccttcagtcttatcctgtggaaacactggtaatgacaaaaacacatatttcaacctaatatacaatagaaacagaatgccagttattcatggaggagaagaatagacttctgtatttaaaataacattttgctctgtgttttaaaatcattcttccttcatcaattgtaagcatcttgactataatttatacacctaaagataaataattcagtagcaatgataactgaaaacaggacacatacaatgaactagctaaattaccatacattctcatccatttcaaaaatagctctgtacttttttcagattttgttagaagaatattcaatacaaatttttattcaatgaacacttcagatgtcaagattgttacccacatggacaacagtaacctaggtaaagattctgcagccaggcgtggtggctcacacctgtaatcccagcactttgggaggctgaggcgggcagatcatgaggtcaggagatcgagactatcctggctaacatggtgaaaccccatctctactaaaaatacaaaaaattagccaggtgtggtgtcatgtgcttgtagtcccagctgctcgggaggctaaggcaggagaatcgcttgaacccgggaggtggaggttgcggtgagccgagattgcaccactgcactccagcctgggtgacagagcgagactctgtctcaaaaaaaaaaaaaaaaaattttatacctgggctctgtgctcaccagcagaaggggtaacatggcttcttaggacaaccttacttgaccatttacttctttgacactaggggtattcttagatcagcaggtccttccctccacttatgcacatgaggctcacagagagtctgggaggcagggaatttatgattggaaacagtatactttttatctaagaaattattaatgtcactgcattcaagtgattaacaccatcaatatcttcaagactaaggggattacatgatgtgtaaaattagaaaactgtcatctactagtggctaggcactttaattatattaagcatgcaacaagagaactcttcaaatgaatccatctctcctctgtattatttccaacccttggatccccatctgtttctgcagacaacagctatgctgctgaatgtcttaatggtttgctgccccaactagcttcaagatactgcaggtcaagcatagcatcttactcttccctgcatctccagcacctctcagaatgttggtcacatagaagatgtttgctgaggagttgaataagaatatgtacaagggacacaattagcattgtttaaaaaagatgtaacaagatagggtaaaggaaagctttggaggataaatctttagaacaatcaataatatcttctcctctgttggttagttgcccttcaatctcagccactgaatcaaatacaacataattactattctgatatgttcttgaatcgaatatccaataataagatattcggatgcatagccatgtctaatatcaaagcccatgcttttcgctattattgtactccatacattagcttccaaatttatttgcaatccaaatattaaaagcaagtcataagcttagtatcgccaatgtgatactaagtatccacttactaaactttattttcaaaatgtggttttatctcagtttaatgaacacggcatgttttaatttacactttcatattatatagtaagggcgtggttacagatatgttaatttcctgtgctgcttcacaatgatggaacataatagcaaatgaaactgttaatttgcagatacccataggcctttggtgtctgaatagaaataaacacacctacaactgagagaggaagcatgtgaagcattccagtgaacagaggccatttattcagtcacagacacaggagaaaaacaacaattaaaaaaaaatctctgatgaaaagttcataaaaagttcactcagtttaagcatatgtcctataactacttaaaatagagttcttcttaaatatcattctttgctgtttttagatttcttctgcctgtatcaaattaatagaacacagcatacttttaatttgctctggtttcttagtggggcatttattaaacacattaaaacaatagtctcagggttttactgctgatgttaaagttctgctttcctacttaccaactgtgtcatcttaaggcacatactttgcctctctctcaaatctcccaaatggagaatgataagaatacgtacctcaattaaagaagctataacaagtagaatgtttggaaaagtgccgggtacaccataagcccactatgagtattggattgtattacctctgaaagctgcagaatggaattctcaaagttatatgtccctaaaatcctcttaagtgacagaaatggagaaattagcagtctgtctaagagagcttttctagagtctgggcatatgtttttaggacaagacagttcagcttcagcttaaaatgagagagcacgtctgtgtccttactcctgggtgccaggtttcttgtccccatcttaagacaaataattttggtggagaagaggcagtctctttgatttcgctctaaaaaccttttctggaggaggtagacactctccacccccgttttgagactcatgcagctgaggatgactggctgagtacaagcaattgttccttctaagcagtttcaattcttataacttgtggagatattcttaagtccaggggattttgtgtatggtggatttttattacaaagtcctgtacttcataggaacaaaataattcaaagtcaggaaccagatcaaagccacaactcagatatggcaccttgagaagttcatttgtatttcacttgcataaaaaccctcaccactgctatctgattttcacaaatcattcaacagctatccatgaagcacccactgtgtgtctggtctctgtgtcagtccctggcttcatgtgtctttccttctgtaccctgactccccaactcatgaacacatgaagtaaaaaaatgaaaatctttttctgacctctcttcaaaatcacttttttcaaaacaaacacctctcacctgctcatcctccagccagtaaatcacaggggcctagaaatgtcacttacaaatattttctgattctgtccctcccttcaagcttgccaacattatcacagtttagggcctgctcatctttcccccaatctccaattagatctctccacaatgcaattctgcacattccctgttacaacccttcaattatttcccagcccatccaaaataaaatctaagcctcttactaacacattcaggaactctgtggcctacggttttctacagactaattttccagcagttgacttccagtgcaagtgaaaacctagtgtcatgcctgcatgatagataaatttgaagctgaagagcccaaatgtatagaccatgccatgaaaggtttatagtcatgacacagtggccctatagtacagtgcttgaagctggctctctactgtcagacagaccacttgccagccatgagacctggggcaaaatgccttaatttttatgtgcctcaagttctcatgtgagatgagaataaaaattacccctatttcataagatttgataaagtgtttagcataatacctcataacaattgcaattcagtggtggttattattataaagaaaagatgattaactttatcttaatgtttaacttgttctgatagttattgatctatagctttgatatggaggtttgagaatgacctggaaagaattggccacaatgattgaagatagtgatacaagaataaaagatgactgcaaaatgtaaacctgcaataacagaaagaatgaagtcactggtctcatgggaactgatatgggagaaaaaaacagatcaaaaggctattcatgttttgggcctctttgtcaaaatggaaatgagaaactggggaataaaaattaaagcaattctagcatctggttttaacataattcttatccctaaaaagaatctataagaaactcccaaaatgacaggcagccgtgggtagcattgcatttcaagtaatcttttaattgttaaaatttaagtttccaacatgaacataaaattttcaacctaaaagaaatgagttccaaatctgagacaagtgaaaaaggataaagcctactagggggtaaattccatctctttagagatctagtacccaatttagcaatgtccaatcaagcctttaactactacatttgaacacctcatcatttcaaaatgttacttaatgatgccaattaactgtacaatgtctctgcatagcacatagccctaaaatgatttgtgcaatgttactgtcagtaaaactgaactacagggaatgctcatattctatgtcattatatacagaaatgcaatatcaataaagtgatatctgttggtattagaaaaaagtgaaaattttcatatctttctattttcttttttcctcaatgggatgctcttgttaaagatagctctgcatagtaaggtttgtataaacattatttagctaaagttaaaaggggtaacatactggttctagcacagatattaaaacaaattagtttgtaggtagggcagcaatcaattatattactaaccatagctttggtccttttatcctttcccatttgattttacacagtgggatgttaaaggttgaatgtctttggtatctataaacttaattgaaagctgttatttgtttgtttaagtctgttgatttttataatcataattttactcctatagatttcttgtaggagtactatatgaatttatgttgcactgaattttgttatgttatacaaattaataggcttttatttatggaaagctactattgatctgtcatttcttaaaaaattactaaaaagtgttaaaactttaaatgttggagagtttatattttaaaagttacatgctagaaaaacatgatgtctgagtatattagaagttatagataattcatctgtcaactataaaactctccaacactgcctttctttaatgaataatatgaaatttagcagtgaaaatgtgacaatgtacaatcctaaataaatcaacaaatttagagatgtacctctaaaaccattgtaaattcaacagtgtaattttccattggactttcacttattcattcattaaacaaatgtttgtgagtgcctgcaatgtatgagacattgtactgaagctaggcagtgtgagttatcatatgggattatcctttaaatacttctgagggcaaaaaaaaaaaaaaaaagaagagaaaaggtgtgaggaaagataaagggttaattcattaaaaaataacacttgaggactgttttctttgcaaggcataaagttatcaccctttcaaacagtagatatttcacatttaggatgcgagactccagttccaacaaagctcattgcacagctgctaccctgattaaactgctacatgaactctgagcaatgtagcatggtagccgcatgcttctgcttgcatgatggttaattccttccattctcattagtgattttctgagctttgaaattctgatggtacctaggatataaagcatatttatctaactgaaaaacagataattagatgtaacataaaatatgaatggctttgtcactttattgtagcagagaatgaatgtgggataaattaaagctgatgctagaacatatgcctattttttagctggaaaatttcaagatttatgtactttgggcttgagaaagaaatggagtttattttttatgcactgacatctctttttttttttttttggaagagctctcttaggaatgaatggtatgtaaatacagtaggaatgtaattatagattttcctgacccagttcctaaataatagatatcatttcagaagtgccccaatacctgaccttttgctccaagccatatcaaagcacacatctagtctacttttcactctcattcctagccactatgacaatactattcagataaaacttctagtcctctacttatgtgactcataccaacttgaccttacgatagtgactgggggtgcatatctaggttcatgctgtttgtccattattatggttttgtgagaaaaggcaaaatttctaggtaaagtgttatgaggacgaataatccaccaggcaaccaactgaccctttcatttgccatcttgtcacttcaaacagctctccagaacctgcagccagcacagaccaaagtcaggtttgtctcctcttctgttgatgaacaaaggttgattccatatcgtggctattgtgaatagtggcagtaaacatggcagtattgtatgaaaatatcacagatagcccttaaatatgtgcaactatgatgatctatcaaaattaaaaattaaaatttatttttaaaagttcagttagaaagcttgtagttcctggcaaactactacctttctcggcaaaagaatttgatatctcttaaatattttctgcctaatgctgatagattgtatttacatattccattaatgcaataaataaaattacaccaaaacatcagcattatttatttccaggggcatctctcaaaataaattcctccaaaattcacaaaaccaaaaccaatgtgaaattgtactcagggatgcaaatgtagcccagtgaagcatttgcccacttgtttggtattattgaagcacaattagaaaaatgtgcaatgtatgcccaaaaattctataataagggccaggcgcggtggctcacacctgtaatctcagcattttgggaggccaaggtgggcaaatcatgaggtcaggagatcgagaccatcctagctaacaccatgaaacccagtctttactaaaaatacaaaaaattggcccagacgtggtggcgggatcctgtagtcccagctactcgggaggctgaggcaggagaatggcatgaacccaggaggcagagtttgcactgagcctactctccagcctgaacgacagagcgagaccccatctcaaaaaaaaaaaccataataagaactttttaatatactatattataatgtaaaaagactagatgtcaaacaaattaggtgatgggaaggaattgagggagaattttagactaagcaattgagcagcacctgtttttcaccacaaatctgttacatgtattgctcaattgtgctgaatccatattgggtcctggtggctatgtaatagtctctttcttggataaatgtttgtcctctcttatggtttactaatggtgtacagaacagcattgaatagtggttatttcctatgacttcctagatatctctctcataatcctgaatgttttaaagatcattcttagatagagtacagctagacacgaaccatagtggaaatcaggtagacaaaatttaaaaggagtcttaattgaaggtcattttattgtcctcagtattaatcttacttaaaacaaacctgtcactgagcagaactcaaaacaccagagccctttgccaaatgtgattttttacaacaggagcgctggcagttgagaggagtattctgtcacacttgagagaattcgagtccctgaagatttatatgaatgcttagctattatcgaaccatctcttcacagatgacttagtaaatgtctgcctttgcatcagataatggcttacaagttaatctcctcttgctccctgttacacacatatacaccttcttcctaaacagctcataaggtgaaagaaagactcagatttctgactatgtaattgataatatcacacggactgcctgctcatcatctgctagtcacattggcagagttgacagttttggagacactgaagacagtgcatatattaggaaataagcagtttcctgatataaattttcttgtagtttataaattacatagcatttattattccctcatattttataacatttaataatagaactgacacatatattcattttaaactcaattgtgtataataactatcatagcaacccttcagtgcctaaatatcaaatcttccattcctcccatgaacatcttgaatatataggtactgtggttagctccaacaagcttttggttagaattcattgcactgatacatagacattgttttaaaggcaatttcaaatcaaagctgtcagctgtgaatcaagcacaccttaaaaagtgacacatttgtcactagattccagcctctcaaattactgacacgcatcctttttatgtaaagatgacattgttctttcctgatatattgcattcctcatgaatttcttatagtcatagaatttttataaaccatttcagaatcgctgaaataaacatcaatatttttaactttttcattctgtcaaaaatattgtatgcagagatattgctgtaagtgtgtatacctgtgcttaagagactagggctgaagagaagtaatcaaccgaaccactggtgtaaatgtgcgtcacatttttagtgactagaaattgaaataattccaacaaatttatgtgctttgggcttgagaattcagactgccttaggctaagataaaaatcttttcctggtactatataccttcttttattgaatgactacctggctctttctattatatatgcagattttgtacctctggtcatctttgtaaatggtgcctaaaagatatttgaagaataagtgaccagcaataagaacaaatgtctatacaaaagcaccctttagttggatgtaattcactactttgagttgttaataacctctaaggatgacagtagctattagttgaataaaccattatgtctattattagaacactagatagtttataagtccaaacaatgcataaaatacctatctcatgttaccattgtttaggttaccagataattgttctgtccaattattccacttaattttttgcttgcccattagctaaatggcaagataaaatttgtcaaacgggggggaatgtattgaaaatgctagacaactacacttaaaatgaaaacaggccaggcgcggtggctcaggcctgtaatcccagcactttgggaggccaaggcgggtggatcacctgaggtcgggagttcaagaccagcttgaccaacatggagaaactccatctctactaaaaatacaaaattagccgggcatggtggcacatacctgtaatcccaactactggggaggctgaggcagaagaatcgtttgaacccaggaggcggtggttgcagtgagccgagattgtgccactgtattctagcctaggcaacatgagcgaaactccatctcaaaaaaaaaaaaaaaaagaaagaaaagaaaacaaatgcataatttgcaaatattatttttatattgtatgttatctagggcttctaaatgcattcttcttataagcctaggtttgcaataacattcatttagaattgagtaattttaaatataatattttataaaataaaatataataatttctcttaattctttgaaaatattaaattaaaagggggttgcaaactctgcattccacatttccatcccaacatttaattttagcaattttgtagtctgcctaaaatgcaatccatcatttactgtttagaaaatagggaatgtacacaaaggcctttcagctttccctgaactccataaaaatctttttgcttctttactgcccccctttgtcaggagttctgaggaactgttttttatcttaagtctcacaaagcatttaggagaatatttaaacttaaattcttttaaaacttatgttcaggacaaagtaacattgtatgcattggtgtcatatgtatttaaattttgaaatttttaatactggcaaaatgaggtttcaattttaatataaattatttaacaatcttaaatcattaaatatattacttaatatatttaatatatctaaacagtcacaattttcccatactaataatcataaaaaatcttacccaatggtcatatagatatacttaatggagttttgggggggtatttttgtatattaaaaaattcatatatttgccttacttagaagaactgattaaatgaaagtataatattaacaaacatattgttattttatatttgcatttgtgataattatatttgaaacgttcaagattttccaatgaatttcttttgcatttgcgtatttgtgcctttttattataaaaataggtggctttttagttccactgcataagtttcaacataggtctacaaatagtgcatctttttgaagttaatcattataatcacaaattgaagttgcctgagctccaattggagtctaaatggatgactgaatcttattattcgaaacccactgttgctacacaatatggccacacaagagagtacacaagacccgtctgattcagcctcagtgccataaatattttaatggtttcgttggaatctggaaatggagctcaccacaggagatgcttcttcctttgactctcattattatttcctttacaaattaattaataaaaacttagatgctaaattagcacttgatgaaaacttatatagccttgacattttgattctgtgagtgaataaaaatacttggagaaataaaaatcctaatcatgttcaggaatacccacaaggtaacaagtacatttttaaactttaaaaacatttattattcatgataaaacatgttgtgtgatttaaatataaatttttattatttgctttaacttatttccggattaaaaagtaaatgtttacctagctgttctaaatggtaatcctcatgattaaaacagcaatttgtcatatttcagttacaaatgatcttttattattagttatagaacataagtttcttcattgactgaggcgatgtttcaagtagataaatctgttaaaaaaattgtggtcatattctgttaaattctcataccaggcaatttgtttgatattcaggaaaaacctagccactgaccaaaaactctacctgccttctcagttgtatcctcttggacttaaaggggactgggaaagttataagatggttcatgatagtccatcaacatcccaagaacaaaaacagatgttgtactgacagcatcatatgatcatatgcatgtaagagcacattcatattgccaaatcagttggaatttttcacggttgaaagttaaatgaaatgcttagatgtatgagtcatcggagttaaagacaattacagccagatttatggctgtgctaaaataaagctagttagaaaacagaccaaattccatgacgataccaagtctgactaatgattcaccttaaatttcggagcaacatttatcctcacttgtttgtttatttgacaatgtgcccttatccattaagtaactaggaggaagggaaaagcactacgtgggtgagtgacaagacactgacactgatttgtgactttggataattcctggatgctgttatctgttttggcatagagatggatctgtaactgctaataattgccgactgtgaccatcccagaggccatttacttaacccaggtatttcagacctgacagcccgaggataaacacgatttccctccatcactaacttcatctgcagggcctaagcctccttcacagtctctccagtgatttattggcatctccaagggtatctcacatgtgctgaagaacaaatctgctcactttcatctgcttggttttcccttttgaaatctgctgctttaaaattactaagggaggaatcatgcctgctgctacccttgccagtgaccttgcagtttgtgccctgattgttccaattaccacaatcaaaacagaagcgtttgcagttactgcagtgctctctctgtggatgtcaggtctgactcagagagccaggctggggaacagccatttccactcttgtacctctgcaaaaggacttccatgttccgtaaacagactcccacctctcattttccccccaagcaaagcatcataaattagagagcatgtaacgggaaagaaaatccattagccatttgggttcagtcagacaagccagctcatggaaagtttatacaggaaggtcacatttcaattgagatcaggagggtgaaagggtccagctgtgtgatgagagagagaatgttcgggaatgtggaacagaggtatccaaggcagaacaaactcgtatatgaaggctttaagggtgtgcaaatctagcatattttatgacataaaagagtcctgattagctagaatatgatgaatgtgagaagaggtgaaggctggagataggaaaaattattccagatcttataagctatagtaagaaatttgcatattatatatagacttgtgggaagccattggattttgtaagaaggagattaacattatcttatttatgttatttgtgatttataaccccaaatgtgccagatacaaacaaaccaaaaataataataataataataagaagaagaacaacaacagcaatggaactgtggtgatggttttggtcacaaaatgcatatatatctatttttcacaatgcaaaaatatttcattatttcaaattttaacataaatgtgggtatgcatgagcttacaaatcttgaagtttattggggaatattggtgagcatggtttttattgcatggtcacaacttactaatgggaaacatctgaatacctattgagttaatgcatgcacatttttattttcctggaatactgagaaaaaggttgctacataatgtcttgatagcttctaagtcatggctcaaaagtgaatgtggaatctgctaatcggaatggactcagattcagccaagttctcaaaaacatttgctttcatagatgtcttcaagaaacaaggagtcttgaatttaaattgtgaagtgtctatcttagaatagagagatttaaaatctgactgtattttgtttaaaaaagcctatataactgtattatataaaattatttatactacagttaaaaaaagaatcccatcctatttgtgcctaaataagtgcctgcttgtagcatgaaaactatttgttgagggtccttagatcctcagagcatgctgtgaaagtaggtacaattgttctttctatataagcctcttaagataacagataattgccagaaatacagcacacagtacaaaattaccttgttttacttttgccacaaaaaacaatttcttttggctttgagcaataaagtccaatgatttttttcctttcaaaatatcttcctccctctccataagttttatatttattcacgaaggaatattccaatatcggatgtttttgtctgtgtctcttcctggaacaaatgttaattaatctctttgggtttgtatgtcaagtggaggggtggggattggggacaggtgatagttgtctagggagttaacttcatctctataggagagtggatagacgctgtatacgaaaagctcttgaaaagggaaatacagcagccacttcctcagggcttccatggtggtcagactccttgattgctttagattaactctggcttttgtccttcggaggccaccagattgggtggatagacattgtccttgctgttcttttgacctacctacttgtactttaggggaaaaaaatgcctgtaataggttaaatgctttctcaaagatcaccaaagtatataacacatggcaaatagacagagaaatgagacagtataatcagtataatttataaaagtaccttacagcaggatcccatgggatatgggttttttttaaaaaaaatctacctaatcttttcattgaactcctattcaggattcattatattgaatatggctcagagacctggaaaattgtttccacctttttaatttattcaccatcatttatggaagttttcaaggacgtttacttacctacctcagttaacagattgtactacttgggaagtctataaatatgagcttaaagcattttctgagttttaaaataatttagattgtgtagaatgttaaaactaaaagaggaaaaaattattcagttcctcagttgaacctagcaatttatcttttcacagtgtgctcaagtatagtttttgaaaagtaaagaagatggtttttatacaaacataaacacatttcaaagattttattcaactaattaattagtagtggagccaataagctggtaagactggtttaaaggaatatctgaggaataaagatttatagaaacagtcaaagaaattctaaagagaattgactaatagatataaatctagtaaatatttgattaataatagcagtaacctatggaattatgttttctactgagcataaatgagcatgaatctctttgggtttgtatgtcaagtggaagggtggggattggggacaagtgatagttgtcaagggagttaacttcatctctataggagagtggatagatgctgtataagaaaagctcttgaaaagggaaataaagcagccactgcacatctgcacatataacctgtagatctgggggctctaataaaaaagttaatggcaatgtcaaaatctggtgttttatcttagataacttcatagtcattgattgagccccttaaaaataacatttaaaggacatgtagtcattctgtttctttattgccaagttttcagcaatttttctcatgagaatgagtgctaagaaacttttggtggagcgtggtggctcaagcctgcagtcttgcactttgggacgccaaggctggccaattacttgagatcagtagtttgagaccaccctggccaacatggtgaaaccttgtctctactaaaaatacaaaaaaaaaaaaaagtgggatgtggtggcatgcgcctgtaatcctggctactctggaggctgaggcacgagagtcacttgaacccgggaggcagaggttgcagtgagccgagatcctgccactgcactccagcctgggctacagagggagactccatctcaaacaaacaaacaaacaaaaaagaaacttttaaaatataacaatagagacattacataggcccacaaaaccacctccaaaaaagcattctatcacctgcaagaaagcatatatatatatctgcttttgtgtatatatatatatatatatatatctgcttttgtgtatatatatatacacacacacacacacatatgtgtgatatcagcatgtgtatttacacatatattttgtgcatgtatatttttaactaaaaatgtgctaggagttagatatgaactgattttggaggaggtgatatgctgtagagagagagaatgggagaatagcagtattataatctctctccattgtattcagtttttttctttgtctgaatttttaatagaagtcagccagaagatgttagtttctgggaaatgtgttgagatttacagtcaaatccagagagaactagaggcttatgagtaaataagtaaaggttatgcagagaaagtattctttttcctgtgtaaacttgaatattggccaggcgcggtggacacctgtaatccagcactttgggaggccaaggcgggtggatcgactgaggtcaggagttcatgaccagcctgtccaacatggtgaaacccattctctaccaaaaatacaaaaattagtgggtgtggtggcaggatcctgtaatcccagctactacggaggctgaggcaggagaattgctttaacctaggaggcggaggttgcagtgagctgagacagcgccattgcactatagctacggcgataagagtgagacttcatctaaaaaaaaaaaagaaaagaaaaccttgaatatttcttgtacttgtgttcaaatcatacagttatgaaagtttacccctagctgttacacttaaaatgtacttctgaaatatacagagagatgatacagactattaatgagttccactaaacttttaatggtttagaaaatacaaatattttcttatttttctggaattccagccattaatgtaaaacattggtttcaacataaataacacactggcatgcacatatgcctaagcatgggcccccacacatacagacattctgaaagaccactttttaaaaatattcagtaccgtatattgtgcattccttctttatccacatacttaagctgctgcaagcatcccattgataacaccagtaataaaagatgggaccatcagtaatgagatttgaaagccccttttgcaagaaagtaaggactagaaggtggaaatcactctgtcttagagtcatatggattggggctttgctagaagtgtgtgctctcagggaaagctgcctttttattttctccagagaaaagcctttttgtcagtaaaagaagatgtatcatccaatgcatatgtaaaattctaaacagcagataaaacaacattcactattaatctctgcaaaagaagatatattgaaaaaatcctcaagtgtccctctttgggtttctttgttatatattaaagcagttatctttagatgcatgagaatcacctgaagaccttatttttaaaattcagattcctgtcagttcactcccaaagattccgattcagtagttaagagacaaagcctaggaatgtgaatttacaatcaacacctcaggtgatagccatgcatgttcttaatgctctactactatctatgcataaaaggaagataaagttttaaaaacttgaaatgtggtataacagtttagtattgaataatatacatttttacttattgtaacaaattatgatatctacttggggcaacagtatcttttattttggatctgaatcctaattttggctaggtatcactgagggattcttagtctaaaacaattaaatggagttagtggttttttttagtaactcttgattttctgtttttttccattggcatcttacaaaatttattcattcatttttccctttttcacttggcattatttgttagacagtggacaaaagaactatagaaagtagagaagcatgtgatgttgtcctgctcttagattctcgcaactcaggagaggacattcgcttacaccaatcatctcaaaacatggcagtttatgctgaactcagtccaatgggagagcatttgactgagcacatagggagagaagttagctctgttgaaggataatcaacgaagaattcttaggaaaggtacagtcattcattgaatatttgctcggcacttactaggtgcatatgtgcactaagatctaaggatgggctgatgaagaacccaggtcccttttcttctagtggacatgcagactggcctaaaaaaaaaaaggtaactggaaaatggataaggaaactgagtcactcggtttatttattatcactcggtttatttgcttttgtttgtattttcattttgacacagcacagtgtcatcttaacgcatcctccaaagtgaaggatggggtggataacactttagttggcatttctgtagccaggagccaggatctttctcccataattgcattaacctgggaaggcaccctctaggtagatttgtatagcaccctggttaatcaattatcagtttacttcttgtctcactaagctttaacaccttacatttatgaagcagtgtaaatataactttagcatcttgatcacagcaagcacctgatttgtatttttttattagctcaagtgaaatcagatcagagaagtacattacaggtcataaaatatgtgcaaatttcataatgacctccttttaaaatgtgcaaaaataagattgttaaggcacattccagagccttggggggtgtgtgtgtgtgtgtgtgtgtgtgtgtgtgcgtgtgtgtgtgtgcttgtcttttgagaatatctgtatatcagaaaatttggctgagaagcaatcttcttcttagtggttctttttctcttttgaaaataaagtactaaaaatacttaaagatgcagaacagcaacctgttcccagtgagactctcgtttaattaatgtggtgatctatatagagaaaagggacaattgcaaaagtccctcaataattatctaaccacagtctttaggtaattacagcagaaagattttcaagacacaaaacaccctggaaaatttgacctcttattttgattcaggcctttcatttcttaaatattttctttaatgttgatgtttatgcttgacaaggtcagcctaatgccagatgaatccctggaactcaaaacattgctgaattcacagttgaaggattttaatataatataccagcttttaaaaatcctacagtgagaataacaggactgaataaaaaaattaagaaatgctcaggtagaaataaatagagaaatttagaaaaaaaataaaacgtattcaaaataagtattaagcattggcaaagaaaaaatagtagcagacaattacatgttccatttgtaaagatgattattaattagtggtcttgcaaaacattggagaaaatttgctgaaccatcacattcataaatattaaaaccacccattagtgaaaatctttttactaaacttcacaactgatagtcaaataatgttcagtttttctccattgcaataaaaaataaaggcttttgccttcagatcagtctctgggccttattaattcagtcagccagaagccacatggaaatattttgttttgttaaaagccagcttgccctcatgatcttttaaaatcttttaaaaatcttccatcagccctctccctgacttgaattatggcagtgctttctaaactggtaaactcaatctccttggtgtgcctcaagatagagtacataaaccctccttagaaattgagctctcaattctaaattgcactctccatgagagcaagcaagaatgctttgctttgtattaagtggtcacaatattaaatataaccatagacagcactgtattttctaaacaccttattttcttttaatgactgacataaattagatcataagtatacaaatgcatatctgttgtatttttcagcaccatgtgtttttttttcttttttctgagttattttcctgctttcggcagccttttctctcaggtgccttgtgatccacagtggtgtgtgttcacactaaccaaagcaatagtcttacctgccagaaatagctgtgacatttaaagagaggtccaggggaaggcacagtgcttaacatccaagtctgaagagctaatagtgaaattggggcatcagctacagagagatttaggggaagtaacaggcaggttaaatattttatggaaatgatttctgttctgtatatgattgcaattaacacatgtcaatctgtttcattaatttgttaactcatctattatgctatgccatgaagaaaataaaattggagttctttatttttttgagatggagtctcactctcttgcccaggctggagtgcagtggcaggatctcagctcactgcaatctccaccacccaggttcaagcgattcttctgcctcagccacctgagtaactgggactacaggtgcgtgcaaccatgcctggctaatttttgtatttttagtagagatggggtttcaccatgtgggccaggctggtcccaaactcctgacctcaagtgatccgcctgtcttggcctcccaaggtgctgggattacaggcgtgagccaccgcgccccgccacaaaactgaagttctaagcttcagtttagatgctcactaaatgcttgttttgcaatacctgactgtaactggcaggaatatgttttgaaagtcctcattttccaggtatgcagatgaaatataggggcattatctactatgtcaaattataatgatttatcagtggcacatgaaagtcgcctcacatttcttaatcagtgatataccattatgtcatgccaccttttaatgtaatatgtttacatctttctttagatgtaagcattcatttagttcatcacggtggctttcacacttactccaagaacgctatgagttcctttgatgtgctcaagtctcctgccccagggagaaagggagtggtgagcaggaatcgctttaatctatttacacagatattttcttttccatttattttaaaggaattttttttaacttaatgagtatgcagtgacggtggtgatgatgatgatactaaggtttaaatgattagatagtcaaatctgggctggaattgtaatactgttttgacttttaatcttagagaagctccagtctgcttattttctgggcataaacacatgagaacaataacacagttctgttatctgaatgttgttatattttgtttgaaacattcagtgactttcaaatattgtatttgcctaagaaaattcaacagagtcagacattctcttccaggttaaatttggtgagtctgctaggaaaataaattttgtgcactggtcattctgatctagtggacgttctaataaaagcacctttgtgctgcctacgtcttcactttaaagataagatacctgggtactcgacaccaaattatagtttgagatctcaaaaatgggatagggaaaccacagctcaaaaacaaaaatactagcactggaaaagatagaactagtgaagatgaatcattctctagactttaaattcagagatatcaaaattaagaaaaagtaggaggaataaaaaaagagggtaagcaaaacaatataagtttgtatagcaagagggtataaagcaaatacaatatttttcagaaaaattaaataaaaatagatttacataacattgtttttaatctcaaagatcaaatttcaattttcatctcattttaaaacccatatgcacagtctcctttatatacatcagttgggtgtcaaagtgacttttttcttgtttccaaatacagttatttttaaaatttaattgtatgatttaggaatttgaaagcaagccagtttgcacacacatatgttattatatgtgtgctttagacttggtttttagttaatgtaacatgacagggccacctgagttatttgtttacaaactagctggaaagccaccctggaggagaaacctggcaacaaaatggtctgcagctttgttattgttatctataggattggatgccattattgctgtaaaatagttcacaagaactcagtctatgggaaagactcaaaaattctttgcctgttaaagaaaaatcaggatattggactggttagtttaactaaaaagtgatgatactcagattctgcttggattcactgcttctcagcagttgttttgtttctttctaattgatattttatttttcagagaacccattataaaactcttcttcttcccttaaaatcacaaccacacaacagcaattaaaacatgctttgacgtaagactgatatggttttaaacccagcttgactatcgaattttttactttaggcaaaacacctctgacatttatgtcttatcgtcagtaaaaaggggtgattaacagttttacaagattattcaataaataaatataaattcctccttttccttcctttcctttcttcatcttcagcatctgcatgccataagctcattttagttctctggactcatgttaacatgtcccacctttcccaaattaaacatcatctctgttattggctccattcttttcctctcatttgagacaattctttatcaaccaacaccctctctgctctgtattgtgaaactctgctcctactacattaacagtctcttggtttctttaaaaagaagacaaaacaattaaagaacagaagcaaaaaatctactcaaatccccaattgttaccctcaaaattaattgtcccacccctagctttctcattgcacaactctttgtcaaaatgttttctaccatcacagccttcaatgatctttctggttcctttatctcctgaagtctgacttctacctccatctttttctggactattcaacacactttgagaaaaaacatacttttgttaaacaggtatgcatccctgaagcataaaatacatagtactgaaagtgcacatgtgtggttcttcccattttttttacagcacttgaaactgacaagtagtagtaccaattacttagtaaaagacctttttcatttcatttctgaaatattgttattttcctttttcatcttccatctctgactacacctccaattttacctctttgctgccttccttcctaagaaagttcttcatgcaatgccatcttgtttttcttcacttgcctctttttctcactttaattttatgaactctgatgacttacctctgtagtgtaactactcaaaatatgtatttctgaagtctcaactccaatctcatattttcaacttatatttatggaggcatctcagactcaacctacctaaaaaatggcttatctgccctaaaatctactttgttctttttttctctactgctaataattatcttcctagttggtcaagctcaaaacctaatcatttttactccttgtccctgtgtcagctgtccacattcaagcagcgtatcatttctgcacatttttcaagcaagtcagtaactgccttttgtttgggactgtcttttcatatagtgaacagccttggaagatagaaatcatttctccttctaaaacaaaaggcaggtgtgcttgcagccttggatagaggtagtgcctctttctaaagcaaagggacatctttactggccattataaaatatccatgtttcctgagctctgcgttcctcttttctaatgcaacccactgagcatgtaggtgtcacctgagcttttctgtgggaattgcggcttgaggaatcagtgcaagaaaatcatgatactcttgctaatgctattaatgtgagtagtaaagttaattgtctctgacccagcactattgtgtctttgcccagcactcaaaagactggcaggcttgcaagtaggacaaaatgttagatttttcacagttcttctgcttataagtacttgttaaaaccaattaaaacacaacttgtagtttgcacctataattttgtagcatttgcttcttatctatgtcactaggatgtgcttagtgacagacccatctatcatctattactcaagtttttggctgtattcctaggcaacagagagaaggggaacaaacaagaggacctgtgcacagtttgagaaaggcaaaacaccgagcttaattgcagacttgaatgtagctagcaaacgaagtaaggcaaaaggttccttttttttttttttagatggagtctcactctgtcgccagtctggagtgcagtggtgctgtctcggctcactgcaacctccgcctcctgggttccagcgattcttctgcctcagcctcccgagtagctgggactacaggcatgtgccaccatgcccagctaacttttgtatttttagtagagacggagtttcaccacgttggccaggatggtctcaatctcttgaccttgtgatccgcccattcggcctcccaaagtgctgagattataggtgtgagcctccgttcccggccaaaagtttccattttttaaatagttgggtttttagtttcgattctttccaaaaaaaggttttcttaaaaaaataaaattagcaataagatgaaatataacaacaatataatcttattaagacaatatatgatatacatttatcaaaatacttatattttcaaaagtgcttaaaataatctagcacatagtagatgctcagtaaatatttgatattatgactgtgcatgggtcattataggctactttatgtatatcatttcatttagtacaacatcactctgaaaaatgttttattgttaccgtttttcagttgaaacatttacgttgctcaagatctcactggtaccatctactattaggtcagtctgccaccaaatctcatgctcttaaatgccctttttctcctgagcttccaacaaatagtgtactgtatataattgttgaagggaggggactgtgagacaaaatatttagagtgaatgtgtagccacaatttcagttcctcaacaaagtgataaaattaggaatcatcctcaatatatattcttccaacacacacacacacatacacacacacacacacacaaataccacaagcccacttgaatgcaccccacctacacattgcaaccatagagacaattgcagcattaaatacagaatattctgtgtgttgtttgtttgttctccctttgctacaaaaatcagaatttctactcaataaacagcaaagggagatacaaatgaaccaaattaaagaaggaaaaaatgttgaaaaaattatatacagaactatgtattgatttattgagagttcagtaatgtaatccagaaataatggatgccttaaaagtaattaaaagaatgcaaataaacatttagtgccaattaaagaaaaagaaatacaacattagacaaaataaaagatattcatttgatgcaatgaggaaataatcttttattcctctttaaattctctgtggaataaggcatggttataaataaataaacatctgccccatggacttaatggatcgttatattttattgcgataatcataatgaaattgttgggagggattagtatctctagtgtaatgctaagaaagataaagcctgtgcccaggcaaaagctttcttggttggtcaaaaggtttgaagacatttcaaactattctaaaacaaacaaacaagcaaacaaacaaaaaacatacaatgtctttgccacatatttaggaaacaaaatgaacaatttatttctgacaacctcatagtctttgttctgtcagaacaataatggaaaggtctaaaccagaaaatgctatgcattgaatttataataaactattttttcctgtaacaaaaaattgataaacttgatatttgcagatttaatgattatgtgtttaaaaaaaatctggtttttgcccttgcaaaaaatcatatatatacacatagatatgtatgtgtgtgtgtgcatagtatatatatatgtatatacatatatatacacacatttatatatataaacatttcctttaacctcctattttattccaataaaaatattggtattagagatagttctgatatttcatcatgaatagttaacattgcatttggaaaggattaatttttttgaaacgtaattttaccttaataagtagcccagcgtaatattttagtaattacacagatttttttttcaagacatttgacaactaatattgcataatagttaagagtgtgggctttggagccagacttcctatctctgttcattcactgataaaatggagacagtagtaacttcctcaaagagttgttttttaagatcaaataatgcatataaaactcttgaaatggtaccaaatacagagtaagcaccaaataaacattaactgttattgttattccatgtccgaataacacagaaaagtaagaattttaatatttcatttgaatgaccttttaaggatacacctagcccattatctttcttgataatcttgtaagatgattccttttttatctccgatctgttgaggcatggatagaggttttcagagaaaacattttctaggtaactgaaagaaagtagcaacaacaaactgtgacaaaacttaacaatgagagaatttacaagatagaataattgcaactccttttgaaatcaaccactatggtcctctggctgggatagctaagcaaagatattccagcctgaaggttgagatctacttgaagagttttctatccagattgtgagggcccctcaaacttcacttagtatctgtttctattagtatggaaacttctggaaccttgtggtatcacattcacttgactactttattcctgctctagctatcttaaagcctttcttaatcttttatcttttagagaagatacttctaggttttaaatccaccgatcttgaagctattgccttcactctctgcttcagagcccatccttttgtatatgagtagtttgttttgcctaaagtactttctcccagtcagattttaagtccagtttctcatctgtttttgagagcaaactcctgggccttggctcactaacatcttgacagcatatttcttctttcctatgggcttttcagcattccctgggtttttctaaaatatgaaagcagactctttatctcttactttgtcaaagcctaccctccccactgatttctcacccagttgctagttttaagacctgcctctggccgggcgcagtggctcacgcctgtaatcccagcactttgggaggccaaggtaggtggatcacgaggtcaggagatcgagaccatcctggctaacacagtgaaaccctgtctctactaaaattacaaaaaaattagccaggcgtggtggtgagcgcctgtagtcccagctactcgggaggctgaagcaggagaatggcgtgatcccgtgaggcagagcttgcagtgagctgagatcgcgccactgcactccagcctgggcgacagagcgagactctgtctcaaaaaaaaaaaaaaaaaaaaaaaaaaaaaaagacctgcctccaaatatcattgtatttgcaaacatgaaatgacttattgattctgagctcagcacaagagcaaacctttctcagcttgacccatcttcacatcgttaatgtcttattcagtcactacccaaggggctgaccttcaagattctaatccatgaaagcttaaaatagtaaacaaatttgaatatagtttaacatacataataaattttatttctagaagaggaggatcagcccttagacatgaaaagtaaaaatagtttattcccagatttccctttgtgcattagtatattcaaccgagtctatccaagtaacaggacaaaaaaagctggcagttgttgctgcgctgtgaagtcttattaggtgagtcagctaattatatggcactaccataaatacagcaggcactgccctgcttgttaggcttgccaaggaaaataaggatttaaagcagcatactacctctttgctatataatgacattttcttcttaaaaatgattttgcaccaattcctgatttatccaccaattattttttaatttatggttgaatgtatttaaacctgaattcagagataaaactagtaaatagctccccaaaataaccccaaatatatttaatatattagctttactctctcctccactgccaaacctttaaaaactgaaataaattgtttttatttcatcttttctctttttctctctctctaaggtgattgccaagactaaagaaacagctagaagggcaaaagacaagaaaatcagtaagatagtaacagattatccaaagtagagcacggctcaggtgcagtggctcatgcctgtaatcccagcactttcggaggctgacgcaggaggatcacttgagtccaggagtttgagaccagcctgggcaacataatgaaacttcatctctataaaaaaaaaaaatttaaatagccgagcatggtggtgtaagcctatagtcccagctatttgggaggctgaggctggaggatcacttgggcccaggagttggagactacagtgagctatgattgtatcactgcattacagcctgggcaatagggcaagaccctgcctctaaacaaaagataaacaaagtagagcataaatggcttctaaatatatgttatttatgtgtaagactgggttctctaaaggtatcatttaattaaaatagatttgcattctcaatctgtaggtatggattatgtataatgtatttaagatatgacttacagcgttcaccaatgtgactattcccaagtgatccagatggctgatgacatagtaatttgtacatttgctgagacctgatctgagtaggtatgtaacataactgagggagagcaagtccatttgccgaaagaaagcctagcatatgacccaggagccacatcttcactcagccttgttgctaggtttggcttagcatatataatagcatagcatgtataatttatgacaaaaaattatactttgcactttttaattagaacattcaaaatgatctcaggaagtggcaccagagatcatcagtggtctactgtacttcgtgtgtatgtgtctgtgagtatgtatgtgtttgtgtgtgttcccacattctaaggcatgtcttttacaggttagtagaaaatgttgatagaaaattatagatttcaacatctaaaacacagtaggtcactacattgttaaaacttggaattttttatcttgttgtaaagtcaggccaaccaaacctaaaatactgctacattgaaatagtgcaaaatattcaaaatactatagttatagatttggtagtaggactgtaccagacctgtcactctatacaagacttatgccttgccctttcacttacctgttcccttttacatctatcttactagatgtaatgctataaattatatttctaatatattataatttatcatgtattataatgtatcaaatattacaaattatgttgcaactccccttacctttcgtctgcatattgcctcagaaagaacagatggatccaacagacttcaaccacaggcccttagtgacaaatagctcttaatgctgggcttgccactttgatgcatttctaaagttatagaatgttaaatgcaccaagtcctttggtcattttatttctaccttagatctaagccataactatactttcccaaaaattaaagtttgaattttaacttaaccatatataattggaaaaggaggttgggttcgttaagtgtaattttatcatgctttattatcctttgggcattggatacagcagaacatgccaatttctatggcttctcatgtgacagaatatacttactaggatgcaattaaatactcctcagagtatgtaaacaataaatgtaatcattacattatttttatattgttctttcttatgcataatagtaagactgaaaatatagtgttatttctgaaatatgcatattgttttgcttttgatgattaaataacattgtccaaagttttaggttttttgaaatcttatattttttaacaaaatatctagcctttccaaaacaagacctcaataattcgtttaagacccagagttgttcctctccacatagatctcttaaaaaggcagaggatttatgacctcaagagaaatcagagtatccaaagtttgctttaattcaatgttttaaaaataaaattccttagattttatcaaaaattgagattagtttgattttgaatcagatgccctttgctccccaccccaaaatggcattatgagcagactaggaattgataatagaaaattgaacatatgaaatatatctttaccttgctttttaacaaggtattcatgtctatcgccttcatttttaagtgcatcaataaaatacatggtaattctcttagtgaaatatactatctacactatgtacacactcccctgtctgaggtagagaagtagagaatattcacatttttgaaacgtctatgctatttttatttaaatacgagttctgggcttgatttcattttggaacacgggtgtgtgcttaagttgaacctttttttcctcttaagtcaaagttcttttttagtttcttcttttatctttttggctactatctctctccttcatcctcctggtgtgagttgttgagtgaaggtattaattccattatttgaggctaagtgacattgttcaataatgcagcaaaacaatggttctacccaaaatatcttcaagtgtaaaagcagtgggcaaaagagaaagtgcgcttctgctgctttgaatgtttaaggctgtgaaagttgatcacacaaattgggtcattcttgttatacccaactaaaacaatcaagaagcctgggaggaaaagcattcaagaaacatcacattgctccaaaagtgtaattttctacaagtccgcatgctgaggctgcctgttgtaacctgggaccaattttttctgtaactgctgaaaaaacttgctgcagctctaggactaattttgcccaccactgtcactcaccaattgaagcttactagctccccagaacctttctagtgccaatgaactttctcaaagagcagcgtgtatcatttctctttttcagaacacctccaacctcctctttgttctttgggtataccaaagaccaaccagccttgaatttcaatttttcttcccacataaaagttttaatttagaaatgtatctctacatttctaactttgacaaagcatagataccagataattgatgaaaccttgctattttaacgatcaccatggattacttcccagtgtcttcagataaccctcaacatttgccaacatttgatggacttcaaaatgagcatatcttttttaaaaaaaattattcacactgacagcaagtacattggtatactctatattaaattataccacagggtttacaaacaattggtgatgtcgggcagtggtttccaaggaacatacttaacaagacactcacaaggccctacaaacctgcatttttaacaagggccctagatgattctagaagagtgtggtttggaaagcaatttttgcctttattatgtgtcattttaaatatatttaaaattaaagttataagtcatagaattgaataaagataatttccttacagaaagtattactaggtatctaaatacaatatggttcaaaacaggaaatttaaaaagattatgtaaattctgtagttgtattcctaaagacagtagctgaaattttttcctacttctccttgtatcacttcccttttccttcactttcacttccctggaattgtacttcccaataagctattagcagtgaaggaagcttcgtctcatgatctgttttatagagcacttcagctgggacgagtacgaaatgataatcagttatatcagctattcaaccctacaggtttatttaaaaagaacttgaataagctttttagggagaaagaggtcagtctcagccatttctgtttcctaatatagcttttaagtctttccttattagcaatgagggtcattccattgtaattttttgataaccatttttctttctgtgtgtcaaatgcagatataagatactgaactgagtctatttcactgttcgtaaaacaatcccatttgaaaaaaaaaagtctacagctattccagggatagggcctagtagagagagaataaaaggtattttcttactatgtctctatatcctaccctgtaggttctcttattaagcatacaggcatataccaaaatccagacgtttttctcatttattttattgccctaacatattctgggttaatataatatcataatgaaaatttgagaaaaaattgattttttcaaaagtgtttaacatttgttatattggtagttttttttcttgtttgtggtaaaaataaatagaaggtgcacttcacaccttcaagtatgattatattttgaaaacaagtcatgaatactcataaaatgcaaattttaatgttctttttttgttacagccaaactatattaggcacagttgtaaattggagttgaaatttaatatttctttatagataacaatgtttttagaaataggtttatgaaacagtaaatatacaggtatagggataaaattgtgtctgatggtcatatgaagtgtttgttgttatattctccttggaatagctgccaaatattttagtatgcttaaaatctacgaatgtgatagagtcaacaaatttagatcacatattcagaaaaacatagttagagaactaactattgaaatgagcatacagcagtcttcctttatctacagggatacattctgaaacccccactaggacacctgaaattgcggatagtagcaaaccctacatatactgttttttccaatgcttatgtacctatgaaaaagtttaatttataaactaggcacagtaagagattaacaacaataactaataacaaaagagaacaattataataatatactgtaataaaagttatgtgggtatggtctcgctttctctttccctctctctctgtctctaaatatcttagtattttggggttgcaattggtggtgggcaactgaaaccatggaaaacaaaaccacggataaaaggagactactgtatatactttttaaaactgatgaaatattaaactcatgtttcttctatatcccacccatttcccccacccaaacctagatagatatcttatttgatctgtaaacatttaattaatttgtaaaagttaagaactttttgaagtaaaactgcaatatatcatcacacctaaagaaataaacaataattcttaaatatcaagtcagtgttcaaatttccccaactacctcatatgtgttttccatttgcttatgtagggttcccaatgagaatgaaataaagttcttaggttgcaattggctaatgctctctcacttctactttaagcggcaggttcccactaacttctttttagttgcaatttacttattgaaattagacgtattctttgtcttgtgtagtttctcacagtgcaaaatttgctgattgtagccactgttgtaagcaatgaacatgtttttcaccaccttatatttgctgtaagttgtcagtgatagttaaatgttaatcaaattcaaattcggatcacgtagggcttttctttttttgttttctttttctatttatatatttatttatttattttgagacggagtctcactccgtcaccaggctggagtgcaatggtgtgatctgggctcactgcaatctccacctcccgggttcaagtgattcccctggctcagtctcccgagtagctgggactataggagaaccaccacgcccggctaactttttgtattttagtagagatggggtttcaccatgttggccaggatgctatagatctcctgacctcaccgatcatgtaggacttcaattgtcgaacaaacgaacctttaatagcagttacaccattaggatgacctgatccaacatcgaggtcgtaaaccctattgtcgatttggactctagaataggattgtgctgtcatccctagtgtagcttgttcccacttgatgaagttattggatcagtgaacaatagcccacttaaactagtacagtcttagtttaagatggtgatgtgtatgtacttccatcagagggcacataatacagtaaatcctcacttaacttcatcaatagtttctggaaactgtgacttgaagcaaaacaacatataacaaaaccagttttaccattggctaattgatataagcaagaattaagtcctatggcaaatttctggacacaaaaacaccatcaaactcctaaataaagataaatcacttctgacattaaacattgaaattaatgtgagctatatatacgtttaagaaagattaatacaaacaagtcaaataacttacctaattatttcggtggaggccgcaggtggttggagcctatcctggcagctcagggagcaatatgggaacccaccccggacaggacgctgttccattactgcagggtgctcttgtacacacccactcacccaggctggaaccatgcagacacacacactcacctaacctacacatctgtgtacatccttcaaagttcagccaaataacatataaacaaatccagtaatatccatcagtcttagttccgtcataacaactcctttttgatcatcaaacaacaaacagggtaggtctgccatatttacttgtctggtccatatcaaaattttctaacaaattatattagaaaatcaaatctctgtcagtttcaaaatcatggaaaaaaatttgccttatttcccttatacttggatatcctaacagtaatctaaatattaatgagaaagttaatgatgtcgtttccttctccctgttgtaaagaaggttttgctgtcccgtttgatcactaagactaattgacactcagaaaaagcataggaaacttctcagcatcacaaaagctctgtcatctagagaagctaggacttgagctcaagtcctgtgacatggaaggccttgtgcctagccatcctgcagcagaggcgtatctaccaagaagtgaaacactacgaaaacagtatgtttactccacattttaaagtgaggtagtttggggtggttcatattttatttaatttatatattatttggattttttttagtttataaaaagggcattggcaagggcagaatgatctgtaagcttctctgcccacctaccataagcatgatctttagtgtgaccttttcttactgttagccattttcttatacttctgcgtccctgtcagtcacttccatgtgaagacatggggaagcttttttacatcagacatgttgttgaaaatcagccgcgttggctgagggattatttgatctctttctccaagtccctttaggctcacattgcctctctgttctttgaattttcacttacctttatcttcttataattactttgctgaaataaatgcaaagcaacaaaaggtatttagtgaagaataccaacaaagccatgaccatttcaggctgagttttgtagtattctttgtctaggaagagatacctagaaaaattttctgaccatgtatttgattattttccttcaatatgtatagtctcagtcttcaaatttcagaaaagaatttgtttcttcattgtcatttaaaattaatgtgttaaatatgtatgcttttacattataagtggttataaaagttaaacacttagaaaaaaagtcaaaataacatacatactatccaacaaaataactttcatattttattgtgttttcttccaaactttttacctttgcgtctgaattctgtgtaggttgtatctataatatagacaacactttatagcctgctaaatattataccataaataggtagttgttacataattctcaggtaatagtaatacaggtctttatcataatctactgagtagttgaatgataattttttttaagacaaggtctccctctgtcacccaggctagaatgcagtggcatgcacatggctcactgtagcctctacctcccaggctcaagtgatcctcctgcctcagcctcccaagtggctgggactgtaggcatgtgccaccatgcccagctatttatttgtatttttagtagagatggggtttcattgtaacagcccaggctggtcttgaactcctggactcaaatgatccacctgcctcagcctcccaaagtgctgaaatcacaggagtgaaccactgcacccagcaataattttttaactcttcattattcattgaacatttagttaacaattctaaaaattttgtttcctgctgtcattgatcttgtgaaaaatatctttggactatagctgtggattatttcctaaatagtaaattacttgagcaaaaagtttacatactttgagggttgataacccatgttgccgcaatgtttccccggaggcattgtggagtttagaatgccagtagtaatattaaggtgtgccattttcaagatccgtggccaacatccctatatgtaagatttttccaaaacatggttctgatttttaaaagtgaaaaatgctacttcatcatgttctttttgtgcttcttactttaaatattagaatgaagaaggagccccacaggaaggaattctggaagatatgcctgtggatcctgacaatgaggcttatgaaatgccttctgaggtaggagtccaagctgaatctttctaacaagacagtaccaaaaacctgtcattgtcacatttctctttcattagtgcttagtgagaatcatttgctctctacatgctcattacgtggacaacttgcaagttaagaatagtttttacatttttaaagggtccttaaaaaaaaagaggaggaggaagatgaagaagaggaagaaaggatgtaaaagaaatcatatgtagtccacatagcttaatatacttactacttgaccctttacaggaaaagtttactaacccctgcattagagaatatatttttagaaactttacattctaaaataaatttctaaatggaaagttagggaaatcaatggaatgccaaaggaaggttattattttttgccatacatgtccaatgggatgacgcatagtaaaataaaagttacccacacaagttatagaataaaaagataaatgcatgatttgcgacaattgatatattccagtataatgttttaaacaacacaatatgattgttaattttattttgattgaaaatgaaagtatctttaatagaaaatgtatcaaaagggaaattagaaaatactgttagatgaataaaactggcccaagaagaaacagtaaatctgaatagatttgtaacacagcgaatagattaaattagtaataaaaaaaaaaacctacctgcaaagaaaatcccaggccgagatggcatcactggtaaattctaccaaacatttaaagaggaattaatactaattagttaacaccaattaatatctcttacaaaacagaagaggagacatttcccaactaattttgtgagaccaatattaccctgataatcaaaaccaaatgaagatatcacaagaaaagaaactatataatggctccattaaaaattgagttcaagtatgttgtagtttggttatgtattattcctcacggcattattaaaaggcatgtcgaggatgggcacagcagttcacacctgtaatcccgcactttgtgagccaaagtggccaggttacttgaggccaggagttggagaccagtctggccaacatggtgaaaccccatctctactaaaaatacaaaaattagccgggcatggtggtacacgcctatggttccagctacttgggaggctgaggcatgagagtcacttgaacccaggaggcagaggttgcagtgagctgagatggcacccctgcactccaatcttggtaacagagcaagactgtctcacacagacacacgaaaggcatattgataataattcaacttatagaaattgagattaaattgtttgtttgcctaataagaatttccaatattttggggtcttttatgcaagacacagtactaaacacaatggaaaactatagagtaattgacattaccaggacataaggagtttacagtctggtaggtttgatgaaaaaaaatagaaattcattcattcatttcttcattatgattcctttaacaaacataattgattgtcttcgatgtaccaggcatcacaggagcaaaaatatataagacatactaaaaagtaaaacattttaaagatctgtttcaatcaatcaggagaagttttattgaggaggtaatgttgatctgggtgggaaaaggtaagagatatagtaggtcaaaacaaacagaggacattctggcacaagggaatatcagaagcaaaggcatgtatgtctgagcatgcaaatggatatgtctgagaacagtgaataattatgactcaagcttaggaacaaggaaaatggtgatagattgaatttgcagctatgggtcaaagacaagttatagagtattaggataatcttgtcatttcagcttgtattctattcagaaaacaacttgagttattgaagttatgcttatttgtttgtttttaagcagaatcctgatattattagagttgctctttaggaggaataatctgatccctttaattaaatccattaatatttgtgttgtggatgctatccagatactgtatggagagcttgaggtttgaaatacaagtaataattgaagccatagatgaagacgaaattttcaactgggagagtgaaagtagggaaaatgtatcttgccttcaaacatcttaatttccttctgagaattagagcatcttagtctggaaaaggctttatagacagcttgattttgttctcacattttacaggtgaagaaactgagaaccagacagtccaacttatttgtcctaccaaactaggtatatgatcattaaatggtgcatccggatcagaacctagatattttaactctgactactactgtaattcacttttatatcagacaagaaagacacaactattaaaaataagataatatttgctgcagaatatttgcaaaaacattgattgtaaattttagtgtaagtggggagccatttcctatctcattggctgtcagtgctgatgcgtaattgaaacttatactaacagtgtgtgctgtctttttgatttttctaatattaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaatatgtatttggtttctgcctccagttcttacaaagagctcctaaaacccttgtaatttcctgagtagtaggggtgctagggtcatcttttgttctaatatttggtctttgactctgctttctgacagagctccttagtccctgggtgagagtagcatcttctcttctaatgaagtgactcttgctgggttcctggatgggggctggtcaccagaaaggtcaagccatgataagaagcttgaagcttttggccccattcacatcttctggggacgggagagaagaggagctggagattgagttaataagcaacaatgcttccatgatgaagactccataaaaatccctaaaagacaggattcagagtgctttgaaataggtgaacatgcagaggtgctgggaattgtggtgtgtccagagaaggcatgcaagctccccacgcctcccccatacctttccctgtgcatctcttccatctggctgttcctgagttgtatccttttataacaaactggtaatctagtaagcaaactgttttcctgaagtctgtgaatcacactagcaaattatcaaacctgaggagagggccgtggagaccttggatttgtagacaagtcaaacagaagctatgagtaacatgaggactcattgcttgtgattgtcatcttcagtgggaaggggaaaaatcttgtaaaactgagtccttaacctgtgggtcaatgctaactccaggtagatagtgtccgatttgaattacgggacacccagttggtagccacaaagaatgggagaattgcttggtgtagaaaacacaccccacacacacatgtggtgtcagaaatgaaccggaaatattgtgttccggaaatattgagtgttgtgagtgagtgtatagaaagaaaaacagcgtttccttttcactactagattaaaacaaacacactcatgcattcacacatctcaaagacaactattaattctcaaagacagtgctgtctaaatccatactgaggaagaaaacacattttcttttcaaatctgtaaacctgacagactgcctctgtccacacactaatggaactctgtgtttcatctgaaatgtgttcatcccactttgttctttctgtcttgggcagggcaagagtgcaacagggctgacattttcatatgagctctgtccctgttattggctatactttagacaaattattatgtgtcaaatatagatgtaagtgatttatcaatattaagtcatttaattctcaaaacaaccttaataggttccattatgattctaattttacacataagccaaaggaggcacccacaggctagataactttcccacggccacacagctagtaagcggcagagccaagaggcccaacattacagcaccacagtctgtgctctcagccccttggccacatagtgtcagagtgaggacacacagctatttaagaaaacttccagaagtctaggaaatggggtgatagccccacttttctaggtataataattagatatttgtttttcttcaggtacctaaagaaaatttactagagtttgagcctttagtaagttttgctagtacatctgtttttcttcaggtgcctgaagacaaacatatacacacacacacacacacaaacacacacaaaatgtgtatctatatatatgtgtacacatatctctcatctctatatatatgtctctgtatatctatatatctataaacatatctatatctatagatacatatagagagatttctttttttttttttttgagatggagtcttgctcttgccacctaggctggagtgcaatggcacaatctcagttcactgcaacctccgcctcccaggttcaagcgattctcctgcctcagcctctcgagtaggtgggattacaggaacacaccaccttagcccgactaatttttgtatttttagtagagacagggttcaccacgttggccaggctggtctcaaactcctgacctcaggtaatccacctacctcggcctcccaaagtgctgggattacaggtgtgagccaccatgcctggccaagatttctaattctaagagaaattagcacctgataggtatttccttgtaaataaaccgggcatatcctgattatagaactaagttaattattttccgtggaagatacgaatgttgatgcaataagagcagcagtctacagtaaggtgggctttgtaattttctgtgttgaatcatggcatgggtacttggcttatgtcaaatagacaaaaaaatataaattaaggtataactgggattgtcaattatacatatttagtaatggaatgaatgaatttataaatagatagtaaagggcatgaattaagaatctataggtataaataatattagcaacttaatattgtataataaagtttgattttctaggtgtagttgattgatgcagtaatgttcgttttatcctttgagtaagcctagaattgaagaacccaaaatgcaatagaatagatataacattgaaactattcctaaatatgattttagttccaatgttctttgtgtaattacctaagcttttctttaatgtttttgctgctactacagtatccttaattatttgaaatcttatattggaagcagttaaaccacattccttcaaagagcccttagtttgagcctctagtaagttttgctagtataatttggttt

SEQ ID NO: 3 - DNA, 3089 bp, Homo sapiens
  source 1..3089: mol_type = genomic DNA
aggagaaggagaaggaggaggactaggaggaggaggacggcgacgaccagaaggggcccaagagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 4 - DNA, 1208 bp, Homo sapiens
  source 1..1208: mol_type = genomic DNA
agagagggggcgagcgaccgagcgccgcgacgcggaagtgaggtgcgtgcgggctgcagcgcagaccccggcccggcccctccgagagcgtcctgggcgctccctcacgccttgccttcaagccttctgcctttccaccctcgtgagcggagaactgggagtggccattcgacgacaggttagcgggtttgcctcccactcccccagcctcgcgtcgccggctcacagcggcctcctctggggacagtcccccccgggtgccgcctccgcccttcctgtgcgctccttttccttcttctttcctattaaatattatttgggaattgtttaaattttttttttaaaaaaaagagagaggcggggaggagtcggagttgtggagaagcagagggactcagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactattttttgttgctgttgaaaaaaaaaaaaaaaaaaaaaaaaaaaaaaaaaaaaaaa

SEQ ID NO: 5 - DNA, 3022 bp, Homo sapiens
  source 1..3022: mol_type = genomic DNA
attctggtgtgatccaggaacagctgtcttccagctctgaaagagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaagaaatatctttgctcccagtttcttgagatctgctgacagatgttccatcctgtacaagtgctcagttccaatgtgcccagtcatgacatttctcaaagtttttacagtgtatctcgaagtcttccatcagcagtgattgaagtatctgtacctgcccccactcagcatttcggtgcttccctttcactgaagtgaatacatggtagcagggtctttgtgtgctgtggattttgtggcttcaatctacgatgttaaaacaaattaaaaacacctaagtgactaccacttatttctaaatcctcactatttttttgttgctgttgttcagaagttgttagtgatttgctatcatatattataagatttttaggtgtcttttaatgatactgtctaagaataatgacgtattgtgaaatttgttaatatatataatacttaaaaatatgtgagcatgaaactatgcacctataaatactaaatatgaaattttaccattttgcgatgtgttttattcacttgtgtttgtatataaatggtgagaattaaaataaaacgttatctcattgcaaaaatattttatttttatcccatctcactttaataataaaaatcatgcttataagcaacatgaattaagaactgacacaaaggacaaaaatataaagttattaatagccatttgaagaaggaggaattttagaagaggtagagaaaatggaacattaaccctacactcggaattccctgaagcaacactgccagaagtgtgttttggtatgcactggttccttaagtggctgtgattaattattgaaagtggggtgttgaagaccccaactactattgtagagtggtctatttctcccttcaatcctgtcaatgtttgctttacgtattttggggaactgttgtttgatgtgtatgtgtttataattgttatacatttttaattgagccttttattaacatatattgttatttttgtctcgaaataattttttagttaaaatctattttgtctgatattggtgtgaatgctgtacctttctgacaataaataatattcgaccatgaataaaaaaaaaaaaaaagtgggttcccgggaactaagcagtgtagaagatgattttgactacaccctccttagagagccataagacacattagcacatattagcacattcaaggctctgagagaatgtggttaactttgtttaactcagcattcctcactttttttttttaatcatcagaaattctctctctctctctctctttttctctcgctctctttttttttttttttttacaggaaatgcctttaaacatcgttggaactaccagagtcaccttaaaggagatcaattctctagactgataaaaatttcatggcctcctttaaatgttgccaaatatatgaattctaggatttttccttaggaaaggtttttctctttcagggaagatctattaactccccatgggtgctgaaaataaacttgatggtgaaaaactctgtataaattaatttaaaaattatttggtttctctttttaattattctggggcatagtcatttctaaaagtcactagtagaaagtataatttcaagacagaatattctagacatgctagcagtttatatgtattcatgagtaatgtgatatatattgggcgctggtgaggaaggaaggaggaatgagtgactataaggatggttaccatagaaacttccttttttacctaattgaagagagactactacagagtgctaagctgcatgtgtcatcttacactagagagaaatggtaagtttcttgttttatttaagttatgtttaagcaaggaaaggatttgttattgaacagtatatttcaggaaggttagaaagtggcggttaggatatattttaaatctacctaaagcagcatattttaaaaatttaaaagtattggtattaaattaagaaatagaggacagaactagactgatagcagtgacctagaacaatttgagattaggaaagttgtgaccatgaatttaaggatttatgtggatacaaattctcctttaaagtgtttcttcccttaatatttatctgacggtaatttttgagcagtgaattactttatatatcttaatagtttatttgggaccaaacacttaaacaaaaagttctttaagtcatataagccttttcaggaagcttgtctcatattcactcccgagacattcacctgccaagtggcctgaggatcaatccagtcctaggtttattttgcagacttacattctcccaagttattcagcctcatatgactccacggtcggctttaccaaaacagttcagagtgcactttggcacacaattgggaacagaacaatctaatgtgtggtttggtattccaagtggggtctttttcagaatctctgcactagtgtgagatgcaaacatgtttcctcatctttctggcttatccagtatgtagctatttgtgacataataaatatatacatatatgaaaata

SEQ ID NO: 6 - DNA, 364 bp, Homo sapiens
  source 1..364: mol_type = genomic DNA
gagaaggagaaggaggaggactaggaggaggaggacggcgacgaccagaaggggcccaagagagggggcgagcgaccgagcgccgcgacgcggaagtgagtgtggtgtaaaggaattcattagccatggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaa

SEQ ID NO: 7 - RNA, 19 nt, synthetic construct
  source 1..19: mol_type = other RNA
tggcagaagcagcaggaaa

SEQ ID NO: 8 - RNA, 21 nt, synthetic construct
  source 1..21: mol_type = other RNA
tccttggttttggagcctaca

SEQ ID NO: 9 - RNA, 17 nt, synthetic construct
  source 1..17: mol_type = other RNA
caaaagagggtgttctc

SEQ ID NO: 10 - RNA, 28 nt, synthetic construct
  source 1..28: mol_type = other RNA
gttgttcagaagttgttagtgatttgct

SEQ ID NO: 11 - RNA, 27 nt, synthetic construct
  source 1..27: mol_type = other RNA
gtgcatagtttcatgctcacatatttt

SEQ ID NO: 12 - RNA, 24 nt, synthetic construct
  source 1..24: mol_type = other RNA
ctgtctaagaataatgacgtattg

SEQ ID NO: 13 - RNA, 19 nt, synthetic construct
  source 1..19: mol_type = other RNA
gaaggtgaaggtcggagtc

SEQ ID NO: 14 - RNA, 20 nt, synthetic construct
  source 1..20: mol_type = other RNA
gaagatggtgatgggatttc

SEQ ID NO: 15 - RNA, 20 nt, synthetic construct
  source 1..20: mol_type = other RNA
caagcttcccgttctcagcc

SEQ ID NO: 16 - RNA, 23 nt, synthetic construct
  source 1..23: mol_type = other RNA
gctgttcctggatcacaccagaa

SEQ ID NO: 17 - RNA, 23 nt, synthetic construct
  source 1..23: mol_type = other RNA
atgaatacatccatggctaatga

SEQ ID NO: 18 - RNA, 23 nt, synthetic construct
  source 1..23: mol_type = other RNA
tctttcagagctggaagacagct

SEQ ID NO: 19 - RNA, 23 nt, synthetic construct
  source 1..23: mol_type = other RNA
gaatggagaagcaagctcctccc

SEQ ID NO: 20 - RNA, 23 nt, synthetic construct
  source 1..23: mol_type = other RNA
tggcctttgaaagtcctttcatg

SEQ ID NO: 21 - RNA, 23 nt, synthetic construct
  source 1..23: mol_type = other RNA
agcagccacaactccctccttgg

SEQ ID NO: 22 - RNA, 23 nt, synthetic construct
  source 1..23: mol_type = other RNA
ccctgtttggttttctcagcagc

SEQ ID NO: 23 - RNA, 23 nt, synthetic construct
  source 1..23: mol_type = other RNA
ttggttttggagcctacatagag

SEQ ID NO: 24 - RNA, 23 nt, synthetic construct
  source 1..23: mol_type = other RNA
gagaacaccctcttttgtctttc

SEQ ID NO: 25 - RNA, 23 nt, synthetic construct
  source 1..23: mol_type = other RNA
ttcctgctgcttctgccacaccc

SEQ ID NO: 26 - RNA, 23 nt, synthetic construct
  source 1..23: mol_type = other RNA
caccatgcaccactccctccttg

SEQ ID NO: 27 - RNA, 23 nt, synthetic construct
  source 1..23: mol_type = other RNA
tttgtcacttgctctttggtctt

SEQ ID NO: 28 - RNA, 23 nt, synthetic construct
  source 1..23: mol_type = other RNA
tggctgctgcaatgctccctgct

SEQ ID NO: 29 - RNA, 23 nt, synthetic construct
  source 1..23: mol_type = other RNA
gctccctccactgtcttctgggc

SEQ ID NO: 30 - RNA, 23 nt, synthetic construct
  source 1..23: mol_type = other RNA
ggctactgctgtcacacccgtca

SEQ ID NO: 31 - RNA, 23 nt, synthetic construct
  source 1..23: mol_type = other RNA
tcaccactgctcctccaacattt

SEQ ID NO: 32 - RNA, 23 nt, synthetic construct
  source 1..23: mol_type = other RNA
cttctcagccactgttgccacac

SEQ ID NO: 33 - RNA, 23 nt, synthetic construct
  source 1..23: mol_type = other RNA
gtcctttttgacaaagccagtgg

SEQ ID NO: 34 - RNA, 23 nt, synthetic construct
  source 1..23: mol_type = other RNA
cttcctcagaaggcatttcataa

SEQ ID NO: 35 - RNA, 23 nt, synthetic construct
  source 1..23: mol_type = other RNA
taagcctcattgtcaggatccac

SEQ ID NO: 36 - RNA, 23 nt, synthetic construct
  source 1..23: mol_type = other RNA
cacaggcatatcttccagaattc

SEQ ID NO: 37 - RNA, 23 nt, synthetic construct
  source 1..23: mol_type = other RNA
ttccttcctgtggggctccttct

SEQ ID NO: 38 - RNA, 23 nt, synthetic construct
  source 1..23: mol_type = other RNA
tcttcattcttgcccaactggtc

SEQ ID NO: 39 - RNA, 23 nt, synthetic construct
  source 1..23: mol_type = other RNA
aggttcgtagtcttgataccctt

SEQ ID NO: 40 - RNA, 23 nt, synthetic construct
  source 1..23: mol_type = other RNA
aatgtcatgactgggcacattgg

SEQ ID NO: 41 - RNA, 23 nt, synthetic construct
  source 1..23: mol_type = other RNA
tggaactgagcacttgtacagga

SEQ ID NO: 42 - RNA, 23 nt, synthetic construct
  source 1..23: mol_type = other RNA
gatctcaagaaactgggagcaaa

SEQ ID NO: 43 - RNA, 23 nt, synthetic construct
  source 1..23: mol_type = other RNA
ggatggaacatctgtcagcagat

SEQ ID NO: 44 - RNA, 23 nt, synthetic construct
  source 1..23: mol_type = other RNA
aaagatatttcttaggcttcagg

SEQ ID NO: 45 - RNA, 23 nt, synthetic construct
  source 1..23: mol_type = other RNA
acactgtaaaaactttgagaaat

SEQ ID NO: 46 - RNA, 23 nt, synthetic construct
  source 1..23: mol_type = other RNA
cctgctaccatgtattcacttca

SEQ ID NO: 47 - RNA, 23 nt, synthetic construct
  source 1..23: mol_type = other RNA
tcagtgaaagggaagcaccgaaa

SEQ ID NO: 48 - RNA, 23 nt, synthetic construct
  source 1..23: mol_type = other RNA
aaatgctgagtgggggcaggtac

SEQ ID NO: 49 - RNA, 23 nt, synthetic construct
  source 1..23: mol_type = other RNA
tacagatacttcaatcactgctg

SEQ ID NO: 50 - RNA, 23 nt, synthetic construct
  source 1..23: mol_type = other RNA
ctgatggaagacttcgagataca

SEQ ID NO: 51 - RNA, 23 nt, synthetic construct
  source 1..23: mol_type = other RNA
atcgtagattgaagccacaaaat

SEQ ID NO: 52 - RNA, 23 nt, synthetic construct
  source 1..23: mol_type = other RNA
ttctgaacaacagcaacaaaaaa

SEQ ID NO: 53 - RNA, 23 nt, synthetic construct
  source 1..23: mol_type = other RNA
aaaatagtgaggatttagaaata

SEQ ID NO: 54 - RNA, 23 nt, synthetic construct
  source 1..23: mol_type = other RNA
ataagtggtagtcacttaggtgt

SEQ ID NO: 55 - RNA, 23 nt, synthetic construct
  source 1..23: mol_type = other RNA
tgtttttaatttgttttaacatc

SEQ ID NO: 56 - RNA, 23 nt, synthetic construct
  source 1..23: mol_type = other RNA
aatccacagcacacaaagaccct

SEQ ID NO: 57 - RNA, 23 nt, synthetic construct
  source 1..23: mol_type = other RNA
gatagcaaatcactaacaacttc

SEQ ID NO: 58 - RNA, 23 nt, synthetic construct
  source 1..23: mol_type = other RNA
ctcacatatttttaagtattata

SEQ ID NO: 59 - RNA, 23 nt, synthetic construct
  source 1..23: mol_type = other RNA
atatatattaacaaatttcacaa

SEQ ID NO: 60 - RNA, 23 nt, synthetic construct
  source 1..23: mol_type = other RNA
caatacgtcattattcttagaca

SEQ ID NO: 61 - RNA, 23 nt, synthetic construct
  source 1..23: mol_type = other RNA
acagtatcattaaaagacaccta

SEQ ID NO: 62 - RNA, 23 nt, synthetic construct
  source 1..23: mol_type = other RNA
ctaaaaatcttataatatatgat

SEQ ID NO: 63 - RNA, 23 nt, synthetic construct
  source 1..23: mol_type = other RNA
tataggtgcatagtttcatgctc

SEQ ID NO: 64 - RNA, 23 nt, synthetic construct
  source 1..23: mol_type = other RNA
tttgcaatgagataacgttttat

SEQ ID NO: 65 - RNA, 23 nt, synthetic construct
  source 1..23: mol_type = other RNA
tattttaattctcaccatttata

SEQ ID NO: 66 - RNA, 23 nt, synthetic construct
  source 1..23: mol_type = other RNA
atatacaaacacaagtgaataaa

SEQ ID NO: 67 - RNA, 23 nt, synthetic construct
  source 1..23: mol_type = other RNA
aaaacacatcgcaaaatggtaaa

SEQ ID NO: 68 - RNA, 23 nt, synthetic construct
  source 1..23: mol_type = other RNA
aaaatttcatatttagtatttat

SEQ ID NO: 69 - RNA, 23 nt, synthetic construct
  source 1..23: mol_type = other RNA
tttttattattaaagtgagatgg

SEQ ID NO: 70 - RNA, 23 nt, synthetic construct
  source 1..23: mol_type = other RNA
tccttcttcaaatggctattaat

SEQ ID NO: 71 - RNA, 23 nt, synthetic construct
  source 1..23: mol_type = other RNA
aataactttatatttttgtcctt

SEQ ID NO: 72 - RNA, 23 nt, synthetic construct
  source 1..23: mol_type = other RNA
ctttgtgtcagttcttaattcat

SEQ ID NO: 73 - RNA, 23 nt, synthetic construct
  source 1..23: mol_type = other RNA
catgttgcttataagcatgattt

SEQ ID NO: 74 - RNA, 23 nt, synthetic construct
  source 1..23: mol_type = other RNA
tgggataaaaataaaatattttt

SEQ ID NO: 75 - RNA, 23 nt, synthetic construct
  source 1..23: mol_type = other RNA
tctacctcttctaaaattcctcc

SEQ ID NO: 76 - RNA, 23 nt, synthetic construct
  source 1..23: mol_type = other RNA
taattaatcacagccacttaagg

SEQ ID NO: 77 - RNA, 23 nt, synthetic construct
  source 1..23: mol_type = other RNA
aggaaccagtgcataccaaaaca

SEQ ID NO: 78 - RNA, 23 nt, synthetic construct
  source 1..23: mol_type = other RNA
acacacttctggcagtgttgctt

SEQ ID NO: 79 - RNA, 23 nt, synthetic construct
  source 1..23: mol_type = other RNA
cttcagggaattccgagtgtagg

SEQ ID NO: 80 - RNA, 23 nt, synthetic construct
  source 1..23: mol_type = other RNA
agggttaatgttccattttctct

SEQ ID NO: 81 - RNA, 23 nt, synthetic construct
  source 1..23: mol_type = other RNA
cttcaacaccccactttcaataa

SEQ ID NO: 82 - RNA, 23 nt, synthetic construct
  source 1..23: mol_type = other RNA
aacagttccccaaaatacgtaaa

SEQ ID NO: 83 - RNA, 23 nt, synthetic construct
  source 1..23: mol_type = other RNA
aaagcaaacattgacaggattga

SEQ ID NO: 84 - RNA, 23 nt, synthetic construct
  source 1..23: mol_type = other RNA
tgaagggagaaatagaccactct

SEQ ID NO: 85 - RNA, 23 nt, synthetic construct
  source 1..23: mol_type = other RNA
tctacaatagtagttggggtctt

SEQ ID NO: 86 - RNA, 23 nt, synthetic construct
  source 1..23: mol_type = other RNA
taaacacatacacatcaaacaac

SEQ ID NO: 87 - RNA, 23 nt, synthetic construct
  source 1..23: mol_type = other RNA
ttaaaaatgtataacaattataa

SEQ ID NO: 88 - RNA, 23 nt, synthetic construct
  source 1..23: mol_type = other RNA
accaatatcagacaaaatagatt

SEQ ID NO: 89 - RNA, 23 nt, synthetic construct
  source 1..23: mol_type = other RNA
attttaactaaaaaattatttcg

SEQ ID NO: 90 - RNA, 23 nt, synthetic construct
  source 1..23: mol_type = other RNA
tcgagacaaaaataacaatatat

SEQ ID NO: 91 - RNA, 23 nt, synthetic construct
  source 1..23: mol_type = other RNA
tatgttaataaaaggctcaatta

SEQ ID NO: 92 - RNA, 23 nt, synthetic construct
  source 1..23: mol_type = other RNA
cagaaaggtacagcattcacacc

SEQ ID NO: 93 - RNA, 23 nt, synthetic construct
  source 1..23: mol_type = other RNA
ttttttttttttttattcatggt

SEQ ID NO: 94 - RNA, 23 nt, synthetic construct
  source 1..23: mol_type = other RNA
gtgctaatgtgtcttatggctct

SEQ ID NO: 95 - RNA, 23 nt, synthetic construct
  source 1..23: mol_type = other RNA
tctctaaggagggtgtagtcaaa

SEQ ID NO: 96 - RNA, 23 nt, synthetic construct
  source 1..23: mol_type = other RNA
ttagttcccgggaacccactttt

SEQ ID NO: 97 - RNA, 23 nt, synthetic construct
  source 1..23: mol_type = other RNA
aaaatcatcttctacactgctta

SEQ ID NO: 98 - RNA, 23 nt, synthetic construct
  source 1..23: mol_type = other RNA
ggtcgaatattatttattgtcag

SEQ ID NO: 99 - RNA, 23 nt, synthetic construct
  source 1..23: mol_type = other RNA
agccttgaatgtgctaatatgtg

SEQ ID NO: 100 - RNA, 23 nt, synthetic construct
  source 1..23: mol_type = other RNA
agagcgagagaaaaagagagaga

SEQ ID NO: 101 - RNA, 23 nt, synthetic construct
  source 1..23: mol_type = other RNA
agagagagagagaatttctgatg

SEQ ID NO: 102 - RNA, 23 nt, synthetic construct
  source 1..23: mol_type = other RNA
atgattaaaaaaaaaaagtgagg

SEQ ID NO: 103 - RNA, 23 nt, synthetic construct
  source 1..23: mol_type = other RNA
aggaatgctgagttaaacaaagt

SEQ ID NO: 104 - RNA, 23 nt, synthetic construct
  source 1..23: mol_type = other RNA
agttaaccacattctctcagagc

SEQ ID NO: 105 - RNA, 23 nt, synthetic construct
  source 1..23: mol_type = other RNA
gtaaaaaaaaaaaaaaaaagaga

SEQ ID NO: 106 - RNA, 23 nt, synthetic construct
  source 1..23: mol_type = other RNA
atgaaatttttatcagtctagag

SEQ ID NO: 107 - RNA, 23 nt, synthetic construct
  source 1..23: mol_type = other RNA
gagaattgatctcctttaaggtg

SEQ ID NO: 108 - RNA, 23 nt, synthetic construct
  source 1..23: mol_type = other RNA
gtgactctggtagttccaacgat

SEQ ID NO: 109 - RNA, 23 nt, synthetic construct
  source 1..23: mol_type = other RNA
gatgtttaaaggcatttcctgta

SEQ ID NO: 110 - RNA, 23 nt, synthetic construct
  source 1..23: mol_type = other RNA
ggcaacatttaaaggaggccatg

SEQ ID NO: 111 - RNA, 23 nt, synthetic construct
  source 1..23: mol_type = other RNA
atcctagaattcatatatttggc

SEQ ID NO: 112 - RNA, 23 nt, synthetic construct
  source 1..23: mol_type = other RNA
aattaatttatacagagtttttc

SEQ ID NO: 113 - RNA, 23 nt, synthetic construct
  source 1..23: mol_type = other RNA
ttcaccatcaagtttattttcag

SEQ ID NO: 114 - RNA, 23 nt, synthetic construct
  source 1..23: mol_type = other RNA
cagcacccatggggagttaatag

SEQ ID NO: 115 - RNA, 23 nt, synthetic construct
  source 1..23: mol_type = other RNA
tagatcttccctgaaagagaaaa

SEQ ID NO: 116 - RNA, 23 nt, synthetic construct
  source 1..23: mol_type = other RNA
aaaacctttcctaaggaaaaatc

SEQ ID NO: 117 - RNA, 23 nt, synthetic construct
  source 1..23: mol_type = other RNA
gtgacttttagaaatgactatgc

SEQ ID NO: 118 - RNA, 23 nt, synthetic construct
  source 1..23: mol_type = other RNA
aatacatataaactgctagcatg

SEQ ID NO: 119 - RNA, 23 nt, synthetic construct
  source 1..23: mol_type = other RNA
tgaaattatactttctactagtg

SEQ ID NO: 120 - RNA, 23 nt, synthetic construct
  source 1..23: mol_type = other RNA
atgtctagaatattctgtcttga

SEQ ID NO: 121 - RNA, 23 nt, synthetic construct
  source 1..23: mol_type = other RNA
gagaaaccaaataatttttaaat

SEQ ID NO: 122 - RNA, 23 nt, synthetic construct
  source 1..23: mol_type = other RNA
tgccccagaataattaaaaagag

SEQ ID NO: 123 - RNA, 23 nt, synthetic construct
  source 1..23: mol_type = other RNA
tatatatcacattactcatgaat

SEQ ID NO: 124 - RNA, 23 nt, synthetic construct
  source 1..23: mol_type = other RNA
tttctatggtaaccatccttata

SEQ ID NO: 125 - RNA, 23 nt, synthetic construct
  source 1..23: mol_type = other RNA
ccttcctcaccagcgcccaatat

SEQ ID NO: 126 - RNA, 23 nt, synthetic construct
  source 1..23: mol_type = other RNA
actctgtagtagtctctcttcaa

SEQ ID NO: 127 - RNA, 23 nt, synthetic construct
  source 1..23: mol_type = other RNA
caattaggtaaaaaaggaagttt

SEQ ID NO: 128 - RNA, 23 nt, synthetic construct
  source 1..23: mol_type = other RNA
atagtcactcattcctccttcct

SEQ ID NO: 129 - RNA, 23 nt, synthetic construct
  source 1..23: mol_type = other RNA
gatgacacatgcagcttagcact

SEQ ID NO: 130 - RNA, 23 nt, synthetic construct
  source 1..23: mol_type = other RNA
tttccttgcttaaacataactta

SEQ ID NO: 131 - RNA, 23 nt, synthetic construct
  source 1..23: mol_type = other RNA
accatttctctctagtgtaagat

SEQ ID NO: 132 - RNA, 23 nt, synthetic construct
  source 1..23: mol_type = other RNA
ttaaataaaacaagaaacttacc

SEQ ID NO: 133 - RNA, 23 nt, synthetic construct
  source 1..23: mol_type = other RNA
tttctaaccttcctgaaatatac

SEQ ID NO: 134 - RNA, 23 nt, synthetic construct
  source 1..23: mol_type = other RNA
tactgttcaataacaaatccttt

SEQ ID NO: 135 - RNA, 23 nt, synthetic construct
  source 1..23: mol_type = other RNA
aaatatatcctaaccgccacttt

SEQ ID NO: 136 - RNA, 23 nt, synthetic construct
  source 1..23: mol_type = other RNA
ttgttctaggtcactgctatcag

SEQ ID NO: 137 - RNA, 23 nt, synthetic construct
  source 1..23: mol_type = other RNA
cagtctagttctgtcctctattt

SEQ ID NO: 138 - RNA, 23 nt, synthetic construct
  source 1..23: mol_type = other RNA
tttcttaatttaataccaatact

SEQ ID NO: 139 - RNA, 23 nt, synthetic construct
  source 1..23: mol_type = other RNA
acttttaaatttttaaaatatgc

SEQ ID NO: 140 - RNA, 23 nt, synthetic construct
  source 1..23: mol_type = other RNA
tgctgctttaggtagatttaaaa

SEQ ID NO: 141 - RNA, 23 nt, synthetic construct
  source 1..23: mol_type = other RNA
aaatccttaaattcatggtcaca

SEQ ID NO: 142 - RNA, 23 nt, synthetic construct
  source 1..23: mol_type = other RNA
aaagtaattcactgctcaaaaat

SEQ ID NO: 143 - RNA, 23 nt, synthetic construct
  source 1..23: mol_type = other RNA
aattaccgtcagataaatattaa

SEQ ID NO: 144 - RNA, 23 nt, synthetic construct
  source 1..23: mol_type = other RNA
aggagaatttgtatccacataaa

SEQ ID NO: 145 - RNA, 23 nt, synthetic construct
  source 1..23: mol_type = other RNA
taagggaagaaacactttaaagg

SEQ ID NO: 146 - RNA, 23 nt, synthetic construct
  source 1..23: mol_type = other RNA
acaactttcctaatctcaaattg

SEQ ID NO: 147 - RNA, 23 nt, synthetic construct
  source 1..23: mol_type = other RNA
aataaactattaagatatataaa

SEQ ID NO: 148 - RNA, 23 nt, synthetic construct
  source 1..23: mol_type = other RNA
atatgacttaaagaactttttgt

SEQ ID NO: 149 - RNA, 23 nt, synthetic construct
  source 1..23: mol_type = other RNA
caagcttcctgaaaaggcttata

SEQ ID NO: 150 - RNA, 23 nt, synthetic construct
  source 1..23: mol_type = other RNA
tgtttaagtgtttggtcccaaat

SEQ ID NO: 151 - RNA, 23 nt, synthetic construct
  source 1..23: mol_type = other RNA
ggccacttggcaggtgaatgtct

SEQ ID NO: 152 - RNA, 23 nt, synthetic construct
  source 1..23: mol_type = other RNA
tctcgggagtgaatatgagacaa

SEQ ID NO: 153 - RNA, 23 nt, synthetic construct
  source 1..23: mol_type = other RNA
taggactggattgatcctcaggc

SEQ ID NO: 154 - RNA, 23 nt, synthetic construct
  source 1..23: mol_type = other RNA
attgtgtgccaaagtgcactctg

SEQ ID NO: 155 - RNA, 23 nt, synthetic construct
  source 1..23: mol_type = other RNA
cgaccgtggagtcatatgaggct

SEQ ID NO: 156 - RNA, 23 nt, synthetic construct
  source 1..23: mol_type = other RNA
ctgaactgttttggtaaagccga

SEQ ID NO: 157 - RNA, 23 nt, synthetic construct
  source 1..23: mol_type = other RNA
gctgaataacttgggagaatgta

SEQ ID NO: 158 - RNA, 23 nt, synthetic construct
  source 1..23: mol_type = other RNA
gtaagtctgcaaaataaacctag

SEQ ID NO: 159 - RNA, 23 nt, synthetic construct
  source 1..23: mol_type = other RNA
cttggaataccaaaccacacatt

SEQ ID NO: 160 - RNA, 23 nt, synthetic construct
  source 1..23: mol_type = other RNA
gctacatactggataagccagaa

SEQ ID NO: 161 - RNA, 23 nt, synthetic construct
  source 1..23: mol_type = other RNA
gaaagatgaggaaacatgtttgc

SEQ ID NO: 162 - RNA, 23 nt, synthetic construct
  source 1..23: mol_type = other RNA
tgcatctcacactagtgcagaga

SEQ ID NO: 163 - RNA, 23 nt, synthetic construct
  source 1..23: mol_type = other RNA
agattctgaaaaagaccccactt

SEQ ID NO: 164 - RNA, 23 nt, synthetic construct
  source 1..23: mol_type = other RNA
attagattgttctgttcccaatt

SEQ ID NO: 165 - RNA, 23 nt, synthetic construct
  source 1..23: mol_type = other RNA
tatttattatgtcacaaatagct

SEQ ID NO: 166 - RNA, 23 nt, synthetic construct
  source 1..23: mol_type = other RNA
tattttcatatatgtatatattt

SEQ ID NO: 167 - RNA, 21 nt, synthetic construct
  source 1..21: mol_type = other RNA
ctggtgtgatccaggaacagc

SEQ ID NO: 168 - RNA, 21 nt, synthetic construct
  source 1..21: mol_type = other RNA
attagccatggatgtattcat

SEQ ID NO: 169 - RNA, 21 nt, synthetic construct
  source 1..21: mol_type = other RNA
ctgtcttccagctctgaaaga

SEQ ID NO: 170 - RNA, 21 nt, synthetic construct
  source 1..21: mol_type = other RNA
gaggagcttgcttctccattc

SEQ ID NO: 171 - RNA, 21 nt, synthetic construct
  source 1..21: mol_type = other RNA
tgaaaggactttcaaaggcca

SEQ ID NO: 172 - RNA, 21 nt, synthetic construct
  source 1..21: mol_type = other RNA
aaggagggagttgtggctgct

SEQ ID NO: 173 - RNA, 21 nt, synthetic construct
  source 1..21: mol_type = other RNA
tgctgagaaaaccaaacaggg

SEQ ID NO: 174 - RNA, 21 nt, synthetic construct
  source 1..21: mol_type = other RNA
ctatgtaggctccaaaaccaa

SEQ ID NO: 175 - RNA, 21 nt, synthetic construct
  source 1..21: mol_type = other RNA
aagacaaaagagggtgttctc

SEQ ID NO: 176 - RNA, 21 nt, synthetic construct
  source 1..21: mol_type = other RNA
gtgtggcagaagcagcaggaa

SEQ ID NO: 177 - RNA, 21 nt, synthetic construct
  source 1..21: mol_type = other RNA
aggagggagtggtgcatggtg

SEQ ID NO: 178 - RNA, 21 nt, synthetic construct
  source 1..21: mol_type = other RNA
gaccaaagagcaagtgacaaa

SEQ ID NO: 179 - RNA, 21 nt, synthetic construct
  source 1..21: mol_type = other RNA
cagggagcattgcagcagcca

SEQ ID NO: 180 - RNA, 21 nt, synthetic construct
  source 1..21: mol_type = other RNA
ccagaagacagtggagggagc

SEQ ID NO: 181 - RNA, 21 nt, synthetic construct
  source 1..21: mol_type = other RNA
acgggtgtgacagcagtagcc

SEQ ID NO: 182 - RNA, 21 nt, synthetic construct
  source 1..21: mol_type = other RNA
atgttggaggagcagtggtga

SEQ ID NO: 183 - RNA, 21 nt, synthetic construct
  source 1..21: mol_type = other RNA
gtggcaacagtggctgagaag

SEQ ID NO: 184 - RNA, 21 nt, synthetic construct
  source 1..21: mol_type = other RNA
actggctttgtcaaaaaggac

SEQ ID NO: 185 - RNA, 21 nt, synthetic construct
  source 1..21: mol_type = other RNA
atgaaatgccttctgaggaag

SEQ ID NO: 186 - RNA, 21 nt, synthetic construct
  source 1..21: mol_type = other RNA
ggatcctgacaatgaggctta

SEQ ID NO: 187 - RNA, 21 nt, synthetic construct
  source 1..21: mol_type = other RNA
attctggaagatatgcctgtg

SEQ ID NO: 188 - RNA, 21 nt, synthetic construct
  source 1..21: mol_type = other RNA
aaggagccccacaggaaggaa

SEQ ID NO: 189 - RNA, 21 nt, synthetic construct
  source 1..21: mol_type = other RNA
ccagttgggcaagaatgaaga

SEQ ID NO: 190 - RNA, 21 nt, synthetic construct
  source 1..21: mol_type = other RNA
gggtatcaagactacgaacct

SEQ ID NO: 191 - RNA, 21 nt, synthetic construct
  source 1..21: mol_type = other RNA
aatgtgcccagtcatgacatt

SEQ ID NO: 192 - RNA, 21 nt, synthetic construct
  source 1..21: mol_type = other RNA
ctgtacaagtgctcagttcca

SEQ ID NO: 193 - RNA, 21 nt, synthetic construct
  source 1..21: mol_type = other RNA
tgctcccagtttcttgagatc

SEQ ID NO: 194 - RNA, 21 nt, synthetic construct
  source 1..21: mol_type = other RNA
ctgctgacagatgttccatcc

SEQ ID NO: 195 - RNA, 21 nt, synthetic construct
  source 1..21: mol_type = other RNA
tgaagcctaagaaatatcttt

SEQ ID NO: 196 - RNA, 21 nt, synthetic construct
  source 1..21: mol_type = other RNA
ttctcaaagtttttacagtgt

SEQ ID NO: 197 - RNA, 21 nt, synthetic construct
  source 1..21: mol_type = other RNA
aagtgaatacatggtagcagg

SEQ ID NO: 198 - RNA, 21 nt, synthetic construct
  source 1..21: mol_type = other RNA
tcggtgcttccctttcactga

SEQ ID NO: 199 - RNA, 21 nt, synthetic construct
  source 1..21: mol_type = other RNA
acctgcccccactcagcattt

SEQ ID NO: 200 - RNA, 21 nt, synthetic construct
  source 1..21: mol_type = other RNA
gcagtgattgaagtatctgta

SEQ ID NO: 201 - RNA, 21 nt, synthetic construct
  source 1..21: mol_type = other RNA
tatctcgaagtcttccatcag

SEQ ID NO: 202 - RNA, 21 nt, synthetic construct
  source 1..21: mol_type = other RNA
tttgtggcttcaatctacgat

SEQ ID NO: 203 - RNA, 21 nt, synthetic construct
  source 1..21: mol_type = other RNA
ttttgttgctgttgttcagaa

SEQ ID NO: 204 - RNA, 21 nt, synthetic construct
  source 1..21: mol_type = other RNA
tttctaaatcctcactatttt

SEQ ID NO: 205 - RNA, 21 nt, synthetic construct
  source 1..21: mol_type = other RNA
acctaagtgactaccacttat

SEQ ID NO: 206 - RNA, 21 nt, synthetic construct
  source 1..21: mol_type = other RNA
tgttaaaacaaattaaaaaca

SEQ ID NO: 207 - RNA, 21 nt, synthetic construct
  source 1..21: mol_type = other RNA
ggtctttgtgtgctgtggatt

SEQ ID NO: 208 - RNA, 21 nt, synthetic construct
  source 1..21: mol_type = other RNA
agttgttagtgatttgctatc

SEQ ID NO: 209 - RNA, 21 nt, synthetic construct
  source 1..21: mol_type = other RNA
taatacttaaaaatatgtgag

SEQ ID NO: 210 - RNA, 21 nt, synthetic construct
  source 1..21: mol_type = other RNA
gtgaaatttgttaatatatat

SEQ ID NO: 211 - RNA, 21 nt, synthetic construct
  source 1..21: mol_type = other RNA
tctaagaataatgacgtattg

SEQ ID NO: 212 - RNA, 21 nt, synthetic construct
  source 1..21: mol_type = other RNA
ggtgtcttttaatgatactgt

SEQ ID NO: 213 - RNA, 21 nt, synthetic construct
  source 1..21: mol_type = other RNA
catatattataagatttttag

SEQ ID NO: 214 - RNA, 21 nt, synthetic construct
  source 1..21: mol_type = other RNA
gcatgaaactatgcacctata

SEQ ID NO: 215 - RNA, 21 nt, synthetic construct
  source 1..21: mol_type = other RNA
aaaacgttatctcattgcaaa

SEQ ID NO: 216 - RNA, 21 nt, synthetic construct
  source 1..21: mol_type = other RNA
taaatggtgagaattaaaata

SEQ ID NO: 217 - RNA, 21 nt, synthetic construct
  source 1..21: mol_type = other RNA
tattcacttgtgtttgtatat

SEQ ID NO: 218 - RNA, 21 nt, synthetic construct
  source 1..21: mol_type = other RNA
taccattttgcgatgtgtttt

SEQ ID NO: 219 - RNA, 21 nt, synthetic construct
  source 1..21: mol_type = other RNA
aaatactaaatatgaaatttt

SEQ ID NO: 220 - RNA, 21 nt, synthetic construct
  source 1..21: mol_type = other RNA
atctcactttaataataaaaa

SEQ ID NO: 221 - RNA, 21 nt, synthetic construct
  source 1..21: mol_type = other RNA
taatagccatttgaagaagga

SEQ ID NO: 222 - RNA, 21 nt, synthetic construct
  source 1..21: mol_type = other RNA
ggacaaaaatataaagttatt

SEQ ID NO: 223 - RNA, 21 nt, synthetic construct
  source 1..21: mol_type = other RNA
gaattaagaactgacacaaag

SEQ ID NO: 224 - RNA, 21 nt, synthetic construct
  source 1..21: mol_type = other RNA
atcatgcttataagcaacatg

SEQ ID NO: 225 - RNA, 21 nt, synthetic construct
  source 1..21: mol_type = other RNA
aaatattttatttttatccca

SEQ ID NO: 226 - RNA, 21 nt, synthetic construct
  source 1..21: mol_type = other RNA
aggaattttagaagaggtaga

SEQ ID NO: 227 - RNA, 21 nt, synthetic construct
  source 1..21: mol_type = other RNA
ttaagtggctgtgattaatta

SEQ ID NO: 228 - RNA, 21 nt, synthetic construct
  source 1..21: mol_type = other RNA
ttttggtatgcactggttcct

SEQ ID NO: 229 - RNA, 21 nt, synthetic construct
  source 1..21: mol_type = other RNA
gcaacactgccagaagtgtgt

SEQ ID NO: 230 - RNA, 21 nt, synthetic construct
  source 1..21: mol_type = other RNA
tacactcggaattccctgaag

SEQ ID NO: 231 - RNA, 21 nt, synthetic construct
  source 1..21: mol_type = other RNA
agaaaatggaacattaaccct

SEQ ID NO: 232 - RNA, 21 nt, synthetic construct
  source 1..21: mol_type = other RNA
attgaaagtggggtgttgaag

SEQ ID NO: 233 - RNA, 21 nt, synthetic construct
  source 1..21: mol_type = other RNA
tacgtattttggggaactgtt

SEQ ID NO: 234 - RNA, 21 nt, synthetic construct
  source 1..21: mol_type = other RNA
aatcctgtcaatgtttgcttt

SEQ ID NO: 235 - RNA, 21 nt, synthetic construct
  source 1..21: mol_type = other RNA
agtggtctatttctcccttca

SEQ ID NO: 236 - RNA, 21 nt, synthetic construct
  source 1..21: mol_type = other RNA
gaccccaactactattgtaga

SEQ ID NO: 237 - RNA, 21 nt, synthetic construct
  source 1..21: mol_type = other RNA
tgtttgatgtgtatgtgttta

SEQ ID NO: 238 - RNA, 21 nt, synthetic construct
  source 1..21: mol_type = other RNA
ataattgttatacatttttaa

SEQ ID NO: 239 - RNA, 21 nt, synthetic construct
  source 1..21: mol_type = other RNA
tctattttgtctgatattggt

SEQ ID NO: 240 - RNA, 21 nt, synthetic construct
  source 1..21: mol_type = other RNA
aaataattttttagttaaaat

SEQ ID NO: 241 - RNA, 21 nt, synthetic construct
  source 1..21: mol_type = other RNA
atattgttatttttgtctcga

SEQ ID NO: 242 - RNA, 21 nt, synthetic construct
  source 1..21: mol_type = other RNA
attgagccttttattaacata

SEQ ID NO: 243 - RNA, 21 nt, synthetic construct
  source 1..21: mol_type = other RNA
tgtgaatgctgtacctttctg

SEQ ID NO: 244 - RNA, 21 nt, synthetic construct
  source 1..21: mol_type = other RNA
catgaataaaaaaaaaaaaaa

SEQ ID NO: 245 - RNA, 21 nt, synthetic construct
  source 1..21: mol_type = other RNA
agccataagacacattagcac

SEQ ID NO: 246 - RNA, 21 nt, synthetic construct
  source 1..21: mol_type = other RNA
tgactacaccctccttagaga

SEQ ID NO: 247 - RNA, 21 nt, synthetic construct
  source 1..21: mol_type = other RNA
aagtgggttcccgggaactaa

SEQ ID NO: 248 - RNA, 21 nt, synthetic construct
  source 1..21: mol_type = other RNA
agcagtgtagaagatgatttt

SEQ ID NO: 249 - RNA, 21 nt, synthetic construct
  source 1..21: mol_type = other RNA
gacaataaataatattcgacc

SEQ ID NO: 250 - RNA, 21 nt, synthetic construct
  source 1..21: mol_type = other RNA
catattagcacattcaaggct

SEQ ID NO: 251 - RNA, 21 nt, synthetic construct
  source 1..21: mol_type = other RNA
tctctctttttctctcgctct

SEQ ID NO: 252 - RNA, 21 nt, synthetic construct
  source 1..21: mol_type = other RNA
tcagaaattctctctctctct

SEQ ID NO: 253 - RNA, 21 nt, synthetic construct
  source 1..21: mol_type = other RNA
tcactttttttttttaatcat

SEQ ID NO: 254 - RNA, 21 nt, synthetic construct
  source 1..21: mol_type = other RNA
tttgtttaactcagcattcct

SEQ ID NO: 255 - RNA, 21 nt, synthetic construct
  source 1..21: mol_type = other RNA
tctgagagaatgtggttaact

SEQ ID NO: 256 - RNA, 21 nt, synthetic construct
  source 1..21: mol_type = other RNA
tctttttttttttttttttac

SEQ ID NO: 257 - RNA, 21 nt, synthetic construct
  source 1..21: mol_type = other RNA
ctagactgataaaaatttcat

SEQ ID NO: 258 - RNA, 21 nt, synthetic construct
  source 1..21: mol_type = other RNA
ccttaaaggagatcaattctc

SEQ ID NO: 259 - RNA, 21 nt, synthetic construct
  source 1..21: mol_type = other RNA
cgttggaactaccagagtcac

SEQ ID NO: 260 - RNA, 21 nt, synthetic construct
  source 1..21: mol_type = other RNA
caggaaatgcctttaaacatc

SEQ ID NO: 261 - RNA, 21 nt, synthetic construct
  source 1..21: mol_type = other RNA
tggcctcctttaaatgttgcc

SEQ ID NO: 262 - RNA, 21 nt, synthetic construct
  source 1..21: mol_type = other RNA
caaatatatgaattctaggat

SEQ ID NO: 263 - RNA, 21 nt, synthetic construct
  source 1..21: mol_type = other RNA
aaaactctgtataaattaatt

SEQ ID NO: 264 - RNA, 21 nt, synthetic construct
  source 1..21: mol_type = other RNA
gaaaataaacttgatggtgaa

SEQ ID NO: 265 - RNA, 21 nt, synthetic construct
  source 1..21: mol_type = other RNA
attaactccccatgggtgctg

SEQ ID NO: 266 - RNA, 21 nt, synthetic construct
  source 1..21: mol_type = other RNA
ttctctttcagggaagatcta

SEQ ID NO: 267 - RNA, 21 nt, synthetic construct
  source 1..21: mol_type = other RNA
tttttccttaggaaaggtttt

SEQ ID NO: 268 - RNA, 21 nt, synthetic construct
  source 1..21: mol_type = other RNA
atagtcatttctaaaagtcac

SEQ ID NO: 269 - RNA, 21 nt, synthetic construct
  source 1..21: mol_type = other RNA
tgctagcagtttatatgtatt

SEQ ID NO: 270 - RNA, 21 nt, synthetic construct
  source 1..21: mol_type = other RNA
ctagtagaaagtataatttca

SEQ ID NO: 271 - RNA, 21 nt, synthetic construct
  source 1..21: mol_type = other RNA
aagacagaatattctagacat

SEQ ID NO: 272 - RNA, 21 nt, synthetic construct
  source 1..21: mol_type = other RNA
ttaaaaattatttggtttctc

SEQ ID NO: 273 - RNA, 21 nt, synthetic construct
  source 1..21: mol_type = other RNA
ctttttaattattctggggca

SEQ ID NO: 274 - RNA, 21 nt, synthetic construct
  source 1..21: mol_type = other RNA
tcatgagtaatgtgatatata

SEQ ID NO: 275 - RNA, 21 nt, synthetic construct
  source 1..21: mol_type = other RNA
taaggatggttaccatagaaa

SEQ ID NO: 276 - RNA, 21 nt, synthetic construct
  source 1..21: mol_type = other RNA
attgggcgctggtgaggaagg

SEQ ID NO: 277 - RNA, 21 nt, synthetic construct
  source 1..21: mol_type = other RNA
gaagagagactactacagagt

SEQ ID NO: 278 - RNA, 21 nt, synthetic construct
  source 1..21: mol_type = other RNA
acttccttttttacctaattg

SEQ ID NO: 279 - RNA, 21 nt, synthetic construct
  source 1..21: mol_type = other RNA
gaaggaggaatgagtgactat

SEQ ID NO: 280 - RNA, 21 nt, synthetic construct
  source 1..21: mol_type = other RNA
tgctaagctgcatgtgtcatc

SEQ ID NO: 281 - RNA, 21 nt, synthetic construct
  source 1..21: mol_type = other RNA
agttatgtttaagcaaggaaa

SEQ ID NO: 282 - RNA, 21 nt, synthetic construct
  source 1..21: mol_type = other RNA
cttacactagagagaaatggt

SEQ ID NO: 283 - RNA, 21 nt, synthetic construct
  source 1..21: mol_type = other RNA
taagtttcttgttttatttaa

SEQ ID NO: 284 - RNA, 21 nt, synthetic construct
  source 1..21: mol_type = other RNA
atatttcaggaaggttagaaa

SEQ ID NO: 285 - RNA, 21 nt, synthetic construct
  source 1..21: mol_type = other RNA
aggatttgttattgaacagta

SEQ ID NO: 286 - RNA, 21 nt, synthetic construct
  source 1..21: mol_type = other RNA
agtggcggttaggatatattt

SEQ ID NO: 287 - RNA, 21 nt, synthetic construct
  source 1..21: mol_type = other RNA
gatagcagtgacctagaacaa

SEQ ID NO: 288 - RNA, 21 nt, synthetic construct
  source 1..21: mol_type = other RNA
atagaggacagaactagactg

SEQ ID NO: 289 - RNA, 21 nt, synthetic construct
  source 1..21: mol_type = other RNA
tattggtattaaattaagaaa

SEQ ID NO: 290 - RNA, 21 nt, synthetic construct
  source 1..21: mol_type = other RNA
atattttaaaaatttaaaagt

SEQ ID NO: 291 - RNA, 21 nt, synthetic construct
  source 1..21: mol_type = other RNA
ttaaatctacctaaagcagca

SEQ ID NO: 292 - RNA, 21 nt, synthetic construct
  source 1..21: mol_type = other RNA
tgaccatgaatttaaggattt

SEQ ID NO: 293 - RNA, 21 nt, synthetic construct
  source 1..21: mol_type = other RNA
ttttgagcagtgaattacttt

SEQ ID NO: 294 - RNA, 21 nt, synthetic construct
  source 1..21: mol_type = other RNA
aatatttatctgacggtaatt

SEQ ID NO: 295 - RNA, 21 nt, synthetic construct
  source 1..21: mol_type = other RNA
tatgtggatacaaattctcct

SEQ ID NO: 296 - RNA, 21 nt, synthetic construct
  source 1..21: mol_type = other RNA
tttaaagtgtttcttccctta

SEQ ID NO: 297 - RNA, 21 nt, synthetic construct
  source 1..21: mol_type = other RNA
atttgagattaggaaagttgt

SEQ ID NO: 298 - RNA, 21 nt, synthetic construct
  source 1..21: mol_type = other RNA
tatatatcttaatagtttatt

SEQ ID NO: 299 - RNA, 21 nt, synthetic construct
  source 1..21: mol_type = other RNA
aaaaagttctttaagtcatat

SEQ ID NO: 300 - RNA, 21 nt, synthetic construct
  source 1..21: mol_type = other RNA
taagccttttcaggaagcttg

SEQ ID NO: 301 - RNA, 21 nt, synthetic construct
  source 1..21: mol_type = other RNA
ttgggaccaaacacttaaaca

SEQ ID NO: 302 - RNA, 21 nt, synthetic construct
  source 1..21: mol_type = other RNA
acattcacctgccaagtggcc

SEQ ID NO: 303 - RNA, 21 nt, synthetic construct
  source 1..21: mol_type = other RNA
gtctcatattcactcccgaga

SEQ ID NO: 304 - RNA, 21 nt, synthetic construct
  source 1..21: mol_type = other RNA
ctgaggatcaatccagtccta

SEQ ID NO: 305 - RNA, 21 nt, synthetic construct
  source 1..21: mol_type = other RNA
gagtgcactttggcacacaat

SEQ ID NO: 306 - RNA, 21 nt, synthetic construct
  source 1..21: mol_type = other RNA
cctcatatgactccacggtcg

SEQ ID NO: 307 - RNA, 21 nt, synthetic construct
  source 1..21: mol_type = other RNA
ggctttaccaaaacagttcag

SEQ ID NO: 308 - RNA, 21 nt, synthetic construct
  source 1..21: mol_type = other RNA
cattctcccaagttattcagc

SEQ ID NO: 309 - RNA, 21 nt, synthetic construct
  source 1..21: mol_type = other RNA
aggtttattttgcagacttac

SEQ ID NO: 310 - RNA, 21 nt, synthetic construct
  source 1..21: mol_type = other RNA
tgtgtggtttggtattccaag

SEQ ID NO: 311 - RNA, 21 nt, synthetic construct
  source 1..21: mol_type = other RNA
ctggcttatccagtatgtagc

SEQ ID NO: 312 - RNA, 21 nt, synthetic construct
  source 1..21: mol_type = other RNA
aaacatgtttcctcatctttc

SEQ ID NO: 313 - RNA, 21 nt, synthetic construct
  source 1..21: mol_type = other RNA
tctgcactagtgtgagatgca

SEQ ID NO: 314 - RNA, 21 nt, synthetic construct
  source 1..21: mol_type = other RNA
gtggggtctttttcagaatct

SEQ ID NO: 315 - RNA, 21 nt, synthetic construct
  source 1..21: mol_type = other RNA
ttgggaacagaacaatctaat

SEQ ID NO: 316 - RNA, 21 nt, synthetic construct
  source 1..21: mol_type = other RNA
ctatttgtgacataataaata

SEQ ID NO: 317 - RNA, 21 nt, synthetic construct
  source 1..21: mol_type = other RNA
atatatacatatatgaaaata

SEQ ID NO: 318 - RNA, 23 nt, synthetic construct
  source 1..23: mol_type = other RNA
tgaattcctttacaccacactct

SEQ ID NO: 319 - RNA, 21 nt, synthetic construct
  source 1..21: mol_type = other RNA
agtgtggtgtaaaggaattca

SEQ ID NO: 320 - RNA, 23 nt, Synthetic construct
  source 1..23: mol_type = other RNA
  modified_base 1: mod_base = OTHER; note = thymine
tcagtgaaagggaagcaccgaaa

SEQ ID NO: 321 - RNA, 23 nt, Synthetic construct
  source 1..23: mol_type = other RNA
  modified_base 1: mod_base = OTHER; note = thymine
tctacctcttctaaaattcctcc

SEQ ID NO: 322 - RNA, 23 nt, Synthetic construct
  source 1..23: mol_type = other RNA
  modified_base 1: mod_base = OTHER; note = thymine
ttatacaaacacaagtgaataaa

SEQ ID NO: 323 - RNA, 23 nt, Synthetic construct
  source 1..23: mol_type = other RNA
  modified_base 1: mod_base = OTHER; note = thymine
taaacacatcgcaaaatggtaaa

SEQ ID NO: 324 - RNA, 23 nt, Synthetic construct
  source 1..23: mol_type = other RNA
  modified_base 1: mod_base = OTHER; note = thymine
ttttgtgtcagttcttaattcat

SEQ ID NO: 325 - RNA, 19 nt, Synthetic construct
  source 1..19: mol_type = other RNA
tatcactgtgtttgtataa

SEQ ID NO: 326 - RNA, 21 nt, Synthetic construct
  source 1..21: mol_type = other RNA
taccattttgcgatgtgttta

SEQ ID NO: 327 - RNA, 21 nt, Synthetic construct
  source 1..21: mol_type = other RNA
gaattaagaactgacacaaaa

SEQ ID NO: 328 - DNA, 15 bp, Synthetic construct
  source 1..15: mol_type = other DNA
tcgccgcttgctgca

SEQ ID NO: 329 - DNA, 17 bp, Synthetic construct
  source 1..17: mol_type = other DNA
atcggccgtgatgtcga

SEQ ID NO: 330 - DNA, 23 bp, Synthetic construct
  source 1..23: mol_type = other DNA
ccatggtcaaccccaccgtgttc

(19) United States
(12) Patent Application Publication
(10) Pub. No.: US 2026/0132401 A1
(43) Pub. Date: May 14, 2026

(54) RNAi AGENTS FOR MODULATING SNCA

(71) Applicant: Ionis Pharmaceuticals, Inc., Carlsbad, CA (US)

(72) Inventors: Susan M. Freier, San Diego, CA (US); Thazha P. Prakash, Carlsbad, CA (US)

(73) Assignee: Ionis Pharmaceuticals, Inc., Carlsbad, CA (US)

(21) Appl. No.: 18/863,476

(22) PCT Filed: May 5, 2023
(86) PCT No.: PCT/US2023/066655

§ 371 Date: Nov. 6, 2024

Related U.S. Application Data
(60) Provisional application No. 63/339,292, filed on May 6, 2022.

(51) Int. Cl. C12N 15/113; A61K 31/7115; A61K 31/712; A61K 31/7125; A61K 47/02; A61K 47/46; A61P 25/16

(52) U.S. Cl. CPC C12N 15/113; A61K 31/7115; A61K 31/712; A61K 31/7125; A61K 47/02; A61K 47/46; A61P 25/16; C12N 2310/14; C12N 2310/315; C12N 2310/321; C12N 2310/322; C12N 2310/3341; C12N 2310/3519

(57) ABSTRACT

Provided are antisense agents, pharmaceutical compositions, and methods of use for reducing the amount or activity of alpha-synuclein (SNCA) RNA in a cell or subject, and in certain instances reducing the amount of alpha-synuclein protein in a cell or subject. In certain embodiments, also provided herein are oligomeric compounds, oligomeric duplexes, and RNAi agents for reducing the amount or activity of SNCA RNA in a cell or subject, and in certain instances reducing the amount of SNCA protein in a cell or subject. Such compounds, methods, and pharmaceutical compositions are useful to ameliorate at least one symptom or hallmark of a neurodegenerative disease. Such neurodegenerative diseases include synucleinopathies, e.g., Parkinson's disease, dementia with Lewy bodies, diffuse Lewy body disease, Parkinson's disease dementia, pure autonomic failure, multiple system atrophy, neuronopathic Gaucher's disease, and Alzheimer's disease.

### SEQUENCE LISTING

The present application is being filed along with a Sequence Listing in electronic format. The Sequence Listing is provided as a file entitled BIOL0450SEQ.xml created Mar. 30, 2023, which is 415 kb in size. The information in the electronic format of the sequence listing is incorporated herein by reference in its entirety.

### FIELD

Provided are antisense agents, pharmaceutical compositions, and methods of use for reducing the amount or activity of alpha-synuclein (SNCA) RNA in a cell or subject, and in certain instances reducing the amount of alpha-synuclein protein in a cell or subject. In certain embodiments, also provided herein are oligomeric compounds and oligomeric duplexes for reducing the amount or activity of SNCA RNA in a cell or subject, and in certain instances reducing the amount of SNCA protein in a cell or subject. In certain embodiments, further provided herein are RNAi agents for reducing the amount or activity of SNCA RNA in a cell or subject, and in certain instances reducing the amount of SNCA protein in a cell or subject. Such compounds, methods, and pharmaceutical compositions are useful to ameliorate at least one symptom or hallmark of a neurodegenerative disease. Such neurodegenerative diseases include synucleinopathies such as Parkinson's disease, dementia with Lewy bodies, diffuse Lewy body disease, Parkinson's disease dementia (PDD), pure autonomic failure, multiple system atrophy (MSA), neuronopathic Gaucher's disease, and Alzheimer's disease. Such symptoms and hallmarks include motor dysfunction, aggregation of alpha-synuclein, neurodegeneration, cognitive decline, dementia, sleep disorders, hyposmia, autonomic failure, ataxia, hallucination, or seizures.

### BACKGROUND

Alpha-synuclein is a small, highly charged 140-amino acid residue protein, predominantly expressed in central nervous system (CNS) neurons, where it is localized at presynaptic terminals in close proximity to synaptic vesicles (Iwai, et al., Neuron. 1995. 14: 467-475). Alpha-synuclein is encoded by the SNCA gene. Alpha-synuclein can associate with lipid membranes by forming amphipathic α-helices, as shown in vitro (Davidson, et al., J. Biol. Chem. 1998. 273: 9443-9449). Several studies suggest that alpha-synuclein is involved in modulating synaptic transmission, the density of synaptic vesicles, and neuronal plasticity (Cabin et al., J. Neurosci. 2002. 22: 8797-8807; Burre et al., Science. 2010. 329: 1663-1667; and Burre et al., J. Neurosci. 2015. 35: 5221-5232). It has also been suggested that alpha-synuclein may have a chaperone function, as indicated by its effectiveness in preventing aggregation of proteins in in vitro assays (Souza et al., FEBS Lett. 2000. 474: 116-119). Moreover, in vivo assays demonstrate that alpha-synuclein chaperone activity is instrumental in promoting the assembly of the SNARE-complex, which is essential for neurotransmitter release in the presynaptic terminals of the brain (Burre et al., Science. 2010, 329: 1663-1667). Decreased SNARE-complex assembly is associated with neurological impairment, thus, indicating a link between presynaptic alpha-synuclein aggregates and neurodegeneration (Kramer and Schulz-Schaeffer, J. Neurosci. 2007. 27: 1405-1410). Knockout mouse models of alpha-synuclein are not lethal, and brain morphology is intact, suggesting that alpha-synuclein is not required for neuronal development and/or that compensatory pathways are present (Abeliovich et al., Neuron. 2000. 25: 239-252).

Misfolding, aggregation, and fibrillation of alpha-synuclein are implicated as critical factors in several neurodegenerative diseases, including, Parkinson's disease, Lewy body variant of Alzheimer's disease, diffuse Lewy body disease, dementia with Lewy bodies, and multiple system atrophy (Schulz-Schaeffer, Acta Neuropathol. 2010. 120: 131-143; Yoshida. Neuropathology. 2007. 27: 484-493). In each of these cases, alpha-synuclein protein is misfolded and assembles in aggregates in Lewy bodies and Lewy neurites (Uversky. J. Neurochem. 2007. 103: 17-37). Several recent studies have shown that lipidic environments that promote alpha-synuclein folding also accelerate alpha-synuclein aggregation, suggesting that the lipid-associated conformation of alpha-synuclein may be relevant to alpha-synuclein misfolding in neurodegenerative diseases (Conway et al., Science. 2001. 294: 6-9; Lee et al., J. Biol. Chem. 2002. 277: 671-678). Mutations at position 53, where alanine is changed to threonine, and at position 30, where alanine is changed to proline, have been shown to cause alpha-synuclein to be in a random coil state, so that aggregation is more likely to occur (Clayton and George, J. Neurosci. 1999. 58: 120-129).

Currently there are few acceptable options for treating neurodegenerative diseases such as synucleinopathies (e.g., Parkinson's disease, dementia with Lewy bodies, diffuse Lewy body disease, Parkinson's disease dementia, pure autonomic failure, multiple system atrophy, neuronopathic Gaucher's disease, and Alzheimer's disease). It is therefore an objective herein to provide compounds, methods, and pharmaceutical compositions for the treatment of such diseases.

### SUMMARY

Provided herein are compounds, pharmaceutical compositions, and methods of use for reducing the amount or activity of SNCA RNA, and in certain embodiments reducing the expression of SNCA protein in a cell or subject. In certain embodiments, the subject has a synucleinopathy. In certain embodiments, the subject has Parkinson's disease, dementia with Lewy bodies, diffuse Lewy body disease, Parkinson's disease dementia, pure autonomic failure, multiple system atrophy, neuronopathic Gaucher's disease, or Alzheimer's disease. In certain embodiments, compounds useful for reducing the amount or activity of SNCA RNA are oligomeric compounds. In certain embodiments, compounds useful for reducing the amount or activity of SNCA RNA are oligomeric duplexes or antisense agents. In certain embodiments, compounds useful for reducing the amount or activity of SNCA RNA are RNAi agents. In certain embodiments, compounds useful for decreasing expression of SNCA protein are oligomeric compounds. In certain embodiments, compounds useful for decreasing expression of SNCA protein are oligomeric duplexes or antisense agents. In certain embodiments, compounds useful for decreasing expression of SNCA protein are RNAi agents.

Also provided are methods useful for ameliorating at least one symptom or hallmark of a neurodegenerative disease. In certain embodiments, the neurodegenerative disease is a synucleinopathy. In certain embodiments, the neurodegenerative disease is Parkinson's disease, dementia with Lewy bodies, diffuse Lewy body disease, Parkinson's disease dementia, pure autonomic failure, multiple system atrophy, neuronopathic Gaucher's disease, or Alzheimer's disease. In certain embodiments, the symptom or hallmark includes motor dysfunction, aggregation of alpha-synuclein, neurodegeneration, cognitive decline, dementia, sleep disorders, hyposmia, autonomic failure, ataxia, hallucination, or seizures. In certain embodiments, amelioration of these symptoms results in improved motor function, a reduced amount or volume of alpha-synuclein aggregates, reduced neurodegeneration, improved cognitive function, delayed onset or progression of dementia, reduced sleep disorders, improved hyposmia, reduced or delayed autonomic failure, reduced ataxia, reduced hallucination, or reduced seizures.

### DETAILED DESCRIPTION OF THE INVENTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

As used herein, “2′-deoxynucleoside” means a nucleoside comprising a 2′-H(H) deoxyribosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside or a nucleoside comprising an unmodified 2′-deoxyribosyl sugar moiety may comprise a modified nucleobase or may comprise an RNA nucleobase (uracil).

As used herein, “2′-MOE” means a 2′-OCH₂CH₂OCH₃ group in place of the 2′-OH group of a ribosyl sugar moiety. A “2′-MOE sugar moiety” means a sugar moiety with a 2′-OCH₂CH₂OCH₃ group in place of the 2′-OH group of a ribosyl sugar moiety. Unless otherwise indicated, a 2′-MOE sugar moiety is in the β-D-ribosyl configuration. “MOE” means O-methoxyethyl.

As used herein, “2′-MOE nucleoside” or “2′-OCH₂CH₂OCH₃ nucleoside” means a nucleoside comprising a 2′-MOE sugar moiety (or 2′-OCH₂CH₂OCH₃ ribosyl sugar moiety).

As used herein, “2′-OMe” means a 2′-OCH₃ group in place of the 2′-OH group of a ribosyl sugar moiety. A “2′-O-methyl sugar moiety” or “2′-OMe sugar moiety” or “2′-O-methylribosyl sugar moiety” or “ribo-2′-OMe sugar moiety” means a sugar moiety with a 2′-OCH₃ group in place of the 2′-OH group of a ribosyl sugar moiety. Unless otherwise indicated, a 2′-OMe sugar moiety is in the β-D-ribosyl configuration.

As used herein, “2′-OMe nucleoside” or “2′-OMe modified nucleoside” means a nucleoside comprising a 2′-OMe sugar moiety.

As used herein, “2′-F” means a 2′-fluoro group in place of the 2′-OH group of a furanosyl sugar moiety. A “ribo-2′-F sugar moiety” means a sugar moiety with a ribo-2′-F group in place of the 2′-OH group of a furanosyl sugar moiety. Unless otherwise indicated, 2′-F sugar moiety is in the β-D-ribosyl configuration.

As used herein, “2′-F nucleoside” or “2′-F modified nucleoside” means a nucleoside comprising a ribo-2′-F modified sugar moiety.

As used herein, “xylo 2′-F” means a 2′-F sugar moiety in the β-D-xylosyl configuration.

As used herein, “2′-substituted nucleoside” means a nucleoside comprising a 2′-substituted furanosyl sugar moiety. As used herein, “2′-substituted” in reference to a sugar moiety means a sugar moiety comprising at least one 2′-substituent group other than H or OH.

As used herein, “3′ target site” refers to the 3′-most nucleotide of a target nucleic acid which is complementary to an antisense oligonucleotide, when the antisense oligonucleotide is hybridized to the target nucleic acid.

As used herein, “5′ target site” refers to the 5′-most nucleotide of a target nucleic acid which is complementary to an antisense oligonucleotide, when the antisense oligonucleotide is hybridized to the target nucleic acid.

As used herein, “5-methylcytosine” means a cytosine modified with a methyl group attached to the 5 position. A 5-methylcytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “alpha-synuclein associated disease”, “disease or disorder associated with SNCA”, or “SNCA associated disease” means any disease associated with any alpha-synuclein nucleic acid or expression product thereof. Such diseases may include a neurodegenerative disease, e.g., a synucleinopathy. Such neurodegenerative diseases may include Parkinson's disease, dementia with Lewy bodies, diffuse Lewy body disease, Parkinson's disease dementia, pure autonomic failure, multiple system atrophy, neuronopathic Gaucher's disease, and Alzheimer's disease.

As used here, “alpha-synuclein RNA” or “SNCA RNA” means any messenger RNA (mRNA) expression product of a DNA sequence encoding alpha-synuclein.

As used herein, “alpha-synuclein nucleic acid” or “SNCA nucleic acid” means any nucleic acid encoding alpha-synuclein. For example, in certain embodiments, an alpha-synuclein nucleic acid includes a DNA sequence encoding alpha-synuclein, an RNA sequence transcribed from DNA encoding alpha-synuclein (including genomic DNA comprising introns and exons), and an mRNA sequence encoding alpha-synuclein. “alpha-synuclein mRNA” means an mRNA encoding an alpha-synuclein protein.

As used herein, “alpha-synuclein protein” or “SNCA protein” means the polypeptide expression product of an alpha-synuclein nucleic acid.

As used herein, “administering” or “administration” means providing a pharmaceutical agent or composition to a subject.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark or the delayed onset or slowing of progression in the severity or frequency of a symptom or hallmark. In certain embodiments, the symptom or hallmark is motor dysfunction, aggregation of alpha-synuclein, neurodegeneration, cognitive decline, dementia, sleep disorders, hyposmia, autonomic failure, ataxia, hallucination, or seizures. The progression or severity of indicators may be determined by subjective or objective measures, which are known to those skilled in the art.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense agent” means an antisense compound and optionally one or more additional features, such as a sense compound.

As used herein, “antisense compound” means an antisense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include, but are not limited to, antisense RNAi oligonucleotides and antisense RNase H oligonucleotides.

As used herein, “sense compound” means a sense oligonucleotide and optionally one or more additional features, such as a conjugate group.

As used herein, “sense oligonucleotide” means an oligonucleotide, including the oligonucleotide portion of an oligomeric compound, that is capable of hybridizing to an antisense oligonucleotide. Sense oligonucleotides include, but are not limited to, sense RNAi oligonucleotides.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl sugar moiety. In certain embodiments, the furanosyl sugar moiety is a ribosyl sugar moiety. In certain embodiments, the bicyclic sugar moiety does not comprise a furanosyl sugar moiety.

As used herein, “blunt” or “blunt ended” in reference to an oligomeric duplex means that there are no terminal unpaired nucleotides (i.e. no overhanging nucleotides). One or both ends of an oligomeric duplex can be blunt.

As used herein, “cell-targeting moiety” means a conjugate group or portion of a conjugate group that is capable of binding to a particular cell type or particular cell types.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) similar to cerebrospinal fluid and is biocompatible with CSF.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide or one or more portions thereof and the nucleobases of another nucleic acid or one or more portions thereof are capable of hydrogen bonding with one another when the nucleobase sequence of the oligonucleotide and the other nucleic acid are aligned in opposing directions.

As used herein, “complementary nucleobases” means nucleobases that are capable of forming hydrogen bonds with one another. Complementary nucleobase pairs include adenine (A) and thymine (T), adenine (A) and uracil (U), cytosine (C) and guanine (G), 5-methylcytosine (^{m}C) and guanine (G). Certain modified nucleobases that pair with natural nucleobases or with other modified nucleobases are known in the art. For example, inosine can pair with adenosine, cytosine, or uracil. Complementary oligonucleotides and/or nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or nucleic acid at each nucleobase of the shorter of the two oligonucleotides, or at each nucleoside if the oligonucleotides are the same length.

As used herein, “complementary region” in reference to an oligonucleotide is the range of nucleobases of the oligonucleotide that is complementary with a second oligonucleotide or target nucleic acid.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or intemucleoside linkages that are immediately adjacent to each other. For example, “contiguous nucleobases” means nucleobases that are immediately adjacent to each other in a sequence.

As used herein, “constrained ethyl” or “cEt” or “cEt sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt sugar moiety.

As used herein, “chirally enriched population” or “chirally enriched” in reference to a population means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom as defined herein. Chirally enriched populations of molecules having multiple chiral centers within each molecule may contain one or more stereorandom chiral centers. In certain embodiments, the molecules are modified oligonucleotides. In certain embodiments, the molecules are oligomeric compounds comprising modified oligonucleotides. In certain embodiments, the chiral center is at the phosphorous atom of a phosphorothioate internucleoside linkage.

As used herein, “diluent” means an ingredient in a composition that lacks pharmacological activity, but is pharmaceutically necessary or desirable. For example, the diluent in an injected composition can be a liquid, e.g. aCSF, PBS, or saline solution.

As used herein, “double-stranded” in reference to a region or an oligonucleotide means a duplex formed by complementary strands of nucleic acids (including, but not limited to oligonucleotides) hybridized to one another. In certain embodiments, the two strands of a double-stranded region are separate molecules. In certain embodiments, the two strands are regions of the same molecule that has folded onto itself (e.g., a hairpin structure).

As used herein, “duplex” or “duplex region” means the structure formed by two oligonucleotides or portions thereof that are hybridized to one another.

As used herein, “hotspot region” is a range of nucleobases on a target nucleic acid that is amenable to antisense agent-mediated reduction of the amount or activity of the target nucleic acid.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an antisense oligonucleotide and a nucleic acid target.

As used herein, “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate internucleoside linkage” is a modified internucleoside linkage in which one of the non-bridging oxygen atoms of a phosphodiester internucleoside linkage is replaced with a sulfur atom.

As used herein, “inverted nucleoside” means a nucleotide having a 3′ to 3′ and/or 5′ to 5′ internucleoside linkage, as shown herein.

As used herein, “inverted sugar moiety” means the sugar moiety of an inverted nucleoside or an abasic sugar moiety having a 3′ to 3′ and/or 5′ to 5′ internucleoside linkage.

As used herein, “lipid nanoparticle” or “LNP” is a vesicle comprising a lipid layer encapsulating a pharmaceutically active molecule, such as a nucleic acid molecule, e.g., an RNAi agent or a plasmid from which an RNAi agent is transcribed. LNPs are described in, for example, U.S. Pat. Nos. 6,858,225, 6,815,432, 8,158,601, and 8,058,069, the entire contents of which are hereby incorporated herein by reference.

As used herein, “linked nucleosides” are nucleosides that are connected in a contiguous sequence (i.e., no additional nucleosides are presented between those that are linked).

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “mismatch” or “non-complementary” means a nucleobase of a first nucleic acid sequence that is not complementary with the corresponding nucleobase of a second nucleic acid sequence when the first and second nucleic acid sequences are aligned in opposing directions.

As used herein, “modified nucleoside” means a nucleoside comprising a modified nucleobase and/or a modified sugar moiety.

As used herein, “motif” means the pattern of unmodified and/or modified sugar moieties, nucleobases, and/or internucleoside linkages, in an oligonucleotide.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methylcytosine” is a modified nucleobase. A universal base is a modified nucleobase that can pair with any one of the five unmodified nucleobases.

As used herein, “nucleobase sequence” means the order of contiguous nucleobases in a target nucleic acid or oligonucleotide, including such nucleobases that are each optionally independently modified or unmodified, and independent of any sugar or internucleoside linkage modification.

As used herein, “the nucleobase sequence of” a reference SEQ ID NO refers only to the nucleobase sequence provided in such SEQ ID NO and therefore, unless otherwise indicated, includes compounds wherein each nucleobase, each sugar moiety, and each internucleoside linkage, independently, may be modified or unmodified, irrespective of the presence or absence of modifications, indicated in the referenced SEQ ID NO.

As used herein, “nucleoside” means a compound, or a fragment of a compound, comprising a nucleobase and a sugar moiety. The nucleobase and sugar moiety are each, independently, unmodified or modified.

As used herein, “nucleoside overhang” refers to unpaired nucleosides at either or both ends of an oligomeric duplex formed by hybridization of two oligonucleotides.

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

As used herein, “oligomeric duplex” means a duplex formed by two oligomeric compounds having complementary nucleobase sequences.

As used herein, “oligonucleotide” means a polymer of linked nucleosides connected via internucleoside linkages, wherein each nucleoside and internucleoside linkage may be modified or unmodified. Unless otherwise indicated, oligonucleotides consist of 8-50 linked nucleosides.

As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “prodrug” means a therapeutic agent in a first form outside the body that is converted to a second form within a subject or cells thereof. Typically, conversion of a prodrug within the subject is facilitated by the action of an enzymes (e.g., endogenous or viral enzyme) or chemicals present in cells or tissues and/or by physiologic conditions. In certain embodiments, the first form of the prodrug is less active than the second form.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “RNA” means an RNA transcript and includes pre-mRNA and mature mRNA unless otherwise specified.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNA (ssRNAi), and microRNA, including microRNA mimics. RNAi agents may comprise conjugate groups and/or terminal groups. In certain embodiments, an RNAi agent modulates the amount, activity, and/or splicing of a target nucleic acid. The term RNAi agent excludes antisense agents that act through RNase H.

As used herein, “antisense RNAi oligonucleotide” means an oligonucleotide comprising a region that is complementary to a target sequence, and which includes at least one chemical modification suitable for RNAi-mediated nucleic acid reduction.

As used herein, “standard in vitro assay” means the assay described in Example 2 and reasonable variations thereof.

As used herein, “stereorandom” or “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center (racemic). The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “stabilized phosphate group” means a 5′-phosphate analog that is metabolically more stable than a 5′-phosphate as naturally occurs on DNA or RNA.

As used herein, “subject” means a human or non-human animal. In certain embodiments, the subject is a human. The terms “subject” and “animal” are used interchangeably herein.

As used herein, “sugar moiety” means an unmodified sugar moiety or a modified sugar moiety.

As used herein, “unmodified sugar moiety” means a 2′-OH(H) β-D-ribosyl sugar moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom” or “hallmark” means any physical feature or test result that indicates the existence or extent of a disease or disorder. In certain embodiments, a symptom is apparent to a subject or to a medical professional examining or testing the subject. In certain embodiments, a hallmark is apparent upon invasive diagnostic testing, including, but not limited to, post-mortem tests. In certain embodiments, a hallmark is apparent on a brain MRI scan. In certain embodiments, symptoms and hallmarks include motor dysfunction, aggregation of alpha-synuclein, neurodegeneration, cognitive decline, dementia, sleep disorders, hyposmia, autonomic failure, ataxia, hallucination, or seizures.

As used herein, “target nucleic acid” and “target RNA” mean a nucleic acid that an antisense compound is designed to affect. Target RNA means an mRNA transcript and includes pre-mRNA and mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “treating” means improving a subject's disease or condition by administering an oligomeric agent, an oligomeric compound, an oligomeric duplex, or an antisense agent described herein.

In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a pharmaceutical agent or composition that provides a therapeutic benefit to an animal. For example, a therapeutically effective amount improves a symptom of a disease.

### CERTAIN EMBODIMENTS

The present disclosure provides the following non-limiting numbered embodiments:
Embodiment 1. An oligomeric compound, wherein the oligomeric compound comprises a modified oligonucleotide consisting of 12 to 50 linked nucleosides, wherein the nucleobase sequence of the modified oligonucleotide comprises at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 16-166, 318, or 320-324 and wherein the modified oligonucleotide comprises at least one modification selected from a modified sugar moiety and a modified internucleoside linkage.
Embodiment 2. The oligomeric compound of claim 1, wherein the nucleobase sequence of the modified oligonucleotide comprises the nucleobase sequence of any of SEQ ID NOs: 16-166, 318, or 320-324.
Embodiment 3. The oligomeric compound of claim 1, wherein the nucleobase sequence of the modified oligonucleotide consists of the nucleobase sequence of any of SEQ ID NOs: 16-166, 318, or 320-324.
Embodiment 4. The oligomeric compound of any of claims 1-3, wherein the nucleobase sequence of the modified oligonucleotide is at least 80%, at least 85%, at least 90%, at least 95%, or 100% complementary to the nucleobase sequence of an equal length portion of any of SEQ ID NOs: 1-6.
Embodiment 5. The oligomeric compound of any of claims 1-4, wherein the modified oligonucleotide consists of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 25, 18 to 30, 18 to 50, 19 to 20, 19 to 25, 19 to 29, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.
Embodiment 6. An oligomeric compound, wherein the oligomeric compound comprises a modified oligonucleotide consisting of 12 to 50 linked nucleosides, wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, at least 21, at least 22, or 23 contiguous nucleobases of
an equal length portion of nucleobases 775-997 of SEQ ID NO: 1;
an equal length portion of nucleobases 1115-1237 of SEQ ID NO: 1; or
an equal length portion of nucleobases 1455-1577 of SEQ ID NO: 1.
Embodiment 7. The oligomeric compound of claim 6, wherein the nucleobase sequence of the modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of a nucleobase sequence selected from:
   SEQ ID NOs: 45, 46, 47, 48, 49, 50, 51, 53, 54, 55, and 56;
   SEQ ID NOs: 58, 63, 65, 66, 67, and 68; and
   SEQ ID NOs: 76, 77, 81, 83, 84, and 85.
Embodiment 8. The oligomeric compound of claim 6, wherein the nucleobase sequence of the modified oligonucleotide comprises or consists of the nucleobase sequence selected from:
   SEQ ID NOs: 45, 46, 47, 48, 49, 50, 51, 53, 54, 55, and 56;
   SEQ ID NOs: 58, 63, 65, 66, 67, and 68; and
   SEQ ID NOs: 76, 77, 81, 83, 84, and 85.
Embodiment 9. An oligomeric compound, wherein the oligomeric compound comprises a modified oligonucleotide consisting of 12 to 50 linked nucleosides, wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, at least 21, at least 22, or 23 contiguous nucleobases of
   an equal length portion of nucleobases 1315-1397 of SEQ ID NO: 1;
   an equal length portion of nucleobases 1475-1577 of SEQ ID NO: 1; or
   an equal length portion of nucleobases 1655-1737 of SEQ ID NO: 1.
Embodiment 10. The oligomeric compound of claim 9, wherein the nucleobase sequence of the modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of a nucleobase sequence selected from:
   SEQ ID NOs: 70, 71, 72 and 75;
   SEQ ID NOs: 76, 81, 83, 84, and 85; and
   SEQ ID NOs: 88, 89, 90, and 92.
Embodiment 11. The oligomeric compound of claim 9, wherein the nucleobase sequence of the modified oligonucleotide comprises or consists of the nucleobase sequence selected from:
   SEQ ID NOs: 70, 71, 72 and 75;
   SEQ ID NOs: 76, 81, 83, 84, and 85; and
   SEQ ID NOs: 88, 89, 90, and 92.
Embodiment 12. An oligomeric compound, wherein the oligomeric compound comprises a modified oligonucleotide consisting of 12 to 50 linked nucleosides, wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, at least 21, at least 22, or 23 contiguous nucleobases of
   an equal length portion of nucleobases 495-517 of SEQ ID NO: 1;
   an equal length portion of nucleobases 1375-1397 of SEQ ID NO: 1;
   an equal length portion of nucleobases 1475-1517 of SEQ ID NO: 1; or
   an equal length portion of nucleobases 1555-1577 of SEQ ID NO: 1.
Embodiment 13. The oligomeric compound of claim 12, wherein the nucleobase sequence of the modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of a nucleobase sequence selected from:
   SEQ ID NO: 29;
   SEQ ID NO: 75;
   SEQ ID NOs: 76 and 81; and
   SEQ ID NO: 83.
Embodiment 14. The oligomeric compound of claim 12, wherein the nucleobase sequence of the modified oligonucleotide comprises or consists of the nucleobase sequence selected from:
   SEQ ID NO: 29;
   SEQ ID NO: 75;
   SEQ ID NOs: 76 and 81; and
   SEQ ID NO: 83.
Embodiment 15. The oligomeric compound of any of claims 1-14, wherein at least one nucleoside of the modified oligonucleotide comprises a modified sugar moiety.
Embodiment 16. The oligomeric compound of claim 15, wherein the modified sugar moiety is a bicyclic sugar moiety.
Embodiment 17. The oligomeric compound of claim 16, wherein the bicyclic sugar moiety comprises a 2′-4′ bridge, wherein the 2′-4′ bridge is selected from —O—CH₂—; and —O—CH(CH₃)—.
Embodiment 18. The oligomeric compound of claim 15, wherein the modified sugar moiety is a non-bicyclic modified sugar moiety.
Embodiment 19. The oligomeric compound of claim 18, wherein the non-bicyclic modified sugar moiety is a 2′-MOE sugar moiety, a 2′-OMe sugar moiety, or a 2′-F sugar moiety.
Embodiment 20. The oligomeric compound of claim 15, wherein the modified sugar moiety is a sugar surrogate.
Embodiment 21. The oligomeric compound of claim 20, wherein the sugar surrogate is selected from morpholino, modified morpholino, glycol nucleic acid (GNA), six-membered tetrahydropyran (THP), and F-hexitol nucleic acid (F-HNA).
Embodiment 22. The oligomeric compound of any of claims 1-21, wherein the modified oligonucleotide comprises at least one modified internucleoside linkage.
Embodiment 23. The oligomeric compound of claim 22, wherein the at least one modified internucleoside linkage is a phosphorothioate internucleoside linkage.
Embodiment 24. The oligomeric compound of claim 22, wherein the at least one modified internucleoside linkage is a mesyl phosphoramidate internucleoside linkage.
Embodiment 25. The oligomeric compound of any of claims 1-24, wherein each internucleoside linkage of the modified oligonucleotide is independently selected from a phosphorothioate internucleoside linkage, a phosphodiester internucleoside linkage, and a mesyl phosphoramidate internucleoside linkage.
Embodiment 26. The oligomeric compound of any of claims 1-25, wherein each internucleoside linkage of the modified oligonucleotide is independently selected from a phosphodiester internucleoside linkage and a phosphorothioate internucleoside linkage.
Embodiment 27. The oligomeric compound of any of claims 1-25, wherein each internucleoside linkage of the modified oligonucleotide is independently selected from a phosphodiester internucleoside linkage and a mesyl phosphoramidate internucleoside linkage.
Embodiment 28. The oligomeric compound of any of claims 1-26, wherein the modified oligonucleotide has an internucleoside linkage motif of 5′-ssooooooooooooooooooss-3′, wherein each “s” is a phosphorothioate internucleoside linkage and each “o” is a phosphodiester internucleoside linkage.
Embodiment 29. The oligomeric compound of any of claims 1-28, wherein the modified oligonucleotide comprises at least one modified nucleobase.
Embodiment 30. The oligomeric compound of claim 29, wherein the modified nucleobase is 5-methylcytosine.
Embodiment 31. The oligomeric compound of claim 29 or claim 30, wherein each cytosine is a 5-methylcytosine.
Embodiment 32. The oligomeric compound of any of claims 1-28, wherein one or more cytosine nucleobases of the modified oligonucleotide are unmodified.
Embodiment 33. The oligomeric compound of any of claims 1-28, wherein all of the cytosine nucleobases of the modified oligonucleotide are unmodified.
Embodiment 34. The oligomeric compound of any of claims 1-33, wherein the modified oligonucleotide has a sugar motif of 5′-yfyfyfyfyfyfyfyfyfyfyyy-3′ or 5′-efyyyfyyyyyyyfyfyyyyyyy-3′, wherein “e” represents a 2′-MOE sugar moiety, each “y” represents a 2′-OMe sugar moiety, and each “f” represents a 2′-F sugar moiety.
Embodiment 35. The oligomeric compound of any of claims 1-34, wherein the oligomeric compound comprises a conjugate group.
Embodiment 36. The oligomeric compound of claim 35, wherein the conjugate group comprises a conjugate moiety and a conjugate linker.
Embodiment 37. The oligomeric compound of claim 36, wherein the conjugate moiety is a lipophilic group.
Embodiment 38. The oligomeric compound of claim 36 or claim 37, wherein the conjugate moiety is a lipophilic group selected from a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, and C5 alkenyl.
Embodiment 39. The oligomeric compound of any of claims 36-38, wherein the conjugate linker consists of a single bond.
Embodiment 40. The oligomeric compound of any of claims 36-39, wherein the conjugate linker is cleavable.
Embodiment 41. The oligomeric compound of any of claims 1-40, comprising a terminal group.
Embodiment 42. The oligomeric compound of claim 41, wherein the terminal group is a 5′-stabilized phosphate group.
Embodiment 43. The oligomeric compound of claim 42, wherein the 5′-stabilized phosphate group is selected from cyclopropylphosphonate and vinylphosphonate.
Embodiment 44. The oligomeric compound of any of claims 1-43, wherein the modified oligonucleotide is an antisense oligonucleotide.
Embodiment 45. The oligomeric compound of any of claims 1-44, wherein the modified oligonucleotide is an antisense RNAi oligonucleotide.
Embodiment 46. An oligomeric duplex, comprising a first oligomeric compound and a second oligomeric compound comprising a second modified oligonucleotide, wherein the first oligomeric compound is an oligomeric compound of any of claims 1-45.
Embodiment 47. The oligomeric duplex of claim 46, wherein the second oligomeric compound comprises a second modified oligonucleotide consisting of 12 to 50 linked nucleosides, and wherein the nucleobase sequence of the second modified oligonucleotide comprises a complementary region of at least 8, at least 9, at least 10, at least 11, at least 12, at least 13, at least 14, at least 15, at least 16, at least 17, at least 18, at least 19, at least 20, or 21 nucleobases that is at least 90% complementary to an equal length portion of the first modified oligonucleotide.
Embodiment 48. The oligomeric duplex of claim 46, wherein the second oligomeric compound comprises a second modified oligonucleotide consisting of 12 to 50 linked nucleosides, and wherein the nucleobase sequence of the second modified oligonucleotide comprises a complementary region of at least 8, at least 9, at least 10, at least 11, at least 12, at least 13, at least 14, at least 15, at least 16, at least 17, at least 18, at least 19, at least 20, or 21 nucleobases that is at least 95% complementary to an equal length portion of the first modified oligonucleotide.
Embodiment 49. The oligomeric duplex of claim 46, wherein the second oligomeric compound comprises a second modified oligonucleotide consisting of 12 to 50 linked nucleosides, and wherein the nucleobase sequence of the second modified oligonucleotide comprises a complementary region of at least 8, at least 9, at least 10, at least 11, at least 12, at least 13, at least 14, at least 15, at least 16, at least 17, at least 18, at least 19, at least 20, or 21 nucleobases that is 100% complementary to an equal length portion of the first modified oligonucleotide.
Embodiment 50. The oligomeric duplex of any of claims 46-49, wherein at least one nucleoside of the second modified oligonucleotide comprises a modified sugar moiety.
Embodiment 51. The oligomeric duplex of claim 50, wherein the modified sugar moiety is a bicyclic sugar moiety.
Embodiment 52. The oligomeric duplex of claim 51, wherein the bicyclic sugar moiety comprises a 2′-4′ bridge, wherein the 2′-4′ bridge is selected from —O—CH₂—; and —O—CH(CH₃)—.
Embodiment 53. The oligomeric duplex of claim 50, wherein the modified sugar moiety is a non-bicyclic modified sugar moiety.
Embodiment 54. The oligomeric duplex of claim 53, wherein the non-bicyclic modified sugar moiety is a 2′-MOE sugar moiety, a 2′-OMe sugar moiety, or a 2′-F sugar moiety.
Embodiment 55. The oligomeric duplex of claim 50, wherein the modified sugar moiety is a sugar surrogate.
Embodiment 56. The oligomeric duplex of claim 55, wherein the sugar surrogate is selected from morpholino, modified morpholino, glycol nucleic acid (GNA), six-membered tetrahydropyran (THP), and F-hexitol nucleic acid (F-HNA).
Embodiment 57. The oligomeric duplex of any of claims 46-56, wherein the second modified oligonucleotide comprises at least one modified internucleoside linkage.
Embodiment 58. The oligomeric duplex of claim 57, wherein the at least one modified internucleoside linkage is a phosphorothioate internucleoside linkage.
Embodiment 59. The oligomeric duplex of claim 57, wherein the at least one modified internucleoside linkage is a mesyl phosphoramidate internucleoside linkage.
Embodiment 60. The oligomeric duplex of any of claims 46-59, wherein each internucleoside linkage of the second modified oligonucleotide is independently selected from a phosphorothioate internucleoside linkage, a phosphodiester internucleoside linkage, and a mesyl phosphoramidate internucleoside linkage.
Embodiment 61. The oligomeric duplex of any of claims 46-60, wherein each internucleoside linkage of the second modified oligonucleotide is independently selected from a phosphodiester internucleoside linkage and a phosphorothioate internucleoside linkage.
Embodiment 62. The oligomeric duplex of any of claims 46-60, wherein each internucleoside linkage of the second modified oligonucleotide is independently selected from a phosphodiester internucleoside linkage and a mesyl phosphoramidate internucleoside linkage.
Embodiment 63. The oligomeric duplex of any of claims 46-61, wherein the second modified oligonucleotide has an internucleoside linkage motif of 5′-ssooooooooooooooooss-3′, wherein each “s” is a phosphorothioate internucleoside linkage and each “o” is a phosphodiester internucleoside linkage.
Embodiment 64. The oligomeric duplex of any of claims 46-63, wherein the second modified oligonucleotide comprises at least one modified nucleobase.
Embodiment 65. The oligomeric duplex of claim 64, wherein the modified nucleobase is 5-methylcytosine.
Embodiment 66. The oligomeric duplex of claim 64 or claim 65, wherein each cytosine is a 5-methylcytosine.
Embodiment 67. The oligomeric duplex of any of claims 46-63, wherein one or more cytosine nucleobases of the second modified oligonucleotide are unmodified.
Embodiment 68. The oligomeric duplex of any of claims 46-63, wherein all of the cytosine nucleobases of the second modified oligonucleotide are unmodified.
Embodiment 69. The oligomeric duplex of any of claims 46-68, wherein the second modified oligonucleotide has a sugar motif of 5′-fyfyfyfyfyfyfyfyfyfyf-3′ or 5′-yyyyyyfyfffyyyyyyyyyy-3′, wherein each “y” represents a 2′-OMe sugar moiety, and each “f” represents a 2′-F sugar moiety.
Embodiment 70. An oligomeric duplex comprising:
   a first oligomeric compound comprising a first modified oligonucleotide consisting of 18 to 30 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 16-166, 318, or 320-324; and
   a second oligomeric compound comprising a second modified oligonucleotide consisting of 15 to 29 linked nucleosides, wherein the nucleobase sequence of the second modified oligonucleotide comprises a complementary region of at least 8 nucleobases that is at least 90% complementary to the nucleobase sequence of an equal length portion of the first modified oligonucleotide.
Embodiment 71. An oligomeric duplex comprising:
   a first oligomeric compound comprising a first modified oligonucleotide consisting of 18 to 30 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 16-166, 318, or 320-324; and
   a second oligomeric compound comprising a second modified oligonucleotide consisting of 15 to 29 linked nucleosides, wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or 21 contiguous nucleobases of the nucleobase sequence of any of SEQ ID NOs: 167-317, 319, or 325-327, wherein the nucleobase sequence of the second modified oligonucleotide is at least 90% complementary to the nucleobase sequence of an equal length portion of the first modified oligonucleotide.
Embodiment 72. An oligomeric duplex comprising:
   a first oligomeric compound comprising a first modified oligonucleotide, wherein the first modified oligonucleotide consists of 23 linked nucleosides and has a nucleobase sequence consisting of the nucleobase sequence of any of SEQ ID NOs: 16-166, 318, or 320-324; and
   a second oligomeric compound comprising a second modified oligonucleotide, wherein the second modified oligonucleotide consists of 21 linked nucleosides, wherein the second modified oligonucleotide has a nucleobase sequence consisting of the nucleobase sequence of any of SEQ ID NOs: 167-317, 319, or 325-327, and wherein the nucleobase sequence of the second modified oligonucleotide is at least 90% complementary to the nucleobase sequence of an equal length portion of the first modified oligonucleotide.
Embodiment 73. The oligomeric duplex of any of claims 70-72, wherein the nucleobase sequence of the second modified oligonucleotide is at least 95% or 100% complementary to the nucleobase sequence of an equal length portion of the first modified oligonucleotide.
Embodiment 74. The oligomeric duplex of any of claims 70-73, wherein the modified oligonucleotide of the first oligomeric compound comprises a 5′-stabilized phosphate group.
Embodiment 75. The oligomeric duplex of claim 74, wherein the 5′-stabilized phosphate group comprises a cyclopropylphosphonate or a vinylphosphonate.
Embodiment 76. The oligomeric duplex of any of claims 70-75, wherein at least one nucleoside of the first modified oligonucleotide and at least one nucleoside of the second modified oligonucleotide each independently comprises a modified sugar moiety.
Embodiment 77. The oligomeric duplex of claim 76, wherein the modified sugar moiety is a bicyclic sugar moiety.
Embodiment 78. The oligomeric duplex of claim 77, wherein the bicyclic sugar moiety comprises a 2′-4′ bridge, wherein the 2′-4′ bridge is selected from —O—CH₂—; and —O—CH(CH₃)—.
Embodiment 79. The oligomeric duplex of any of claims 76-78, wherein at least one nucleoside of the first modified oligonucleotide and at least one nucleoside of the second modified oligonucleotide each independently comprises a non-bicyclic modified sugar moiety.
Embodiment 80. The oligomeric duplex of claim 79, wherein the non-bicyclic modified sugar moiety is a 2′-MOE sugar moiety, a 2′-OMe sugar moiety, or a 2′-F sugar moiety.
Embodiment 81. The oligomeric duplex of any of claims 76-80, wherein at least one nucleoside of the first modified oligonucleotide or the second modified oligonucleotide each independently comprises a sugar surrogate.
Embodiment 82. The oligomeric duplex of claim 81, wherein the sugar surrogate is selected from morpholino, modified morpholino, glycol nucleic acid (GNA), six-membered tetrahydropyran (THP), and F-hexitol nucleic acid (F-HNA).
Embodiment 83. The oligomeric duplex of any of claims 70-82, wherein the first modified oligonucleotide comprises at least one modified internucleoside linkage.
Embodiment 84. The oligomeric duplex of any of claims 70-83, wherein the second modified oligonucleotide comprises at least one modified internucleoside linkage.
Embodiment 85. The oligomeric duplex of claim 83 or claim 84, wherein the at least one modified internucleoside linkage is a phosphorothioate internucleoside linkage.
Embodiment 86. The oligomeric duplex of claim 83 or claim 84, wherein the at least one modified internucleoside linkage is a mesyl phosphoramidate internucleoside linkage.
Embodiment 87. The oligomeric duplex of claim 83 or claim 85, wherein each internucleoside linkage of the first modified oligonucleotide is independently selected from a phosphodiester internucleoside linkage and a phosphorothioate internucleoside linkage.
Embodiment 88. The oligomeric duplex of claim 83 or claim 86, wherein each internucleoside linkage of the first modified oligonucleotide is independently selected from a phosphodiester internucleoside linkage and a mesyl phosphoramidate internucleoside linkage.
Embodiment 89. The oligomeric duplex of any of claims 83, 84, 85, 87, or 88, wherein each internucleoside linkage of the second modified oligonucleotide is independently selected from a phosphodiester internucleoside linkage and a phosphorothioate internucleoside linkage.
Embodiment 90. The oligomeric duplex of any of claims 83, 84, 86, 87, or 88, wherein each internucleoside linkage of the second modified oligonucleotide is independently selected from a phosphodiester internucleoside linkage and a mesyl phosphoramidate internucleoside linkage.
Embodiment 91. The oligomeric duplex of any of claims 70-90, wherein the first modified oligonucleotide has an internucleoside linkage motif of 5′-ssooooooooooooooooooss-3′, wherein each “s” is a phosphorothioate internucleoside linkage and each “o” is a phosphodiester internucleoside linkage.
Embodiment 92. The oligomeric duplex of any of claims 70-91, wherein the second modified oligonucleotide has an internucleoside linkage motif of 5′-ssooooooooooooooooss-3′, wherein each “s” is a phosphorothioate internucleoside linkage and each “o” is a phosphodiester internucleoside linkage.
Embodiment 93. The oligomeric duplex of any of claims 70-92, wherein the first modified oligonucleotide and the second modified oligonucleotide each independently comprises at least one modified nucleobase.
Embodiment 94. The oligomeric duplex of claim 93, wherein the at least one modified nucleobase is 5-methylcytosine.
Embodiment 95. The oligomeric duplex of claim 93 or claim 94, wherein each cytosine is a 5-methylcytosine.
Embodiment 96. The oligomeric duplex of any of claims 70-92, wherein one or more cytosine nucleobases of the first modified oligonucleotide and/or of the second modified oligonucleotide are unmodified.
Embodiment 97. The oligomeric duplex of any of claims 70-92, wherein cytosine nucleobases of the first modified oligonucleotide or of the second modified oligonucleotide are unmodified.
Embodiment 98. The oligomeric duplex of any of claims 70-92, wherein cytosine nucleobases of the first modified oligonucleotide and of the second modified oligonucleotide are unmodified.
Embodiment 99. The oligomeric duplex of any of claims 70-98, wherein at least one nucleoside of the first modified oligonucleotide comprises a 2′-F sugar moiety and the at least one nucleoside is at:
position 2 or 14 from the 5′ end;
position 2, 6, or 14 from the 5′ end;
position 2, 14, or 16 from the 5′ end;
position 2, 6, 14, or 16 from the 5′ end; or position 2, 6, 8, 9, 14, or 16 from the 5′ end.
Embodiment 100. The oligomeric duplex of any of claims 70-99, wherein the nucleosides of the first modified oligonucleotide each comprises a 2′-F sugar moiety and the nucleosides are at:
positions 2 and 14 from the 5′ end;
positions 2, 6, and 14 from the 5′ end;
positions 2, 14, and 16 from the 5′ end;
positions 2, 6, 14, and 16 from the 5′ end; or
positions 2, 6, 8, 9, 14, and 16 from the 5′ end.
Embodiment 101. The oligomeric duplex of any of claims 70-100, wherein at least one nucleoside of the second modified oligonucleotide comprises a 2′-F sugar moiety and the at least one nucleoside is at:
position 9, 10, or 11 from the 5′ end;
position 7, 9, 10, or 11 from the 5′ end;
position 11, 12, or 15 from the 5′ end; or position 7, 9, 10, 11, 12, or 15 from the 5′ end.
Embodiment 102. The oligomeric duplex of any of claims 70-101, wherein the nucleosides of the second modified oligonucleotide each comprises a 2′-F sugar moiety and the nucleosides are at:
positions 9, 10, and 11 from the 5′ end; or
positions 7, 9, 10, and 11 from the 5′ end.
Embodiment 103. The oligomeric duplex of claim 99 or claim 100, wherein the nucleosides at the remaining positions of the first modified oligonucleotide each comprises a 2′-OMe sugar moiety.
Embodiment 104. The oligomeric duplex of any of claims 99-103, wherein the nucleosides at the remaining positions of the second modified oligonucleotide each comprises a 2′-OMe sugar moiety.
Embodiment 105. The oligomeric duplex of any of claims 70-98, wherein the first modified oligonucleotide has a sugar motif of 5′-yfyfyfyfyfyfyfyfyfyfyyy-3′ or 5′-efyyyfyyyyyyyfyfyyyyyyy-3′, wherein “e” represents a 2′-MOE sugar moiety, each “y” represents a 2′-OMe sugar moiety, and each “f” represents a 2′-F sugar moiety.
Embodiment 106. The oligomeric duplex of any of claims 70-98 or 105, wherein the second modified oligonucleotide has a sugar motif of 5′-fyfyfyfyfyfyfyfyfyfyf-3′ or 5′-yyyyyyfyfffyyyyyyyyyy-3′, wherein each “y” represents a 2′-OMe sugar moiety, and each “f” represents a 2′-F sugar moiety.
Embodiment 107. The oligomeric duplex of any of claims 70-106, wherein the second modified oligonucleotide comprises a conjugate group.
Embodiment 108. The oligomeric duplex of claim 107, wherein the conjugate group comprises a conjugate linker and a conjugate moiety.
Embodiment 109. The oligomeric duplex of claim 107 or claim 108, wherein the conjugate group is attached to the second modified oligonucleotide at the 5′-end of the second modified oligonucleotide.
Embodiment 110. The oligomeric duplex of claim 107 or claim 108, wherein the conjugate group is attached to the second modified oligonucleotide at the 3′-end of the second modified oligonucleotide.
Embodiment 111. The oligomeric duplex of claim 107 or claim 108, wherein the conjugate group is attached to the second modified oligonucleotide at the 2′ position of a furanosyl sugar moiety.
Embodiment 112. The oligomeric duplex of claim 107 or claim 108, wherein the conjugate group is attached to the second modified oligonucleotide through a modified internucleoside linkage.
Embodiment 113. The oligomeric duplex of any of claims 107-112, wherein the conjugate group comprises a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.
Embodiment 114. The oligomeric duplex of any of claims 45-113, wherein the second modified oligonucleotide comprises a terminal group.
Embodiment 115. The oligomeric duplex of claim 114, wherein the terminal group is an abasic sugar moiety.
Embodiment 116. The oligomeric duplex of any of claims 46-115, wherein the second modified oligonucleotide consists of 10 to 25, 10 to 30, 12 to 20, 12 to 25, 12 to 30, 13 to 20, 13 to 25, 13 to 30, 14 to 20, 14 to 25, 14 to 30, 15 to 20, 15 to 25, 15 to 30, 16 to 18, 16 to 20, 16 to 25, 16 to 30, 17 to 20, 17 to 25, 17 to 30, 18 to 20, 18 to 25, 18 to 30, 19 to 20, 19 to 25, 19 to 30, 20 to 25, 20 to 30, 21 to 25, 21 to 30, 22 to 25, 22 to 30, 23 to 25, or 23 to 30 linked nucleosides.
Embodiment 117. The oligomeric duplex of any of claims 46-71 or 73-116, wherein the first modified oligonucleotide consists of 23 linked nucleosides and the second modified oligonucleotide consists of 21 linked nucleosides.
Embodiment 118. An antisense agent, wherein the antisense agent is the oligomeric duplex of any of claims 46-117.
Embodiment 119. The antisense agent of claim 118, wherein the antisense agent is an RNAi agent capable of reducing the amount of SNCA through the activation of RISC/Ago2.
Embodiment 120. A population of oligomeric duplexes of claims 46-117, wherein the population is chirally enriched for modified oligonucleotides comprising at least one particular phosphorothioate internucleoside linkage having a particular stereochemical configuration.
Embodiment 121. The population of claim 120, wherein the population is chirally enriched for modified oligonucleotides having a particular, independently selected stereochemical configuration at each phosphorothioate internucleoside linkage.
Embodiment 122. The population of claim 120, wherein the population is chirally enriched for modified oligonucleotides having the (Rp) configuration at one particular phosphorothioate internucleoside linkage and the (Sp) configuration at each of the remaining phosphorothioate internucleoside linkages, or the population is chirally enriched for modified oligonucleotides having the (Sp) configuration at each phosphorothioate internucleoside linkage or for modified oligonucleotides having the (Rp) configuration at each phosphorothioate internucleoside linkage.
Embodiment 123. A population of oligomeric compounds comprising modified oligonucleotides of any of claims 1-45, wherein all of the phosphorothioate internucleoside linkages of the modified oligonucleotides are stereorandom.
Embodiment 124. A population of oligomeric duplexes of any of claims 46-117, wherein all of the phosphorothioate internucleoside linkages of the modified oligonucleotides of the first oligomeric compound are stereorandom.
Embodiment 125. The population of oligomeric duplexes of claim 124, wherein all of the phosphorothioate internucleoside linkages of the modified oligonucleotides of the second oligomeric compound are stereorandom.
Embodiment 126. A pharmaceutical composition comprising the oligomeric compound of any of claims 1-45, the oligomeric duplex of any of claims 46-117, the antisense agent of any of claims 118-119, or the population of any of claims 120-125, and a pharmaceutically acceptable diluent or carrier.
Embodiment 127. The pharmaceutical composition of claim 126, wherein the pharmaceutically acceptable diluent is phosphate buffered saline (PBS) or artificial cerebrospinal fluid (aCSF).
Embodiment 128. The pharmaceutical composition of claim 127, wherein the pharmaceutical composition consists essentially of the oligomeric compound of any of claims 1-45, the oligomeric duplex of any of claims 46-117, the antisense agent of any of claims 118-119, or the population of any of claims 120-125, and aCSF.
Embodiment 129. The pharmaceutical composition of claim 127, wherein the pharmaceutical composition consists essentially of the oligomeric compound of any of claims 1-45, the oligomeric duplex of any of claims 46-117, the antisense agent of any of claims 118-119, or the population of any of claims 120-125, and PBS.
Embodiment 130. A method comprising administering to a subject the oligomeric compound of any of claims 1-45, the oligomeric duplex of any of claims 46-117, the antisense agent of any of claims 118-119, the population of any of claims 120-125, or the pharmaceutical composition of any of claims 126-129.
Embodiment 131. The method of claim 130, wherein the subject has a disease or disorder associated with SNCA.
Embodiment 132. The method of claim 130, wherein the subject has a synucleinopathy.
Embodiment 133. The method of claim 130, wherein the subject has Parkinson's disease.
Embodiment 134. The method of claim 130, wherein the subject has dementia with Lewy bodies, diffuse Lewy body disease, Parkinson's disease dementia, pure autonomic failure, multiple system atrophy, neuronopathic Gaucher's disease, or Alzheimer's disease.
Embodiment 135. A method of treating a disease or disorder associated with SNCA comprising administering to a subject having or at risk of developing a disease or disorder associated with SNCA a therapeutically effective amount of the oligomeric compound of any of claims 1-45, the oligomeric duplex of any of claims 46-117, the antisense agent of any of claims 118-119, the population of any of claims 120-125, or the pharmaceutical composition of any of claims 126-129, thereby treating the disease or disorder associated with SNCA.
Embodiment 136. The method of claim 135, wherein the disease or disorder associated with SNCA is a neurodegenerative disease.
Embodiment 137. The method of claim 135 or claim 136, wherein the disease or disorder associated with SNCA is a synucleinopathy.
Embodiment 138. The method of claim 136 or claim 137, wherein the neurodegenerative disease or synucleinopathy is any of Parkinson's disease, dementia with Lewy bodies, diffuse Lewy body disease, Parkinson's disease dementia, pure autonomic failure, multiple system atrophy, neuronopathic Gaucher's disease, and Alzheimer's disease.
Embodiment 139. The method of claim 135 or claim 136, wherein the disease or disorder associated with SNCA is Parkinson's disease.
Embodiment 140. The method of any of claims 135-139, wherein at least one symptom or hallmark is ameliorated.
Embodiment 141. The method of claim 140, wherein the at least one symptom or hallmark is motor dysfunction, aggregation of alpha-synuclein, neurodegeneration, cognitive decline, dementia, sleep disorders, hyposmia, autonomic failure, ataxia, hallucination, or seizures.
Embodiment 142. The method of claim 140 or claim 141, wherein administering the oligomeric compound of any of claims 1-45, the oligomeric duplex of any of claims 46-117, the antisense agent of any of claims 118-119, the population of any of claims 120-125, or the pharmaceutical composition of any of claims 126-129 improves motor function, reduces the amount or volume of alpha-synuclein aggregates, reduces or delays neurodegeneration, improves cognitive function, delays the onset or progression of dementia, reduces sleep disorders, improves hyposmia, reduces or delays autonomic failure, reduces ataxia, reduces hallucination, or reduces seizures.
Embodiment 143. The method of any of claims 130-142, wherein the oligomeric compound of any of claims 1-45, the oligomeric duplex of any of claims 46-117, the antisense agent of any of claims 118-119, the population of any of claims 120-125, or the pharmaceutical composition of any of claims 126-129 is administered to the central nervous system or systemically.
Embodiment 144. The method of any of claims 130-143, wherein the oligomeric compound of any of claims 1-45, the oligomeric duplex of any of claims 46-117, the antisense agent of any of claims 118-119, the population of any of claims 120-125, or the pharmaceutical composition of any of claims 126-129 is administered intrathecally.
Embodiment 145. The method of any of claims 130-144, wherein the subject is a human.
Embodiment 146. A method of reducing SNCA RNA in a cell comprising contacting the cell with the oligomeric compound of any of claims 1-45, the oligomeric duplex of any of claims 46-117, the antisense agent of any of claims 118-119, the population of any of claims 120-125, or the pharmaceutical composition of any of claims 126-129.
Embodiment 147. A method of reducing alpha-synuclein protein in a cell comprising contacting the cell with the oligomeric compound of any of claims 1-45, the oligomeric duplex of any of claims 46-117, the antisense agent of any of claims 118-119, the population of any of claims 120-125, or the pharmaceutical composition of any of claims 126-129.
Embodiment 148. The method of claim 146 or claim 147, wherein the cell is a central nervous system cell.
Embodiment 149. The method of claim 146 or claim 147, wherein the cell is a brain cell.
Embodiment 150. The method of claim 146 or claim 147, wherein the cell is a neuron or an oligodendrocyte.
Embodiment 151. The method of any of claims 146-150, wherein the cell is a human cell.
Embodiment 152. Use of the oligomeric compound of any of claims 1-45, the oligomeric duplex of any of claims 46-117, the antisense agent of any of claims 118-119, the population of any of claims 120-125, or the pharmaceutical composition of any of claims 126-129 for treating a disease or disorder associated with SNCA.
Embodiment 153. Use of the oligomeric compound of any of claims 1-45, the oligomeric duplex of any of claims 46-117, the antisense agent of any of claims 118-119, the population of any of claims 120-125, or the pharmaceutical composition of any of claims 126-129 for the manufacture of a medicament for treating a disease or disorder associated with SNCA.
Embodiment 154. The use of claim 152 or claim 153, wherein the disease or disorder associated with SNCA is a neurodegenerative disease.
Embodiment 155. The use of any of claims 152-154, wherein the disease or disorder associated with SNCA is a synucleinopathy.
Embodiment 156. The use of any of claims 152-155, wherein the disease or disorder associated with SNCA is Parkinson's disease.
Embodiment 157. The use of any of claims 152-155, wherein the disease or disorder associated with SNCA is dementia with Lewy bodies, diffuse Lewy body disease, Parkinson's disease dementia, pure autonomic failure, multiple system atrophy, neuronopathic Gaucher's disease, or Alzheimer's disease.
Embodiment 158. The use of any of claims 152-157, wherein at least one symptom or hallmark is ameliorated.
Embodiment 159. The use of claim 158, wherein the at least one symptom or hallmark is motor dysfunction, aggregation of alpha-synuclein, neurodegeneration, cognitive decline, dementia, sleep disorders, hyposmia, autonomic failure, ataxia, hallucination, or seizures.
Embodiment 160. The use of claim 158 or claim 159, wherein the use of the oligomeric compound of any of claims 1-45, the oligomeric duplex of any of claims 46-117, the antisense agent of any of claims 118-119, the population of any of claims 120-125, or the pharmaceutical composition of any of claims 126-129 improves motor function, reduces the amount or volume of alpha-synuclein aggregates, reduces or delays neurodegeneration, improves cognitive function, delays the onset or progression of dementia, reduce sleep disorders, improves hyposmia, reduces or delays autonomic failure, reduces ataxia, reduces hallucination, or reduces seizures.

### I. Certain Oligonucleotides

Provided herein are oligomeric compounds comprising antisense oligonucleotides complementary to a SNCA RNA and optionally, sense oligonucleotides complementary to the antisense oligonucleotides. Antisense oligonucleotides and sense oligonucleotides typically comprise at least one modified nucleoside and/or at least one modified internucleoside linkage. Certain modified nucleosides and modified internucleoside linkages suitable for use in antisense oligonucleotides and/or sense oligonucleotides are described below.

### A. Certain Modified Nucleosides

Modified nucleosides comprise a modified sugar moiety or a modified nucleobase or both a modified sugar moiety and a modified nucleobase. Modified nucleosides comprising the following modified sugar moieties and/or the following modified nucleobases may be incorporated into antisense oligonucleotides and/or sense oligonucleotides.

### 1. Modified Sugar Moieties

In certain embodiments, sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including, but not limited, to substituents at the 2′, 3′, 4′, and/or 5′ positions. In certain embodiments, the furanosyl sugar moiety is a ribosyl sugar moiety.

In certain embodiments, one or more acyclic substituent of non-bicyclic modified sugar moieties is branched.

In certain embodiments, non-bicyclic modified sugar moieties comprise a substituent group at the 2′-position. Examples of substituent groups suitable for the 2′-position of modified sugar moieties include but are not limited to: —F, —OCH₃ (“OMe” or “O-methyl”), and OCH₂CH₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂ON(CH₃)₂ (“DMAOE”), 2′-O(CH₂)₂O(CH₂)₂N(CH₃)₂ (“DMAEOE”), and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted sugar moiety of a modified nucleoside comprises 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, O(CH₂)₂ON(CH₃)₂ (“DMAOE”), O(CH₂)₂O(CH₂)₂N(CH₃)₂ (“DMAEOE”), and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted sugar moiety of a modified nucleoside comprises 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. Modified furanosyl sugar moieties described herein are in the β-D-ribosyl isomeric configuration unless otherwise specified.

In certain embodiments, non-bicyclic modified sugar moieties comprise a substituent group at the 4′-position. Examples of substituent groups suitable for the 4′-position of modified sugar moieties include but are not limited to alkoxy (e.g., methoxy), alkyl, and those described in Manoharan et al., WO 2015/106128.

In certain embodiments, non-bicyclic modified sugar moieties comprise a substituent group at the 3′-position. Examples of substituent groups suitable for the 3′-position of modified sugar moieties include but are not limited to alkoxy (e.g., methoxy), alkyl (e.g., methyl, ethyl).

In certain embodiments, non-bicyclic modified sugar moieties comprise a substituent group at the 5′-position. Examples of substituent groups suitable for the 5′-position of modified sugar moieties include but are not limited to vinyl, alkoxy (e.g., methoxy), alkyl (e.g., methyl (R or S), ethyl).

In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In naturally occurring nucleic acids, sugars are linked to one another 3′ to 5′. In certain embodiments, oligonucleotides include one or more nucleoside or sugar moiety linked at an alternative position, for example at the 2′ position or inverted 5′ to 3′. For example, where the linkage is at the 2′ position, the 2′-substituent groups may instead be at the 3′-position.

Certain modified sugar moieties comprise a substituent that bridges two atoms of the furanosyl ring to form a second ring, resulting in a bicyclic sugar moiety. In certain embodiments, the bicyclic sugar moiety comprises a bridge between the 4′ and the 2′ furanose ring atoms. Examples of such 4′ to 2′ bridging sugar substituents include, but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C1-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ, —[C(Rₐ)(R_{b})]ₙO—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Wahlestedt et al., Proc. Natl. Acad. Sci. U.S.A, 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Elayadi et al., Curr. Opinion Invens. Drugs, 2001, 2, 558-561; Braasch et al., Chem. Biol., 2001, 8, 1-7; Orum et al., Curr. Opinion Mol. Ther., 2001, 3,239-243; Wengel et al., U.S. Pat. No. 7,053,207, Imanishi et al., U.S. Pat. No. 6,268,490, Imanishi et al. U.S. Pat. No. 6,770,748, Imanishi et al., U.S. RE44,779; Wengel et al., U.S. Pat. No. 6,794,499, Wengel et al., U.S. Pat. No. 6,670,461; Wengel et al., U.S. Pat. No. 7,034,133, Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191, Torsten et al., WO 2004/106356, Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; Allerson et al., US2008/0039618; and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

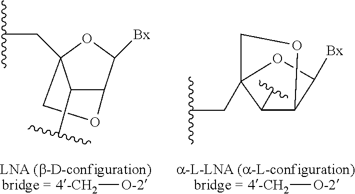

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mal Canc Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include, but are not limited to, hexitol nucleic acid (“HNA”), anitol nucleic acid (“ANA”), manitol nucleic acid (“MNA”) (see e.g., Leumann, CJ. Bioorg. & Med. Chem. 2002, 10, 841-854), fluoro HNA:

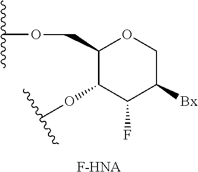

(“F-HNA”, see e.g., Swayze et al., U.S. Pat. No. 8,088,904; Swayze et al., U.S. Pat. No. 8,440,803; and Swayze et al., U.S. Pat. No. 9,005,906) F-HNA can also be referred to as a F-THP or 3′-fluoro tetrahydropyran, and nucleosides comprising additional modified THP compounds having the formula:

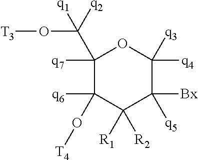

wherein, independently, for each of said modified THP nucleoside:
Bx is a nucleobase moiety;
T₃ and T₄ are each, independently, an intemucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C1-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

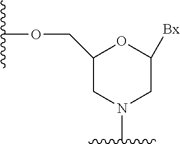

In certain embodiments, morpholinos may be modified, for example, by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378. Representative U.S. patents that teach the preparation of PNA compounds include, but are not limited to, U.S. Pat. Nos. 5,539,082; 5,714,331; and 5,719,262. Additional PNA compounds suitable for use in the RNAi oligonucleotides are described in, for example, in Nielsen et al., Science, 1991, 254, 1497-1500.

In certain embodiments, sugar surrogates are the “unlocked” sugar structure of UNA (unlocked nucleic acid) nucleosides. UNA is a nucleoside wherein any of the bonds of the sugar moiety has been removed, forming an unlocked sugar surrogate. Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

In certain embodiments, sugar surrogates are the glycerol as found in GNA (glycol nucleic acid) nucleosides as depicted below:

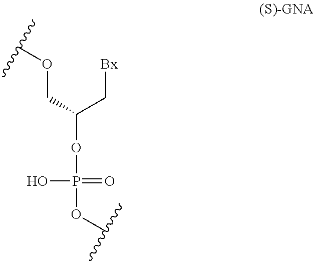

where Bx represents any nucleobase.

Many other modified sugar moieties and sugar surrogates are known in the art that can be used in modified nucleosides.

### 2. Certain Modified Nucleobases

In certain embodiments, oligonucleotides comprise one or more nucleoside comprising a modified nucleobase. In certain embodiments, oligonucleotides comprise one or more inosine nucleosides (i.e., nucleosides comprising a hypoxantine nucleobase).

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C═C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angew. Chem., Int. Ed., 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases, as well as other modified nucleobases include without limitation, Manoharan et al., US2003/0158403, Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., U.S. Pat. No. 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphodiesters, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS-P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphodiester internucleoside linkages, can alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO/2021/030778, incorporated by reference herein. In certain embodiments, a modified internucleoside linkage comprises the formula:

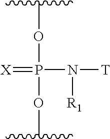

wherein independently for each internucleoside linking group of the modified oligonucleotide:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.

In certain embodiments, a modified internucleoside linkage comprises a mesyl phosphoramidate linking group having a formula:

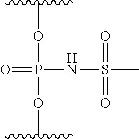 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

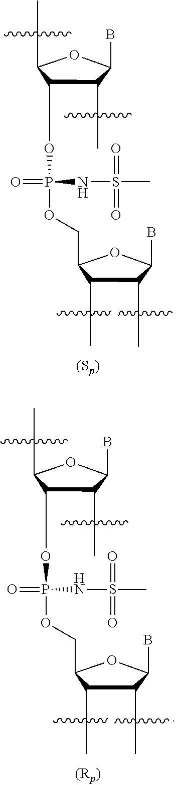

Representative internucleoside linkages having a chiral center include but are not limited to alkylphosphonates, mesyl phosphoramidates, and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate or other linkages containing chiral centers in particular stereochemical configurations. In certain embodiments, populations of modified oligonucleotides comprise phosphorothioate internucleoside linkages wherein all of the phosphorothioate internucleoside linkages are stereorandom. In certain embodiments, populations of modified oligonucleotides comprise mesyl phosphoramidate internucleoside linkages wherein all of the mesyl phosphoramidate internucleoside linkages are stereorandom. Such modified oligonucleotides can be generated using synthetic methods that result in random selection of the stereochemical configuration of each phosphorothioate or mesyl phosphoramidate linkage. Nonetheless, each individual phosphorothioate or mesyl phosphoramidate of each individual oligonucleotide molecule has a defined stereoconfiguration. In certain embodiments, populations of modified oligonucleotides are enriched for modified oligonucleotides comprising one or more particular phosphorothioate or mesyl phosphoramidate internucleoside linkages in a particular, independently selected stereochemical configuration. In certain embodiments, the particular configuration of the particular phosphorothioate or mesyl phosphoramidate linkage is present in at least 65% of the molecules in the population. In certain embodiments, the particular configuration of the particular phosphorothioate or mesyl phosphoramidate linkage is present in at least 70% of the molecules in the population. In certain embodiments, the particular configuration of the particular phosphorothioate or mesyl phosphoramidate linkage is present in at least 80% of the molecules in the population. In certain embodiments, the particular configuration of the particular phosphorothioate or mesyl phosphoramidate linkage is present in at least 90% of the molecules in the population. In certain embodiments, the particular configuration of the particular phosphorothioate or mesyl phosphoramidate linkage is present in at least 99% of the molecules in the population. Such chirally enriched populations of modified oligonucleotides can be generated using synthetic methods known in the art, e.g., methods described in Oka et al., JACS 2003, 125, 8307, Wan et al. Nucleic Acids Res. 2014, 42, 13456, and WO 2017/015555. In certain embodiments, a population of modified oligonucleotides is enriched for modified oligonucleotides having at least one indicated phosphorothioate or mesyl phosphoramidate in the (Sp) configuration. In certain embodiments, a population of modified oligonucleotides is enriched for modified oligonucleotides having at least one phosphorothioate or mesyl phosphoramidate in the (Rp) configuration. In certain embodiments, modified oligonucleotides comprising (Rp) and/or (Sp) phosphorothioates comprise one or more of the following formulas, respectively, wherein “B” indicates a nucleobase:

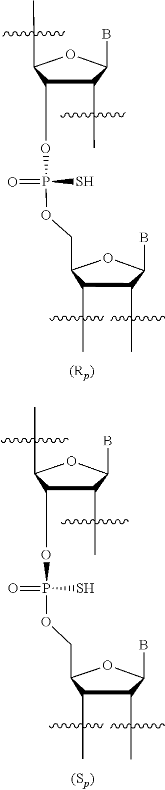

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See, for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, oligonucleotides (such as antisense oligonucleotides and/or sense oligonucleotides) comprise one or more inverted nucleoside, as shown below:

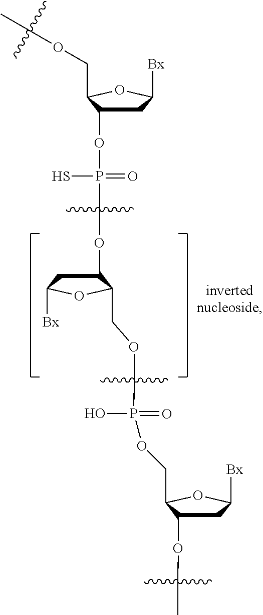

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

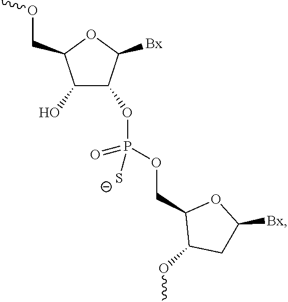

wherein each Bx represents any nucleobase.

### B. Antisense Oligonucleotides

In certain embodiments, antisense oligonucleotides comprise a number of linked nucleosides, wherein certain nucleosides and/or linkages are modified.

### 1. Certain Lengths

In certain embodiments, antisense oligonucleotides consist of 12-30 linked nucleosides. In certain embodiments, antisense oligonucleotides consist of 17-25 linked nucleosides. In certain embodiments, antisense oligonucleotides consist of 17-23 linked nucleosides. In certain embodiments, antisense oligonucleotides consist of 17-21 linked nucleosides. In certain embodiments, antisense oligonucleotides consist of 18-30 linked nucleosides. In certain embodiments, antisense oligonucleotides consist of 20-30 linked nucleosides. In certain embodiments, antisense oligonucleotides consist of 21-30 linked nucleosides. In certain embodiments, antisense oligonucleotides consist of 23-30 linked nucleosides. In certain embodiments, antisense oligonucleotides consist of 18-25 linked nucleosides. In certain embodiments, antisense oligonucleotides consist of 20-22 linked nucleosides. In certain embodiments, antisense oligonucleotides consist of 21-23 linked nucleosides. In certain embodiments, antisense oligonucleotides consist of 23-24 linked nucleosides. In certain embodiments, antisense oligonucleotides consist of 20 linked nucleosides. In certain embodiments, antisense oligonucleotides consist of 21 linked nucleosides. In certain embodiments, antisense oligonucleotides consist of 22 linked nucleosides. In certain embodiments, antisense oligonucleotides consist of 23 linked nucleosides.

### 2. Certain Sugar Motifs

In certain embodiments, the sugar moiety of at least one nucleoside of an antisense oligonucleotide is a modified sugar moiety.

In certain embodiments, at least one nucleoside comprises a 2′-OMe sugar moiety. In certain embodiments, at least 2 nucleosides comprise 2′-OMe sugar moieties. In certain embodiments, at least 5 nucleosides comprise 2′-OMe sugar moieties. In certain embodiments, at least 8 nucleosides comprise 2′-OMe sugar moieties. In certain embodiments, at least 10 nucleosides comprise 2′-OMe sugar moieties. In certain embodiments, at least 12 nucleosides comprise 2′-OMe sugar moieties. In certain embodiments, at least 13 nucleosides comprise 2′-OMe sugar moieties. In certain embodiments, at least 14 nucleosides comprise 2′-OMe sugar moieties. In certain embodiments, at least 15 nucleosides comprise 2′-OMe sugar moieties. In certain embodiments, at least 17 nucleosides comprise 2′-OMe sugar moieties. In certain such embodiments, at least 18 nucleosides comprise 2′-OMe sugar moieties. In certain such embodiments, at least 20 nucleosides comprise 2′-OMe sugar moieties. In certain such embodiments, at least 21 nucleosides comprise 2′-OMe sugar moieties.

In certain embodiments, at least one nucleoside comprises a 2′-F sugar moiety. In certain embodiments, at least 2 nucleosides comprise 2′-F sugar moieties. In certain embodiments, at least 3 nucleosides comprise 2′-F sugar moieties. In certain embodiments, at least 4 nucleosides comprise 2′-F sugar moieties. In certain embodiments, at least 6 nucleosides comprise 2′-F sugar moieties. In certain embodiments, at least 8 nucleosides comprise 2′-F sugar moieties. In certain embodiments, at least 10 nucleosides comprise 2′-F sugar moieties. In certain embodiments, at least 11 nucleosides comprise 2′-F sugar moieties. In certain embodiments, at least 12 nucleosides comprise 2′-F sugar moieties. In certain embodiments, one, but not more than one nucleoside comprises a 2′-F sugar moiety. In certain embodiments, 1 or 2 nucleosides comprise 2′-F sugar moieties. In certain embodiments, 1-3 nucleosides comprise 2′-F sugar moieties. In certain embodiments, at least 1-4 nucleosides comprise 2′-F sugar moieties. In certain embodiments, antisense oligonucleotides have a block of 2-4 contiguous 2′-F modified nucleosides. In certain embodiments, 4 nucleosides of an antisense oligonucleotide are 2′-F modified nucleosides and 3 of those 2′-F modified nucleosides are contiguous. In certain such embodiments the remainder of the nucleosides are 2′OMe modified.

In certain embodiments, one nucleoside of an antisense oligonucleotide is a UNA.

In certain embodiments, one nucleoside of an antisense oligonucleotide is a GNA.

In certain embodiments, 1-4 nucleosides of an antisense oligonucleotide is/are DNA. In certain such embodiments, the 1-4 DNA nucleosides are at one or both ends of the antisense oligonucleotide.

### 3. Certain Internucleoside Linkages

In certain embodiments, at least one linkage of the antisense oligonucleotide is a modified linkage. In certain embodiments, the 5′-most linkage (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain embodiments, the modified linkage is a phosphorothioate linkage. In certain embodiments, the modified linkage is a mesyl phosphoramidate linkage. In certain embodiments, the remaining linkages are all unmodified phosphodiester linkages.

In certain embodiments, at least one linkage of the antisense oligonucleotide is an inverted linkage.

### C. Sense Oligonucleotides

In certain embodiments, sense oligonucleotides comprise a number of linked nucleosides, wherein certain nucleosides and/or linkages are modified.

### 1. Certain Lengths

In certain embodiments, sense oligonucleotides consist of 12-30 linked nucleosides. In certain embodiments, sense oligonucleotides consist of 17-25 linked nucleosides. In certain embodiments, sense oligonucleotides consist of 17-23 linked nucleosides. In certain embodiments, sense oligonucleotides consist of 17-21 linked nucleosides. In certain embodiments, sense oligonucleotides consist of 18-30 linked nucleosides. In certain embodiments, sense oligonucleotides consist of 20-30 linked nucleosides. In certain embodiments, sense oligonucleotides consist of 21-30 linked nucleosides. In certain embodiments, sense oligonucleotides consist of 23-30 linked nucleosides. In certain embodiments, sense oligonucleotides consist of 18-25 linked nucleosides. In certain embodiments, sense oligonucleotides consist of 20-22 linked nucleosides. In certain embodiments, sense oligonucleotides consist of 21-23 linked nucleosides. In certain embodiments, sense oligonucleotides consist of 23-24 linked nucleosides. In certain embodiments, sense oligonucleotides consist of 19 linked nucleosides. In certain embodiments, sense oligonucleotides consist of 20 linked nucleosides. In certain embodiments, sense oligonucleotides consist of 21 linked nucleosides. In certain embodiments, sense oligonucleotides consist of 22 linked nucleosides. In certain embodiments, sense oligonucleotides consist of 23 linked nucleosides. In certain embodiments, sense oligonucleotides consist of 25 linked nucleosides.

### 2. Certain Sugar Motifs

In certain embodiments, the sugar moiety of at least one nucleoside of a sense oligonucleotides is a modified sugar moiety.

In certain embodiments, at least one nucleoside comprises a 2′-OMe sugar moiety. In certain embodiments, at least 2 nucleosides comprise 2′-OMe sugar moieties. In certain embodiments, at least 5 nucleosides comprise 2′-OMe sugar moieties. In certain embodiments, at least 8 nucleosides comprise 2′-OMe sugar moieties. In certain embodiments, at least 10 nucleosides comprise 2′-OMe sugar moieties. In certain embodiments, at least 12 nucleosides comprise 2′-OMe sugar moieties. In certain embodiments, at least 14 nucleosides comprise 2′-OMe sugar moieties. In certain embodiments, at least 15 nucleosides comprise 2′-OMe sugar moieties. In certain embodiments, at least 17 nucleosides comprise 2′-OMe sugar moieties. In certain such embodiments, at least 18 nucleosides comprise 2′-OMe sugar moieties. In certain such embodiments, at least 20 nucleosides comprise 2′-OMe sugar moieties. In certain such embodiments, at least 21 nucleosides comprise 2′-OMe sugar moieties.

In certain embodiments, at least one nucleoside comprises a 2′-F sugar moiety. In certain embodiments, at least 2 nucleosides comprise 2′-F sugar moieties. In certain embodiments, at least 3 nucleosides comprise 2′-F sugar moieties. In certain embodiments, at least 4 nucleosides comprise 2′-F sugar moieties. In certain embodiments, at least 6 nucleosides comprise 2′-F sugar moieties. In certain embodiments, at least 8 nucleosides comprise 2′-F sugar moieties. In certain embodiments, at least 10 nucleosides comprise 2′-F sugar moieties. In certain embodiments, at least 11 nucleosides comprise 2′-F sugar moieties. In certain embodiments, at least 12 nucleosides comprise 2′-F sugar moieties. In certain embodiments, one, but not more than one nucleoside comprises a 2′-F sugar moiety. In certain embodiments, 1 or 2 nucleosides comprise 2′-F sugar moieties. In certain embodiments, 1-3 nucleosides comprise 2′-F sugar moieties. In certain embodiments, at least 1-4 nucleosides comprise 2′-F sugar moieties. In certain embodiments, sense oligonucleotides have a block of 2-4 contiguous 2′-F modified nucleosides. In certain embodiments, 4 nucleosides of a sense oligonucleotide are 2′-F modified nucleosides and 3 of those 2′-F modified nucleosides are contiguous. In certain such embodiments the remainder of the nucleosides are 2′OMe modified.

In certain embodiments, one nucleoside of a sense oligonucleotide is a UNA.

In certain embodiments, one nucleoside of a sense oligonucleotide is a GNA.

In certain embodiments, 1-4 nucleosides of a sense oligonucleotide is/are DNA. In certain such embodiments, the 1-4 DNA nucleosides are at one or both ends of the sense oligonucleotide.

### 3. Certain Internucleoside Linkages

In certain embodiments, at least one linkage of the sense oligonucleotides is a modified linkage. In certain embodiments, the 5′-most linkage (i.e., linking the first nucleoside from the 5′-end to the second nucleoside from the 5′-end) is modified. In certain embodiments, the two 5′-most linkages are modified. In certain embodiments, the first one or 2 linkages from the 3′-end are modified. In certain embodiments, the modified linkage is a phosphorothioate linkage. In certain embodiments, the modified linkage is a mesyl phosphoramidate linkage. In certain embodiments, the remaining linkages are all unmodified phosphodiester linkages.

In certain embodiments, at least one linkage of the sense oligonucleotides is an inverted linkage.

### II. Oligomeric Duplexes

In certain embodiments, an oligomeric compound described herein comprises an oligonucleotide, having a nucleobase sequence complementary to that of a target nucleic acid, is paired with a second oligomeric compound to form an oligomeric duplex. Such oligomeric duplexes comprise a first oligomeric compound having a portion complementary to a target nucleic acid and a second oligomeric compound having a portion complementary to the first oligomeric compound. In certain embodiments, the first oligomeric compound of an oligomeric duplex comprises or consists of (1) a first modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides. In certain embodiments, the two oligonucleotides have at least one mismatch relative to one another. In certain embodiments, the oligomeric duplex is an antisense agent.

In certain embodiments, an oligomeric duplex comprises:
a first oligomeric compound comprising a first modified oligonucleotide consisting of 15 to 30 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of the nucleobase sequence of any of SEQ ID NOs: 16-166, 318, or 320-324; and
a second oligomeric compound comprising a second modified oligonucleotide consisting of 15 to 29 linked nucleosides, wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or at least 21 contiguous nucleobases of the nucleobase sequence of any of SEQ ID NOs: 167-317, 319, or 325-327, wherein the nucleobase sequence of the second modified oligonucleotide is at least 90% complementary to the nucleobase sequence of an equal length portion of the first modified oligonucleotide.
   In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent.

In certain embodiments, an oligomeric duplex comprises:
a first oligomeric compound comprising a first modified oligonucleotide consisting of 18 to 30 linked nucleosides, wherein the nucleobase sequence of the first modified oligonucleotide comprises at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 16-166, 318, or 320-324; and
a second oligomeric compound comprising a second modified oligonucleotide consisting of 15 to 29 linked nucleosides, wherein the nucleobase sequence of the second modified oligonucleotide comprises a complementary region of at least 8 nucleobases that is at least 90% complementary to the nucleobase sequence of an equal length portion of the first modified oligonucleotide.
   In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the nucleobase sequence of the second modified oligonucleotide comprises a complementary region of at least 9, at least 10, at least 11, at least 12, at least 13, at least 14, at least 15, at least 16, at least 17, at least 18, at least 19, at least 20, or 21 nucleobases that is at least 90% complementary to the nucleobase sequence of an equal portion of the first modified oligonucleotide. In certain embodiments, the nucleobase sequence of the second modified oligonucleotide comprises a complementary region of at least 8, at least 9, at least 10, at least 11, at least 12, at least 13, at least 14, at least 15, at least 16, at least 17, at least 18, at least 19, at least 20, or 21 nucleobases that is at least 95% complementary to the nucleobase sequence of an equal portion of the first modified oligonucleotide. In certain embodiments, the nucleobase sequence of the second modified oligonucleotide comprises a complementary region of at least 8, at least 9, at least 10, at least 11, at least 12, at least 13, at least 14, at least 15, at least 16, at least 17, at least 18, at least 19, at least 20, or 21 nucleobases that is 100% complementary to the nucleobase sequence of an equal portion of the first modified oligonucleotide. In certain embodiments, the oligomeric duplex is an antisense agent.

In certain embodiments, an oligomeric duplex comprises:
a first oligomeric compound comprising a first modified oligonucleotide consisting of 18 to 30 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 16-166, 318, or 320-324; and
a second oligomeric compound comprising a second modified oligonucleotide consisting of 15 to 29 linked nucleosides, wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or 21 contiguous nucleobases of the nucleobase sequence of any of SEQ ID NOs: 167-317, 319, or 325-327, wherein the nucleobase sequence of the second modified oligonucleotide is at least 90% complementary to the nucleobase sequence of an equal length portion of the first modified oligonucleotide.
   In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent.

In certain embodiments, an oligomeric duplex comprises:
a first oligomeric compound comprising a first modified oligonucleotide, wherein the first modified oligonucleotide consists of 23 linked nucleosides and has a nucleobase sequence of consisting of the nucleobase sequence of any of SEQ ID NOs: 16-166, 318, or 320-324; and
a second oligomeric compound comprising a second modified oligonucleotide wherein the second modified oligonucleotide consists of 21 linked nucleosides, wherein the second modified oligonucleotide has a nucleobase sequence consisting of the nucleobase sequence of any of SEQ ID NOs: 167-317, 319, or 325-327, and wherein the nucleobase sequence of the second modified oligonucleotide is at least 90% complementary to the nucleobase sequence of an equal length portion of the first modified oligonucleotide.
   In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the nucleobase sequence of the second modified oligonucleotide is at least 95% or 100% complementary to the nucleobase sequence of an equal length portion of the first modified oligonucleotide. In certain embodiments, the oligomeric duplex is an antisense agent.

In certain embodiments, an oligomeric duplex comprises a first oligomeric compound comprising a first modified oligonucleotide, wherein the first modified oligonucleotide consists of 15 to 30 linked nucleosides and a second oligomeric compound comprising a second modified oligonucleotide, wherein the second modified oligonucleotide consists of 15 to 29 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 16-166, 318, or 320-324 and the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or 21 contiguous nucleobases of the nucleobase sequence of any of SEQ ID NOs: 167-317, 319, or 325-327. In certain embodiments, the nucleobase sequence of the second modified oligonucleotide is at least 90%, at least 95%, or 100% complementary to the nucleobase sequence of an equal length portion of the first modified oligonucleotide. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide, wherein the first modified oligonucleotide consists of 18 to 30 linked nucleosides and a second oligomeric compound comprising a second modified oligonucleotide, wherein the second modified oligonucleotide consists of 15 to 29 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 16-166, 318, or 320-324 and the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or 21 contiguous nucleobases of the nucleobase sequence of any of SEQ ID NOs: 167-317, 319, or 325-327. In certain embodiments, the nucleobase sequence of the second modified oligonucleotide is at least 90%, at least 95%, or 100% complementary to the nucleobase sequence of an equal length portion of the first modified oligonucleotide. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide, wherein the first modified oligonucleotide consists of 18 to 30 linked nucleosides and a second oligomeric compound comprising a second modified oligonucleotide, wherein the second modified oligonucleotide consists of 15 to 29 linked nucleosides, wherein the nucleobase sequence of the first modified oligonucleotide comprises the nucleobase sequence of any of SEQ ID NOs: 16-166, 318, or 320-324 and the nucleobase sequence of the second modified oligonucleotide comprises the nucleobase sequence of any of SEQ ID NOs: 167-317, 319, or 325-327. In certain embodiments, the nucleobase sequence of the second modified oligonucleotide is at least 90%, at least 95%, or 100% complementary to the nucleobase sequence of an equal length portion of the first modified oligonucleotide. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide, wherein the first modified oligonucleotide consists of 23 linked nucleosides and a second oligomeric compound comprising a second modified oligonucleotide, wherein the second modified oligonucleotide consists of 21 linked nucleosides, wherein the nucleobase sequence of the first modified oligonucleotide comprises the nucleobase sequence of any of SEQ ID NOs: 16-166, 318, or 320-324 and the nucleobase sequence of the second modified oligonucleotide comprises the nucleobase sequence of any of SEQ ID NOs: 167-317, 319, or 325-327. In certain embodiments, the nucleobase sequence of the second modified oligonucleotide is at least 90%, at least 95%, or 100% complementary to the nucleobase sequence of an equal length portion of the first modified oligonucleotide. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide consisting of 19 to 29 linked nucleosides and a second oligomeric compound comprising a second modified oligonucleotide consisting of 15 to 29 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of any of the nucleobase sequences recited in SEQ ID NOs: 70, 71, 72, 75, 76, 81, 83, 84, 85, 88, 89, 90, or 92.

In certain embodiments, an oligomeric duplex comprises a first oligomeric compound comprising a first modified oligonucleotide consisting of 19 to 29 linked nucleosides and a second oligomeric compound comprising a second modified oligonucleotide consisting of 15 to 29 linked nucleosides, wherein the nucleobase sequence of the first modified oligonucleotide and the nucleobase sequence of the second modified oligonucleotide each comprises at least 8, at least 9, at least 10, at least 11, at least 12, at least 13, at least 14, at least 15, at least 16, at least 17, at least 18, at least 19, at least 20, at least 21, at least 22, or at least 23 contiguous nucleobases of any of the following pairs of nucleobase sequences recited in SEQ ID NOs: 16/167, 17/168, 18/169, 19/170, 20/171, 21/172, 22/173, 23/174, 24/175, 25/176, 26/177, 27/178, 28/179, 29/180, 30/181, 31/182, 32/183, 33/184, 34/185, 35/186, 36/187, 37/188, 38/189, 39/190, 40/191, 41/192, 42/193, 43/194, 44/195, 45/196, 46/197, 47/198, 48/199, 49/200, 50/201, 51/202, 52/203, 53/204, 54/205, 55/206, 56/207, 57/208, 58/209, 59/210, 60/211, 61/212, 62/213, 63/214, 64/215, 65/216, 66/217, 67/218, 68/219, 69/220, 70/221, 71/222, 72/223, 73/224, 74/225, 75/226, 76/227, 77/228, 78/229, 79/230, 80/231, 81/232, 82/233, 83/234, 84/235, 85/236, 86/237, 87/238, 88/239, 89/240, 90/241, 91/242, 92/243, 93/244, 94/245, 95/246, 96/247, 97/248, 98/249, 99/250, 100/251, 101/252, 102/253, 103/254, 104/255, 105/256, 106/257, 107/258, 108/259, 109/260, 110/261, 111/262, 112/263, 113/264, 114/265, 115/266, 116/267, 117/268, 118/269, 119/270, 120/271, 121/272, 122/273, 123/274, 124/275, 125/276, 126/277, 127/278, 128/279, 129/280, 130/281, 131/282, 132/283, 133/284, 134/285, 135/286, 136/287, 137/288, 138/289, 139/290, 140/291, 141/292, 142/293, 143/294, 144/295, 145/296, 146/297, 147/298, 148/299, 149/300, 150/301, 151/302, 152/303, 153/304, 154/305, 155/306, 156/307, 157/308, 158/309, 159/310, 160/311, 161/312, 162/313, 163/314, 164/315, 165/316, 166/317, 318/319, 320/198, 321/226, 322/325, 323/326, and 324/327, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide consisting of 19 to 29 linked nucleosides and a second oligomeric compound comprising a second modified oligonucleotide consisting of 15 to 29 linked nucleosides, wherein the nucleobase sequences of the first modified oligonucleotide and second modified oligonucleotide comprise any of the following pairs of nucleobase sequences recited in SEQ ID NOs: 16/167, 17/168, 18/169, 19/170, 20/171, 21/172, 22/173, 23/174, 24/175, 25/176, 26/177, 27/178, 28/179, 29/180, 30/181, 31/182, 32/183, 33/184, 34/185, 35/186, 36/187, 37/188, 38/189, 39/190, 40/191, 41/192, 42/193, 43/194, 44/195, 45/196, 46/197, 47/198, 48/199, 49/200, 50/201, 51/202, 52/203, 53/204, 54/205, 55/206, 56/207, 57/208, 58/209, 59/210, 60/211, 61/212, 62/213, 63/214, 64/215, 65/216, 66/217, 67/218, 68/219, 69/220, 70/221, 71/222, 72/223, 73/224, 74/225, 75/226, 76/227, 77/228, 78/229, 79/230, 80/231, 81/232, 82/233, 83/234, 84/235, 85/236, 86/237, 87/238, 88/239, 89/240, 90/241, 91/242, 92/243, 93/244, 94/245, 95/246, 96/247, 97/248, 98/249, 99/250, 100/251, 101/252, 102/253, 103/254, 104/255, 105/256, 106/257, 107/258, 108/259, 109/260, 110/261, 111/262, 112/263, 113/264, 114/265, 115/266, 116/267, 117/268, 118/269, 119/270, 120/271, 121/272, 122/273, 123/274, 124/275, 125/276, 126/277, 127/278, 128/279, 129/280, 130/281, 131/282, 132/283, 133/284, 134/285, 135/286, 136/287, 137/288, 138/289, 139/290, 140/291, 141/292, 142/293, 143/294, 144/295, 145/296, 146/297, 147/298, 148/299, 149/300, 150/301, 151/302, 152/303, 153/304, 154/305, 155/306, 156/307, 157/308, 158/309, 159/310, 160/311, 161/312, 162/313, 163/314, 164/315, 165/316, 166/317, 318/319, 320/198, 321/226, 322/325, 323/326, and 324/327, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide consisting of 23 linked nucleosides and a second oligomeric compound comprising a second modified oligonucleotide consisting of 21 linked nucleosides, wherein the nucleobase sequences of the first modified oligonucleotide and second modified oligonucleotide consist of any of the following pairs of nucleobase sequences recited in SEQ ID NOs: 16/167, 17/168, 18/169, 19/170, 20/171, 21/172, 22/173, 23/174, 24/175, 25/176, 26/177, 27/178, 28/179, 29/180, 30/181, 31/182, 32/183, 33/184, 34/185, 35/186, 36/187, 37/188, 38/189, 39/190, 40/191, 41/192, 42/193, 43/194, 44/195, 45/196, 46/197, 47/198, 48/199, 49/200, 50/201, 51/202, 52/203, 53/204, 54/205, 55/206, 56/207, 57/208, 58/209, 59/210, 60/211, 61/212, 62/213, 63/214, 64/215, 65/216, 66/217, 67/218, 68/219, 69/220, 70/221, 71/222, 72/223, 73/224, 74/225, 75/226, 76/227, 77/228, 78/229, 79/230, 80/231, 81/232, 82/233, 83/234, 84/235, 85/236, 86/237, 87/238, 88/239, 89/240, 90/241, 91/242, 92/243, 93/244, 94/245, 95/246, 96/247, 97/248, 98/249, 99/250, 100/251, 101/252, 102/253, 103/254, 104/255, 105/256, 106/257, 107/258, 108/259, 109/260, 110/261, 111/262, 112/263, 113/264, 114/265, 115/266, 116/267, 117/268, 118/269, 119/270, 120/271, 121/272, 122/273, 123/274, 124/275, 125/276, 126/277, 127/278, 128/279, 129/280, 130/281, 131/282, 132/283, 133/284, 134/285, 135/286, 136/287, 137/288, 138/289, 139/290, 140/291, 141/292, 142/293, 143/294, 144/295, 145/296, 146/297, 147/298, 148/299, 149/300, 150/301, 151/302, 152/303, 153/304, 154/305, 155/306, 156/307, 157/308, 158/309, 159/310, 160/311, 161/312, 162/313, 163/314, 164/315, 165/316, 166/317, 318/319, 320/198, 321/226, 322/325, 323/326, and 324/327, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In any of the oligomeric duplexes described herein, at least one nucleoside of the first modified oligonucleotide and/or the second modified oligonucleotide can comprise a modified sugar moiety. Examples of suitable modified sugar moieties include, but are not limited to, a bicyclic sugar moiety, such as a 2′-4′ bridge selected from —O—CH₂—; and —O—CH(CH₃)—, and a non-bicyclic sugar moiety, such as a 2′-MOE sugar moiety, a 2′-F sugar moiety, a 2′-OMe sugar moiety, or a 2′-NMA sugar moiety. In certain embodiments, at least one nucleoside of the first modified oligonucleotide and/or the second modified oligonucleotide can comprise an unmodified 2′-deoxyribosyl sugar moiety. In certain embodiments, at least 80%, at least 90%, or 100% of the nucleosides of the first modified oligonucleotide and/or the second modified oligonucleotide comprises a modified sugar moiety selected from 2′-F and 2′-OMe. In certain embodiments, one or more 2′-F sugar moieties have a confirmation other than 2′-β-D-ribosyl. In certain embodiments, one or more 2′-F sugar moieties is in the 2′-β-D-xylosyl conformation.

In any of the oligomeric duplexes described herein, at least one nucleoside of the first modified oligonucleotide and/or the second modified oligonucleotide can comprise a sugar surrogate. Examples of suitable sugar surrogates include, but are not limited to, morpholino, hexitol nucleic acid (HNA), fluoro-hexitol nucleic acid (F-HNA), the sugar surrogates of glycol nucleic acid (GNA), and unlocked nucleic acid (UNA). In certain embodiments, at least one nucleoside of the first modified oligonucleotide comprises a sugar surrogate, which can be a GNA.

In certain embodiments, an oligomeric duplex comprises a first oligomeric compound comprising a first modified oligonucleotide consisting of 19 to 29 linked nucleosides and a second oligomeric compound comprising a second modified oligonucleotide consisting of 15 to 29 linked nucleosides, wherein at least one nucleoside of the first modified oligonucleotide and/or the second modified oligonucleotide comprises a modified sugar moiety. In certain embodiments, the modified sugar moiety is a non-bicyclic sugar moiety. In certain embodiment, the non-bicyclic sugar moiety is selected from a 2′-F sugar moiety and a 2′-OMe sugar moiety. In certain embodiments, the first modified oligonucleotide comprises two, three, four, five, six, or more nucleosides comprising non-bicyclic sugar moieties selected from a 2′-F sugar moiety and a 2′-OMe sugar moiety. In certain embodiments, the second modified oligonucleotide comprises two, three, four, or more nucleosides comprising non-bicyclic sugar moieties selected from a 2′-F sugar moiety and a 2′-OMe sugar moiety. In certain embodiments, at least one nucleoside at position 2, 6, 8, 9, 14, or 16 from the 5′ end of the first modified oligonucleotide comprises a 2′-F sugar moiety. In certain embodiments, at least one nucleoside at position 2, 6, 14, or 16 from the 5′ end of the first modified oligonucleotide comprises a 2′-F sugar moiety. In certain embodiments, at least one nucleoside at position 2, 6, or 14 from the 5′ end of the first modified oligonucleotide comprises a 2′-F sugar moiety. In certain embodiments, at least one nucleoside at position 2, 14, or 16 from the 5′ end of the first modified oligonucleotide comprises a 2′-F sugar moiety. In certain embodiments, the nucleoside at position 2 or 14 from the 5′ end of the first modified oligonucleotide comprises a 2′-F sugar moiety. In certain embodiments, the nucleosides at positions 2, 6, 8, 9, 14, and 16 from the 5′ end of the first modified oligonucleotide each comprises a 2′-F sugar moiety. In certain embodiments, the nucleosides at positions 2, 6, 14, and 16 from the 5′ end of the first modified oligonucleotide each comprises a 2′-F sugar moiety. In certain embodiments, the nucleosides at positions 2, 6, and 14 from the 5′ end of the first modified oligonucleotide each comprises a 2′-F sugar moiety. In certain embodiments, the nucleosides at positions 2, 14, and 16 from the 5′ end of the first modified oligonucleotide each comprises a 2′-F sugar moiety. In certain embodiments, the nucleosides at positions 2 and 14 from the 5′ end of the first modified oligonucleotide each comprises a 2′-F sugar moiety. In certain embodiments, at least one nucleoside from the remaining positions of the first modified oligonucleotide comprises a 2′-OMe sugar moiety. In certain embodiments, the nucleosides at the remaining positions of the first modified oligonucleotide each comprises a 2′-OMe sugar moiety. In certain embodiments, at least one nucleoside at position 7, 9, 10, 11, 12, or 15 from the 5′ end of the second modified oligonucleotide comprises a 2′-F sugar moiety. In certain embodiments, at least one nucleoside at position 7, 9, 10, or 11 from the 5′ end of the second modified oligonucleotide comprises a 2′-F sugar moiety. In certain embodiments, at least one nucleoside at position 9, 10, or 11 from the 5′ end of the second modified oligonucleotide comprises a 2′-F sugar moiety. In certain embodiments, at least one nucleoside at position 11, 12, or 15 from the 5′ end of the second modified oligonucleotide comprises a 2′-F sugar moiety. In certain embodiments, the nucleosides at positions 7, 9, 10, and 11 from the 5′ end of the second modified oligonucleotide each comprises a 2′-F sugar moiety. In certain embodiments, the nucleosides at positions 9, 10, and 11 from the 5′ end of the second modified oligonucleotide each comprises a 2′-F sugar moiety. In certain embodiments, at least one nucleoside from the remaining positions of the second modified oligonucleotide comprises a 2′-OMe sugar moiety. In certain embodiments, the nucleosides at the remaining positions of the second modified oligonucleotide each comprises a 2′-OMe sugar moiety. In certain embodiments, the nucleobase sequences of the first modified oligonucleotide and second modified oligonucleotide consist of any of the following pairs of nucleobase sequences recited in SEQ ID NOs: 16/167, 17/168, 18/169, 19/170, 20/171, 21/172, 22/173, 23/174, 24/175, 25/176, 26/177, 27/178, 28/179, 29/180, 30/181, 31/182, 32/183, 33/184, 34/185, 35/186, 36/187, 37/188, 38/189, 39/190, 40/191, 41/192, 42/193, 43/194, 44/195, 45/196, 46/197, 47/198, 48/199, 49/200, 50/201, 51/202, 52/203, 53/204, 54/205, 55/206, 56/207, 57/208, 58/209, 59/210, 60/211, 61/212, 62/213, 63/214, 64/215, 65/216, 66/217, 67/218, 68/219, 69/220, 70/221, 71/222, 72/223, 73/224, 74/225, 75/226, 76/227, 77/228, 78/229, 79/230, 80/231, 81/232, 82/233, 83/234, 84/235, 85/236, 86/237, 87/238, 88/239, 89/240, 90/241, 91/242, 92/243, 93/244, 94/245, 95/246, 96/247, 97/248, 98/249, 99/250, 100/251, 101/252, 102/253, 103/254, 104/255, 105/256, 106/257, 107/258, 108/259, 109/260, 110/261, 111/262, 112/263, 113/264, 114/265, 115/266, 116/267, 117/268, 118/269, 119/270, 120/271, 121/272, 122/273, 123/274, 124/275, 125/276, 126/277, 127/278, 128/279, 129/280, 130/281, 131/282, 132/283, 133/284, 134/285, 135/286, 136/287, 137/288, 138/289, 139/290, 140/291, 141/292, 142/293, 143/294, 144/295, 145/296, 146/297, 147/298, 148/299, 149/300, 150/301, 151/302, 152/303, 153/304, 154/305, 155/306, 156/307, 157/308, 158/309, 159/310, 160/311, 161/312, 162/313, 163/314, 164/315, 165/316, 166/317, 318/319, 320/198, 321/226, 322/325, 323/326, and 324/327, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide consisting of 23 linked nucleosides and a second oligomeric compound comprising a second modified oligonucleotide consisting of 21 linked nucleosides, wherein at least one nucleoside of the first modified oligonucleotide and/or the second modified oligonucleotide comprises a modified sugar moiety. In certain embodiments, the modified sugar moiety is a non-bicyclic sugar moiety. In certain embodiment, the non-bicyclic sugar moiety is selected from a 2′-F sugar moiety and a 2′-OMe sugar moiety. In certain embodiments, the first modified oligonucleotide comprises two, three, four, five, six, or more nucleosides comprising non-bicyclic sugar moieties selected from a 2′-F sugar moiety and a 2′-OMe sugar moiety. In certain embodiments, the second modified oligonucleotide comprises two, three, four, or more nucleosides comprising non-bicyclic sugar moieties selected from a 2′-F sugar moiety and a 2′-OMe sugar moiety. In certain embodiments, at least one nucleoside at position 2, 6, 8, 9, 14, or 16 from the 5′ end of the first modified oligonucleotide comprises a 2′-F sugar moiety. In certain embodiments, at least one nucleoside at position 2, 6, 14, or 16 from the 5′ end of the first modified oligonucleotide comprises a 2′-F sugar moiety. In certain embodiments, at least one nucleoside at position 2, 6, or 14 from the 5′ end of the first modified oligonucleotide comprises a 2′-F sugar moiety. In certain embodiments, at least one nucleoside at position 2, 14, or 16 from the 5′ end of the first modified oligonucleotide comprises a 2′-F sugar moiety. In certain embodiments, the nucleoside at position 2 or 14 from the 5′ end of the first modified oligonucleotide comprises a 2′-F sugar moiety. In certain embodiments, the nucleosides at positions 2, 6, 8, 9, 14, and 16 from the 5′ end of the first modified oligonucleotide each comprises a 2′-F sugar moiety. In certain embodiments, the nucleosides at positions 2, 6, 14, and 16 from the 5′ end of the first modified oligonucleotide each comprises a 2′-F sugar moiety. In certain embodiments, the nucleosides at positions 2, 6, and 14 from the 5′ end of the first modified oligonucleotide each comprises a 2′-F sugar moiety. In certain embodiments, the nucleosides at positions 2, 14, and 16 from the 5′ end of the first modified oligonucleotide each comprises a 2′-F sugar moiety. In certain embodiments, the nucleosides at positions 2 and 14 from the 5′ end of the first modified oligonucleotide each comprises a 2′-F sugar moiety. In certain embodiments, at least one nucleoside from the remaining positions of the first modified oligonucleotide comprises a 2′-OMe sugar moiety. In certain embodiments, the nucleosides at the remaining positions of the first modified oligonucleotide each comprises a 2′-OMe sugar moiety. In certain embodiments, at least one nucleoside at position 7, 9, 10, 11, 12, or 15 from the 5′ end of the second modified oligonucleotide comprises a 2′-F sugar moiety. In certain embodiments, at least one nucleoside at position 7, 9, 10, or 11 from the 5′ end of the second modified oligonucleotide comprises a 2′-F sugar moiety. In certain embodiments, at least one nucleoside at position 9, 10, or 11 from the 5′ end of the second modified oligonucleotide comprises a 2′-F sugar moiety. In certain embodiments, at least one nucleoside at position 11, 12, or 15 from the 5′ end of the second modified oligonucleotide comprises a 2′-F sugar moiety. In certain embodiments, the nucleosides at positions 7, 9, 10, and 11 from the 5′ end of the second modified oligonucleotide each comprises a 2′-F sugar moiety. In certain embodiments, the nucleosides at positions 9, 10, and 11 from the 5′ end of the second modified oligonucleotide each comprises a 2′-F sugar moiety. In certain embodiments, at least one nucleoside from the remaining positions of the second modified oligonucleotide comprises a 2′-OMe sugar moiety. In certain embodiments, the nucleosides at the remaining positions of the second modified oligonucleotide each comprises a 2′-OMe sugar moiety. In certain embodiments, the nucleobase sequences of the first modified oligonucleotide and second modified oligonucleotide consist of any of the following pairs of nucleobase sequences recited in SEQ ID NOs: 16/167, 17/168, 18/169, 19/170, 20/171, 21/172, 22/173, 23/174, 24/175, 25/176, 26/177, 27/178, 28/179, 29/180, 30/181, 31/182, 32/183, 33/184, 34/185, 35/186, 36/187, 37/188, 38/189, 39/190, 40/191, 41/192, 42/193, 43/194, 44/195, 45/196, 46/197, 47/198, 48/199, 49/200, 50/201, 51/202, 52/203, 53/204, 54/205, 55/206, 56/207, 57/208, 58/209, 59/210, 60/211, 61/212, 62/213, 63/214, 64/215, 65/216, 66/217, 67/218, 68/219, 69/220, 70/221, 71/222, 72/223, 73/224, 74/225, 75/226, 76/227, 77/228, 78/229, 79/230, 80/231, 81/232, 82/233, 83/234, 84/235, 85/236, 86/237, 87/238, 88/239, 89/240, 90/241, 91/242, 92/243, 93/244, 94/245, 95/246, 96/247, 97/248, 98/249, 99/250, 100/251, 101/252, 102/253, 103/254, 104/255, 105/256, 106/257, 107/258, 108/259, 109/260, 110/261, 111/262, 112/263, 113/264, 114/265, 115/266, 116/267, 117/268, 118/269, 119/270, 120/271, 121/272, 122/273, 123/274, 124/275, 125/276, 126/277, 127/278, 128/279, 129/280, 130/281, 131/282, 132/283, 133/284, 134/285, 135/286, 136/287, 137/288, 138/289, 139/290, 140/291, 141/292, 142/293, 143/294, 144/295, 145/296, 146/297, 147/298, 148/299, 149/300, 150/301, 151/302, 152/303, 153/304, 154/305, 155/306, 156/307, 157/308, 158/309, 159/310, 160/311, 161/312, 162/313, 163/314, 164/315, 165/316, 166/317, 318/319, 320/198, 321/226, 322/325, 323/326, and 324/327, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide consisting of 19 to 29 linked nucleosides and a second oligomeric compound comprising a second modified oligonucleotide consisting of 15 to 29 linked nucleosides, wherein at least one nucleoside of the first modified oligonucleotide and/or the second modified oligonucleotide comprises a modified sugar moiety selected from a 2′-F sugar moiety and a 2′-OMe sugar moiety. In certain embodiments, each nucleoside of the first modified oligonucleotide comprises a modified sugar moiety selected from a 2′-F sugar moiety and a 2′-OMe sugar moiety. In certain embodiments, the nucleosides of the first modified oligonucleotide have an alternating 2′-F/2′-OMe sugar motif with the nucleoside at position 1 from the 5′ end comprising a 2′-OMe sugar moiety. In certain embodiments, each nucleoside of the second modified oligonucleotide comprises a modified sugar moiety selected from a 2′-F sugar moiety and a 2′-OMe sugar moiety. In certain embodiments, the nucleosides of the second modified oligonucleotide have an alternating 2′-F/2′-OMe sugar motif with the nucleoside at position 1 from the 5′ end comprising a 2′-F sugar moiety. In certain embodiments, the nucleobase sequences of the first modified oligonucleotide and second modified oligonucleotide consist of any of the following pairs of nucleobase sequences recited in SEQ ID NOs: 16/167, 17/168, 18/169, 19/170, 20/171, 21/172, 22/173, 23/174, 24/175, 25/176, 26/177, 27/178, 28/179, 29/180, 30/181, 31/182, 32/183, 33/184, 34/185, 35/186, 36/187, 37/188, 38/189, 39/190, 40/191, 41/192, 42/193, 43/194, 44/195, 45/196, 46/197, 47/198, 48/199, 49/200, 50/201, 51/202, 52/203, 53/204, 54/205, 55/206, 56/207, 57/208, 58/209, 59/210, 60/211, 61/212, 62/213, 63/214, 64/215, 65/216, 66/217, 67/218, 68/219, 69/220, 70/221, 71/222, 72/223, 73/224, 74/225, 75/226, 76/227, 77/228, 78/229, 79/230, 80/231, 81/232, 82/233, 83/234, 84/235, 85/236, 86/237, 87/238, 88/239, 89/240, 90/241, 91/242, 92/243, 93/244, 94/245, 95/246, 96/247, 97/248, 98/249, 99/250, 100/251, 101/252, 102/253, 103/254, 104/255, 105/256, 106/257, 107/258, 108/259, 109/260, 110/261, 111/262, 112/263, 113/264, 114/265, 115/266, 116/267, 117/268, 118/269, 119/270, 120/271, 121/272, 122/273, 123/274, 124/275, 125/276, 126/277, 127/278, 128/279, 129/280, 130/281, 131/282, 132/283, 133/284, 134/285, 135/286, 136/287, 137/288, 138/289, 139/290, 140/291, 141/292, 142/293, 143/294, 144/295, 145/296, 146/297, 147/298, 148/299, 149/300, 150/301, 151/302, 152/303, 153/304, 154/305, 155/306, 156/307, 157/308, 158/309, 159/310, 160/311, 161/312, 162/313, 163/314, 164/315, 165/316, 166/317, 318/319, 320/198, 321/226, 322/325, 323/326, and 324/327, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide consisting of 23 linked nucleosides and a second oligomeric compound comprising a second modified oligonucleotide consisting of 21 linked nucleosides, wherein at least one nucleoside of the first modified oligonucleotide and/or the second modified oligonucleotide comprises a modified sugar moiety selected from a 2′-F sugar moiety and a 2′-OMe sugar moiety. In certain embodiments, each nucleoside of the first modified oligonucleotide comprises a modified sugar moiety selected from a 2′-F sugar moiety and a 2′-OMe sugar moiety. In certain embodiments, the nucleosides of the first modified oligonucleotide have an alternating 2′-F/2′-OMe sugar motif with the nucleoside at position 1 from the 5′ end comprising a 2′-OMe sugar moiety. In certain embodiments, each nucleoside of the second modified oligonucleotide comprises a modified sugar moiety selected from a 2′-F sugar moiety and a 2′-OMe sugar moiety. In certain embodiments, the nucleosides of the second modified oligonucleotide have an alternating 2′-F/2′-OMe sugar motif with the nucleoside at position 1 from the 5′ end comprising a 2′-F sugar moiety. In certain embodiments, the nucleobase sequences of the first modified oligonucleotide and second modified oligonucleotide consist of any of the following pairs of nucleobase sequences recited in SEQ ID NOs: 16/167, 17/168, 18/169, 19/170, 20/171, 21/172, 22/173, 23/174, 24/175, 25/176, 26/177, 27/178, 28/179, 29/180, 30/181, 31/182, 32/183, 33/184, 34/185, 35/186, 36/187, 37/188, 38/189, 39/190, 40/191, 41/192, 42/193, 43/194, 44/195, 45/196, 46/197, 47/198, 48/199, 49/200, 50/201, 51/202, 52/203, 53/204, 54/205, 55/206, 56/207, 57/208, 58/209, 59/210, 60/211, 61/212, 62/213, 63/214, 64/215, 65/216, 66/217, 67/218, 68/219, 69/220, 70/221, 71/222, 72/223, 73/224, 74/225, 75/226, 76/227, 77/228, 78/229, 79/230, 80/231, 81/232, 82/233, 83/234, 84/235, 85/236, 86/237, 87/238, 88/239, 89/240, 90/241, 91/242, 92/243, 93/244, 94/245, 95/246, 96/247, 97/248, 98/249, 99/250, 100/251, 101/252, 102/253, 103/254, 104/255, 105/256, 106/257, 107/258, 108/259, 109/260, 110/261, 111/262, 112/263, 113/264, 114/265, 115/266, 116/267, 117/268, 118/269, 119/270, 120/271, 121/272, 122/273, 123/274, 124/275, 125/276, 126/277, 127/278, 128/279, 129/280, 130/281, 131/282, 132/283, 133/284, 134/285, 135/286, 136/287, 137/288, 138/289, 139/290, 140/291, 141/292, 142/293, 143/294, 144/295, 145/296, 146/297, 147/298, 148/299, 149/300, 150/301, 151/302, 152/303, 153/304, 154/305, 155/306, 156/307, 157/308, 158/309, 159/310, 160/311, 161/312, 162/313, 163/314, 164/315, 165/316, 166/317, 318/319, 320/198, 321/226, 322/325, 323/326, and 324/327, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide consisting of 19 to 29 linked nucleosides and a second oligomeric compound comprising a second modified oligonucleotide consisting of 15 to 29 linked nucleosides, wherein at least one nucleoside of the first modified oligonucleotide and/or the second modified oligonucleotide comprises a modified sugar moiety selected from a 2′-F sugar moiety and a 2′-OMe sugar moiety. In certain embodiments, each nucleoside of the first modified oligonucleotide comprises a modified sugar moiety selected from a 2′-F sugar moiety and a 2′-OMe sugar moiety. In certain embodiments, the nucleosides at positions 1, 3, 5, 7, 9, 11, 13, 15, 17, 19, 21, 22, and 23 from the 5′ end of the first modified oligonucleotide each comprises a 2′-OMe sugar moiety. In certain embodiments, the nucleosides at the remaining positions of the first modified oligonucleotide each comprises a 2′-F sugar moiety. In certain embodiments, each nucleoside of the second modified oligonucleotide comprises a modified sugar moiety selected from a 2′-F sugar moiety and a 2′-OMe sugar moiety. In certain embodiments, the nucleosides of the second modified oligonucleotide have an alternating 2′-F/2′-OMe sugar motif with the nucleoside at position 1 from the 5′ end comprising a 2′-F sugar moiety. In certain embodiments, the nucleobase sequences of the first modified oligonucleotide and second modified oligonucleotide consist of any of the following pairs of nucleobase sequences recited in SEQ ID NOs: 16/167, 17/168, 18/169, 19/170, 20/171, 21/172, 22/173, 23/174, 24/175, 25/176, 26/177, 27/178, 28/179, 29/180, 30/181, 31/182, 32/183, 33/184, 34/185, 35/186, 36/187, 37/188, 38/189, 39/190, 40/191, 41/192, 42/193, 43/194, 44/195, 45/196, 46/197, 47/198, 48/199, 49/200, 50/201, 51/202, 52/203, 53/204, 54/205, 55/206, 56/207, 57/208, 58/209, 59/210, 60/211, 61/212, 62/213, 63/214, 64/215, 65/216, 66/217, 67/218, 68/219, 69/220, 70/221, 71/222, 72/223, 73/224, 74/225, 75/226, 76/227, 77/228, 78/229, 79/230, 80/231, 81/232, 82/233, 83/234, 84/235, 85/236, 86/237, 87/238, 88/239, 89/240, 90/241, 91/242, 92/243, 93/244, 94/245, 95/246, 96/247, 97/248, 98/249, 99/250, 100/251, 101/252, 102/253, 103/254, 104/255, 105/256, 106/257, 107/258, 108/259, 109/260, 110/261, 111/262, 112/263, 113/264, 114/265, 115/266, 116/267, 117/268, 118/269, 119/270, 120/271, 121/272, 122/273, 123/274, 124/275, 125/276, 126/277, 127/278, 128/279, 129/280, 130/281, 131/282, 132/283, 133/284, 134/285, 135/286, 136/287, 137/288, 138/289, 139/290, 140/291, 141/292, 142/293, 143/294, 144/295, 145/296, 146/297, 147/298, 148/299, 149/300, 150/301, 151/302, 152/303, 153/304, 154/305, 155/306, 156/307, 157/308, 158/309, 159/310, 160/311, 161/312, 162/313, 163/314, 164/315, 165/316, 166/317, 318/319, 320/198, 321/226, 322/325, 323/326, and 324/327, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide consisting of 23 linked nucleosides and a second oligomeric compound comprising a second modified oligonucleotide consisting of 21 linked nucleosides, wherein at least one nucleoside of the first modified oligonucleotide and/or the second modified oligonucleotide comprises a modified sugar moiety selected from a 2′-F sugar moiety and a 2′-OMe sugar moiety. In certain embodiments, each nucleoside of the first modified oligonucleotide comprises a modified sugar moiety selected from a 2′-F sugar moiety and a 2′-OMe sugar moiety. In certain embodiments, the nucleosides at positions 1, 3, 5, 7, 9, 11, 13, 15, 17, 19, 21, 22, and 23 from the 5′ end of the first modified oligonucleotide each comprises a 2′-OMe sugar moiety. In certain embodiments, the nucleosides at the remaining positions of the first modified oligonucleotide each comprises a 2′-F sugar moiety. In certain embodiments, each nucleoside of the second modified oligonucleotide comprises a modified sugar moiety selected from a 2′-F sugar moiety and a 2′-OMe sugar moiety. In certain embodiments, the nucleosides of the second modified oligonucleotide have an alternating 2′-F/2′-OMe sugar motif with the nucleoside at position 1 from the 5′ end comprising a 2′-F sugar moiety. In certain embodiments, the nucleobase sequences of the first modified oligonucleotide and second modified oligonucleotide consist of any of the following pairs of nucleobase sequences recited in SEQ ID NOs: 16/167, 17/168, 18/169, 19/170, 20/171, 21/172, 22/173, 23/174, 24/175, 25/176, 26/177, 27/178, 28/179, 29/180, 30/181, 31/182, 32/183, 33/184, 34/185, 35/186, 36/187, 37/188, 38/189, 39/190, 40/191, 41/192, 42/193, 43/194, 44/195, 45/196, 46/197, 47/198, 48/199, 49/200, 50/201, 51/202, 52/203, 53/204, 54/205, 55/206, 56/207, 57/208, 58/209, 59/210, 60/211, 61/212, 62/213, 63/214, 64/215, 65/216, 66/217, 67/218, 68/219, 69/220, 70/221, 71/222, 72/223, 73/224, 74/225, 75/226, 76/227, 77/228, 78/229, 79/230, 80/231, 81/232, 82/233, 83/234, 84/235, 85/236, 86/237, 87/238, 88/239, 89/240, 90/241, 91/242, 92/243, 93/244, 94/245, 95/246, 96/247, 97/248, 98/249, 99/250, 100/251, 101/252, 102/253, 103/254, 104/255, 105/256, 106/257, 107/258, 108/259, 109/260, 110/261, 111/262, 112/263, 113/264, 114/265, 115/266, 116/267, 117/268, 118/269, 119/270, 120/271, 121/272, 122/273, 123/274, 124/275, 125/276, 126/277, 127/278, 128/279, 129/280, 130/281, 131/282, 132/283, 133/284, 134/285, 135/286, 136/287, 137/288, 138/289, 139/290, 140/291, 141/292, 142/293, 143/294, 144/295, 145/296, 146/297, 147/298, 148/299, 149/300, 150/301, 151/302, 152/303, 153/304, 154/305, 155/306, 156/307, 157/308, 158/309, 159/310, 160/311, 161/312, 162/313, 163/314, 164/315, 165/316, 166/317, 318/319, 320/198, 321/226, 322/325, 323/326, and 324/327, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide consisting of 19 to 29 linked nucleosides and a second oligomeric compound comprising a second modified oligonucleotide consisting of 15 to 29 linked nucleosides, wherein at least one nucleoside of the first modified oligonucleotide and/or the second modified oligonucleotide comprises a modified sugar moiety selected from a 2′-F sugar moiety and a 2′-OMe sugar moiety. In certain embodiments, each nucleoside of the first modified oligonucleotide comprises a modified sugar moiety selected from a 2′-F sugar moiety and a 2′-OMe sugar moiety. In certain embodiments, the nucleosides of the first modified oligonucleotide have an alternating 2′-F/2′-OMe sugar motif with the nucleoside at position 1 from the 5′ end comprising a 2′-OMe sugar moiety. In certain embodiments, each nucleoside of the second modified oligonucleotide comprises a modified sugar moiety selected from a 2′-F sugar moiety and a 2′-OMe sugar moiety. In certain embodiments, at least one nucleoside at position of 9, 10, or 11 from the 5′ end of the second modified oligonucleotide comprises a 2′-F sugar moiety. In certain embodiments, the nucleosides at positions of 9, 10, and 11 from the 5′ end of the second modified oligonucleotide each comprises a 2′-F sugar moiety. In certain embodiments, the remaining nucleosides of the second modified oligonucleotide each comprises a 2′-OMe sugar moiety. In certain embodiments, the nucleobase sequences of the first modified oligonucleotide and second modified oligonucleotide consist of any of the following pairs of nucleobase sequences recited in SEQ ID NOs: 16/167, 17/168, 18/169, 19/170, 20/171, 21/172, 22/173, 23/174, 24/175, 25/176, 26/177, 27/178, 28/179, 29/180, 30/181, 31/182, 32/183, 33/184, 34/185, 35/186, 36/187, 37/188, 38/189, 39/190, 40/191, 41/192, 42/193, 43/194, 44/195, 45/196, 46/197, 47/198, 48/199, 49/200, 50/201, 51/202, 52/203, 53/204, 54/205, 55/206, 56/207, 57/208, 58/209, 59/210, 60/211, 61/212, 62/213, 63/214, 64/215, 65/216, 66/217, 67/218, 68/219, 69/220, 70/221, 71/222, 72/223, 73/224, 74/225, 75/226, 76/227, 77/228, 78/229, 79/230, 80/231, 81/232, 82/233, 83/234, 84/235, 85/236, 86/237, 87/238, 88/239, 89/240, 90/241, 91/242, 92/243, 93/244, 94/245, 95/246, 96/247, 97/248, 98/249, 99/250, 100/251, 101/252, 102/253, 103/254, 104/255, 105/256, 106/257, 107/258, 108/259, 109/260, 110/261, 111/262, 112/263, 113/264, 114/265, 115/266, 116/267, 117/268, 118/269, 119/270, 120/271, 121/272, 122/273, 123/274, 124/275, 125/276, 126/277, 127/278, 128/279, 129/280, 130/281, 131/282, 132/283, 133/284, 134/285, 135/286, 136/287, 137/288, 138/289, 139/290, 140/291, 141/292, 142/293, 143/294, 144/295, 145/296, 146/297, 147/298, 148/299, 149/300, 150/301, 151/302, 152/303, 153/304, 154/305, 155/306, 156/307, 157/308, 158/309, 159/310, 160/311, 161/312, 162/313, 163/314, 164/315, 165/316, 166/317, 318/319, 320/198, 321/226, 322/325, 323/326, and 324/327, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In certain embodiments, an oligomeric duplex comprises a first oligomeric compound comprising a first modified oligonucleotide consisting of 23 linked nucleosides and a second oligomeric compound comprising a second modified oligonucleotide consisting of 21 linked nucleosides, wherein at least one nucleoside of the first modified oligonucleotide and/or the second modified oligonucleotide comprises a modified sugar moiety selected from a 2′-F sugar moiety and a 2′-OMe sugar moiety. In certain embodiments, each nucleoside of the first modified oligonucleotide comprises a modified sugar moiety selected from a 2′-F sugar moiety and a 2′-OMe sugar moiety. In certain embodiments, the nucleosides of the first modified oligonucleotide have an alternating 2′-F/2′-OMe sugar motif with the nucleoside at position 1 from the 5′ end comprising a 2′-OMe sugar moiety. In certain embodiments, each nucleoside of the second modified oligonucleotide comprises a modified sugar moiety selected from a 2′-F sugar moiety and a 2′-OMe sugar moiety. In certain embodiments, at least one nucleoside at position of 9, 10, or 11 from the 5′ end of the second modified oligonucleotide comprises a 2′-F sugar moiety. In certain embodiments, the nucleosides at positions of 9, 10, and 11 from the 5′ end of the second modified oligonucleotide each comprises a 2′-F sugar moiety. In certain embodiments, the remaining nucleosides of the second modified oligonucleotide each comprises a 2′-OMe sugar moiety. In certain embodiments, the nucleobase sequences of the first modified oligonucleotide and second modified oligonucleotide consist of any of the following pairs of nucleobase sequences recited in SEQ ID NOs: 16/167, 17/168, 18/169, 19/170, 20/171, 21/172, 22/173, 23/174, 24/175, 25/176, 26/177, 27/178, 28/179, 29/180, 30/181, 31/182, 32/183, 33/184, 34/185, 35/186, 36/187, 37/188, 38/189, 39/190, 40/191, 41/192, 42/193, 43/194, 44/195, 45/196, 46/197, 47/198, 48/199, 49/200, 50/201, 51/202, 52/203, 53/204, 54/205, 55/206, 56/207, 57/208, 58/209, 59/210, 60/211, 61/212, 62/213, 63/214, 64/215, 65/216, 66/217, 67/218, 68/219, 69/220, 70/221, 71/222, 72/223, 73/224, 74/225, 75/226, 76/227, 77/228, 78/229, 79/230, 80/231, 81/232, 82/233, 83/234, 84/235, 85/236, 86/237, 87/238, 88/239, 89/240, 90/241, 91/242, 92/243, 93/244, 94/245, 95/246, 96/247, 97/248, 98/249, 99/250, 100/251, 101/252, 102/253, 103/254, 104/255, 105/256, 106/257, 107/258, 108/259, 109/260, 110/261, 111/262, 112/263, 113/264, 114/265, 115/266, 116/267, 117/268, 118/269, 119/270, 120/271, 121/272, 122/273, 123/274, 124/275, 125/276, 126/277, 127/278, 128/279, 129/280, 130/281, 131/282, 132/283, 133/284, 134/285, 135/286, 136/287, 137/288, 138/289, 139/290, 140/291, 141/292, 142/293, 143/294, 144/295, 145/296, 146/297, 147/298, 148/299, 149/300, 150/301, 151/302, 152/303, 153/304, 154/305, 155/306, 156/307, 157/308, 158/309, 159/310, 160/311, 161/312, 162/313, 163/314, 164/315, 165/316, 166/317, 318/319, 320/198, 321/226, 322/325, 323/326, and 324/327, wherein the nucleobase sequence of the first modified oligonucleotide comprises the nucleobase sequence of the first SEQ ID NO recited in the pair and the nucleobase sequence of the second modified oligonucleotide comprises the nucleobase sequence of the second SEQ ID NO recited in the pair. In certain embodiments, the first oligomeric compound is an antisense compound. In certain embodiments, the first modified oligonucleotide is an antisense oligonucleotide. In certain embodiments, the second oligomeric compound is a sense compound. In certain embodiments, the second modified oligonucleotide is a sense oligonucleotide. In certain embodiments, the first modified oligonucleotide is an antisense RNAi oligonucleotide. In certain embodiments, the second modified oligonucleotide is a sense RNAi oligonucleotide. In certain embodiments, the oligomeric duplex is an antisense agent.

In any of the oligomeric duplexes described herein, at least one internucleoside linkage of the first modified oligonucleotide and/or the second modified oligonucleotide can comprise a modified internucleoside linkage. In certain embodiments, the modified internucleoside linkage is a phosphorothioate internucleoside linkage. In certain embodiments, at least one of the first, second, or third internucleoside linkages from the 5′ end and/or the 3′ end of the first modified oligonucleotide comprises a phosphorothioate linkage. In certain embodiments, at least one of the first, second, or third internucleoside linkages from the 5′ end and/or the 3′ end of the second modified oligonucleotide comprises a phosphorothioate linkage. In certain embodiments, the modified internucleoside linkage is a mesyl phosphoramidate internucleoside linkage. In certain embodiments, at least one of the first or second internucleoside linkages from the 5′ end and/or the 3′ end of the first modified oligonucleotide comprises a mesyl phosphoramidate internucleoside linkage. In certain embodiments, at least one of the first or second internucleoside linkages from the 5′ end and/or the 3′ end of the second modified oligonucleotide comprises a mesyl phosphoramidate internucleoside linkage.

In any of the oligomeric duplexes described herein, at least one internucleoside linkage of the first modified oligonucleotide and/or the second modified oligonucleotide can comprise a phosphodiester internucleoside linkage.

In any of the oligomeric duplexes described herein, each internucleoside linkage of the first modified oligonucleotide and/or the second modified oligonucleotide can be independently selected from a phosphodiester, a phosphorothioate, or a mesyl phosphoramidate internucleoside linkage.

In any of the oligomeric duplexes described herein, each internucleoside linkage of the first modified oligonucleotide and/or the second modified oligonucleotide can be independently selected from a phosphodiester or a phosphorothioate internucleoside linkage.

In any of the oligomeric duplexes described herein, each internucleoside linkage of the first modified oligonucleotide and/or the second modified oligonucleotide can be independently selected from a phosphodiester or a mesyl phosphoramidate internucleoside linkage.

In any of the oligomeric duplexes described herein, the internucleoside linkage motif of the first modified oligonucleotide can be 5′-ssooooooooooooooooooss-3′, wherein each “s” is a phosphorothioate internucleoside linkage and each “o” is a phosphodiester internucleoside linkage. In any of the oligomeric duplexes described herein, the internucleoside linkage motif of the second modified oligonucleotide can be 5′-ssooooooooooooooooss-3′, wherein each “s” is a phosphorothioate internucleoside linkage and each “o” is a phosphodiester internucleoside linkage.

In any of the oligomeric duplexes described herein, at least one nucleobase of the first modified oligonucleotide and/or the second modified oligonucleotide can be modified nucleobase. In certain embodiments, the modified nucleobase is 5-methylcytosine.

In any of the oligomeric duplexes described herein, the first modified oligonucleotide can comprise a stabilized phosphate group attached to the 5′ position of the 5′-most nucleoside. In certain embodiments, the stabilized phosphate group comprises a cyclopropyl phosphonate or an (E)-vinyl phosphonate.

In any of the oligomeric duplexes described herein, the first modified oligonucleotide can comprise a conjugate group. In certain embodiments, the conjugate group comprises a conjugate linker and a conjugate moiety. In certain embodiments, the conjugate group is attached to the first modified oligonucleotide at the 5′-end of the first modified oligonucleotide. In certain embodiments, the conjugate group is attached to the first modified oligonucleotide at the 3′-end of the modified oligonucleotide. In certain embodiments, the conjugate group is attached to the first modified oligonucleotide at an internal position. In certain embodiments, the conjugate group is attached to the first modified oligonucleotide through a 2′-modification of a furanosyl sugar moiety. In certain embodiments, the conjugate group is attached to the first modified oligonucleotide through a modified internucleoside linkage. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In any of the oligomeric duplexes described herein, the second modified oligonucleotide can comprise a conjugate group. In certain embodiments, the conjugate group comprises a conjugate linker and a conjugate moiety. In certain embodiments, the conjugate group is attached to the second modified oligonucleotide at the 5′-end of the second modified oligonucleotide. In certain embodiments, the conjugate group is attached to the second modified oligonucleotide at the 3′-end of the modified oligonucleotide. In certain embodiments, the conjugate group is attached to the second modified oligonucleotide at an internal position. In certain embodiments, the conjugate group is attached to the second modified oligonucleotide through a 2′-modification of a furanosyl sugar moiety. In certain embodiments, the conjugate group is attached to the second modified oligonucleotide through a modified intemucleoside linkage. In certain embodiments, the conjugate group comprises N-acetyl galactosamine. In certain embodiments, the conjugate group comprises a cell-targeting moiety having an affinity for transferrin receptor (TfR), also known as TfR1 and CD71. In certain embodiments, the conjugate group comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, conjugate groups may be selected from any of a C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C17 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl. In certain embodiments, conjugate groups may be selected from any of C22 alkyl, C20 alkyl, C16 alkyl, C10 alkyl, C21 alkyl, C19 alkyl, C18 alkyl, C17 alkyl, C15 alkyl, C14 alkyl, C13 alkyl, C12 alkyl, C11 alkyl, C9 alkyl, C8 alkyl, C7 alkyl, C6 alkyl, and C5 alkyl, where the alkyl chain has one or more unsaturated bonds.

In certain embodiments, an antisense agent comprises an antisense compound, which comprises an oligomeric compound or an oligomeric duplex described herein. In certain embodiments, an antisense agent, which can comprise an oligomeric compound or an oligomeric duplex described herein, is an RNAi agent capable of reducing the amount of SNCA RNA through the activation of RISC/Ago2.

Certain embodiments provide an oligomeric agent comprising two or more oligomeric duplexes. In certain embodiments, an oligomeric agent comprises two or more of any of the oligomeric duplexes described herein. In certain embodiments, an oligomeric agent comprises two or more of the same oligomeric duplex, which can be any of the oligomeric duplexes described herein. In certain embodiments, the two or more oligomeric duplexes are linked together. In certain embodiments, the two or more oligomeric duplexes are covalently linked together. In certain embodiments, the second modified oligonucleotides of two or more oligomeric duplexes are covalently linked together. In certain embodiments, the second modified oligonucleotides of two or more oligomeric duplexes are covalently linked together at their 3′ ends. In certain embodiments, the two or more oligomeric duplexes are covalently linked together by a glycol linker, such as a tetraethylene glycol linker. Certain such compounds are described in, e.g., Alterman, et al., Nature Biotech., 2019, 37:844-894.

### Certain Terminal Groups

In certain embodiments, oligomeric compounds comprise a terminal group. In certain such embodiments, oligomeric compounds comprise a phosphorus-containing group at the 5′-end of the antisense oligonucleotide and/or the sense oligonucleotide. In certain embodiments, the terminal group is a phosphate stabilized phosphate group. The 5′-end phosphorus-containing group can be 5′-end phosphate (5′-P), 5′-end phosphorothioate (5′-PS), 5′-end phosphorodithioate (5′-PS₂), 5′-end vinylphosphonate (5′-VP), 5′-end methylphosphonate (MePhos) or 5′-deoxy-5′-C-malonyl. When the 5′-end phosphorus-containing group is 5′-end vinylphosphonate, the 5′VP can be either 5′-E-VP isomer (i.e., trans-vinylphosphonate), 5′-Z-VP isomer (i.e., cis-vinylphosphonate), or mixtures thereof. Although such phosphate group can be attached to either the antisense oligonucleotide or the sense oligonucleotide, it will typically be attached to the antisense oligonucleotide as that has been shown to improve activity of certain RNAi agents. See, e.g., Prakash et al., Nucleic Acids Res., 2015, 43(6):2993-3011; Elkayam, et al., Nucleic Acids Res., 2017, 45(6):3528-3536; Parmar, et al. ChemBioChem, 2016, 17(11):985-989; and Harastzi, et al., Nucleic Acids Res., 2017, 45(13):7581-7592. In certain embodiments, the phosphate stabilizing group is 5′-cyclopropyl phosphonate. See e.g., WO/2018/027106.

### Certain Conjugated Oligomeric Compounds

In certain embodiments, the oligomeric compounds comprise one or more conjugate groups. Conjugate groups consist of one or more conjugate moiety and a conjugate linker which links the conjugate moiety to an oligonucleotide of an oligomeric compound. Conjugate groups may be attached to either or both ends and/or at any internal position of an oligonucleotide. In certain embodiments, conjugate groups modify one or more properties of oligomeric compound, including, but not limited to, pharmacodynamics, pharmacokinetics, stability, binding, absorption, tissue distribution, cellular distribution, cellular uptake, charge, and clearance.

Conjugation of one or more carbohydrate moieties to an oligomeric compound can optimize one or more properties of the oligomeric compound. In certain embodiments, the carbohydrate moiety is attached to a modified subunit of the oligomeric compound. For example, the ribose sugar of one or more ribonucleotide subunits of an oligomeric compound can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds.

### A. Certain Specific Conjugate Groups

Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucleic Acids Res., 1992, 20, 533-538), an aliphatic chain, e.g., do-decan-diol or undecyl residues (Saison-Behmoaras et al., EMBO J, 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucleic Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937), a tocopherol group (Nishina et al., Mol. Ther. Nucleic Acids, 2015, 4, e220; doi:10.1038/mtna.2014.72 and Nishina et al., Mol. Ther., 2008, 16, 734-740), or a GalNAc cluster (e.g., WO2014/179620).

### 1. Conjugate Moieties

Conjugate moieties include, without limitation, intercalators, reporter molecules, polyamines, polyamides, peptides, carbohydrates (e.g., GalNAc), antibodies, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial, or an antibiotic.

### 2. Conjugate Linkers

Conjugate moieties are attached to an oligomeric compound through conjugate linkers. In certain embodiments, a conjugate group is a single chemical bond (i.e. conjugate moiety is attached to an oligonucleotide via a conjugate linker through a single bond). In certain embodiments, the conjugate linker comprises a chain structure, such as a hydrocarbyl chain, or an oligomer of repeating units, such as ethylene glycol, nucleosides, or amino acid units.

In certain embodiments, a conjugate linker comprises a pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a compound and the other is selected to bind to a conjugate group. Examples of functional groups used in a bifunctional linking moiety include, but are not limited to, electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include, but are not limited to, pyrrolidine, 8-amino-3,6-dioxaoctanoic acid (ADO), succinimidyl 4-(N-maleimidomethyl)cyclohexane-1-carboxylate (SMCC) and 6-aminohexanoic acid (AHEX or AHA). Other conjugate linkers include, but are not limited to, substituted or unsubstituted C₁-C₁₀ alkyl, substituted or unsubstituted C₂-C₁₀ alkenyl, or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl, and alkynyl.

In certain embodiments, conjugate linkers comprise 1-5 linker-nucleosides. In certain embodiments, such linker-nucleosides are modified nucleosides. In certain embodiments, such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the compound after it reaches a target tissue. Accordingly, linker-nucleosides are typically linked to one another and to the remainder of the compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises two oligonucleotides each consisting of a specified number or range of linked nucleosides and the antisense oligonucleotide having a specified percent complementarity to a reference nucleic acid, and the oligomeric compound also comprises a conjugate group comprising a conjugate linker comprising linker-nucleosides, those linker-nucleosides are not counted toward the length of the oligonucleotides of an oligomeric compound and are not used in determining the percent complementarity of the antisense oligonucleotide with the reference nucleic acid. Unless otherwise indicated, conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligomeric compound. For example, in certain circumstances, oligomeric compounds comprising a particular conjugate moiety are better taken up by a particular cell type, but once the oligomeric compound has been taken up, it is desirable that the conjugate group be cleaved to release the unconjugated or parent oligomeric compound. Thus, certain conjugates may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the compound through cleavable bonds. In certain embodiments, such cleavable bonds are unmodified phosphodiester bonds. In certain embodiments, a cleavable moiety is 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### 3. Certain Cell-Targeting Conjugate Moieties

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, the cell-targeting moiety targets neurons. In certain embodiments, the cell-targeting moiety targets a neurotransmitter receptor. In certain embodiments, the cell targeting moiety targets a neurotransmitter transporter. In certain embodiments, the cell targeting moiety targets a GABA transporter. See e.g., WO 2011/131693, WO 2014/064257.

### Certain Motifs

Oligomeric duplexes can be described by motif or by specific features. In certain embodiments, an oligomeric duplex having a motif or specific feature described herein is an antisense agent.

In certain embodiments, the oligomeric duplexes described herein comprise:
(a) a sense oligonucleotide having:
   (i) a length of 21 nucleosides;
   (ii) a conjugate attached to the 3′-end;
   (iii) 2′-F modifications at positions 1, 3, 5, 7, 9, 11, 13, 15, 17, 19, and 21, and 2′-OMe modifications at positions 2, 4, 6, 8, 10, 12, 14, 16, 18, and 20 (counting from the 5′ end); and
   (iv) phosphorothioate internucleoside linkages between nucleoside positions 1 and 2, 2 and 3, 19 and 20, and 20 and 21 (counting from the 5′ end);
and
(b) an antisense oligonucleotide having:
   (i) a length of 23 nucleosides;
   (ii) 2′-OMe modifications at positions 1, 3, 5, 9, 11, 13, 15, 17, 19, and 21 to 23, and 2′F modifications at positions 2, 4, 6, 8, 10, 12, 14, 16, 18, and 20 (counting from the 5′ end); and
   (iii) phosphorothioate internucleoside linkages between nucleoside positions 1 and 2, 2 and 3, 21 and 22, and 22 and 23 (counting from the 5′ end);
wherein the two nucleosides at the 3′end of the antisense oligonucleotide are overhanging nucleosides, and the end of the oligomeric duplex constituting the 5′-end of the antisense oligonucleotide and the 3′-end of the sense oligonucleotide is blunt (i.e., neither oligonucleotide has overhang nucleoside at that end and instead the hybridizing region of the sense oligonucleotide includes the 3′-most nucleoside of the sense oligonucleotide and that nucleoside hybridizes with the 5′-most nucleoside of the antisense oligonucleotide).

In certain embodiments, the oligomeric duplexes described herein comprise:
(a) a sense oligonucleotide having:
   (i) a length of 21 nucleosides;
   (ii) a conjugate attached to the 3′-end; and
   (iii) 2′-F modifications at positions 1, 3, 5, 7, 9 to 11, 13, 17, 19, and 21, and 2′-OMe modifications at positions 2, 4, 6, 8, 12, 14 to 16, 18, and 20 (counting from the 5′ end);
and
(b) an antisense oligonucleotide having:
   (i) a length of 23 nucleosides;
   (ii) 2′-OMe modifications at positions 1, 3, 5, 9, 11 to 13, 15, 17, 19, 21, and 23, and 2′-F modifications at positions 2, 4, 6 to 8, 10, 14, 16, 18, 20, and 22 (counting from the 5′ end); and
   (iii) phosphorothioate internucleoside linkages between nucleoside positions 21 and 22, and between nucleoside positions 22 and 23 (counting from the 5′ end);
   wherein the two nucleosides at the 3′end of the antisense oligonucleotide are overhanging nucleosides, and the end of the oligomeric duplex constituting the 5′-end of the antisense oligonucleotide and the 3′-end of the sense oligonucleotide is blunt (i.e., neither oligonucleotide has overhang nucleoside at that end and instead the hybridizing region of the sense oligonucleotide includes the 3′-most nucleoside of the sense oligonucleotide and that nucleoside hybridizes with the 5′-most nucleoside of the antisense oligonucleotide).

In certain embodiments, the oligomeric duplexes described herein comprise:
(a) a sense oligonucleotide having:
   (i) a length of 21 nucleosides;
   (ii) a conjugate attached to the 3′-end;
   (iii) 2′-F modifications at positions 1, 3, 5, 7, 9 to 11, 13, 17, 19, and 21, and 2′-OMe modifications at positions 2, 4, 6, 8, 12, 14, 16, 18, and 20 (counting from the 5′ end); and
   (iv) phosphorothioate internucleoside linkages between nucleoside positions 1 and 2, and between nucleoside positions 2 and 3 (counting from the 5′ end);
and
(b) an antisense oligonucleotide having:
   (i) a length of 23 nucleosides;
   (ii) 2′-OMe modifications at positions 1, 3, 5, 7, 9, 11 to 13, 15, 17, 19, and 21 to 23, and 2′F modifications at positions 2, 4, 6, 8, 10, 14, 16, 18, and 20 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the oligomeric duplex includes a two nucleoside overhang at the 3′end of the antisense oligonucleotide, and a blunt end at the 5′-end of the antisense oligonucleotide.

In certain embodiments, the oligomeric duplexes described herein comprise:
(a) a sense oligonucleotide having:
   (i) a length of 21 nucleosides;
   (ii) a conjugate attached to the 3′-end;
   (iii) 2′-OMe modifications at positions 1 to 6, 8, 10, and 12 to 21, and 2′-F modifications at positions 7 and 9, and a deoxynucleoside at position 11 (counting from the 5′ end); and
   (iv) phosphorothioate internucleoside linkages between nucleoside positions 1 and 2, and between nucleoside positions 2 and 3 (counting from the 5′ end);
and
(b) an antisense oligonucleotide having:
   (i) a length of 23 nucleosides;
   (ii) 2′-OMe modifications at positions 1, 3, 7, 9, 11, 13, 15, 17, and 19 to 23, and 2′-F modifications at positions 2, 4 to 6, 8, 10, 12, 14, 16, and 18 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the oligomeric duplex has a two nucleoside overhang at the 3′end of the antisense oligonucleotide, and a blunt end at the 5′-end of the antisense oligonucleotide.

In certain embodiments, the oligomeric duplexes described herein comprise:
(a) a sense oligonucleotide having:
   (i) a length of 21 nucleosides;
   (ii) a conjugate attached to the 3′-end;
   (iii) 2′-OMe modifications at positions 1 to 6, 8, and 12 to 21, and 2′-F modifications at positions 7, and 9 to 11; and
   (iv) phosphorothioate internucleoside linkages between nucleoside positions 1 and 2, and between nucleoside positions 2 and 3 (counting from the 5′ end);
and
(b) an antisense oligonucleotide having:
   (i) a length of 23 nucleosides;
   (ii) 2′-OMe modifications at positions 1, 3 to 5, 7, 8, 10 to 13, 15, and 17 to 23, and 2′-F modifications at positions 2, 6, 9, 14, and 16 (counting from the 5′ end); and
   (iii) phosphorothioate internucleoside linkages between nucleoside positions 1 and 2, between nucleoside positions 2 and 3, between nucleoside positions 21 and 22, and between nucleoside positions 22 and 23 (counting from the 5′ end);
   wherein the oligomeric duplex has a two nucleotide overhang at the 3′end of the antisense oligonucleotide, and a blunt end at the 5′-end of the antisense oligonucleotide.

In certain embodiments, the oligomeric duplexes described herein comprise:
(a) a sense oligonucleotide having:
   (i) a length of 21 nucleosides;
   (ii) a conjugate attached to the 3′-end;
   (iii) 2′-OMe modifications at positions 1 to 6, 8, and 12 to 21, and 2′-F modifications at positions 7, and 9 to 11; and
   (iv) phosphorothioate internucleoside linkages between nucleoside positions 1 and 2, and between nucleoside positions 2 and 3 (counting from the 5′ end);
and
(b) an antisense oligonucleotide having:
   (i) a length of 23 nucleosides;
   (ii) 2′-OMe modifications at positions 1, 3 to 5, 7, 10 to 13, 15, and 17 to 23, and 2′-F modifications at positions 2, 6, 8, 9, 14, and 16 (counting from the 5′ end); and
   (iii) phosphorothioate internucleoside linkages between nucleoside positions 1 and 2, between nucleoside positions 2 and 3, between nucleotide positions 21 and 22, and between nucleoside positions 22 and 23 (counting from the 5′ end);
   wherein the oligomeric duplex has a two nucleoside overhang at the 3′end of the antisense oligonucleotide, and a blunt end at the 5′-end of the antisense oligonucleotide.

In certain embodiments, the oligomeric duplexes described herein comprise:
(a) a sense oligonucleotide having:
   (i) a length of 19 nucleosides;
   (ii) an conjugate attached to the 3′-end;
   (iii) 2′-OMe modifications at positions 1 to 4, 6, and 10 to 19, and 2′-F modifications at positions 5, and 7 to 9; and
   (iv) phosphorothioate internucleoside linkages between nucleoside positions 1 and 2, and between nucleoside positions 2 and 3 (counting from the 5′ end);
and
(b) an antisense oligonucleotide having:
   (i) a length of 21 nucleosides;
   (ii) 2′-OMe modifications at positions 1, 3 to 5, 7, 10 to 13, 15, and 17 to 21, and 2′-F modifications at positions 2, 6, 8, 9, 14, and 16 (counting from the 5′ end); and
   (iii) phosphorothioate internucleoside linkages between nucleoside positions 1 and 2, between nucleoside positions 2 and 3, between nucleoside positions 19 and 20, and between nucleoside positions 20 and 21 (counting from the 5′ end);
   wherein the oligomeric duplex has a two nucleoside overhang at the 3′end of the antisense oligonucleotide, and a blunt end at the 5′-end of the antisense oligonucleotide.

In any of the above embodiments, the conjugate at the 3′-end of the sense oligonucleotide may comprise a targeting moiety. In certain such embodiments, the targeting moiety targets a neurotransmitter receptor. In certain embodiments, the cell targeting moiety targets a neurotransmitter transporter. In certain embodiments, the cell targeting moiety targets a GABA transporter.

In certain embodiments, the oligomeric duplex comprises a sense oligonucleotide consisting of 21 nucleosides and an antisense oligonucleotide consisting of 23 nucleosides, wherein the sense oligonucleotide contains at least one motif of three contiguous 2′-F modified nucleosides at positions 9, 10, 11 from the 5′-end; the antisense oligonucleotide contains at least one motif of three 2′-O-methyl modifications on three consecutive nucleosides at positions 11, 12, 13 from the 5′ end, wherein one end of the oligomeric duplex is blunt, while the other end comprises a 2 nucleotide overhang. Preferably, the 2 nucleotide overhang is at the 3′-end of the antisense oligonucleotide.

In certain embodiments, when the 2 nucleotide overhang is at the 3′-end of the antisense oligonucleotide, there may be two phosphorothioate internucleoside linkages between the terminal three nucleotides, wherein two of the three nucleotides are the overhang nucleotides, and the third nucleotide is a paired nucleotide next to the overhang nucleotide. In certain embodiments, the oligomeric duplex additionally has two phosphorothioate internucleoside linkages between the terminal three nucleotides at both the 5′-end of the sense oligonucleotide and at the 5′-end of the antisense oligonucleotide. In certain embodiments, every nucleoside in the sense oligonucleotide and the antisense oligonucleotide of the oligomeric duplex is a modified nucleoside. In certain embodiments, each nucleoside is independently modified with a 2′-O-methyl or 3′-fluoro, e.g. in an alternating motif. Optionally, the oligomeric duplex comprises a conjugate.

In certain embodiments, every nucleotide in the sense oligonucleotide and antisense oligonucleotide of the oligomeric duplex, including the nucleotides that are part of the motifs, may be modified. Each nucleotide may be modified with the same or different modification, which can include one or more alteration of one or both of the non-linking phosphate oxygens; alteration of a constituent of the ribose sugar, e.g., of the 2′ hydroxyl on the ribose sugar; wholesale replacement of the phosphate moiety with “dephospho” linkers; modification or replacement of a naturally occurring base; and replacement or modification of the ribose-phosphate backbone.

In certain embodiments, each nucleoside of the sense oligonucleotide and antisense oligonucleotide is independently modified with LNA, cEt, UNA, HNA, CeNA, 2′-MOE, 2′-OMe, 2′-O-allyl, 2′-C-allyl, 2′-deoxy, 2′-hydroxyl, or 2′-fluoro. The oligomeric duplex can contain more than one modification. In one embodiment, each nucleoside of the sense oligonucleotide and antisense oligonucleotide is independently modified with 2′-O-methyl or 2′-F. In certain embodiments, the modification is a 2′-NMA modification.

The term “alternating motif” as used herein refers to a motif having one or more modifications, each modification occurring on alternating nucleosides of one oligonucleotide. The alternating nucleoside may refer to one per every other nucleoside or one per every three nucleosides, or a similar pattern. For example, if A, B and C each represent one type of modification to the nucleoside, the alternating motif can be “ABABABABABAB . . . ,” “AABBAABBAABB . . . ,” “AABAABAABAAB . . . ,” “AAABAAABAAAB . . . ,” “AAABBBAAABBB . . . ,” or “ABCABCABCABC . . . ,” etc.

The type of modifications contained in the alternating motif may be the same or different. For example, if A, B, C, D each represent one type of modification on the nucleoside, the alternating pattern, i.e., modifications on every other nucleoside, may be the same, but each of the sense oligonucleotide or antisense oligonucleotide can be selected from several possibilities of modifications within the alternating motif such as “ABABAB . . . ”, “ACACAC . . . ” “BDBDBD . . . ” or “CDCDCD . . . ,” etc.

In certain embodiments, the modification pattern for the alternating motif on the sense oligonucleotide relative to the modification pattern for the alternating motif on the antisense oligonucleotide is shifted. The shift may be such that the group of modified nucleotide of the sense oligonucleotide corresponds to a group of differently modified nucleotides of the antisense oligonucleotide and vice versa. For example, the sense oligonucleotide when paired with the antisense oligonucleotide in the oligomeric duplex, the alternating motif in the sense oligonucleotide may start with “ABABAB” from 5′-3′ of the oligonucleotide and the alternating motif in the antisense oligonucleotide may start with “BABABA” from 5′-3′ of the oligonucleotide within the duplex region. As another example, the alternating motif in the sense oligonucleotide may start with “AABBAABB” from 5′-3′ of the oligonucleotide and the alternating motif in the antisense oligonucleotide may start with “BBAABBAA” from 5′-3′ of the oligonucleotide within the duplex region, so that there is a complete or partial shift of the modification 10 patterns between the sense oligonucleotide and the antisense oligonucleotide.

In certain embodiments, the oligomeric duplex comprising the pattern of the alternating motif of 2′-O-methyl modification and 2′-F modification on the sense oligonucleotide initially has a shift relative to the pattern of the alternating motif of 2′-O-methyl modification and 2′-F modification on the antisense oligonucleotide initially, i.e., the 2′-O-methyl modified nucleotide on the sense oligonucleotide base pairs with a 2′-F modified nucleotides on the antisense oligonucleotide and vice versa. The 1 position of the sense oligonucleotide may start with the 2′-F modification, and the 1 position of the antisense oligonucleotide may start with a 2′-O-methyl modification.

The introduction of one or more motifs of three identical modifications on three consecutive nucleotides to the sense oligonucleotide and/or antisense oligonucleotide interrupts the initial modification pattern present in the sense oligonucleotide and/or antisense oligonucleotide. This interruption of the modification pattern of the sense and/or antisense oligonucleotide by introducing one or more motifs of three identical modifications on three consecutive nucleotides to the sense and/or antisense oligonucleotide surprisingly enhances the gene silencing activity to the target gene. In one embodiment, when the motif of three identical modifications on three consecutive 25 nucleotides is introduced to any of the oligonucleotides, the modification of the nucleotide next to the motif is a different modification than the modification of the motif. For example, the portion of the sequence containing the motif is “ . . . NaYYYNb . . . ,” where “Y” represents the modification of the motif of three identical modifications on three consecutive nucleotide, and “Na” and “Nb” represent a modification to the nucleotide next to the motif “YYY” that is different than the modification of Y, and where Na and Nb can be the same or different modifications. Alternatively, Na and/or Nb may be present or absent when there is a wing modification present.

In certain embodiments, the sense oligonucleotide may be represented by formula (I):

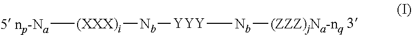

wherein:
i and j are each independently 0 or 1;
p and q are each independently 0-6;
each Nₐ independently represents 0-25 linked nucleosides comprising at least two differently modified nucleosides;
each N_{b} independently represents 0-10 linked nucleosides;
each nₚ and n_{q} independently represent an overhanging nucleoside;
wherein N_{b} and Y do not have the same modification; and
XXX, YYY and ZZZ each independently represent modified nucleosides where each X nucleoside has the same modification; each Y nucleoside has the same modification; and each Z nucleoside has the same modification. In certain embodiments, each Y comprises a 2′-F modification.

In certain embodiments, the Nₐ and N_{b} comprise modifications of alternating patterns.

In certain embodiments, the YYY motif occurs at or near the cleavage site of the target nucleic acid. For example, when the oligomeric duplex has a duplex region of 17-23 nucleotides in length, the YYY motif can occur at or near the vicinity of the cleavage site (e.g., can occur at positions 6, 7, 8; 7, 8, 9; 8, 9, 10; 9, 10, 11; 10, 11, 12; or 11, 12, 13) of the sense oligonucleotide, the count starting from the 1^{st} nucleotide from the 5′-end; or optionally, the count starting at the 1′ paired nucleotide within the duplex region, from the 5′-end.

In certain embodiments, the antisense oligonucleotide of the oligomeric duplex may be represented by the formula:

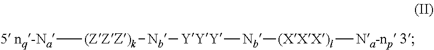

wherein:
k and l are each independently 0 or 1;
p‘ and q’ are each independently 0-6;
each Nₐ′ independently represents 0-25 linked nucleotides comprising at least two differently modified nucleotides;
each N_{b}′ independently represents 0-10 linked nucleotides;
each nₚ′ and n_{q}′ independently represent an overhanging nucleoside;
wherein N_{b}′ and Y′ do not have the same modification; and
X′X′X′, Y′Y′Y′ and Z′Z′Z′ each independently represent modified nucleosides where each X′ nucleoside has the same modification; each Y′ nucleoside has the same modification; and each Z′ nucleoside has the same modification. In certain embodiments, each Y′ comprises a 2′-F modification. In certain embodiments, each Y′ comprises a 2′-OMe modification.

In certain embodiments, the Nₐ′ and/or N_{b}′ comprise modifications of alternating patterns.

In certain embodiments, the Y′Y′Y′ motif occurs at or near the cleavage site of the target nucleic acid. For example, when the oligomeric duplex has a duplex region of 17-23 nucleotides in length, the Y′Y′Y′ motif can occur at positions 9, 10, 11; 10, 11, 12; 11, 12, 13; 12, 13, 14; or 13, 14, 15 of the antisense oligonucleotide, with the count starting from the 1′ nucleotide from the 5′-end; or, optionally, the count starting at the 1^{st} paired nucleotide within the duplex region, from the 5′-end. Preferably, the Y′Y′Y′ motif occurs at positions 11, 12, 13.

In certain embodiments, k is 1 and l is 0, or k is 0 and l is 1, or both k and l are 1.

The antisense oligonucleotide can therefore be represented by the following formulas:

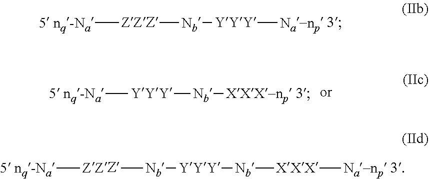

When the antisense oligonucleotide is represented by formula IIb, N_{b}′ represents 0-10, 0-7, 0-5, 0-4, 0-2, or 0 linked nucleosides. Each Nₐ′ independently represents 2-20, 2-15, or 2-10 linked nucleosides.

When the antisense oligonucleotide is represented by formula IIc, N_{b}′ represents 0-10, 0-7, 0-5, 0-4, 0-2, or 0 linked nucleosides. Each Nₐ′ independently represents 2-20, 2-15, or 2-10 linked nucleosides.

When the antisense oligonucleotide is represented by formula IId, N_{b}′ represents 0-10, 0-7, 0-5, 0-4, 0-2, or 0 linked nucleosides. Each Nₐ′ independently represents 2-20, 2-15, or 2-10 linked nucleosides. Preferably, N_{b}′ is 0, 1, 2, 3, 4, 5, or 6.

In certain embodiments, k is 0 and l is 0 and the antisense oligonucleotide may be represented by the formula:

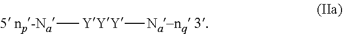

When the antisense oligonucleotide is represented by formula IIa, each Nₐ′ independently represents 2-20, 2-15, or 2-10 linked nucleosides.

Each X′, Y′, and Z′ may be the same or different from each other.

Each nucleoside of the sense oligonucleotide and antisense oligonucleotide may be independently modified with LNA, UNA, cEt, HNA, CeNA, 2′-O-methoxyethyl, 2′-O-methyl, 2′-O-allyl, 2′-C-allyl, 2′-hydroxyl, or 2′-fluoro. For example, each nucleoside of the sense oligonucleotide and antisense oligonucleotide is independently modified with, 2′-O-methyl or 2′-fluoro. Each X, Y, Z, X′, Y′, and Z′, in particular, may represent a 2′-O-methyl modification or 2′-fluoro modification. In certain embodiments, the modification is a 2′-NMA modification.

In certain embodiments, the sense oligonucleotide of the oligomeric duplex may contain YYY motif occurring at 9, 10, and 11 positions of the oligonucleotide when the duplex region is 21 nucleotides, the count starting from the 1′ nucleotide from the 5′-end, or optionally, the count starting at the 1′ paired nucleotide within the duplex region, from the 5′-end; and Y represents 2′-F modification.

The sense oligonucleotide may additionally contain XXX motif or ZZZ motifs as wing modifications at the opposite end of the duplex region; and XXX and ZZZ each independently represents a 2′-O-methyl modification or 2′-fluoro modification.

In certain embodiments, the antisense oligonucleotide may contain Y′Y′Y′ motif occurring at positions 11, 12, 13 of the oligonucleotide, the count starting from the 1′ nucleotide from the 5′-end, or optionally, the count starting at the 1′ paired nucleotide within the duplex region, from the 5′-end; and Y′ represents 2′-O-methyl modification. The antisense oligonucleotide may additionally contain X′X′X′ motif or Z′Z′Z′ motif as wing modifications at the opposite end of the duplex region; and X′X′X′ or Z′Z′Z′ each independently represents a 2′-O-methyl modification or 2′-fluoro modification.

The sense oligonucleotide represented by any one of the above formulas Ia, Ib, Ic, and Id forms a duplex with an antisense oligonucleotide being represented by any one of the formulas IIa, IIb, IIc, and IId, respectively.

Accordingly, the oligomeric duplexes described herein may comprise a sense oligonucleotide and an antisense oligonucleotide, each oligonucleotide having 14 to 30 nucleotides, the oligomeric duplex represented by formula (III):

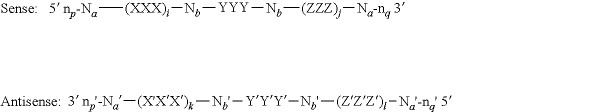

wherein:
i, j, k, and 1 are each independently 0 or 1;
p, p′, q, and q′ are each independently 0-6;
each Nₐ and Nₐ′ independently represents 0-25 linked nucleosides, each sequence comprising at least two differently modified nucleotides;
each N_{b} and N_{b}′ independently represents 0-10 linked nucleosides;
wherein each nₚ′, nₚ, n_{q}′ and n_{q}, each of which may or may not be present, independently represents an overhang nucleotide; and
XXX, YYY, X′X′X′, Y′Y′Y′, and Z′Z′Z′ each independently represent one motif of three identical modifications on three consecutive nucleotides.

In certain embodiments, i is 0 and j is 0; or i is 1 and j is 0; or i is 0 and j is 1; or both i and j are 0; or both i and j are 1. In another embodiment, k is 0 and l is 0; or k is l and l is 0, or k is 0 and l is 1; or both k and l are 0; or both k and l are 1.

Exemplary combinations of the sense oligonucleotide and antisense oligonucleotide forming a oligomeric duplex include the formulas below:

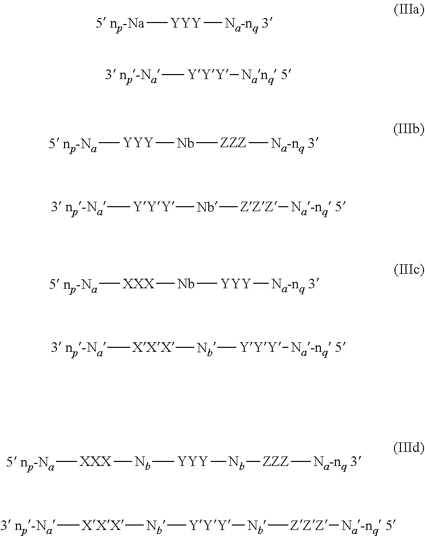

When the oligomeric duplex is represented with formula IIIa, each Nₐ independently represents 2-20, 2-15, or 2-10 linked nucleosides.

When the oligomeric duplex is represented with formula IIIb, each N_{b} independently represents 1-10, 1-7, 1-5, or 1-4 linked nucleosides. Each Nₐ independently represents 2-20, 2-15, or 2-10 linked nucleosides.

When the oligomeric duplex is represented with formula IIIc, each N_{b}, N_{b}′ independently represents 0-10, 0-7, 0-10, 0-7, 0-5, 0-4, 0-2, or 0 linked nucleosides. Each Nₐ independently represents 2-20, 2-15, or 2-10 linked nucleosides.

When the oligomeric duplex is represented with formula IIId, each N_{b}, N_{b}′ independently represents 0-10, 0-7, 0-10, 0-7, 0-5, 0-4, 0-2, or 0 linked nucleosides. Each Nₐ, Nₐ′ independently 2-20, 2-15, or 2-10 linked nucleosides. Each Nₐ, Nₐ′, N_{b}, N_{b}′ independently comprises modifications of alternating pattern.

Each of X, Y, and Z in formulas III, IIIa, IIIb, IIIc, and IIId may be the same or different from each other.

When the oligomeric duplex is represented by formula III, IIIa, IIIb, IIIc, and/or IIId, at least one of the Y nucleotides may form a base pair with one of the Y′ nucleotides. Alternatively, at least two of the Y nucleotides may form base pairs with the corresponding Y′ nucleotides; or all three of the Y nucleotides may form base pairs with the corresponding Y′ nucleotides.

When the oligomeric duplex is represented by formula IIIb or IIId, at least one of the Z nucleotides may form a base pair with one of the Z′ nucleotides. Alternatively, at least two of the Z nucleotides may form base pairs with the corresponding Z′ nucleotides; or all three of the Z nucleotides may form base pairs with the corresponding Z′ nucleotides.

When the oligomeric duplex is represented by formula IIIc or IIId, at least one of the X nucleotides may form a base pair with one of the X′ nucleotides. Alternatively, at least two of the X nucleotides may form base pairs with the corresponding X′ nucleotides; or all three of the X nucleotides may form base pairs with the corresponding X′ nucleotides.

In certain embodiments, the modification of the Y nucleotide is different than the modification on the Y′ nucleotide, the modification on the Z nucleotide is different than the modification on the Z′ nucleotide, and/or the modification on the X nucleotide is different than the modification on the X′ nucleotide.

In certain embodiments, when the oligomeric duplex is represented by the formula IIId, the Nₐ modifications are 2′-O-methyl or 2′-fluoro modifications. In another embodiment, when the oligomeric duplex is represented by formula IIId, the Nₐ modifications are 2′-O-methyl or 2′-fluoro modifications and nₚ′>0 and at least one nₚ′ is linked to a neighboring nucleotide via phosphorothioate linkage. In other embodiments, when the oligomeric duplex is represented by formula IIId, the Nₐ modifications are 2′-O-methyl or 2′-fluoro modifications, nₚ′>0 and at least one nₚ′ is linked to a neighboring nucleotide via phosphorothioate linkage, and the sense oligonucleotide is conjugated to one or more cell targeting group attached through a bivalent or trivalent branched linker. In certain embodiments, when the oligomeric duplex is represented by formula IIId, the Nₐ modifications are 2′-O-methyl or 2′-fluoro modifications, nₚ′>0 and at least one nₚ′ is linked to a neighboring nucleotide via phosphorothioate linkage, the sense oligonucleotide comprises at least one phosphorothioate linkage and the sense oligonucleotide is conjugated to one or more cell targeting group attached through a bivalent or trivalent branched linker.

In certain embodiments, when the oligomeric duplex is represented by the formula IIIa, the Nₐ modifications are 2′-O-methyl or 2′-fluoro modifications and nₚ′>0 and at least one nₚ′ is linked to a neighboring nucleotide via phosphorothioate linkage, the sense oligonucleotide comprises at least one phosphorothioate linkage and the sense oligonucleotide is conjugated to one or more cell targeting group attached through a bivalent or trivalent branched linker.

In certain embodiments, the modification is a 2′-NMA modification.

### III. Antisense Activity

In certain embodiments, oligomeric compounds and oligomeric duplexes are capable of hybridizing to a target nucleic acid, resulting in at least one antisense activity. Such oligomeric compounds and oligomeric duplexes are antisense agents. In certain antisense activities, an antisense agent or a portion of an antisense agent is loaded into an RNA-induced silencing complex (RISC), ultimately resulting in cleavage of the target nucleic acid. For example, certain antisense compounds result in cleavage of the target nucleic acid by Argonaute. Antisense agents having antisense oligonucleotides that are loaded into RISC are RNAi agents. RNAi agents may be double-stranded (siRNA or dsRNAi) or single-stranded (ssRNA).

In certain embodiments, RNAi agents are capable of RISC-mediated modulation of a target nucleic acid in a cell. In certain embodiments, such compounds reduce or inhibit the amount or activity of a target nucleic acid by 25% or more in the standard in vitro assay described in Example 2. In certain embodiments, RNAi agents selectively affect more than one target nucleic acid. Such RNAi agents comprise a nucleobase sequence that hybridizes to more than one target nucleic acid, resulting in more than one desired antisense activity. In certain embodiments, an RNAi agent does not hybridize to one or more non-target nucleic acid or does not hybridize to one or more non-target nucleic acid in such a way that results in significant undesired antisense activity.

Antisense activities may be observed directly or indirectly. In certain embodiments, observation or detection of an RNAi activity involves observation or detection of a change in an amount of a target nucleic acid or protein encoded by such target nucleic acid, a change in the ratio of splice variants of a nucleic acid or protein and/or a phenotypic change in a cell or subject.

### IV. Certain Target Nucleic Acids

In certain embodiments, antisense agents comprise an antisense oligonucleotide comprising a region that is complementary to a target nucleic acid. In certain embodiments, oligomeric compounds or oligomeric duplexes comprise an antisense oligonucleotide comprising a region that is complementary to a target nucleic acid. In certain embodiments, the target nucleic acid is an endogenous RNA molecule. In certain embodiments, the target nucleic acid encodes a protein. In certain embodiments, the oligomeric compound or oligomeric duplex is an RNAi agent.

### A. Target/Duplex Complementarity

In certain embodiments, an antisense agent comprises an antisense oligonucleotide comprising a region that is complementary to a target nucleic acid. In certain embodiments, an oligomeric compound or an oligomeric duplex comprises an antisense oligonucleotide comprising a region that is complementary to a target nucleic acid. In certain embodiments, antisense oligonucleotides are 99%, 95%, 90%, 85%, or 80% complementary to the target nucleic acid. In certain embodiments, antisense oligonucleotides are at least 80% complementary to the target nucleic acid over the entire length of the antisense oligonucleotides and comprise a region that is 100% or fully complementary to a target nucleic acid. In certain embodiments, the region of full complementarity is from 6 to 20, 10 to 18, or 18 to 20 nucleobases in length.

In certain embodiments, antisense oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the antisense oligonucleotides is improved.

In certain embodiments, antisense oligonucleotides comprise a region complementary to the target nucleic acid. In certain embodiments, the complementary region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleobases. In certain embodiments, the complementary region comprises or consists of at least 8, at least 9, at least 10, at least 11, at least 12, at least 13, at least 14, at least 15, at least 16, at least 17, at least 18, at least 19, at least 20, at least 21, at least 22, or 23 contiguous nucleobases. In certain embodiments, the complementary region constitutes 70%, 80%, 85%, 90%, or 95% of the nucleosides of the antisense oligonucleotide. In certain embodiments, the complementary region constitutes all of the nucleosides of the antisense oligonucleotide. In certain embodiments, the complementary region of the antisense oligonucleotide is at least 99%, 95%, 90%, 85%, or 80% complementary to the target nucleic acid. In certain embodiments, the complementary region of the antisense oligonucleotide is 100% complementary to the target nucleic acid.

In certain embodiments, an oligomeric duplex comprises a sense oligonucleotide. In certain embodiments, an antisense agent comprises a sense oligonucleotide. In such embodiments, the sense oligonucleotide comprises a region complementary to the antisense oligonucleotide. In certain embodiments, the complementary region comprises or consists of at least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 contiguous nucleobases. In certain embodiments, the complementary region comprises or consists of at least 8, at least 9, at least 10, at least 11, at least 12, at least 13, at least 14, at least 15, at least 16, at least 17, at least 18, at least 19, at least 20, or 21 contiguous nucleobases. In certain embodiments, the complementary region constitutes 70%, 80%, 85%, 90%, or 95% of the nucleosides of the sense oligonucleotide. In certain embodiments, the complementary region constitutes all of the nucleosides of the sense oligonucleotide. In certain embodiments, the complementary region of the sense oligonucleotide is at least 99%, 95%, 90%, 85%, or 80% complementary to the antisense oligonucleotide. In certain embodiments, the complementary region of the sense oligonucleotide is 100% complementary to the antisense oligonucleotide.

The complementary region of a sense oligonucleotide hybridizes with the antisense oligonucleotide to form a duplex region. In certain embodiments, such duplex region consists of 7 hybridized pairs of nucleosides (one of each pair being on the antisense oligonucleotide and the other of each pair being on the sense oligonucleotide). In certain embodiments, a duplex region comprises least 8, at least 9, at least 10, at least 11, at least 12, at least 13, at least 14, at least 15, at least 16, at least 17, at least 18, at least 19, at least 20, at least 21, at least 22, at least 23, at least 24, or at least 25 hybridized pairs. In certain embodiments, a duplex region comprises at least 8, at least 9, at least 10, at least 11, at least 12, at least 13, at least 14, at least 15, at least 16, at least 17, at least 18, at least 19, at least 20, or 21 hybridized pairs. In certain embodiments, each nucleoside of antisense oligonucleotide is paired in the duplex region (i.e., the antisense oligonucleotide has no overhanging nucleosides). In certain embodiments, the antisense oligonucleotide includes unpaired nucleosides at the 3′-end and/or the 5′end (overhanging nucleosides). In certain embodiments, each nucleoside of sense oligonucleotide is paired in the duplex region (i.e., the sense oligonucleotide has no overhanging nucleosides). In certain embodiments, the sense oligonucleotide includes unpaired nucleosides at the 3′-end and/or the 5′end (overhanging nucleosides). In certain embodiments, duplexes formed by the antisense oligonucleotide and the sense oligonucleotide do not include any overhangs at one or both ends. Such ends without overhangs are referred to as blunt. In certain embodiments wherein the antisense oligonucleotide has overhanging nucleosides, one or more of those overhanging nucleosides are complementary to the target nucleic acid. In certain embodiments wherein the antisense oligonucleotide has overhanging nucleosides, one or more of those overhanging nucleosides are not complementary to the target nucleic acid.

### B. SNCA

In certain embodiments, antisense agents disclosed herein comprise an antisense oligonucleotide comprising a region that is complementary to a target nucleic acid, wherein the target nucleic acid is SNCA RNA. In certain embodiments, oligomeric compounds or oligomeric duplexes disclosed herein comprise an antisense oligonucleotide comprising a region that is complementary to a target nucleic acid, wherein the target nucleic acid is SNCA RNA. In each of the embodiments described above, the oligomeric compound, oligomeric duplex, or antisense agent may target SNCA RNA. In certain embodiments, the oligomeric compound or oligomeric duplex is an RNAi agent. In certain embodiments, the antisense agent is an RNAi agent. In certain embodiments, SNCA RNA has the sequence of SEQ ID NO: 1 (GENBANK Accession No. NM_000345.3), SEQ ID NO: 2 (GENBANK Accession No: NT_016354.20 TRUNC 30800000-30919000), SEQ ID NO: 3 (GENBANK Accession No: JN709863.1), SEQ ID NO: 4 (GENBANK Accession No: BC013293.2), SEQ ID NO: 5 (GENBANK Accession No: NM_001146055.1), and SEQ ID NO: 6 (GENBANK Accession No: HQ830269.1). In certain embodiments, contacting a cell with an oligomeric compound comprising or consisting of an antisense oligonucleotide complementary to SEQ ID NO: 1, SEQ ID NO: 2, SEQ ID NO: 3, SEQ ID NO: 4, SEQ ID NO: 5, or SEQ ID NO: 6 reduces the amount of SNCA RNA, and in certain embodiments, reduces the amount of SNCA protein. In certain embodiments, contacting a cell with an oligomeric duplex comprising an oligomeric compound, in which the oligomeric compound comprises or consists of an antisense oligonucleotide complementary to SEQ ID NO: 1, SEQ ID NO: 2, SEQ ID NO: 3, SEQ ID NO: 4, SEQ ID NO: 5, or SEQ ID NO: 6, reduces the amount of SNCA RNA, and in certain embodiments reduces the amount of SNCA protein. In certain embodiments, the oligomeric duplex is an antisense agent, and the antisense agent comprises an oligomeric compound that comprises or consists of an antisense oligonucleotide complementary to SEQ ID NO: 1, SEQ ID NO: 2, SEQ ID NO: 3, SEQ ID NO: 4, SEQ ID NO: 5, or SEQ ID NO: 6. In certain embodiments, the oligomeric compound, the oligomeric duplex, or the antisense agent comprises a conjugate group. In certain embodiments, the oligomeric compounds, the oligomeric duplex, or the antisense agent comprises more than one conjugate group.

In certain embodiments, contacting a cell in a subject with an oligomeric compound disclosed herein comprising or consisting of an antisense oligonucleotide complementary to SEQ ID NO: 1, SEQ ID NO: 2, SEQ ID NO: 3, SEQ ID NO: 4, SEQ ID NO: 5, or SEQ ID NO: 6 ameliorates one or more symptoms or hallmarks of a disease associated with SNCA In certain embodiments, the one or more symptoms or hallmarks is motor dysfunction, aggregation of alpha-synuclein, neurodegeneration, cognitive decline, dementia, sleep disorders, hyposmia, autonomic failure, ataxia, hallucination, or seizures. In certain embodiments, contacting a cell in a subject with an oligomeric compound disclosed herein comprising or consisting of an antisense oligonucleotide complementary to SEQ ID NO: 1, SEQ ID NO: 2, SEQ ID NO: 3, SEQ ID NO: 4, SEQ ID NO: 5, or SEQ ID NO: 6 ameliorates one or more symptoms or hallmarks of a neurodegenerative disease. In certain embodiments, the symptom or hallmark is motor dysfunction, aggregation of alpha-synuclein, neurodegeneration, cognitive decline, dementia, sleep disorders, hyposmia, autonomic failure, ataxia, hallucination, or seizures. In certain embodiments, contacting a cell in a subject with an RNAi agent disclosed herein comprising or consisting of an antisense oligonucleotide complementary to SEQ ID NO: 1, SEQ ID NO: 2, SEQ ID NO: 3, SEQ ID NO: 4, SEQ ID NO: 5, or SEQ ID NO: 6 improves motor function, reduces the amount or volume of alpha-synuclein aggregates, reduces or delays neurodegeneration, improves cognitive function, delays the onset or progression of dementia, reduce sleep disorders, improves hyposmia, reduces or delays autonomic failure, reduces ataxia, reduces hallucination, or reduces seizures.

In certain embodiments, contacting a cell in a subject with an oligomeric duplex disclosed herein comprising an antisense oligonucleotide complementary to SEQ ID NO: 1, SEQ ID NO: 2, SEQ ID NO: 3, SEQ ID NO: 4, SEQ ID NO: 5, or SEQ ID NO: 6 ameliorates one or more symptoms or hallmarks of a disease associated with SNCA. In certain embodiments, the one or more symptoms or hallmarks is motor dysfunction, aggregation of alpha-synuclein, neurodegeneration, cognitive decline, dementia, sleep disorders, hyposmia, autonomic failure, ataxia, hallucination, or seizures. In certain embodiments, contacting a cell in a subject with an oligomeric duplex disclosed herein comprising an antisense oligonucleotide complementary to SEQ ID NO: 1, SEQ ID NO: 2, SEQ ID NO: 3, SEQ ID NO: 4, SEQ ID NO: 5, or SEQ ID NO: 6 improves motor function, reduces the amount or volume of alpha-synuclein aggregates, reduces or delays neurodegeneration, improves cognitive function, delays the onset or progression of dementia, reduce sleep disorders, improves hyposmia, reduces or delays autonomic failure, reduces ataxia, reduces hallucination, or reduces seizures.

In certain embodiments, contacting a cell in a subject with an antisense agent disclosed herein comprising an antisense oligonucleotide complementary to SEQ ID NO: 1, SEQ ID NO: 2, SEQ ID NO: 3, SEQ ID NO: 4, SEQ ID NO: 5, or SEQ ID NO: 6 ameliorates one or more symptoms or hallmarks of a disease associated with SNCA. In certain embodiments, the one or more symptoms or hallmarks is motor dysfunction, aggregation of alpha-synuclein, neurodegeneration, cognitive decline, dementia, sleep disorders, hyposmia, autonomic failure, ataxia, hallucination, or seizures. In certain embodiments, contacting a cell in a subject with an antisense agent disclosed herein comprising an antisense oligonucleotide complementary to SEQ ID NO: 1, SEQ ID NO: 2, SEQ ID NO: 3, SEQ ID NO: 4, SEQ ID NO: 5, or SEQ ID NO: 6 improves motor function, reduces the amount or volume of alpha-synuclein aggregates, reduces or delays neurodegeneration, improves cognitive function, delays the onset or progression of dementia, reduce sleep disorders, improves hyposmia, reduces or delays autonomic failure, reduces ataxia, reduces hallucination, or reduces seizures.

### C. Certain Target Nucleic Acids in Certain Tissues

In certain embodiments, oligomeric compounds comprise or consist of an antisense oligonucleotide comprising a region that is complementary to a target nucleic acid, wherein the target nucleic acid is expressed in a pharmacologically relevant tissue. In certain embodiments, oligomeric duplexes comprise an antisense oligonucleotide comprising a region that is complementary to a target nucleic acid, wherein the target nucleic acid is expressed in a pharmacologically relevant tissue. In certain embodiments, the pharmacologically relevant tissues are the cells and tissues that comprise the central nervous system (CNS). Such tissues include the brain and spinal cord. In certain embodiments, the pharmacologically relevant tissues are the cells and tissues that comprise the central nervous system (CNS). Such cells and tissues include motor cortex, frontal cortex, caudate, amygdala, pons, substantia nigra, putamen, cerebellar peduncle, corpus collosum, deep cerebellar nuclei, entorhinal cortex (Ent Cortex), hippocampus, insular cortex, medulla oblongata, central gray matter, pulvinar, occipital cortex, cerebral cortex, temporal cortex, globus pallidus, superior colliculi, and basal forebrain nuclei. In certain embodiments, the cells are brain cells. In certain embodiments, the cells are neuronal cells. In certain embodiments, the cells are non-neuronal cells. In certain embodiments, the cells include neurons and oligodendrocytes. In certain embodiments, the oligomeric compounds or oligomeric duplexes are antisense agents.

### V. Certain Methods and Uses

Certain embodiments provided herein relate to methods of inhibiting SNCA RNA expression or activity, which can be useful for treating or ameliorating a disease or disorder associated with SNCA. In certain embodiments, the disease or disorder associated with SNCA is a neurodegenerative disease. In certain embodiments, the disease or disorder associated with SNCA is a synucleinopathy. In certain embodiments, the disease or disorder associated with SNCA is Parkinson's disease, dementia with Lewy bodies, diffuse Lewy body disease, Parkinson's disease dementia, pure autonomic failure, multiple system atrophy (MSA), neuronopathic Gaucher's disease, or Alzheimer's disease.

In certain embodiments, a method comprises administering to a subject an oligomeric compound or an oligomeric duplex, any of which having a nucleobase sequence complementary to SNCA. In certain embodiments, the subject has or is at risk for developing a disease or disorder associated with SNCA. In certain embodiments, the subject has or is at risk for developing a neurodegenerative disease. In certain embodiments, the subject has or is at risk for developing a synucleinopathy. In certain embodiments, the subject has or is at risk for developing Parkinson's disease, dementia with Lewy bodies, diffuse Lewy body disease, Parkinson's disease dementia, pure autonomic failure, multiple system atrophy (MSA), neuronopathic Gaucher's disease, or Alzheimer's disease. In certain embodiments, the oligomeric compound or oligomeric duplex is an antisense agent.

In certain embodiments, a method of treating a disease or disorder associated with SNCA comprises administering to a subject an oligomeric compound, an oligomeric duplex, or an antisense agent, any of which having a nucleobase sequence complementary to SNCA. In certain embodiments, the subject has or is at risk for developing a neurodegenerative disease. In certain embodiments, the subject has or is at risk for developing a synucleinopathy. In certain embodiments, the subject has or is at risk for developing Parkinson's disease, dementia with Lewy bodies, diffuse Lewy body disease, Parkinson's disease dementia, pure autonomic failure, multiple system atrophy (MSA), neuronopathic Gaucher's disease, or Alzheimer's disease. In certain embodiments, at least one symptom or hallmark of the disease or disorder associated with SNCA is ameliorated. In certain embodiments, the at least one symptom or hallmark is motor dysfunction, aggregation of alpha-synuclein, neurodegeneration, cognitive decline, dementia, sleep disorders, hyposmia, autonomic failure, ataxia, hallucination, or seizures. In certain embodiments, administration of the oligomeric compound, the oligomeric duplex, or the antisense agent to the subject improves motor function, reduces the amount or volume of alpha-synuclein aggregates, reduces or delays neurodegeneration, improves cognitive function, delays the onset or progression of dementia, reduce sleep disorders, improves hyposmia, reduces or delays autonomic failure, reduces ataxia, reduces hallucination, or reduces seizures.

In certain embodiments, a method of reducing expression of SNCA or reducing SNCA protein in a cell comprises contacting the cell with an oligomeric compound, an oligomeric duplex, or an antisense agent, any of which having a nucleobase sequence complementary to SNCA. In certain embodiments, the cell is a central nervous system cell. In certain embodiments, the cell is a brain cell. In certain embodiments, the cell is a neuronal cell. In certain embodiments, the cell is a non-neuronal cell. In certain embodiments, the cell is an oligodendrocyte. In certain embodiments, the cell is a human cell.

Certain embodiments are drawn to an oligomeric compound, an oligomeric duplex, or an antisense agent, any of which having a nucleobase sequence complementary to SNCA, for use in treating a disease or disorder associated with SNCA or for use in the manufacture of a medicament for treating a disease or disorder associated with SNCA. In certain embodiments, the disease or disorder associated with SNCA is a neurodegenerative disease. In certain embodiments, the disease or disorder associated with SNCA is a synucleinopathy. In certain embodiments, the disease or disorder associated with SNCA is Parkinson's disease, dementia with Lewy bodies, diffuse Lewy body disease, Parkinson's disease dementia, pure autonomic failure, multiple system atrophy, neuronopathic Gaucher's disease, or Alzheimer's disease.

In any of the methods or uses described herein, the oligomeric compound, the oligomeric duplex, or the antisense agent can be any described herein.

### VI. Certain Pharmaceutical Compositions

Oligomeric compounds, oligomeric duplex, or antisense agents described herein may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered. In certain embodiments, the oligomeric compound or oligomeric duplex is an RNAi agent.

Certain embodiments provide pharmaceutical compositions comprising one or more oligomeric compounds, oligomeric duplexes, or antisense agents, or a salt thereof. In certain such embodiments, the pharmaceutical composition comprises a suitable pharmaceutically acceptable diluent or carrier. In certain embodiments, a pharmaceutical composition comprises a sterile saline solution and one or more oligomeric compounds, oligomeric duplexes, or antisense agents. In certain embodiments, such pharmaceutical composition consists of a sterile saline solution and one or more oligomeric compounds, oligomeric duplexes, or antisense agents. In certain embodiments, the sterile saline is pharmaceutical grade saline. In certain embodiments, a pharmaceutical composition comprises one or more oligomeric compounds, oligomeric duplexes, or antisense agents, and sterile water. In certain embodiments, a pharmaceutical composition consists of one or more oligomeric compounds, oligomeric duplexes, or antisense agents, and sterile water. In certain embodiments, the sterile water is pharmaceutical grade water. In certain embodiments, a pharmaceutical composition comprises one or more oligomeric compounds, oligomeric duplexes, or antisense agents, and phosphate-buffered saline (PBS). In certain embodiments, a pharmaceutical composition consists of one or more oligomeric compounds, oligomeric duplexes, or antisense agents, and sterile PBS. In certain embodiments, the sterile PBS is pharmaceutical grade PBS. In certain embodiments, a pharmaceutical composition consists of cerebrospinal fluid (CSF) and one or more oligomeric compounds, oligomeric duplexes, or antisense agents. In certain embodiments, the oligomeric duplexes or antisense agents comprise a sense oligonucleotide and an antisense oligonucleotide. In certain embodiments, the CSF is artificial CSF (aCSF). Compositions and methods for the formulation of pharmaceutical compositions are dependent upon a number of criteria, including, but not limited to, route of administration, extent of disease, or dose to be administered.

In certain embodiments, a pharmaceutical composition comprises one or more oligomeric compounds, oligomeric duplexes, or antisense agents and artificial cerebrospinal fluid (aCSF). In certain embodiments, a pharmaceutical composition consists of one or more oligomeric compounds, oligomeric duplexes, or antisense agents and artificial cerebrospinal fluid. In certain embodiments, a pharmaceutical composition consists essentially of one or more oligomeric compounds, oligomeric duplexes, or antisense agents and artificial cerebrospinal fluid. In certain embodiments, the artificial cerebrospinal fluid is pharmaceutical grade.

In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric compounds, oligomeric duplexes, or antisense agents, and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds, oligomeric duplexes, or antisense agents may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

Pharmaceutical compositions comprising oligomeric compounds, oligomeric duplexes, or antisense agents provided herein encompass any pharmaceutically acceptable salts, esters, or salts of such esters, which, upon administration to an animal, including a human, is capable of providing (directly or indirectly) the biologically active metabolite or residue thereof. Accordingly, for example, the disclosure is also drawn to pharmaceutically acceptable salts of compounds, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts.

A prodrug can include the incorporation of additional nucleosides at one or both ends of an oligomeric compound, oligomeric duplex, or antisense agent, which are cleaved by endogenous nucleases within the body, to form the active compound.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents comprising an oligomeric compound, an oligomeric duplex, or an antisense agent provided herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

In certain embodiments, oligomeric compounds, oligomeric duplexes, or antisense agents are in aqueous solution with sodium. In certain embodiments, oligomeric compounds, oligomeric duplexes, or antisense agents are in aqueous solution with potassium. In certain embodiments, oligomeric compounds, oligomeric duplexes, or antisense agents are in PBS. In certain embodiments, oligomeric compounds, oligomeric duplexes, or antisense agents are in water. In certain embodiments, oligomeric compounds, oligomeric duplexes, or antisense agents are in aCSF. In certain such embodiments, the pH of the solution is adjusted with NaOH and/or HCl to achieve a desired pH.

### VII. Certain Hotspot Regions

### 1. Nucleobases 775-997 of SEO ID NO: 1

In certain embodiments, nucleobases 775-997 of SEQ ID NO: 1 comprise a hotspot region. In certain embodiments, oligomeric compounds, oligomeric duplexes, or antisense agents comprise antisense oligonucleotides complementary to a portion of nucleobases 775-997 of SEQ ID NO: 1. In certain embodiments, the antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length. In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyfyfyfyfyfyfyfyfyfyyy-3′, wherein each “y” represents a 2′-OMe sugar moiety, and each “f” represents a 2′-F sugar moiety.

The nucleobase sequences of SEQ ID NOs: 45, 46, 47, 48, 49, 50, 51, 53, 54, 55, and 56 are complementary to nucleobases 775-997 of SEQ ID NO: 1. The nucleobase sequences of the antisense oligonucleotides of Compound Nos: 1518800, 1518801, 1518802, 1518803, 1518804, 1518805, 1518818, 1518820, 1518821, 1518822, and 1518823 are complementary to nucleobases 775-997 of SEQ ID NO: 1.

In certain embodiments, oligomeric compounds, oligomeric duplexes, or antisense agents comprising antisense oligonucleotides complementary to a portion of nucleobases 775-997 of SEQ ID NO: 1 achieve at least 45% reduction of SNCA RNA in the standard in vitro assay. In certain embodiments, oligomeric compounds, oligomeric duplexes, or antisense agents comprising antisense oligonucleotides complementary to a portion of nucleobases 775-997 of SEQ ID NO: 1 achieve an average of 55.6% reduction of SNCA RNA in the standard in vitro assay. In certain embodiments, oligomeric duplexes comprising antisense oligonucleotides complementary to a portion of nucleobases 775-997 of SEQ ID NO: 1 achieve at least 65% reduction of SNCA RNA in the standard in vitro assay. In certain embodiments, oligomeric duplexes comprising antisense oligonucleotides complementary to a portion of nucleobases 775-997 of SEQ ID NO: 1 achieve an average of 78.6% reduction of SNCA RNA in the standard in vitro assay.

### 2. Nucleobases 1115-1237 of SEO ID NO: 1

In certain embodiments, nucleobases 1115-1237 of SEQ ID NO: 1 comprise a hotspot region. In certain embodiments, oligomeric compounds, oligomeric duplexes, or antisense agents comprise antisense oligonucleotides complementary to a portion of nucleobases 1115-1237 of SEQ ID NO: 1. In certain embodiments, the antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length. In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyfyfyfyfyfyfyfyfyfyyy-3′, wherein each “y” represents a 2′-OMe sugar moiety, and each “f” represents a 2′-F sugar moiety.

The nucleobase sequences of SEQ ID NOs: 58, 63, 65, 66, 67, and 68 are complementary to nucleobases 1115-1237 of SEQ ID NO: 1. The nucleobase sequences of the antisense oligonucleotides of Compound Nos: 1518837, 1518854, 1518856, 1518857, 1518858, and 1518859 are complementary to nucleobases 1115-1237 of SEQ ID NO: 1.

In certain embodiments, oligomeric compounds, oligomeric duplexes, or antisense agents comprising antisense oligonucleotides complementary to a portion of nucleobases 1115-1237 of SEQ ID NO: 1 achieve at least 53% reduction of SNCA RNA in the standard in vitro assay. In certain embodiments, oligomeric compounds, oligomeric duplexes, or antisense agents comprising antisense oligonucleotides complementary to a portion of nucleobases 1115-1237 of SEQ ID NO: 1 achieve an average of 62.8% reduction of SNCA RNA in the standard in vitro assay. In certain embodiments, oligomeric duplexes comprising antisense oligonucleotides complementary to a portion of nucleobases 1115-1237 of SEQ ID NO: 1 achieve at least 70% reduction of SNCA RNA in the standard in vitro assay. In certain embodiments, oligomeric duplexes comprising antisense oligonucleotides complementary to a portion of nucleobases 1115-1237 of SEQ ID NO: 1 achieve an average of 75.3% reduction of SNCA RNA in the standard in vitro assay.

### 3. Nucleobases 1455-1577 of SEO ID NO: 1

In certain embodiments, nucleobases 1455-1577 of SEQ ID NO: 1 comprise a hotspot region. In certain embodiments, oligomeric compounds, oligomeric duplexes, or antisense agents comprise antisense oligonucleotides complementary to a portion of nucleobases 1455-1577 of SEQ ID NO: 1. In certain embodiments, the antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length. In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyfyfyfyfyfyfyfyfyfyyy-3′, wherein each “y” represents a 2′-OMe sugar moiety, and each “f” represents a 2′-F sugar moiety.

The nucleobase sequences of SEQ ID NOs: 76, 77, 81, 83, 84, and 85 are complementary to nucleobases 1455-1577 of SEQ ID NO: 1. The nucleobase sequences of the antisense oligonucleotides of Compound Nos: 1518891, 1518892, 1518908, 1518910, 1518911, and 1518912 are complementary to nucleobases 1455-1577 of SEQ ID NO: 1.

In certain embodiments, oligomeric compounds, oligomeric duplexes, or antisense agents comprising antisense oligonucleotides complementary to a portion of nucleobases 1455-1577 of SEQ ID NO: 1 achieve at least 50% reduction of SNCA RNA in the standard in vitro assay. In certain embodiments, oligomeric compounds, oligomeric duplexes, or antisense agents comprising antisense oligonucleotides complementary to a portion of nucleobases 1455-1577 of SEQ ID NO: 1 achieve an average of 55.3% reduction of SNCA RNA in the standard in vitro assay. In certain embodiments, oligomeric duplexes comprising antisense oligonucleotides complementary to a portion of nucleobases 1455-1577 of SEQ ID NO: 1 achieve at least 58% reduction of SNCA RNA in the standard in vitro assay. In certain embodiments, oligomeric duplexes comprising antisense oligonucleotides complementary to a portion of nucleobases 1455-1577 of SEQ ID NO: 1 achieve an average of 63.5% reduction of SNCA RNA in the standard in vitro assay.

### 4. Nucleobases 1315-1397 of SEO ID NO: 1

In certain embodiments, nucleobases 1315-1397 of SEQ ID NO: 1 comprise a hotspot region. In certain embodiments, oligomeric compounds, oligomeric duplexes, or antisense agents comprise antisense oligonucleotides complementary to a portion of nucleobases 1315-1397 of SEQ ID NO: 1. In certain embodiments, the antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length. In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyfyfyfyfyfyfyfyfyfyyy-3′, wherein each “y” represents a 2′-OMe sugar moiety, and each “f” represents a 2′-F sugar moiety.

The nucleobase sequences of SEQ ID NOs: 70, 71, 72, and 75 are complementary to nucleobases 1315-1397 of SEQ ID NO: 1. The nucleobase sequences of the antisense oligonucleotides of Compound Nos: 1518875, 1518874, 1518873, and 1518890 are complementary to nucleobases 1315-1397 of SEQ ID NO: 1.

In certain embodiments, oligomeric compounds, oligomeric duplexes, or antisense agents comprising antisense oligonucleotides complementary to a portion of nucleobases 1315-1397 of SEQ ID NO: 1 achieve at least 33% reduction of SNCA RNA in the standard in vitro assay. In certain embodiments, oligomeric compounds, oligomeric duplexes, or antisense agents comprising antisense oligonucleotides complementary to a portion of nucleobases 1315-1397 of SEQ ID NO: 1 achieve an average of 58.8% reduction of SNCA RNA in the standard in vitro assay. In certain embodiments, oligomeric duplexes comprising antisense oligonucleotides complementary to a portion of nucleobases 1315-1397 of SEQ ID NO: 1 achieve at least 45% reduction of SNCA RNA in the standard in vitro assay. In certain embodiments, oligomeric duplexes comprising antisense oligonucleotides complementary to a portion of nucleobases 1315-1397 of SEQ ID NO: 1 achieve an average of 67.3% reduction of SNCA RNA in the standard in vitro assay.

### 5. Nucleobases 1475-1577 of SEO ID NO: 1

In certain embodiments, nucleobases 1475-1577 of SEQ ID NO: 1 comprise a hotspot region. In certain embodiments, oligomeric compounds, oligomeric duplexes, or antisense agents comprise antisense oligonucleotides complementary to a portion of nucleobases 1475-1577 of SEQ ID NO: 1. In certain embodiments, the antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length. In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyfyfyfyfyfyfyfyfyfyyy-3′, wherein each “y” represents a 2′-OMe sugar moiety, and each “f” represents a 2′-F sugar moiety.

The nucleobase sequences of SEQ ID NOs: 76, 81, 83, 84, and 85 are complementary to nucleobases 1475-1577 of SEQ ID NO: 1. The nucleobase sequences of the antisense oligonucleotides of Compound Nos: 1518891, 1518908, 1518912, 1518911, and 1518910 are complementary to nucleobases 1475-1577 of SEQ ID NO: 1.

In certain embodiments, oligomeric compounds, oligomeric duplexes, or antisense agents comprising antisense oligonucleotides complementary to a portion of nucleobases 1475-1577 of SEQ ID NO: 1 achieve at least 50% reduction of SNCA RNA in the standard in vitro assay. In certain embodiments, oligomeric compounds, oligomeric duplexes, or antisense agents comprising antisense oligonucleotides complementary to a portion of nucleobases 1475-1577 of SEQ ID NO: 1 achieve an average of 54% reduction of SNCA RNA in the standard in vitro assay. In certain embodiments, oligomeric duplexes comprising antisense oligonucleotides complementary to a portion of nucleobases 1475-1577 of SEQ ID NO: 1 achieve at least 58% reduction of SNCA RNA in the standard in vitro assay. In certain embodiments, oligomeric duplexes comprising antisense oligonucleotides complementary to a portion of nucleobases 1475-1577 of SEQ ID NO: 1 achieve an average of 61.6% reduction of SNCA RNA in the standard in vitro assay.

### 6. Nucleobases 1655-1737 of SEO ID NO: 1

In certain embodiments, nucleobases 1655-1737 of SEQ ID NO: 1 comprise a hotspot region. In certain embodiments, oligomeric compounds, oligomeric duplexes, or antisense agents comprise antisense oligonucleotides complementary to a portion of nucleobases 1655-1737 of SEQ ID NO: 1. In certain embodiments, the antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length. In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyfyfyfyfyfyfyfyfyfyyy-3′, wherein each “y” represents a 2′-OMe sugar moiety, and each “f” represents a 2′-F sugar moiety.

The nucleobase sequences of SEQ ID NOs: 88, 89, 90, and 92 are complementary to nucleobases 1655-1737 of SEQ ID NO: 1. The nucleobase sequences of the antisense oligonucleotides of Compound Nos: 1518929, 1518928, 1518927, and 1518931 are complementary to nucleobases 1655-1737 of SEQ ID NO: 1.

In certain embodiments, oligomeric compounds, oligomeric duplexes, or antisense agents comprising antisense oligonucleotides complementary to a portion of nucleobases 1655-1737 of SEQ ID NO: 1 achieve at least 17% reduction of SNCA RNA in the standard in vitro assay. In certain embodiments, oligomeric compounds, oligomeric duplexes, or antisense agents comprising antisense oligonucleotides complementary to a portion of nucleobases 1655-1737 of SEQ ID NO: 1 achieve an average of 45.8% reduction of SNCA RNA in the standard in vitro assay. In certain embodiments, oligomeric duplexes comprising antisense oligonucleotides complementary to a portion of nucleobases 1655-1737 of SEQ ID NO: 1 achieve at least 38% reduction of SNCA RNA in the standard in vitro assay. In certain embodiments, oligomeric duplexes comprising antisense oligonucleotides complementary to a portion of nucleobases 1655-1737 of SEQ ID NO: 1 achieve an average of 64.5% reduction of SNCA RNA in the standard in vitro assay.

### 7. Nucleobases 495-517 of SEO ID NO: 1

In certain embodiments, nucleobases 495-517 of SEQ ID NO: 1 comprise a hotspot region. In certain embodiments, oligomeric compounds, oligomeric duplexes, or antisense agents comprise antisense oligonucleotides complementary to a portion of nucleobases 495-517 of SEQ ID NO: 1. In certain embodiments, the antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length. In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyfyfyfyfyfyfyfyfyfyyy-3′, wherein each “y” represents a 2′-OMe sugar moiety, and each “f” represents a 2′-F sugar moiety.

The nucleobase sequences of SEQ ID NO: 29 is complementary to nucleobases 495-517 of SEQ ID NO: 1. The nucleobase sequences of the antisense oligonucleotides of Compound No. 1518748 is complementary to nucleobases 495-517 of SEQ ID NO: 1.

### 8. Nucleobases 1375-1397 of SEO ID NO: 1

In certain embodiments, nucleobases 1375-1397 of SEQ ID NO: 1 comprise a hotspot region. In certain embodiments, oligomeric compounds, oligomeric duplexes, or antisense agents comprise antisense oligonucleotides complementary to a portion of nucleobases 1375-1397 of SEQ ID NO: 1. In certain embodiments, the antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length. In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyfyfyfyfyfyfyfyfyfyyy-3′, wherein each “y” represents a 2′-OMe sugar moiety, and each “f” represents a 2′-F sugar moiety.

The nucleobase sequences of SEQ ID NO: 75 is complementary to nucleobases 1375-1397 of SEQ ID NO: 1. The nucleobase sequences of the antisense oligonucleotides of Compound No. 1518890 is complementary to nucleobases 1375-1397 of SEQ ID NO: 1.

### 9. Nucleobases 1475-1517 of SEO ID NO: 1

In certain embodiments, nucleobases 1475-1517 of SEQ ID NO: 1 comprise a hotspot region. In certain embodiments, oligomeric compounds, oligomeric duplexes, or antisense agents comprise antisense oligonucleotides complementary to a portion of nucleobases 1475-1517 of SEQ ID NO: 1. In certain embodiments, the antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length. In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyfyfyfyfyfyfyfyfyfyyy-3′, wherein each “y” represents a 2′-OMe sugar moiety, and each “f” represents a 2′-F sugar moiety.

The nucleobase sequences of SEQ ID NOs: 76 and 81 are complementary to nucleobases 1475-1517 of SEQ ID NO: 1. The nucleobase sequences of the antisense oligonucleotides of Compound Nos: 1518891 and 1518908 are complementary to nucleobases 1475-1517 of SEQ ID NO: 1.

### 10. Nucleobases 1555-1577 of SEO ID NO: 1

In certain embodiments, nucleobases 1555-1577 of SEQ ID NO: 1 comprise a hotspot region. In certain embodiments, oligomeric compounds, oligomeric duplexes, or antisense agents comprise antisense oligonucleotides complementary to a portion of nucleobases 1555-1577 of SEQ ID NO: 1. In certain embodiments, the antisense oligonucleotides are 15 to 30 nucleobases in length. In certain embodiments, the antisense oligonucleotides are 17 to 30, 18 to 30, 18 to 25, or 20 to 23 nucleobases in length. In certain embodiments, the antisense oligonucleotides are 23 nucleobases in length. In certain embodiments, the antisense oligonucleotide has an internucleoside linkage motif of 5′-ssooooooooooooooooooss-3′, wherein each “s” is a phosphorothioate internucleoside linkage and each “o” is a phosphodiester internucleoside linkage. In certain embodiments, the antisense oligonucleotide has a sugar motif of 5′-yfyfyfyfyfyfyfyfyfyfyyy-3′, wherein each “y” represents a 2′-OMe sugar moiety, and each “f” represents a 2′-F sugar moiety.

The nucleobase sequences of SEQ ID NO: 83 is complementary to nucleobases 1555-1577 of SEQ ID NO: 1. The nucleobase sequences of the antisense oligonucleotides of Compound No. 1518910 is complementary to nucleobases 1555-1577 of SEQ ID NO: 1.

### Nonlimiting Disclosure and Incorporation by Reference

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, unless otherwise stated, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such oligomeric compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position. Finally, for clarity, unless otherwise indicated, the phrase “nucleobase sequence of SEQ ID NO: X”, refers only to the sequence of nucleobases in that SEQ ID NO: X, independent of any sugar or internucleoside linkage modifications also described in such SEQ ID NO.

While effort has been made to accurately describe compounds in the accompanying sequence listing, should there be any discrepancies between a description in this specification and in the accompanying sequence listing, the description in the specification and not in the sequence listing is the accurate description.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or f such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ²C, ¹⁵N in place of ¹⁴N, ¹⁷O or ¹⁸O in place of ¹⁶O, and ³³, ³⁴S, ³S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1: Design of Oligomeric Duplexes that Target a Human SNCA Nucleic Acid

Oligomeric duplexes comprising antisense oligonucleotides complementary to a human SNCA nucleic acid, and sense oligonucleotides complementary to the antisense oligonucleotides were designed as follows.

The antisense oligonucleotide in each case is 23 nucleosides in length; has a sugar motif (from 5′ to 3′) of: yfyfyfyfyfyfyfyfyfyfyyy; wherein each ‘y’ represents a ribo-2′-OMe sugar moiety and each “f” represents a 2′-F sugar moiety; and an internucleoside linkage motif (from 5′ to 3′) of: ssooooooooooooooooooss; wherein each ‘o’ represents a phosphodiester internucleoside linkage and each ‘s’ represents a phosphorothioate internucleoside linkage. Each cytosine residue is a non-methylated cytosine. Each antisense oligonucleotide has a terminal phosphate at the 5′-end. The antisense oligonucleotides are listed below in Tables 1 and 3.

Each antisense oligonucleotides is complementary to the target nucleic acid (SNCA), and each sense oligonucleotides is complementary to the first of the 21 nucleosides of the antisense oligonucleotide (from 5′ to 3′) wherein the last two 3′-nucleosides of the antisense oligonucleotides are not paired with the sense oligonucleotide (are overhanging nucleosides). The sense oligonucleotides and oligomeric duplexes are listed in Tables 2 and 4 below.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified antisense oligonucleoside listed in Tables 1 and 3 below is 100% complementary to SEQ ID NO: 1 (GENBANK Accession No: NM_000345.3), to SEQ ID NO: 2 (the complement of GENBANK Accession No: NT_016354.20 TRUNC 30800000-30919000), to SEQ NO: 5 (GENBANK Accession No: NM_001146055.1), or to both SEQ ID NOs 1 and 2. ‘N/A’ indicates that the modified oligonucleotide is not 100% complementary to that particular target nucleic acid sequence.

TABLE 1
Design of antisense modified oligonucleotides targeted to human SNCA

| | SEQ ID | SEQ ID | SEQ ID | SEQ ID | | |
|---|---|---|---|---|---|---|
| Antisense | NO: 1 | NO: 1 | NO: 2 | NO: 2 | | |
| oligo. | Antisense | Antisense | Antisense | Antisense | | SEQ |
| Compound | oligo. | oligo. | oligo. | oligo. | Antisense Oligo. | ID |
| No. | Start Site | Stop Site | Start Site | Stop Site | Sequence (5′ to 3′) | NO |
| 1518724 | N/A | N/A | 2087 | 2109 | GCUGUUCCUGGAUCACACCAGAA | 16 |
| 1518723 | 255 | 277 | 4706 | 4728 | AUGAAUACAUCCAUGGCUAAUGA | 17 |
| 1518722 | N/A | N/A | 2107 | 2129 | UCUUUCAGAGCUGGAAGACAGCU | 18 |
| 1518727 | N/A | N/A | 2067 | 2089 | GAAUGGAGAAGCAAGCUCCUCCC | 19 |
| 1518726 | 275 | 297 | 4726 | 4748 | UGGCCUUUGAAAGUCCUUUCAUG | 20 |
| 1518741 | 295 | 317 | 4746 | 4768 | AGCAGCCACAACUCCCUCCUUGG | 21 |
| 1518740 | 315 | 337 | 4766 | 4788 | CCCUGUUUGGUUUUCUCAGCAGC | 22 |
| 1518742 | 375 | 397 | N/A | N/A | UUGGUUUUGGAGCCUACAUAGAG | 23 |
| 1518745 | 355 | 377 | 4806 | 4828 | GAGAACACCCUCUUUUGUCUUUC | 24 |
| 1518744 | 335 | 357 | 4786 | 4808 | UUCCUGCUGCUUCUGCCACACCC | 25 |
| 1518743 | 395 | 417 | 12208 | 12230 | CACCAUGCACCACUCCCUCCUUG | 26 |
| 1518760 | 435 | 457 | 18002 | 18024 | UUUGUCACUUGCUCUUUGGUCUU | 27 |
| 1518759 | 515 | 537 | 18082 | 18104 | UGGCUGCUGCAAUGCUCCCUGCU | 28 |
| 1518761 | 495 | 517 | 18062 | 18084 | GCUCCCUCCACUGUCUUCUGGGC | 29 |
| 1518758 | 475 | 497 | 18042 | 18064 | GGCUACUGCUGUCACACCCGUCA | 30 |
| 1518763 | 455 | 477 | 18022 | 18044 | UCACCACUGCUCCUCCAACAUUU | 31 |
| 1518762 | 415 | 437 | N/A | N/A | CUUCUCAGCCACUGUUGCCACAC | 32 |
| 1518779 | 535 | 557 | 18102 | 18124 | GUCCUUUUUGACAAAGCCAGUGG | 33 |
| 1518777 | 635 | 657 | N/A | N/A | CUUCCUCAGAAGGCAUUUCAUAA | 34 |
| 1518780 | 615 | 637 | 111150 | 111172 | UAAGCCUCAUUGUCAGGAUCCAC | 35 |
| 1518775 | 595 | 617 | 111130 | 111152 | CACAGGCAUAUCUUCCAGAAUUC | 36 |
| 1518781 | 575 | 597 | 111110 | 111132 | UUCCUUCCUGUGGGGCUCCUUCU | 37 |
| 1518778 | 555 | 577 | N/A | N/A | UCUUCAUUCUUGCCCAACUGGUC | 38 |
| 1518796 | 655 | 677 | 113723 | 113745 | AGGUUCGUAGUCUUGAUACCCUU | 39 |
| 1518797 | 755 | 777 | 113823 | 113845 | AAUGUCAUGACUGGGCACAUUGG | 40 |
| 1518794 | 735 | 757 | 113803 | 113825 | UGGAACUGAGCACUUGUACAGGA | 41 |
| 1518798 | 695 | 717 | 113763 | 113785 | GAUCUCAAGAAACUGGGAGCAAA | 42 |
| 1518795 | 715 | 737 | 113783 | 113805 | GGAUGGAACAUCUGUCAGCAGAU | 43 |
| 1518799 | 675 | 697 | 113743 | 113765 | AAAGAUAUUUCUUAGGCUUCAGG | 44 |
| 1518816 | 775 | 797 | 113843 | 113865 | ACACUGUAAAAACUUUGAGAAAU | 45 |
| 1518813 | 875 | 897 | 113943 | 113965 | CCUGCUACCAUGUAUUCACUUCA | 46 |
| 1518814 | 855 | 877 | 113923 | 113945 | UCAGUGAAAGGGAAGCACCGAAA | 47 |
| 1518815 | 835 | 857 | 113903 | 113925 | AAAUGCUGAGUGGGGGCAGGUAC | 48 |
| 1518812 | 815 | 837 | 113883 | 113905 | UACAGAUACUUCAAUCACUGCUG | 49 |
| 1518817 | 795 | 817 | 113863 | 113885 | CUGAUGGAAGACUUCGAGAUACA | 50 |
| 1518835 | 915 | 937 | 113983 | 114005 | AUCGUAGAUUGAAGCCACAAAAU | 51 |
| 1518834 | 995 | 1017 | 114063 | 114085 | UUCUGAACAACAGCAACAAAAAA | 52 |
| 1518831 | 975 | 997 | 114043 | 114065 | AAAAUAGUGAGGAUUUAGAAAUA | 53 |
| 1518832 | 955 | 977 | 114023 | 114045 | AUAAGUGGUAGUCACUUAGGUGU | 54 |
| 1518830 | 935 | 957 | 114003 | 114025 | UGUUUUUAAUUUGUUUUAACAUC | 55 |
| 1518833 | 895 | 917 | 113963 | 113985 | AAUCCACAGCACACAAAGACCCU | 56 |
| 1518848 | 1015 | 1037 | 114083 | 114105 | GAUAGCAAAUCACUAACAACUUC | 57 |
| 1518852 | 1115 | 1137 | 114183 | 114205 | CUCACAUAUUUUUAAGUAUUAUA | 58 |
| 1518850 | 1095 | 1117 | 114163 | 114185 | AUAUAUAUUAACAAAUUUCACAA | 59 |
| 1518853 | 1075 | 1097 | 114143 | 114165 | CAAUACGUCAUUAUUCUUAGACA | 60 |
| 1518849 | 1055 | 1077 | 114123 | 114145 | ACAGUAUCAUUAAAAGACACCUA | 61 |
| 1518851 | 1035 | 1057 | 114103 | 114125 | CUAAAAAUCUUAUAAUAUAUGAU | 62 |
| 1518871 | 1135 | 1157 | 114203 | 114225 | UAUAGGUGCAUAGUUUCAUGCUC | 63 |
| 1518868 | 1235 | 1257 | 114303 | 114325 | UUUGCAAUGAGAUAACGUUUUAU | 64 |
| 1518870 | 1215 | 1237 | 114283 | 114305 | UAUUUUAAUUCUCACCAUUUAUA | 65 |
| 1518869 | 1195 | 1217 | 114263 | 114285 | AUAUACAAACACAAGUGAAUAAA | 66 |
| 1518867 | 1175 | 1197 | 114243 | 114265 | AAAACACAUCGCAAAAUGGUAAA | 67 |
| 1518866 | 1155 | 1177 | 114223 | 114245 | AAAAUUUCAUAUUUAGUAUUUAU | 68 |
| 1518884 | 1275 | 1297 | 114343 | 114365 | UUUUUAUUAUUAAAGUGAGAUGG | 69 |
| 1518888 | 1355 | 1377 | 114423 | 114445 | UCCUUCUUCAAAUGGCUAUUAAU | 70 |
| 1518885 | 1335 | 1357 | 114403 | 114425 | AAUAACUUUAUAUUUUUGUCCUU | 71 |
| 1518887 | 1315 | 1337 | 114383 | 114405 | CUUUGUGUCAGUUCUUAAUUCAU | 72 |
| 1518889 | 1295 | 1317 | 114363 | 114385 | CAUGUUGCUUAUAAGCAUGAUUU | 73 |
| 1518886 | 1255 | 1277 | 114323 | 114345 | UGGGAUAAAAAUAAAAUAUUUUU | 74 |
| 1518902 | 1375 | 1397 | 114443 | 114465 | UCUACCUCUUCUAAAAUUCCUCC | 75 |
| 1518906 | 1475 | 1497 | 114543 | 114565 | UAAUUAAUCACAGCCACUUAAGG | 76 |
| 1518904 | 1455 | 1477 | 114523 | 114545 | AGGAACCAGUGCAUACCAAAACA | 77 |
| 1518907 | 1435 | 1457 | 114503 | 114525 | ACACACUUCUGGCAGUGUUGCUU | 78 |
| 1518905 | 1415 | 1437 | 114483 | 114505 | CUUCAGGGAAUUCCGAGUGUAGG | 79 |
| 1518903 | 1395 | 1417 | 114463 | 114485 | AGGGUUAAUGUUCCAUUUUCUCU | 80 |
| 1518921 | 1495 | 1517 | 114563 | 114585 | CUUCAACACCCCACUUUCAAUAA | 81 |
| 1518920 | 1575 | 1597 | 114643 | 114665 | AACAGUUCCCCAAAAUACGUAAA | 82 |
| 1518925 | 1555 | 1577 | 114623 | 114645 | AAAGCAAACAUUGACAGGAUUGA | 83 |
| 1518923 | 1535 | 1557 | 114603 | 114625 | UGAAGGGAGAAAUAGACCACUCU | 84 |
| 1518922 | 1515 | 1537 | 114583 | 114605 | UCUACAAUAGUAGUUGGGGUCUU | 85 |
| 1518924 | 1595 | 1617 | 114663 | 114685 | UAAACACAUACACAUCAAACAAC | 86 |
| 1518939 | 1615 | 1637 | 114683 | 114705 | UUAAAAAUGUAUAACAAUUAUAA | 87 |
| 1518938 | 1695 | 1717 | 114763 | 114785 | ACCAAUAUCAGACAAAAUAGAUU | 88 |
| 1518940 | 1675 | 1697 | 114743 | 114765 | AUUUUAACUAAAAAAUUAUUUCG | 89 |
| 1518941 | 1655 | 1677 | 114723 | 114745 | UCGAGACAAAAAUAACAAUAUAU | 90 |
| 1518943 | 1635 | 1657 | 114703 | 114725 | UAUGUUAAUAAAAGGCUCAAUUA | 91 |
| 1518942 | 1715 | 1737 | 114783 | 114805 | CAGAAAGGUACAGCAUUCACACC | 92 |
| 1518958 | 1755 | 1777 | 114823 | 114845 | UUUUUUUUUUUUUUAUUCAUGGU | 93 |
| 1518956 | 1835 | 1857 | 114903 | 114925 | GUGCUAAUGUGUCUUAUGGCUCU | 94 |
| 1518959 | 1815 | 1837 | 114883 | 114905 | UCUCUAAGGAGGGUGUAGUCAAA | 95 |
| 1518960 | 1775 | 1797 | 114843 | 114865 | UUAGUUCCCGGGAACCCACUUUU | 96 |
| 1518961 | 1795 | 1817 | 114863 | 114885 | AAAAUCAUCUUCUACACUGCUUA | 97 |
| 1518957 | 1735 | 1757 | 114803 | 114825 | GGUCGAAUAUUAUUUAUUGUCAG | 98 |
| 1518977 | 1855 | 1877 | 114923 | 114945 | AGCCUUGAAUGUGCUAAUAUGUG | 99 |
| 1518974 | 1955 | 1977 | 115023 | 115045 | AGAGCGAGAGAAAAAGAGAGAGA | 100 |
| 1518978 | 1935 | 1957 | 115003 | 115025 | AGAGAGAGAGAGAAUUUCUGAUG | 101 |
| 1518975 | 1915 | 1937 | 114983 | 115005 | AUGAUUAAAAAAAAAAAGUGAGG | 102 |
| 1518976 | 1895 | 1917 | 114963 | 114985 | AGGAAUGCUGAGUUAAACAAAGU | 103 |
| 1518979 | 1875 | 1897 | 114943 | 114965 | AGUUAACCACAUUCUCUCAGAGC | 104 |
| 1518992 | 1975 | 1997 | 115043 | 115065 | GUAAAAAAAAAAAAAAAAAGAGA | 105 |
| 1518993 | 2055 | 2077 | 115123 | 115145 | AUGAAAUUUUUAUCAGUCUAGAG | 106 |
| 1518994 | 2035 | 2057 | 115103 | 115125 | GAGAAUUGAUCUCCUUUAAGGUG | 107 |
| 1518996 | 2015 | 2037 | 115083 | 115105 | GUGACUCUGGUAGUUCCAACGAU | 108 |
| 1518995 | 1995 | 2017 | 115063 | 115085 | GAUGUUUAAAGGCAUUUCCUGUA | 109 |
| 1518997 | 2075 | 2097 | 115143 | 115165 | GGCAACAUUUAAAGGAGGCCAUG | 110 |
| 1519015 | 2095 | 2117 | 115163 | 115185 | AUCCUAGAAUUCAUAUAUUUGGC | 111 |
| 1519011 | 2195 | 2217 | 115263 | 115285 | AAUUAAUUUAUACAGAGUUUUUC | 112 |
| 1519014 | 2175 | 2197 | 115243 | 115265 | UUCACCAUCAAGUUUAUUUUCAG | 113 |
| 1519013 | 2155 | 2177 | 115223 | 115245 | CAGCACCCAUGGGGAGUUAAUAG | 114 |
| 1519010 | 2135 | 2157 | 115203 | 115225 | UAGAUCUUCCCUGAAAGAGAAAA | 115 |
| 1519012 | 2115 | 2137 | 115183 | 115205 | AAAACCUUUCCUAAGGAAAAAUC | 116 |
| 1519032 | 2255 | 2277 | 115323 | 115345 | GUGACUUUUAGAAAUGACUAUGC | 117 |
| 1519029 | 2315 | 2337 | 115383 | 115405 | AAUACAUAUAAACUGCUAGCAUG | 118 |
| 1519030 | 2275 | 2297 | 115343 | 115365 | UGAAAUUAUACUUUCUACUAGUG | 119 |
| 1519028 | 2295 | 2317 | 115363 | 115385 | AUGUCUAGAAUAUUCUGUCUUGA | 120 |
| 1519033 | 2215 | 2237 | 115283 | 115305 | GAGAAACCAAAUAAUUUUUAAAU | 121 |
| 1519031 | 2235 | 2257 | 115303 | 115325 | UGCCCCAGAAUAAUUAAAAAGAG | 122 |
| 1519047 | 2335 | 2357 | 115403 | 115425 | UAUAUAUCACAUUACUCAUGAAU | 123 |
| 1519048 | 2395 | 2417 | 115463 | 115485 | UUUCUAUGGUAACCAUCCUUAUA | 124 |
| 1519050 | 2355 | 2377 | 115423 | 115445 | CCUUCCUCACCAGCGCCCAAUAU | 125 |
| 1519049 | 2435 | 2457 | 115503 | 115525 | ACUCUGUAGUAGUCUCUCUUCAA | 126 |
| 1519051 | 2415 | 2437 | 115483 | 115505 | CAAUUAGGUAAAAAAGGAAGUUU | 127 |
| 1519046 | 2375 | 2397 | 115443 | 115465 | AUAGUCACUCAUUCCUCCUUCCU | 128 |
| 1519064 | 2455 | 2477 | 115523 | 115545 | GAUGACACAUGCAGCUUAGCACU | 129 |
| 1519065 | 2515 | 2537 | 115583 | 115605 | UUUCCUUGCUUAAACAUAACUUA | 130 |
| 1519069 | 2475 | 2497 | 115543 | 115565 | ACCAUUUCUCUCUAGUGUAAGAU | 131 |
| 1519066 | 2495 | 2517 | 115563 | 115585 | UUAAAUAAAACAAGAAACUUACC | 132 |
| 1519067 | 2555 | 2577 | 115623 | 115645 | UUUCUAACCUUCCUGAAAUAUAC | 133 |
| 1519068 | 2535 | 2557 | 115603 | 115625 | UACUGUUCAAUAACAAAUCCUUU | 134 |
| 1519082 | 2575 | 2597 | 115643 | 115665 | AAAUAUAUCCUAACCGCCACUUU | 135 |
| 1519086 | 2675 | 2697 | 115743 | 115765 | UUGUUCUAGGUCACUGCUAUCAG | 136 |
| 1519083 | 2655 | 2677 | 115723 | 115745 | CAGUCUAGUUCUGUCCUCUAUUU | 137 |
| 1519084 | 2635 | 2657 | 115703 | 115725 | UUUCUUAAUUUAAUACCAAUACU | 138 |
| 1519085 | 2615 | 2637 | 115683 | 115705 | ACUUUUAAAUUUUUAAAAUAUGC | 139 |
| 1519087 | 2595 | 2617 | 115663 | 115685 | UGCUGCUUUAGGUAGAUUUAAAA | 140 |
| 1519100 | 2715 | 2737 | 115783 | 115805 | AAAUCCUUAAAUUCAUGGUCACA | 141 |
| 1519103 | 2795 | 2817 | 115863 | 115885 | AAAGUAAUUCACUGCUCAAAAAU | 142 |
| 1519105 | 2775 | 2797 | 115843 | 115865 | AAUUACCGUCAGAUAAAUAUUAA | 143 |
| 1519102 | 2735 | 2757 | 115803 | 115825 | AGGAGAAUUUGUAUCCACAUAAA | 144 |
| 1519101 | 2755 | 2777 | 115823 | 115845 | UAAGGGAAGAAACACUUUAAAGG | 145 |
| 1519104 | 2695 | 2717 | 115763 | 115785 | ACAACUUUCCUAAUCUCAAAUUG | 146 |
| 1519119 | 2815 | 2837 | 115883 | 115905 | AAUAAACUAUUAAGAUAUAUAAA | 147 |
| 1519118 | 2855 | 2877 | 115923 | 115945 | AUAUGACUUAAAGAACUUUUUGU | 148 |
| 1519121 | 2875 | 2897 | 115943 | 115965 | CAAGCUUCCUGAAAAGGCUUAUA | 149 |
| 1519120 | 2835 | 2857 | 115903 | 115925 | UGUUUAAGUGUUUGGUCCCAAAU | 150 |
| 1519123 | 2915 | 2937 | 115983 | 116005 | GGCCACUUGGCAGGUGAAUGUCU | 151 |
| 1519122 | 2895 | 2917 | 115963 | 115985 | UCUCGGGAGUGAAUAUGAGACAA | 152 |
| 1519141 | 2935 | 2957 | 116003 | 116025 | UAGGACUGGAUUGAUCCUCAGGC | 153 |
| 1519137 | 3035 | 3057 | 116103 | 116125 | AUUGUGUGCCAAAGUGCACUCUG | 154 |
| 1519138 | 2995 | 3017 | 116063 | 116085 | CGACCGUGGAGUCAUAUGAGGCU | 155 |
| 1519139 | 3015 | 3037 | 116083 | 116105 | CUGAACUGUUUUGGUAAAGCCGA | 156 |
| 1519140 | 2975 | 2997 | 116043 | 116065 | GCUGAAUAACUUGGGAGAAUGUA | 157 |
| 1519136 | 2955 | 2977 | 116023 | 116045 | GUAAGUCUGCAAAAUAAACCUAG | 158 |
| 1519155 | 3075 | 3097 | 116143 | 116165 | CUUGGAAUACCAAACCACACAUU | 159 |
| 1519158 | 3155 | 3177 | 116223 | 116245 | GCUACAUACUGGAUAAGCCAGAA | 160 |
| 1519157 | 3135 | 3157 | 116203 | 116225 | GAAAGAUGAGGAAACAUGUUUGC | 161 |
| 1519156 | 3115 | 3137 | 116183 | 116205 | UGCAUCUCACACUAGUGCAGAGA | 162 |
| 1519159 | 3095 | 3117 | 116163 | 116185 | AGAUUCUGAAAAAGACCCCACUU | 163 |
| 1519154 | 3055 | 3077 | 116123 | 116145 | AUUAGAUUGUUCUGUUCCCAAUU | 164 |
| 1519164 | 3175 | 3197 | 116243 | 116265 | UAUUUAUUAUGUCACAAAUAGCU | 165 |
| 1519165 | 3193 | 3215 | 116261 | 116283 | UAUUUUCAUAUAUGUAUAUAUUU | 166 |

The sense oligonucleotide in each case is 21 nucleosides in length; has a sugar motif (from 5′ to 3′) of: fyfyfyfyfyfyfyfyfyfyf, wherein each ‘y’ represents a ribo-2′-OMe sugar moiety and each “f” represents a 2′-F sugar moiety; and an intemucleoside linkage motif (from 5′ to 3′) of: ssooooooooooooooooss; wherein each ‘o’ represents a phosphodiester intemucleoside linkage and each ‘s’ represents a phosphorothioate internucleoside linkage. The sense oligonucleotides are listed below in Tables 2 and 4.

The oligomeric duplex compound numbers with the corresponding sense oligonucleotide (“Sense oligo.”) compound numbers and antisense oligonucleotide (“Antisense oligo.”) compound numbers, together with the sense oligonucleotide sequences are listed in Tables 2 and 4 below.

TABLE 2
Design of sense oligonucleotides and design of
oligomeric duplexes targeted to human SNCA

| Oligomeric | Antisense | Sense | | |
|---|---|---|---|---|
| Duplex | oligo. | oligo. | | SEQ |
| Compound | Compound | Compound | Sense Oligo. | ID |
| Number | No. | No. | Sequence (5′ to 3′) | NO |
| 1518710 | 1518724 | 1518718 | CUGGUGUGAUCCAGGAACAGC | 167 |
| 1518711 | 1518723 | 1518717 | AUUAGCCAUGGAUGUAUUCAU | 168 |
| 1518712 | 1518722 | 1518716 | CUGUCUUCCAGCUCUGAAAGA | 169 |
| 1518713 | 1518727 | 1518720 | GAGGAGCUUGCUUCUCCAUUC | 170 |
| 1518715 | 1518726 | 1518721 | UGAAAGGACUUUCAAAGGCCA | 171 |
| 1518728 | 1518741 | 1518735 | AAGGAGGGAGUUGUGGCUGCU | 172 |
| 1518729 | 1518740 | 1518736 | UGCUGAGAAAACCAAACAGGG | 173 |
| 1518730 | 1518742 | 1518734 | CUAUGUAGGCUCCAAAACCAA | 174 |
| 1518731 | 1518745 | 1518737 | AAGACAAAAGAGGGUGUUCUC | 175 |
| 1518732 | 1518744 | 1518738 | GUGUGGCAGAAGCAGCAGGAA | 176 |
| 1518733 | 1518743 | 1518739 | AGGAGGGAGUGGUGCAUGGUG | 177 |
| 1518746 | 1518760 | 1518755 | GACCAAAGAGCAAGUGACAAA | 178 |
| 1518747 | 1518759 | 1518754 | CAGGGAGCAUUGCAGCAGCCA | 179 |
| 1518748 | 1518761 | 1518753 | CCAGAAGACAGUGGAGGGAGC | 180 |
| 1518749 | 1518758 | 1518752 | ACGGGUGUGACAGCAGUAGCC | 181 |
| 1518750 | 1518763 | 1518757 | AUGUUGGAGGAGCAGUGGUGA | 182 |
| 1518751 | 1518762 | 1518756 | GUGGCAACAGUGGCUGAGAAG | 183 |
| 1518764 | 1518779 | 1518771 | ACUGGCUUUGUCAAAAAGGAC | 184 |
| 1518765 | 1518777 | 1518772 | AUGAAAUGCCUUCUGAGGAAG | 185 |
| 1518766 | 1518780 | 1518773 | GGAUCCUGACAAUGAGGCUUA | 186 |
| 1518767 | 1518775 | 1518770 | AUUCUGGAAGAUAUGCCUGUG | 187 |
| 1518768 | 1518781 | 1518776 | AAGGAGCCCCACAGGAAGGAA | 188 |
| 1518769 | 1518778 | 1518774 | CCAGUUGGGCAAGAAUGAAGA | 189 |
| 1518782 | 1518796 | 1518788 | GGGUAUCAAGACUACGAACCU | 190 |
| 1518783 | 1518797 | 1518789 | AAUGUGCCCAGUCAUGACAUU | 191 |
| 1518784 | 1518794 | 1518790 | CUGUACAAGUGCUCAGUUCCA | 192 |
| 1518785 | 1518798 | 1518793 | UGCUCCCAGUUUCUUGAGAUC | 193 |
| 1518786 | 1518795 | 1518792 | CUGCUGACAGAUGUUCCAUCC | 194 |
| 1518787 | 1518799 | 1518791 | UGAAGCCUAAGAAAUAUCUUU | 195 |
| 1518800 | 1518816 | 1518808 | UUCUCAAAGUUUUUACAGUGU | 196 |
| 1518801 | 1518813 | 1518806 | AAGUGAAUACAUGGUAGCAGG | 197 |
| 1518802 | 1518814 | 1518809 | UCGGUGCUUCCCUUUCACUGA | 198 |
| 1518803 | 1518815 | 1518811 | ACCUGCCCCCACUCAGCAUUU | 199 |
| 1518804 | 1518812 | 1518807 | GCAGUGAUUGAAGUAUCUGUA | 200 |
| 1518805 | 1518817 | 1518810 | UAUCUCGAAGUCUUCCAUCAG | 201 |
| 1518818 | 1518835 | 1518824 | UUUGUGGCUUCAAUCUACGAU | 202 |
| 1518819 | 1518834 | 1518826 | UUUUGUUGCUGUUGUUCAGAA | 203 |
| 1518820 | 1518831 | 1518827 | UUUCUAAAUCCUCACUAUUUU | 204 |
| 1518821 | 1518832 | 1518828 | ACCUAAGUGACUACCACUUAU | 205 |
| 1518822 | 1518830 | 1518829 | UGUUAAAACAAAUUAAAAACA | 206 |
| 1518823 | 1518833 | 1518825 | GGUCUUUGUGUGCUGUGGAUU | 207 |
| 1518836 | 1518848 | 1518845 | AGUUGUUAGUGAUUUGCUAUC | 208 |
| 1518837 | 1518852 | 1518847 | UAAUACUUAAAAAUAUGUGAG | 209 |
| 1518838 | 1518850 | 1518843 | GUGAAAUUUGUUAAUAUAUAU | 210 |
| 1518839 | 1518853 | 1518846 | UCUAAGAAUAAUGACGUAUUG | 211 |
| 1518840 | 1518849 | 1518844 | GGUGUCUUUUAAUGAUACUGU | 212 |
| 1518841 | 1518851 | 1518842 | CAUAUAUUAUAAGAUUUUUAG | 213 |
| 1518854 | 1518871 | 1518864 | GCAUGAAACUAUGCACCUAUA | 214 |
| 1518855 | 1518868 | 1518862 | AAAACGUUAUCUCAUUGCAAA | 215 |
| 1518856 | 1518870 | 1518865 | UAAAUGGUGAGAAUUAAAAUA | 216 |
| 1518857 | 1518869 | 1518861 | UAUUCACUUGUGUUUGUAUAU | 217 |
| 1518858 | 1518867 | 1518863 | UACCAUUUUGCGAUGUGUUUU | 218 |
| 1518859 | 1518866 | 1518860 | AAAUACUAAAUAUGAAAUUUU | 219 |
| 1518872 | 1518884 | 1518881 | AUCUCACUUUAAUAAUAAAAA | 220 |
| 1518873 | 1518888 | 1518879 | UAAUAGCCAUUUGAAGAAGGA | 221 |
| 1518874 | 1518885 | 1518878 | GGACAAAAAUAUAAAGUUAUU | 222 |
| 1518875 | 1518887 | 1518880 | GAAUUAAGAACUGACACAAAG | 223 |
| 1518876 | 1518889 | 1518883 | AUCAUGCUUAUAAGCAACAUG | 224 |
| 1518877 | 1518886 | 1518882 | AAAUAUUUUAUUUUUAUCCCA | 225 |
| 1518890 | 1518902 | 1518899 | AGGAAUUUUAGAAGAGGUAGA | 226 |
| 1518891 | 1518906 | 1518901 | UUAAGUGGCUGUGAUUAAUUA | 227 |
| 1518892 | 1518904 | 1518896 | UUUUGGUAUGCACUGGUUCCU | 228 |
| 1518893 | 1518907 | 1518900 | GCAACACUGCCAGAAGUGUGU | 229 |
| 1518894 | 1518905 | 1518898 | UACACUCGGAAUUCCCUGAAG | 230 |
| 1518895 | 1518903 | 1518897 | AGAAAAUGGAACAUUAACCCU | 231 |
| 1518908 | 1518921 | 1518915 | AUUGAAAGUGGGGUGUUGAAG | 232 |
| 1518909 | 1518920 | 1518914 | UACGUAUUUUGGGGAACUGUU | 233 |
| 1518910 | 1518925 | 1518916 | AAUCCUGUCAAUGUUUGCUUU | 234 |
| 1518911 | 1518923 | 1518919 | AGUGGUCUAUUUCUCCCUUCA | 235 |
| 1518912 | 1518922 | 1518918 | GACCCCAACUACUAUUGUAGA | 236 |
| 1518913 | 1518924 | 1518917 | UGUUUGAUGUGUAUGUGUUUA | 237 |
| 1518926 | 1518939 | 1518934 | AUAAUUGUUAUACAUUUUUAA | 238 |
| 1518927 | 1518938 | 1518933 | UCUAUUUUGUCUGAUAUUGGU | 239 |
| 1518928 | 1518940 | 1518932 | AAAUAAUUUUUUAGUUAAAAU | 240 |
| 1518929 | 1518941 | 1518935 | AUAUUGUUAUUUUUGUCUCGA | 241 |
| 1518930 | 1518943 | 1518936 | AUUGAGCCUUUUAUUAACAUA | 242 |
| 1518931 | 1518942 | 1518937 | UGUGAAUGCUGUACCUUUCUG | 243 |
| 1518944 | 1518958 | 1518952 | CAUGAAUAAAAAAAAAAAAAA | 244 |
| 1518945 | 1518956 | 1518950 | AGCCAUAAGACACAUUAGCAC | 245 |
| 1518946 | 1518959 | 1518953 | UGACUACACCCUCCUUAGAGA | 246 |
| 1518947 | 1518960 | 1518954 | AAGUGGGUUCCCGGGAACUAA | 247 |
| 1518948 | 1518961 | 1518955 | AGCAGUGUAGAAGAUGAUUUU | 248 |
| 1518949 | 1518957 | 1518951 | GACAAUAAAUAAUAUUCGACC | 249 |
| 1518962 | 1518977 | 1518969 | CAUAUUAGCACAUUCAAGGCU | 250 |
| 1518963 | 1518974 | 1518968 | UCUCUCUUUUUCUCUCGCUCU | 251 |
| 1518964 | 1518978 | 1518971 | UCAGAAAUUCUCUCUCUCUCU | 252 |
| 1518965 | 1518975 | 1518973 | UCACUUUUUUUUUUUAAUCAU | 253 |
| 1518966 | 1518976 | 1518970 | UUUGUUUAACUCAGCAUUCCU | 254 |
| 1518967 | 1518979 | 1518972 | UCUGAGAGAAUGUGGUUAACU | 255 |
| 1518980 | 1518992 | 1518986 | UCUUUUUUUUUUUUUUUUUAC | 256 |
| 1518981 | 1518993 | 1518987 | CUAGACUGAUAAAAAUUUCAU | 257 |
| 1518982 | 1518994 | 1518988 | CCUUAAAGGAGAUCAAUUCUC | 258 |
| 1518983 | 1518996 | 1518989 | CGUUGGAACUACCAGAGUCAC | 259 |
| 1518984 | 1518995 | 1518990 | CAGGAAAUGCCUUUAAACAUC | 260 |
| 1518985 | 1518997 | 1518991 | UGGCCUCCUUUAAAUGUUGCC | 261 |
| 1518998 | 1519015 | 1519006 | CAAAUAUAUGAAUUCUAGGAU | 262 |
| 1518999 | 1519011 | 1519008 | AAAACUCUGUAUAAAUUAAUU | 263 |
| 1519000 | 1519014 | 1519009 | GAAAAUAAACUUGAUGGUGAA | 264 |
| 1519001 | 1519013 | 1519007 | AUUAACUCCCCAUGGGUGCUG | 265 |
| 1519002 | 1519010 | 1519005 | UUCUCUUUCAGGGAAGAUCUA | 266 |
| 1519003 | 1519012 | 1519004 | UUUUUCCUUAGGAAAGGUUUU | 267 |
| 1519016 | 1519032 | 1519026 | AUAGUCAUUUCUAAAAGUCAC | 268 |
| 1519017 | 1519029 | 1519025 | UGCUAGCAGUUUAUAUGUAUU | 269 |
| 1519018 | 1519030 | 1519027 | CUAGUAGAAAGUAUAAUUUCA | 270 |
| 1519019 | 1519028 | 1519023 | AAGACAGAAUAUUCUAGACAU | 271 |
| 1519020 | 1519033 | 1519024 | UUAAAAAUUAUUUGGUUUCUC | 272 |
| 1519021 | 1519031 | 1519022 | CUUUUUAAUUAUUCUGGGGCA | 273 |
| 1519034 | 1519047 | 1519045 | UCAUGAGUAAUGUGAUAUAUA | 274 |
| 1519035 | 1519048 | 1519040 | UAAGGAUGGUUACCAUAGAAA | 275 |
| 1519036 | 1519050 | 1519041 | AUUGGGCGCUGGUGAGGAAGG | 276 |
| 1519037 | 1519049 | 1519043 | GAAGAGAGACUACUACAGAGU | 277 |
| 1519038 | 1519051 | 1519044 | ACUUCCUUUUUUACCUAAUUG | 278 |
| 1519039 | 1519046 | 1519042 | GAAGGAGGAAUGAGUGACUAU | 279 |
| 1519052 | 1519064 | 1519058 | UGCUAAGCUGCAUGUGUCAUC | 280 |
| 1519053 | 1519065 | 1519059 | AGUUAUGUUUAAGCAAGGAAA | 281 |
| 1519054 | 1519069 | 1519061 | CUUACACUAGAGAGAAAUGGU | 282 |
| 1519055 | 1519066 | 1519060 | UAAGUUUCUUGUUUUAUUUAA | 283 |
| 1519056 | 1519067 | 1519063 | AUAUUUCAGGAAGGUUAGAAA | 284 |
| 1519057 | 1519068 | 1519062 | AGGAUUUGUUAUUGAACAGUA | 285 |
| 1519070 | 1519082 | 1519076 | AGUGGCGGUUAGGAUAUAUUU | 286 |
| 1519071 | 1519086 | 1519078 | GAUAGCAGUGACCUAGAACAA | 287 |
| 1519072 | 1519083 | 1519081 | AUAGAGGACAGAACUAGACUG | 288 |
| 1519073 | 1519084 | 1519077 | UAUUGGUAUUAAAUUAAGAAA | 289 |
| 1519074 | 1519085 | 1519079 | AUAUUUUAAAAAUUUAAAAGU | 290 |
| 1519075 | 1519087 | 1519080 | UUAAAUCUACCUAAAGCAGCA | 291 |
| 1519088 | 1519100 | 1519097 | UGACCAUGAAUUUAAGGAUUU | 292 |
| 1519089 | 1519103 | 1519096 | UUUUGAGCAGUGAAUUACUUU | 293 |
| 1519090 | 1519105 | 1519099 | AAUAUUUAUCUGACGGUAAUU | 294 |
| 1519091 | 1519102 | 1519095 | UAUGUGGAUACAAAUUCUCCU | 295 |
| 1519092 | 1519101 | 1519094 | UUUAAAGUGUUUCUUCCCUUA | 296 |
| 1519093 | 1519104 | 1519098 | AUUUGAGAUUAGGAAAGUUGU | 297 |
| 1519106 | 1519119 | 1519112 | UAUAUAUCUUAAUAGUUUAUU | 298 |
| 1519107 | 1519118 | 1519113 | AAAAAGUUCUUUAAGUCAUAU | 299 |
| 1519108 | 1519121 | 1519116 | UAAGCCUUUUCAGGAAGCUUG | 300 |
| 1519109 | 1519120 | 1519114 | UUGGGACCAAACACUUAAACA | 301 |
| 1519110 | 1519123 | 1519115 | ACAUUCACCUGCCAAGUGGCC | 302 |
| 1519111 | 1519122 | 1519117 | GUCUCAUAUUCACUCCCGAGA | 303 |
| 1519124 | 1519141 | 1519135 | CUGAGGAUCAAUCCAGUCCUA | 304 |
| 1519125 | 1519137 | 1519131 | GAGUGCACUUUGGCACACAAU | 305 |
| 1519126 | 1519138 | 1519133 | CCUCAUAUGACUCCACGGUCG | 306 |
| 1519127 | 1519139 | 1519130 | GGCUUUACCAAAACAGUUCAG | 307 |
| 1519128 | 1519140 | 1519132 | CAUUCUCCCAAGUUAUUCAGC | 308 |
| 1519129 | 1519136 | 1519134 | AGGUUUAUUUUGCAGACUUAC | 309 |
| 1519142 | 1519155 | 1519152 | UGUGUGGUUUGGUAUUCCAAG | 310 |
| 1519143 | 1519158 | 1519151 | CUGGCUUAUCCAGUAUGUAGC | 311 |
| 1519144 | 1519157 | 1519150 | AAACAUGUUUCCUCAUCUUUC | 312 |
| 1519145 | 1519156 | 1519148 | UCUGCACUAGUGUGAGAUGCA | 313 |
| 1519146 | 1519159 | 1519149 | GUGGGGUCUUUUUCAGAAUCU | 314 |
| 1519147 | 1519154 | 1519153 | UUGGGAACAGAACAAUCUAAU | 315 |
| 1519160 | 1519164 | 1519162 | CUAUUUGUGACAUAAUAAAUA | 316 |
| 1519161 | 1519165 | 1519163 | AUAUAUACAUAUAUGAAAAUA | 317 |

TABLE 3
Design of antisense modified oligonucleotides
targeted to human SNCA

| | SEQ ID | SEQ ID | | |
|---|---|---|---|---|
| Antisense | NO: 5 | NO: 5 | Antisense | |
| oligo. | Antisense | Antisense | Oligo. | SEQ |
| Compound | oligo. | oligo. | Sequence | ID |
| No. | Start Site | Stop Site | (5′ to 3′) | NO |
| 1518725 | 42 | 64 | UGAAUUCCUUUA | 318 |
| | | | CACCACACUCU | |

TABLE 4
Design of sense modified oligonucleotides
and design of oligomeric duplexes
targeted to human SNCA

| Oligomeric | Antisense | Sense | Sense | |
|---|---|---|---|---|
| Duplex | oligo. | oligo. | Oligo. | SEQ |
| Compound | Compound | Compound | Sequence | ID |
| Number | No. | No. | (5′ to 3′) | NO |
| 1518714 | 1518725 | 1518719 | AGUGUGGUGUA | 319 |
| | | | AAGGAAUUCA | |

### Example 2: Effect of Oligomeric Duplexes on Human SNCA RNA In Vitro, Single Dose

Oligomeric duplexes described above are tested in a series of experiments under the same culture conditions.

Cultured SH-SY5Y cells were treated with oligomeric duplexes at a concentration of 40 nM by LipofectAMINE 2000 at a cell density of 15,000 cells per well. After a treatment period of approximately 24 hours, total RNA was isolated from the cells and SNCA RNA levels were measured by quantitative real-time RT-PCR. SNCA RNA levels were measured by the human primer-probe set hSNCA_LTS00672_MGB (forward sequence TGGCAGAAGCAGCAGGAAA, designated herein as SEQ ID NO: 7; reverse sequence TCCTTGGTTTTGGAGCCTACA, designated herein as SEQ ID NO: 8; probe sequence CAAAAGAGGGTGTTCTC, designated herein as SEQ ID NO: 9) and human primer probe set RTS4787_MGB (forward sequence GTTGTTCAGAAGTTGTTAGTGATTTGCT, designated herein as SEQ ID NO: 10; reverse sequence GTGCATAGTTTCATGCTCACATATTTT, designated herein as SEQ ID NO: 11; probe sequence CTGTCTAAGAATAATGACGTATTG, designated herein as SEQ ID NO: 12) as indicated in the tables below. SNCA RNA levels were normalized to human GAPDH. Human GAPDH was amplified using human primer probe set RTS104 (forward sequence GAAGGTGAAGGTCGGAGTC, designated herein as SEQ ID NO: 13; reverse sequence GAAGATGGTGATGGGATTTC, designated herein as SEQ ID NO: 14; probe sequence CAAGCTTCCCGTTCTCAGCC, designated herein as SEQ ID NO: 15). Reduction of SNCA RNA is presented in the tables below as percent SNCA RNA, relative to the amount in untreated control cells (% UTC). The values marked with a “t” indicate that the modified oligonucleotide is complementary to the amplicon region of the primer probe set. Additional assays may be used to measure the potency and efficacy of the modified oligonucleotides complementary to the amplicon region.

TABLE 5
Reduction of SNCA RNA by oligomeric
duplexes in SH-SY5Y cells

| Compound | SNCA (% UTC) | SNCA (% UTC) |
|---|---|---|
| No. | hSNCA_LTS00672_MGB | RTS4787_MGB |
| 1518944 | 102 | 52 |
| 1518945 | 113 | 74 |
| 1518946 | 82 | 92 |
| 1518947 | 74 | 57 |
| 1518948 | 91 | 87 |
| 1518949 | 97 | 71 |
| 1518962 | 90 | 75 |
| 1518963 | 105 | 83 |
| 1518964 | 92 | 73 |
| 1518965 | 84 | 62 |
| 1518966 | 106 | 78 |
| 1518967 | 75 | 59 |
| 1518980 | 85 | 86 |
| 1518981 | 92 | 64 |
| 1518982 | 88 | 72 |
| 1518983 | 73 | 69 |
| 1518984 | 121 | 83 |
| 1518985 | 161 | 131 |
| 1518998 | 97 | 91 |
| 1518999 | 104 | 81 |
| 1519000 | 106 | 100 |
| 1519001 | 94 | 74 |
| 1519002 | 96 | 59 |
| 1519003 | 73 | 61 |
| 1519016 | 77 | 61 |
| 1519017 | 95 | 99 |
| 1519018 | 94 | 68 |
| 1519019 | 101 | 94 |
| 1519020 | 97 | 72 |
| 1519021 | 126 | 84 |
| 1519034 | 90 | 78 |
| 1519035 | 97 | 77 |
| 1519036 | 90 | 77 |
| 1519037 | 87 | 65 |
| 1519038 | 69 | 62 |
| 1519039 | 76 | 62 |
| 1519052 | 98 | 91 |
| 1519053 | 78 | 60 |
| 1519054 | 138 | 103 |
| 1519055 | 103 | 89 |
| 1519056 | 166 | 131 |
| 1519057 | 112 | 92 |
| 1519070 | 105 | 70 |
| 1519071 | 141 | 98 |
| 1519072 | 79 | 69 |
| 1519073 | 103 | 75 |
| 1519074 | 97 | 86 |
| 1519075 | 91 | 75 |
| 1519088 | 110 | 94 |
| 1519089 | 90 | 61 |
| 1519090 | 102 | 74 |
| 1519091 | 97 | 78 |
| 1519092 | 79 | 58 |
| 1519093 | 101 | 79 |
| 1519106 | 90 | 80 |
| 1519107 | 124 | 107 |
| 1519108 | 92 | 97 |
| 1519109 | 86 | 85 |
| 1519110 | 101 | 79 |
| 1519111 | 90 | 77 |
| 1519124 | 113 | 99 |
| 1519125 | 85 | 72 |
| 1519126 | 119 | 105 |
| 1519127 | 92 | 76 |
| 1519128 | 105 | 81 |
| 1519129 | 94 | 80 |
| 1519142 | 92 | 76 |
| 1519143 | 96 | 79 |
| 1519144 | 80 | 67 |
| 1519145 | 111 | 85 |
| 1519146 | 98 | 69 |
| 1519147 | 72 | 84 |
| 1519160 | 87 | 71 |
| 1519161 | 75 | 74 |

TABLE 6
Reduction of SNCA RNA by oligomeric
duplexes in SH-SY5Y cells

| Compound | SNCA (% UTC) | SNCA (% UTC) |
|---|---|---|
| No. | hSNCA_LTS00672_MGB | RTS4787_MGB |
| 1518710 | 63 | 80 |
| 1518711 | 99 | 106 |
| 1518712 | 71 | 82 |
| 1518713 | 78 | 82 |
| 1518714 | 78 | 90 |
| 1518715 | 95 | 86 |
| 1518728 | 94 | 73 |
| 1518729 | 99 | 83 |
| 1518730 | 83† | 74 |
| 1518731 | 86† | 79 |
| 1518732 | 69† | 63 |
| 1518733 | 105† | 118 |
| 1518746 | 103 | 90 |
| 1518747 | 86 | 75 |
| 1518748 | 77 | 62 |
| 1518749 | 92 | 72 |
| 1518750 | 96 | 72 |
| 1518751 | 89 | 85 |
| 1518764 | 68 | 41 |
| 1518765 | 87 | 69 |
| 1518766 | 102 | 86 |
| 1518767 | 96 | 84 |
| 1518768 | 86 | 65 |
| 1518769 | 66 | 66 |
| 1518782 | 54 | 36 |
| 1518783 | 61 | 58 |
| 1518784 | 47 | 25 |
| 1518785 | 38 | 14 |
| 1518786 | 65 | 36 |
| 1518787 | 59 | 36 |
| 1518800 | 46 | 19 |
| 1518801 | 40 | 18 |
| 1518802 | 40 | 6 |
| 1518803 | 43 | 14 |
| 1518804 | 36 | 12 |
| 1518805 | 47 | 23 |
| 1518818 | 47 | 30 |
| 1518819 | 39 | 21† |
| 1518820 | 39 | 21 |
| 1518821 | 42 | 31 |
| 1518822 | 53 | 26 |
| 1518823 | 55 | 35 |
| 1518836 | 45 | 35† |
| 1518837 | 36 | 21† |
| 1518838 | 50 | 32† |
| 1518839 | 60 | 41† |
| 1518840 | 86 | 77† |
| 1518841 | 37 | 23† |
| 1518854 | 36 | 25† |
| 1518855 | 98 | 80 |
| 1518856 | 47 | 27 |
| 1518857 | 33 | 21 |
| 1518858 | 29 | 30 |
| 1518859 | 42 | 21 |
| 1518872 | 55 | 38 |
| 1518873 | 67 | 55 |
| 1518874 | 35 | 21 |
| 1518875 | 31 | 21 |
| 1518876 | 61 | 55 |
| 1518877 | 94 | 71 |
| 1518890 | 32 | 34 |
| 1518891 | 48 | 38 |
| 1518892 | 38 | 27 |
| 1518893 | 60 | 43 |
| 1518894 | 78 | 73 |
| 1518895 | 92 | 69 |
| 1518908 | 40 | 42 |
| 1518909 | 117 | 99 |
| 1518910 | 47 | 40 |
| 1518911 | 50 | 42 |
| 1518912 | 45 | 30 |
| 1518913 | 58 | 47 |
| 1518926 | 107 | 91 |
| 1518927 | 83 | 62 |
| 1518928 | 41 | 25 |
| 1518929 | 55 | 33 |
| 1518930 | 114 | 85 |
| 1518931 | 38 | 22 |

### Example 3: Design of Oligomeric Duplexes that Target a Human SNCA Nucleic Acid

Oligomeric duplexes comprising antisense oligonucleotides complementary to a human SNCA nucleic acid, and sense oligonucleotides complementary to the antisense oligonucleotides were designed as follows.

The antisense oligonucleotide in each case is 23 nucleosides in length; has a sugar motif (from 5′ to 3′) of: efyyyfyyyyyyyfyfyyyyyyy; wherein ‘e’ represents a ribo-2′-MOE sugar moiety, ‘y’ represents a ribo-2′-OMe sugar moiety, and each “f” represents a ribo-2′-F sugar; and an internucleoside linkage motif (from 5′ to 3′) of: ssooooooooooooooooooss; wherein each ‘o’ represents a phosphodiester internucleoside linkage and each ‘s’ represents a phosphorothioate internucleoside linkage. Each cytosine residue is a non-methylated cytosine. Each antisense oligonucleotide has a vinyl phosphonate at the 5′-end. The antisense oligonucleotides are listed below in the table below.

“Start site” indicates the 5′-most nucleoside to which the antisense oligonucleotide is complementary in the target nucleic acid sequence. “Stop site” indicates the 3′-most nucleoside to which the antisense oligonucleotide is complementary in the target nucleic acid sequence. Each antisense oligonucleotide listed in the table below is 100% complementary to SEQ ID NO: 1 (described herein above), to SEQ ID NO: 2 (described herein above), or to both.

TABLE 7
Design of antisense oligonucleotides
(antisense oligo.) targeted to human SNCA

| | SEQ ID | SEQ ID | SEQ ID | SEQ ID | | |
|---|---|---|---|---|---|---|
| Antisense | NO: 1 | NO: 1 | NO: 2 | NO: 2 | Antisense | |
| Oligo. | Antisense | Antisense | Antisense | Antisense | Strand | SEQ |
| Compound | Oligo. | Oligo. | Oligo. | Oligo. | Sequence | ID |
| No. | Start Site | Stop Site | Start Site | Stop Site | (5′ to 3′) | NO |
| 1634332 | 855 | 877 | 113923 | 113945 | TCAGUGAAAGGG | 320 |
| | | | | | AAGCACCGAAA | |
| 1637244 | 1375 | 1397 | 114443 | 114465 | TCUACCUCUUCU | 321 |
| | | | | | AAAAUUCCUCC | |

“Start site” indicates the 5′-most nucleoside to which the antisense oligonucleotide is complementary in the target nucleic acid sequence. “Stop site” indicates the 3′-most nucleoside to which the antisense oligonucleotide is complementary in the target nucleic acid sequence. Each antisense oligonucleotide listed in the table below is complementary to SEQ ID NO: 1 (described herein above), to SEQ ID NO: 2 (described herein above), or to both, with a single mismatch at position 1 of the 5′ end.

TABLE 8
Design of antisense oligonucleotides (antisense oligo.) targeted to human SNCA

| | SEQ ID | SEQ ID | SEQ ID | SEQ ID | | |
|---|---|---|---|---|---|---|
| Antisense | NO: 1 | NO: 1 | NO: 2 | NO: 2 | | |
| Oligo. | Antisense | Antisense | Antisense | Antisense | | SEQ |
| Compound | Oligo. | Oligo. | Oligo. | Oligo. | Antisense Strand | ID |
| No. | Start Site | Stop Site | Start Site | Stop Site | Sequence (5′ to 3′) | NO |
| 1637238 | 1195 | 1216 | 114263 | 114284 | TUAUACAAACACAAGUGAAUAAA | 322 |
| 1637240 | 1175 | 1196 | 114243 | 114264 | TAAACACAUCGCAAAAUGGUAAA | 323 |
| 1637242 | 1315 | 1336 | 114383 | 114404 | TUUUGUGUCAGUUCUUAAUUCAU | 324 |

The sense oligonucleotide in each case is 21 nucleosides in length; has a sugar motif (from 5′ to 3′) of: yyyyyyfyfffyyyyyyyyyy; wherein each “y” represents a ribo-2′-OMe sugar and each “f” represents a ribo-2′-F sugar; and an intemucleoside linkage motif (from 5′ to 3′) of: ssooooooooooooooooss; wherein each ‘o’ represents a phosphodiester intemucleoside linkage and each ‘s’ represents a phosphorothioate internucleoside linkage. The sense oligonucleotides are listed in the table below. Each cytosine residue is a non-methylated cytosine. Each sense oligonucleotide in the table below is conjugated to a 2-(hydroxymethyl)-6-palmitamidohexyl phosphoryl conjugate group attached to the 3′-OH of the oligonucleotide. The structure for the C16 conjugate group is:

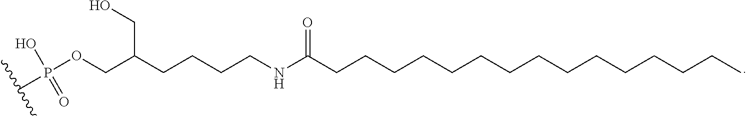 [SMILES: *P(=O)(O)OCC(CO)CCCCNC(=O)CCCCCCCCCCCCCCC]

Each antisense oligonucleotide is complementary to the target nucleic acid (SNCA), and each sense oligonucleotide is complementary to the first 21 nucleosides of the respective antisense oligonucleotide (from 5′ to 3′) wherein the last two 3′-nucleosides of the antisense oligonucleotides are not paired with the sense oligonucleotide (are overhanging nucleosides). The oligomeric duplex compound numbers with the corresponding sense oligonucleotide (sense oligo.) compound numbers and antisense oligonucleotide (antisense oligo.) compound numbers, together with the sense oligonucleotide sequences are listed in the table below.

TABLE 9
Design of sense oligonucleotides and design of oligomeric
duplexes targeted to human SNCA with C16 conjugates

| Oligomeric | Antisense | Sense | | |
|---|---|---|---|---|
| Duplex | Oligo. | Oligo. | | SEQ |
| Compound | Compound | Compound | Sense Oligo. | ID |
| Number | No. | No. | Sequence (5′ to 3′) | NO |
| 1657448 | 1634332 | 1634333 | UCGGUGCUUCCCUUUCACUGA | 198 |
| 1657605 | 1637238 | 1637239 | UAUUCACUUGUGUUUGUAUAA | 325 |
| 1657625 | 1637240 | 1637241 | UACCAUUUUGCGAUGUGUUUA | 326 |
| 1657626 | 1637242 | 1637243 | GAAUUAAGAACUGACACAAAA | 327 |
| 1657627 | 1637244 | 1637245 | AGGAAUUUUAGAAGAGGUAGA | 226 |

### Example 4: Effect of Oligomeric Duplexes on Human SCNA RNA in Human SNCA Transgenic Mice, 2-Weeks

Oligomeric duplexes described above were tested in the FVB/N-Tg(SNCA)Mjfa transgenic mouse model expressing wildtype full-length SNCA gene directed by the human SNCA promotor/enhancer regions on the BAC transgene (JAX strain 018442).

The SNCA transgenic mice were divided into groups of 4 mice each. Each mouse received a single ICV bolus of 300 g of oligomeric duplex. A group of 4 mice received a single ICV bolus of PBS as a negative control.

Two weeks post treatment, mice were sacrificed and RNA was extracted from spinal cord, cortical brain tissue, dorsal root ganglion (DRG), and liver for real-time PCR analysis of measurement of mRNA expression of SNCA using human primer probe set hSNCA_LTS00672_MGB (forward sequence TGGCAGAAGCAGCAGGAAA, designated herein as SEQ ID NO: 7; reverse sequence TCCTTGGTTTTGGAGCCTACA, designated herein as SEQ ID NO: 8; probe sequence CAAAAGAGGGTGTTCTC, designated herein as SEQ ID NO: 9). SNCA RNA levels were normalized to mouse PPIA. Mouse PPIA was amplified using primer probe set m_cyclo24 (forward sequence TCGCCGCTTGCTGCA, designated herein as SEQ ID NO: 328; reverse sequence ATCGGCCGTGATGTCGA, designated herein as SEQ ID NO: 329; probe sequence CCATGGTCAACCCCACCGTGTTC, designated herein as SEQ ID NO: 330). Results are presented as percent human SNCA RNA relative to the amount of SNCA RNA in PBS treated animals, (% control).

TABLE 10
Reduction of SNCA RNA by oligomeric
duplexes in SNCA transgenic mice

| Compound | SNCA RNA (% control) | | |
|---|---|---|---|
| No. | Spinal Cord | Cortex | DRG |
| PBS | 100 | 100 | 100‡ |
| 1657448 | 51 | 16 | 21 |
| 1657605 | 113 | 105 | 112 |
| 1657625 | 67 | 70 | 42 |
| 1657626 | 78 | 88 | 109 |
| 1657627 | 80 | 82 | 49 |

‡indicates fewer than 4 samples available

1.-8. (canceled)

9. An oligomeric compound, wherein the oligomeric compound comprises a modified oligonucleotide consisting of 12 to 50 linked nucleosides, wherein the nucleobase sequence of the modified oligonucleotide is complementary to at least 8, at least 9, at least 10, at least 11, at least 12, at least 13, at least 14, at least 15, at least 16, at least 17, at least 18, at least 19, at least 20, at least 21, at least 22, or 23 contiguous nucleobases of
an equal length portion of nucleobases 1315-1397 of SEQ ID NO: 1;
an equal length portion of nucleobases 1475-1577 of SEQ ID NO: 1;
an equal length portion of nucleobases 1655-1737 of SEQ ID NO: 1;
an equal length portion of nucleobases 775-997 of SEQ ID NO: 1;
an equal length portion of nucleobases 1115-1237 of SEQ ID NO: 1;
an equal length portion of nucleobases 1455-1577 of SEQ ID NO: 1;
an equal length portion of nucleobases 495-517 of SEQ ID NO: 1;
an equal length portion of nucleobases 1375-1397 of SEQ ID NO: 1;
an equal length portion of nucleobases 1475-1517 of SEQ ID NO: 1; or
an equal length portion of nucleobases 1555-1577 of SEQ ID NO: 1;
wherein at least one nucleoside of the modified oligonucleotide comprises a modified sugar moiety or a modified internucleoside linkage.

10. The oligomeric compound of claim 9, wherein the nucleobase sequence of the modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of a nucleobase sequence selected from:
SEQ ID NOs: 70, 71, 72 and 75;
SEQ ID NOs: 76, 81, 83, 84, and 85;
SEQ ID NOs: 88, 89, 90, and 92;
SEQ ID NOs: 45, 46, 47, 48, 49, 50, 51, 53, 54, 55, and 56;
SEQ ID NOs: 58, 63, 65, 66, 67, and 68;
SEQ ID NOs: 76, 77, 81, 83, 84, and 85
SEQ ID NO: 29;
SEQ ID NO: 75;
SEQ ID NOs: 76 and 81; and
SEQ ID NO: 83.

11.-15. (canceled)

16. The oligomeric compound of claim 9, wherein the modified sugar moiety is a bicyclic sugar moiety.

17. The oligomeric compound of claim 16, wherein the bicyclic sugar moiety comprises a 2′-4′ bridge, wherein the 2′-4′ bridge is selected from —O—CH₂—; and —O—CH(CH₃)—.

18. The oligomeric compound of claim 9, wherein the modified sugar moiety is a non-bicyclic modified sugar moiety.

19. The oligomeric compound of claim 18, wherein the non-bicyclic modified sugar moiety is a 2′-MOE sugar moiety, a 2′-OMe sugar moiety, or a 2′-F sugar moiety.

20. The oligomeric compound of claim 9, wherein the modified sugar moiety is a sugar surrogate.

21. The oligomeric compound of claim 20, wherein the sugar surrogate is selected from morpholino, modified morpholino, glycol nucleic acid (GNA), six-membered tetrahydropyran (THP), and F-hexitol nucleic acid (F-HNA).

22. (canceled)

23. The oligomeric compound of claim 9, wherein the at least one modified internucleoside linkage is a phosphorothioate internucleoside linkage.

24. The oligomeric compound of claim 9, wherein the at least one modified internucleoside linkage is a mesyl phosphoramidate internucleoside linkage.

25. The oligomeric compound of claim 9, wherein each internucleoside linkage of the modified oligonucleotide is independently selected from a phosphorothioate internucleoside linkage, a phosphodiester internucleoside linkage, and a mesyl phosphoramidate internucleoside linkage.

26.-28. (canceled)

29. The oligomeric compound of claim 9, wherein the modified oligonucleotide comprises at least one modified nucleobase.

30. The oligomeric compound of claim 29, wherein the modified nucleobase is 5-methylcytosine.

31.-70. (canceled)

71. An oligomeric duplex comprising:
a first oligomeric compound comprising a first modified oligonucleotide consisting of 18 to 30 linked nucleosides, wherein the nucleobase sequence of the first modified oligonucleotide comprises at least 8, at least 9, at least 10, at least 11, at least 12, at least 13, at least 14, at least 15, at least 16, at least 17, at least 18, at least 19, at least 20, at least 21, at least 22, or 23 contiguous nucleobases of the nucleobase sequence of any of SEQ ID NOs: 16-166, 318, or 320-324; and
a second oligomeric compound comprising a second modified oligonucleotide consisting of 15 to 29 linked nucleosides, wherein the nucleobase sequence of the second modified oligonucleotide comprises at least 8, at least 9, at least 10, at least 11, at least 12, at least 13, at least 14, at least 15, at least 16, at least 17, at least 18, at least 19, at least 20, or 21 contiguous nucleobases of the nucleobase sequence of any of SEQ ID NOs: 167-317, 319, or 325-327, wherein the nucleobase sequence of the second modified oligonucleotide is at least 90% complementary to the nucleobase sequence of an equal length portion of the first modified oligonucleotide;
wherein at least one nucleoside of the first modified oligonucleotide and at least one nucleoside of the second modified oligonucleotide each independently comprises a modified sugar moiety or a modified internucleoside linkage.

72. (canceled)

73. The oligomeric duplex of claim 71, wherein the nucleobase sequence of the second modified oligonucleotide is at least 95% or 100% complementary to the nucleobase sequence of an equal length portion of the first modified oligonucleotide.

74. The oligomeric duplex of claim 71, wherein the first modified oligonucleotide of the first oligomeric compound comprises a 5′-stabilized phosphate group.

75. The oligomeric duplex of claim 74, wherein the 5′-stabilized phosphate group comprises a cyclopropylphosphonate or a vinylphosphonate.

76. (canceled)

77. The oligomeric duplex of claim 71, wherein the modified sugar moiety is a bicyclic sugar moiety or a non-bicyclic modified sugar moiety.

78. The oligomeric duplex of claim 77, wherein the bicyclic sugar moiety comprises a 2′-4′ bridge, wherein the 2′-4′ bridge is selected from —O—CH₂—; and —O—CH(CH₃)—.

79. (canceled)

80. The oligomeric duplex of claim 77, wherein the non-bicyclic modified sugar moiety is a 2′-MOE sugar moiety, a 2′-OMe sugar moiety, or a 2′-F sugar moiety.

81. The oligomeric duplex of claim 71, wherein the modified sugar moiety is a sugar surrogate.

82. (canceled)

83. (canceled)

84. (canceled)

85. The oligomeric duplex of claim 71, wherein the modified internucleoside linkage is a phosphorothioate internucleoside linkage.

86. The oligomeric duplex of claim 71, wherein the modified internucleoside linkage is a mesyl phosphoramidate internucleoside linkage.

87.-92. (canceled)

93. The oligomeric duplex of claim 71, wherein the first modified oligonucleotide and the second modified oligonucleotide each independently comprises at least one modified nucleobase.

94. The oligomeric duplex of claim 93, wherein the at least one modified nucleobase is 5-methylcytosine.

95.-106. (canceled)

107. The oligomeric duplex of claim 71, wherein the second modified oligonucleotide comprises a conjugate group.

108. The oligomeric duplex of claim 107, wherein the conjugate group comprises a conjugate linker and a conjugate moiety.

109. The oligomeric duplex of claim 107, wherein the conjugate group is attached to the second modified oligonucleotide at the 5′-end of the second modified oligonucleotide, at the 3′-end of the second modified oligonucleotide, or at the 2′ position of a furanosyl sugar moiety.

110.-122. (canceled)

123. A population of oligomeric compounds comprising modified oligonucleotides of claim 9, wherein all of the phosphorothioate internucleoside linkages of the modified oligonucleotides are stereorandom.

124. (canceled)

125. (canceled)

126. A pharmaceutical composition comprising the oligomeric compound of claim 9 and a pharmaceutically acceptable diluent or carrier.

127. The pharmaceutical composition of claim 126, wherein the pharmaceutically acceptable diluent is phosphate buffered saline (PBS) or artificial cerebrospinal fluid (aCSF).

128. The pharmaceutical composition of claim 127, wherein the pharmaceutical composition consists essentially of the oligomeric compound and aCSF.

129. The pharmaceutical composition of claim 127, wherein the pharmaceutical composition consists essentially of the oligomeric compound and PBS.

130. A method comprising administering to a subject the oligomeric compound of claim 9.

131. (canceled)

132. The method of claim 130, wherein the subject has a synucleinopathy.

133. The method of claim 130, wherein the subject has Parkinson's disease, dementia with Lewy bodies, diffuse Lewy body disease, Parkinson's disease dementia, pure autonomic failure, multiple system atrophy, neuronopathic Gaucher's disease, or Alzheimer's disease.

134. (canceled)

135. A method of treating a disease or disorder associated with SNCA comprising administering to a subject having or at risk of developing a disease or disorder associated with SNCA a therapeutically effective amount of the oligomeric compound of claim 9, thereby treating the disease or disorder associated with SNCA.

136. (canceled)

137. The method of claim 135, wherein the disease or disorder associated with SNCA is a synucleinopathy.

138. The method of claim 136, wherein the neurodegenerative disease or synucleinopathy is any of Parkinson's disease, dementia with Lewy bodies, diffuse Lewy body disease, Parkinson's disease dementia, pure autonomic failure, multiple system atrophy, neuronopathic Gaucher's disease, and Alzheimer's disease.

139.-145. (canceled)

146. A method of reducing SNCA RNA in a cell or reducing alpha-synuclein protein in a cell comprising contacting the cell with the oligomeric compound of claim 9.

147. (canceled)

148. The method of claim 146, wherein the cell is a central nervous system cell.

149.-160. (canceled)
